(19) United States
(12) Patent Application Publication
(10) Pub. No.: US 2013/0203709 A1
(43) Pub. Date: Aug. 8, 2013

(54) ACYLSULFONAMIDES AND PROCESSES FOR PRODUCING THE SAME

(71) Applicants: Roman Manetsch, Tampa, FL (US); Sameer Kulkarni, Tampa, FL (US); Iredia D. Iyamu, Tampa, FL (US); Hong-Gang Wang, Palmyra, PA (US); Kenichiro Doi, Hummelstown, PA (US); Wayne Guida, St. Petersburg Beach, FL (US); Daniel Santiago, Pinellas Park, FL (US); Courtney du Boulay, Temple Terrace, FL (US)

(72) Inventors: Roman Manetsch, Tampa, FL (US); Sameer Kulkarni, Tampa, FL (US); Iredia D. Iyamu, Tampa, FL (US); Hong-Gang Wang, Palmyra, PA (US); Kenichiro Doi, Hummelstown, PA (US); Wayne Guida, St. Petersburg Beach, FL (US); Daniel Santiago, Pinellas Park, FL (US); Courtney du Boulay, Temple Terrace, FL (US)

(73) Assignees: THE PENN STATE RESEARCH FOUNDATION, University Park, PA (US); UNIVERSITY OF SOUTH FLORIDA, Tampa, FL (US)

(21) Appl. No.: 13/814,765

(22) PCT Filed: Aug. 9, 2011
(86) PCT No.: PCT/US11/47028

§ 371 Date: Apr. 15, 2013

Related U.S. Application Data
(60) Provisional application No. 61/372,009, filed on Aug. 9, 2010.

(51) Int. Cl. C07C 311/37; C07D 277/56; C07D 405/12; C07D 307/68; C07D 261/18; C07D 211/14; C07D 217/04

(52) U.S. Cl. CPC C07C 311/37; C07D 211/14; C07D 277/56; C07D 217/04; C07D 307/68; C07D 261/18; C07D 405/12
USPC 514/150; 546/206; 514/325; 552/5; 548/200; 514/365; 564/85; 514/603; 546/146; 514/307; 549/487; 514/471; 548/248; 514/378; 544/369; 514/254.02; 546/140; 514/308; 549/441; 514/466; 506/12

(57) ABSTRACT

The present disclosure relates to acylsulfonamides and processes for their preparation. The processes involve a target-guided synthesis approach, whereby a thioacid and a sulfonyl azide are reacted in the presence of a biological target protein, a Bcl-2 family protein, to form the acylsulfonamide.

### CROSS-REFERENCE TO RELATED APPLICATIONS

This application claims the benefit of U.S. Provisional Application Ser. No. 61/372,009, filed Aug. 9, 2010, which are hereby incorporated by reference in their entirety, including any figures, tables, and drawings.

### STATEMENT OF GOVERNMENT RIGHTS

This invention was made with United States government support under Grant No. 07KN-08 awarded by the James and Esther King Biomedical Research Program and Grant No. P01CA118210 awarded by the National Cancer Institute, National Institutes of Health. The United States government has certain rights in the invention.

### BACKGROUND

The present disclosure generally relates to acylsulfonamides and processes for their preparation. The disclosure also relates to a kinetically controlled target-guided synthesis approach for the discovery and development of small molecules.

Combinatorial chemistry and parallel synthesis are the tools commonly utilized for lead compound identification and optimization. However, even though in the last two decades combinatorial chemistry and parallel synthesis have gone hand in hand with the dramatic advances of technology for rapid production, handling and screening of large numbers of compounds, they are often accompanied by challenges such as the efficiency of library synthesis, the purity of each library member, and the unambiguous identification of lead compounds in the screening of each library member against a particular biological target. In the last decade, fragment-based lead compound discovery or target-guided synthesis (TGS) approaches have been developed in which the biological target is actively engaged in the design and the synthesis of its own enzyme inhibitory compounds. To date, target-guided synthesis has exclusively been applied for enzymatic targets only. See, e.g., Manetsch et al., Journal of the American Chemical Society 2004, 126, 12809-12818; Sharpless et al., Expert Opin. Drug Discovery 2006, 1, 525-538; and Kolb et al., U.S. Patent Publication No. 2006/0269942.

Among a variety of proteins, the Bcl-2 family of proteins, which consists of both anti- and pro-apoptotic molecules, in particular, can play an important role in the regulation of the intrinsic (mitochondrial) pathway of apoptosis. The anti-apoptotic Bcl-2 family proteins (e.g., Bcl-2, Bcl-X_{L}, Mcl-1) inhibit the release of certain pro-apoptotic factors from mitochondria, whereas pro-apoptotic Bcl-2 family members, which can be further separated into two subgroups, the multidomain BH1-3 proteins (Bax and Bak) and the BH3-only proteins (e.g., Bad, Bim, and Noxa), induce the release of mitochondrial apoptogenic molecules into the cytosol. Although the precise biochemical mechanisms by which Bcl-2 family proteins exert their influence on cell life and death remains far from clear, the relative ratios of pro- and anti-apoptotic Bcl-2 family proteins determine the ultimate sensitivity or resistance of cells to a wide variety of apoptotic signals.

Evidence has accumulated that the majority of human cancers overexpress the pro-survival Bcl-2 family proteins, which not only contribute to cancer progression by preventing normal cell turnover, but also render cancer cells resistant to current cancer treatments. For example, high levels of Bcl-2 are found in ˜30% to 60% of prostate cancer, ˜60% to 90% of breast cancer, ˜20% to 40% of non-small cell lung cancer, ˜60% to 80% of small cell lung cancer, ˜50% to 100% of colorectal cancer, ˜65% of melanoma, ˜30% of neuroblastomas, and ˜80% of B cell lymphomas. Similarly, Bcl-X_{L} is overexpressed in ˜100% of hormone-refractory prostate cancer, ˜40% to 60% of breast cancer, ˜80% of colorectal cancer, ˜90% of melanoma, ˜90% of pancreatic cancer, and ˜80% of hepatocellular carcinoma. It has been shown that overexpression of Bcl-2 and/or Bcl-X_{L} renders cancer cells resistant to most of the currently available chemotherapeutic drugs as well as radiation therapy. Therefore, it is an attractive strategy to design and develop a new class of anticancer drugs that specifically target the anti- and pro-apoptotic functions of the Bcl-2 family proteins.

Over the last few years, several small-molecule Bcl-2 inhibitors have been synthesized and some of these molecules have entered clinical trials. Although Bcl-2 and Bcl-X_{L} have been the primary focus for the design of small-molecule inhibitors, recent studies have demonstrated that Mcl-1 also plays an important role for cancer cell survival and that Bcl-2, Bcl-X_{L} and Mcl-1 must be simultaneously neutralized for apoptosis induction in many types of cancer cells. Obatoclax (GX15-070) is a pan-Bcl-2 inhibitor but seems to have additional targets besides anti-apoptotic Bcl-2 family proteins and thus may lead to unpredicted, non-mechanism based toxicity. Clearly, the more specific the inhibitor for individual Bcl-2 family members, the less non-mechanism based toxicity may be expected. To date, the most potent and selective small-molecule Bcl-2 inhibitors are ABT-737 and its orally active analog ABT-263, which only inhibit Bcl-2, Bcl-X_{L} and Bcl-w but do not target Mcl-1 or A1. Thus, these agents generally lack efficacy in tumors with elevated Mcl-1 or A1 and in many instances this resistance can be overcome by down-regulation of Mcl-1. For example, knockdown of Mcl-1 or overexpression of Noxa, a BH3-only protein that selectively binds to and inhibits Mcl-1, sensitizes MCF-7 breast cancer cells to ABT-737. Similarly, it has been demonstrated that suppression of Mcl-1 expression allows ABT-737 to promote anoikis (detachment-induced apoptosis) in MDA-MB-231 breast cancer cells. More recently, it was shown in a non-Hodgkin's lymphoma model that acquired resistance was developed after the cells were exposed to ABT-737 for three weeks and transcriptional upregulation of Mcl-1 was detected.

### SUMMARY OF THE DISCLOSURE

Among the various aspects of the present disclosure is the provision of a target-guided synthesis approach for the discovery and development of small molecules, and in particular acylsulfonamides.

Briefly, therefore, the present disclosure is directed to a process for the preparation of an acylsulfonamide (3), the process comprising reacting a thioacid (1) with a sulfonyl azide (2) in the presence of a protein of the Bcl-2 family, wherein the thioacid (1), the sulfonyl azide (2), and the acylsulfonamide (3) correspond to Formulae (1), (2), and (3):

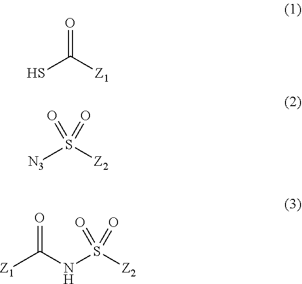

Z₁ is hydrocarbyl, substituted hydrocarbyl, heteroaryl, or heterocyclo; and

Z₂ is hydrocarbyl, substituted hydrocarbyl, heteroaryl, or heterocyclo.

Another aspect of the disclosure is directed to an acylsulfonamide (3) having the formula:

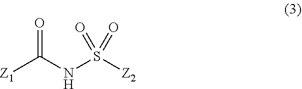

wherein

Z₁ has the formula:

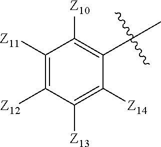

Z₂ has the formula:

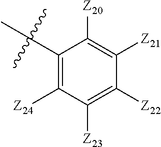

Z₁₁ and Z₁₃ are alkyl, substituted alkyl, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heterocyclo, and halo, among others, wherein each occurrence of R_{Z} is substituted or unsubstituted alkyl, substituted or unsubstituted aryl, or substituted or unsubstituted aralkyl;

Z₂₂ is —N(Z₂₂₀)(Z₂₂₁) or —CH₂—N(Z₂₂₀)(Z₂₂₁), wherein Z₂₂₀ and Z₂₂₁ are independently hydrogen, alkyl, substituted alkyl, aryl, substituted aryl, or Z₂₂₀ and Z₂₂₁ together with the nitrogen atom to which they are attached, form a substituted or unsubstituted alicyclic, bicyclic, aryl, or heterocyclic moiety; and

Z₁₀, Z₁₂, Z₁₄, Z₂₀, Z₂₁, Z₂₃, and Z₂₄ are hydrogen.

Another aspect of the present disclosure relates to a compound for inhibiting a Bcl-2 family protein, the Bcl-2 family protein selected from one or more of Bcl-2, Bcl-X_{L}, Bcl-w, Mcl-1, and A1/Bfl-1, wherein the compound corresponds to Formula (3):

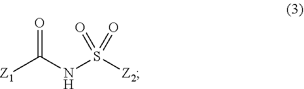

Z₁ is hydrocarbyl, substituted hydrocarbyl, heteroaryl, or heterocyclo; and

Z₂ is hydrocarbyl, substituted hydrocarbyl, heteroaryl, or heterocyclo.

Another aspect of the present disclosure relates to a method of treating or preventing cancer. The method comprises administering a compound described herein to a patient in need of such treatment.

Another aspect of the present disclosure relates to a method of screening for cancer therapies using the compounds described herein.

Other aspects and features will be in part apparent and in part pointed out hereinafter.

### BRIEF DESCRIPTION OF THE DRAWINGS

FIG. 1 illustrates the ribbon structure of a Bcl-X_{L}-Bak complex and the surface representation of the binding pocket of Bcl-X_{L} bound to the Bak peptide.

FIG. 2 illustrates exemplary steps of conventional lead discovery and target-guided synthesis protocols.

FIG. 3 illustrates the binding pockets of the Bcl-X_{L}-Bak complex.

FIG. 4 is an LC/MS trace illustrating the comparison between incubations of (SZ4 and TA2) measured by LC/MS-SIM Mode and LC/MS-Scan Mode. A) Incubation of (SZ4) and (TA2) without Bcl-X_{L} measured by LC/MS-SIM mode; B) Incubation of (SZ4) and (TA2) with 2 μM Bcl-X_{L} measured by LC/MS-SIM mode; C) Incubation of (SZ4) and (TA2) with 2 μM Bcl-X_{L} measured by LC/MS-Scan mode.

FIG. 5 is an LC/MS trace illustrating the incubations of (SZ1) and (TA2) and incubations of (SZ2) and (TA2). A) Incubation of (SZ1) and (TA2) without Bcl-X_{L}; B) Incubation of (SZ1) and (TA2) with 2 μM Bcl-X_{L}; C) Incubation of (SZ5) and (TA2) without Bcl-X_{L}; D) Incubation of (SZ5) and (TA2) with 2 μM Bcl-X_{L}.

FIG. 6 is an LC/MS trace illustrating the incubations of (SZ4) and (TA2) with bovine erythrocyte carbonic anhydrase II, concanavalin A and mAChE. A) Incubation of (SZ4) and (TA2) without proteins; B) Incubation of (SZ4) and (TA2) with 2 μM of bCAII; C) Incubation of (SZ4) and (TA2) with 2 μM of ConA. D) Incubation of (SZ4) and (TA2) with 2 μM of mAChE. E) Incubation of (SZ4) and (TA2) with 2 μM of Bcl-X_{L}.

FIG. 7 is an LC/MS trace illustrating the incubations of (SZ4) and (TA2) with Bak BH3 peptide for 24 hours. A) Incubation of (SZ4) and (TA2) without Bcl-X_{L} or Bak BH3 peptide; B) Incubation of (SZ4) and (TA2) with 20 μM Bak BH3 peptide and without no Bcl-X_{L}.

FIG. 8 is an LC/MS trace illustrating Bcl-X_{L}-templated incubations containing Bim, mutant Bim and mutant Bak. A) Incubation of (SZ4) and (TA2) without Bcl-X_{L}; B) Incubation of (SZ4) and (TA2) with 2 μM Bcl-X_{L}; C) Incubation of (SZ4) and (TA2) with 2 μM Bcl-X_{L} and 20 μM Bak BH3 peptide; D) Incubation of (SZ4) and (TA2) with 2 μM Bcl-X_{L} and 20 μM of mutant Bak; E) Incubation of (SZ4) and (TA2) with 2 μM Bcl-X_{L} and 20 μM of Bim; F) Incubation of (SZ4) and (TA2) with 2 μM Bcl-X_{L} and 20 μM of mutant Bim.

FIG. 9 is an LC/MS trace illustrating incubations of (SZ4) and (TA2) with Bim, mutant Bim and mutant Bak (no Bcl-X_{L}). A) Incubation of (SZ4) and (TA2) without peptides; B) Incubation of (SZ4) and (TA2) with 20 μM Bim; C) Incubation of (SZ4) and (TA2) with 20 μM of mutant Bim; D) Incubation of (SZ4) and (TA2) with 20 μM of mutant Bak.

FIG. 10 is an LC/MS trace illustrating the suppression of Bcl-X_{L}-templated incubations with Bak BH3 Peptide. Incubation samples were kept for six hours at 37° C. A) Incubation of (SZ4) and (TA2) without Bcl-X_{L}; B) Incubation of (SZ4) and (TA2) with 2 μM Bcl-X_{L}; C) Incubation of (SZ4) and (TA2) with 2 μM Bcl-X_{L} and 20 μM Bak BH3 peptide; D) Incubation of (SZ4) and (TA2) with 2 μM Bcl-X_{L} and 10 μM Bak BH3 peptide; E) Incubation of (SZ4) and (TA2) with 2 μM Bcl-X_{L} and 5 μM Bak BH3 peptide; F) Incubation of (SZ4) and (TA2) with 2 μM Bcl-X_{L} and 2 μM Bak BH3 peptide.

FIG. 11 is an LC/MS trace illustrating Bcl-X_{L} incubations containing sulfonyl azides (SZ1)-(SZ6) and thioacid (TA2). A) Incubation of (SZ1)-(SZ6) and (TA2) without Bcl-X_{L}; B) Incubation of (SZ1)-(SZ6) and (TA2) with 404 Bcl-X_{L}; C) Synthesized compound (SZ4TA2) as reference.

FIGS. 12-19 are LC/MS trace illustrating other sulfonyl azide and thioacid combinations: (SZ7) and (TA2) (FIG. 12); (SZ9) and (TA5) (FIG. 13); (SZ10) and (TA2) (FIG. 14); (SZ15) and (TA3) (FIG. 15); (SZ15) and (TA8) (FIG. 16); (SZ16) and (TA4) (FIG. 17); (SZ16) and (TA8) (FIG. 18); and (SZ17) and (TA7).

FIG. 20 is a schematic representation of kinetic TGS, which generally utilizes substantially irreversible reactions for the assembly of bidentate ligands.

FIG. 21 is a reaction scheme illustrating a representative amidation reaction.

FIG. 22 illustrates the reactive fragments utilized in kinetic TGS targeting Bcl-X_{L} and Mcl-1. The studies with Bcl-X_{L} as the template were conducted with SZ1-SZ9 and thioacids TA1-TA9 (R=H). Kinetic TGS targeting Mcl-1 was conducted with fluorenylmethyl-protected thioacids TA1-TA14 (R=Fm), which were deprotected shortly prior to their incubations with Mcl-1 and sulfonyl azides SZ1-SZ31. With the exception of the designated fragments, all TAs and SZs demonstrated at 100 μM concentrations less than 5% inhibition (Bcl-X_{L} and Mcl-1) in the FP assay.

FIG. 23 depicts four representative compounds identified via kinetic TGS screening against Bcl-X_{L} and their IC₅₀s against Bcl-X_{L}.

FIG. 24 depicts exemplary optimization possibilities for a representative compound described herein.

### DETAILED DESCRIPTION

Among other things, the present disclosure relates to a fragment-based lead compound discovery method, in which the biological target, e.g., a member of the Bcl-2 family of proteins, is directly involved in the assembly of its own bidentate ligand from two or more smaller reactive fragments or scaffolds. The methods described herein are versatile target-guided synthesis approaches for probing adaptive regions on/in biological targets, and in particular Bcl-2 family protein targets (e.g., Bcl-X_{L} and/or Mcl-1), and can be exploited as an innovative means to identify and optimize small molecules interacting with such biological targets. The target-guided synthesis methods are successful, in part, due to: (a) the nature of the chemical reaction combining the two fragments or scaffold compounds into a single molecule; and (b) the use of reactive fragments showing low to high affinity towards binding pockets or surfaces of the biological targets.

Another key component of the processes described herein is the reactivity of the utilized reactions; specifically, the functionalities on the building block or scaffold compounds can be tuned not only to the particular biological target, but also to speed up or slow down reactivity with the biological target, improving the formation of bidentate ligand(s) displaying good affinity to the biological target. Among other things, the processes described herein address certain limitations of the target-guided synthesis methods reported thus far; compared to the reported target-guided synthesis methods for the screening of enzymes, the discovery of protein interactions is more challenging because biological target/interfaces have relatively shallow binding sites on their surfaces, thus permitting only weak binding affinity for reactive fragments. This often translates to short residence times for these fragments within the binding cavities. For these and other reasons, previously reported target-guided synthesis methods poorly succeed or even fail in discovery attempts.

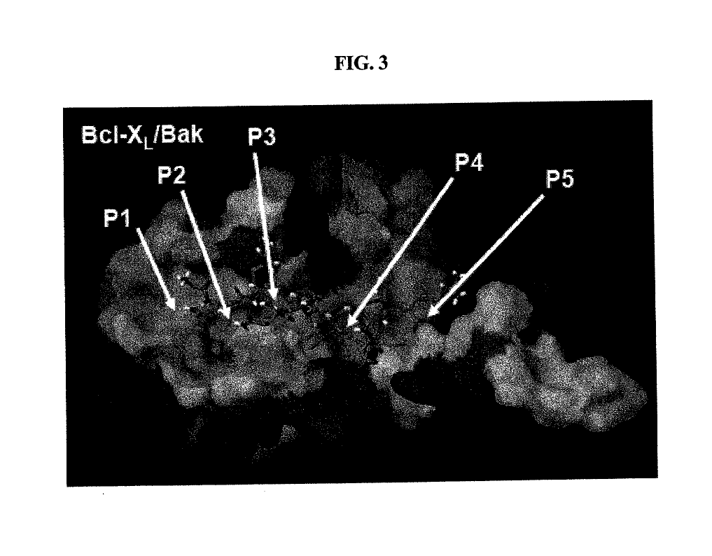

As noted above, the processes described herein utilize certain structural moieties or scaffolds having activity against Bcl-2 family protein interactions (also referred to as protein-protein interaction modulation (PPIM)). PPIM activity can be achieved as described herein by compound design including one or two of the aforementioned structural moieties in the same compound. Each scaffold portion is designed to bind to one or more subpockets of a biological target, e.g., a Bcl-2 family protein. In a particular embodiment, the compounds prepared by the target-guided synthesis methods described herein are acylsulfonamide compounds that are capable of binding to one or more of the subpockets of a Bcl-2 family (e.g., Bcl-X_{L}, the binding subpockets of which are designated as P1, P2, P3, P4, and P5) (see, e.g., FIG. 3)). In a particular embodiment, the acylsulfonamide compounds target the P4 and/or P5 region of Bcl-X_{L}. In another particular embodiment, the acylsulfonamide compounds target Mcl-1; more preferably in this embodiment, the acylsulfonamide compounds have a specificity for Mcl-1 over Bcl-X_{L} of at least two fold, more preferably at least three fold, more preferably at least four fold, more preferably at least five fold, or more preferably at least six fold.

Compared to the previously reported target-guided synthesis screening methods for enzyme inhibitors, the target-guided synthesis approaches described herein utilize reactions with superior reactivity profiles, enabling the use of traditionally weak affinity small molecules as relatively reactive fragments for the discovery and optimization of ligands and compounds. The enhanced reactivity is due, in part, to the use of more reactive functionalities for the chemical reaction(s) that combines the two fragments into a larger molecule.

Among other things, the present disclosure relates to the preparation of acylsulfonamides. According to the processes described herein, at least one (and typically two or more) thioacid(s) is incubated or reacted with at least one (and typically two or more) sulfonyl azide(s) in the presence of a protein of the Bcl-2 family (e.g., Bcl-X_{L} and/or Mcl-1) to form an acylsulfonamide. In certain embodiments, the protein is Bcl-X_{L}. In certain other embodiments, the protein is Mcl-1. In general, the reaction involves an amidation reaction between electron-poor thioacids and sulfonyl azides or between thioacids and electron-rich sulfonyl azides. See, e.g., Shangguan et al. J. Am. Chem. Soc. 2003, 125, 7754-7755.

One particular aspect of the present disclosure relates to the identification of multiple compounds displaying greater specificity in binding to Mcl-1 over other Bcl-2 family proteins such as Bcl-X_{L} or Bcl-2. Though a selective inhibitory compound is desired, there are very few compounds known to display such a selectivity for Mcl-1 over Bcl-X_{L} as the compounds described herein. Using kinetic target-guided synthesis, for example, multiple compounds were identified and optimized that selectively inhibit the proteins of the Bcl-2 family (such as Bcl-X_{L} and/or Mcl-1). In one cell-free assay, for example, compounds were identified which are specifically inhibiting Mcl-1 while showing relatively low interaction to Bcl-X_{L}. Thus, the compounds described herein can be considered to be highly specific Mcl-1 inhibitors. The compounds described herein also show similar selectivity in a cellular assay, as compared to the selectivity in the cell-free assay.

The acylsulfonamide-forming reaction described herein is generally illustrated in Reaction Scheme (1), wherein Z₁ and Z₂ are described in connection with Formulae (1), (2), and (3) below:

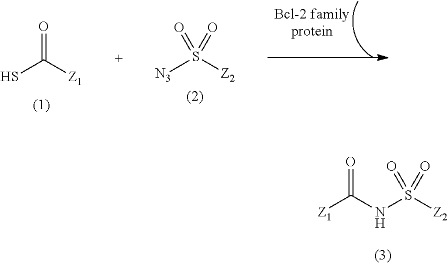

As shown, the thioacid (1) is reacted with a sulfonyl azide (2) in the presence of a Bcl-2 family protein (such as Bcl-X_{L}, Mcl-1, or other related proteins). Usually, the reaction involves a pool or library of two or more thioacids (1), and a corresponding pool or library of two or more sulfonyl azides (2). The reaction is typically carried out at relatively ambient or slightly higher temperatures, which can serve to enhance the rate of the ligation reaction. The acylsulfonamide-forming reaction is typically carried out at a temperature of at least 20° C., preferably at least 25° C., and more preferably 30-40° C. Reaction times can range from about 1 hour to several days; e.g., from about 1 hour to about 48 hours (e.g., 6-12 hours, 12-36 hours, or 24-72 hours).

The reaction mixture for preparing the acylsulfonamide (3) according to the methods described herein typically comprises the thioacid (1) (or a library thereof), the sulfonyl azide (2) (or a library thereof), the biological target, and an aqueous buffer medium, which may be optimized depending on the particular thioacid(s) (1), sulfonyl azide(s) (2), and Bcl-2 family protein selected for the reaction. Preferably, the buffer is an aqueous physiological buffer that is compatible with biological materials. Buffers useful in the preparation of acylsulfonamides according to the processes described herein include but are not limited to phosphate-, citrate-, sulfosalicylate-, and acetate-based buffers, or other organic acid-based buffers. Still other buffers include ADA buffer, ACES buffer, BES buffer, BIS TRIS buffer, DIPSO buffer, HEPES buffer, MOPS buffer, MOPSO buffer, PIPES buffer, TES buffer, Tris buffer, Tricine buffer, TRISMA buffer, and the like. A more complete list can be found in the United States Pharmacopeia. In one embodiment, the buffer is a phosphate buffer (e.g., sodium phosphate, potassium phosphate, and the like). In certain preferred embodiments the buffering agent will be present in an amount sufficient to provide a pH ranging from about 6.0 to 9.5, more preferably pH 7.4. Other agents that may be present in the buffer medium include chelating agents, such as EDTA, EGTA, and the like.

Thioacids

In accordance with the present methods, a thioacid (or a library of thioacids) is reacted with a sulfonyl azide (or a library of sulfonyl azides) in the presence of a biological target molecule; in preferred embodiments, the biological target molecule is a Bcl-2 family protein. In general, the Bcl-2 family protein acts as a template for the formation of the acylsulfonamide. As noted above in connection with Reaction Scheme (1), the thioacid corresponds to Formula (1):

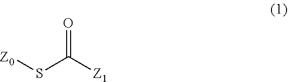

wherein

Z₀ is hydrogen or a thiol protecting group; and

Z₁ is hydrocarbyl, substituted hydrocarbyl, heteroaryl, or heterocyclo. In one embodiment, the thiol protecting group is fluorenylmethyl oxycarbonyl (Fmoc).

Typically, such hydrocarbyl substituents for Z₁ contain from 1 to 20 carbon atoms and may be linear, branched, or cyclic, and said substituted hydrocarbyl, heteroaryl, and heterocyclo moieties for Z₁ may be substituted with one or more of ═O, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heteroaryl, heterocyclo, and halo (including F, Cl, Br and I), among others, wherein each occurrence of R_{Z} may be hydrocarbyl or substituted hydrocarbyl (e.g., substituted or unsubstituted alkyl, substituted or unsubstituted aryl, or substituted or unsubstituted aralkyl), heteroaryl, or heterocyclo.

Although Z₁ is hydrocarbyl, substituted hydrocarbyl, heteroaryl, or heterocyclo, in certain embodiments Z₁ is aryl, substituted aryl, or heteroaryl. In the embodiments in which Z₁ is aryl or substituted aryl, for example, Z₁ may have the formula:

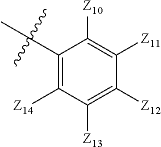

wherein Z₁₀, Z₁₁, Z₁₂, Z₁₃, and Z₁₄ are independently hydrogen, hydroxyl, protected hydroxyl, halo, hydrocarbyl, substituted hydrocarbyl, heterocyclo, heteroaryl, alkoxy, alkenoxy, alkynoxy, aryloxy, arylalkoxy (heterocyclo)alkoxy, trihaloalkoxy, amino, amido, or cyano, or two of Z₁₀, Z₁₁, Z₁₂, Z₁₃, and Z₁₄, together with the carbon atoms to which they are attached, form a fused carbocyclic (e.g., napthyl) or heterocyclic ring. In one embodiment, Z₁ corresponds to the aryl or substituted aryl structure illustrated above and Z₁₀, Z₁₁, Z₁₂, Z₁₃, and Z₁₄ are independently hydrogen, hydrocarbyl, substituted hydrocarbyl, amino, alkoxy, nitro, or trihalomethoxy (e.g., trifluoromethoxy); more preferably in this embodiment, Z₁₀ and Z₁₄ are hydrogen and Z₁₁, Z₁₂, and Z₁₃ are independently hydrogen, hydrocarbyl, substituted hydrocarbyl, amino, alkoxy, nitro, or trihalomethoxy. In one particular embodiment, Z₁ is phenyl, substituted phenyl, or napthyl moiety, optionally with substituents in the ortho-, para-, and/or meta-positions; more preferably in this embodiment, Z₁ is phenyl, para- and/or meta-substituted phenyl, or napthyl moiety; thus, for example, at least Z₁₁ and Z₁₃ in the above structure are substituted with alkyl, substituted alkyl, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heteroaryl, heterocyclo, and halo (including F, Cl, Br and I), among others, wherein each occurrence of R_{Z} may be hydrocarbyl, substituted hydrocarbyl (e.g., substituted or unsubstituted alkyl, substituted or unsubstituted aryl, or substituted or unsubstituted aralkyl), heteroaryl, or heterocyclo. Typically in this embodiment, Z₁₀, Z₁₂, and Z₁₄ are hydrogen. In another embodiment, Z₁₀ and Z₁₄ are hydrogen and at least one of Z₁₁, Z₁₂, and Z₁₃ is other than hydrogen. In this embodiment, for example, one or more of Z₁₁, Z₁₂, and Z₁₃ may be alkyl, substituted alkyl, alkoxy, halo, or nitro.

In the embodiments in which Z₁ corresponds to the aryl or substituted aryl structure illustrated above and where one or more of Z₁₀, Z₁₁, Z₁₂, Z₁₃, and Z₁₄ are hydrocarbyl, for example, they may be independently alkyl, alkenyl, alkynyl, aryl, alkaryl, or aralkyl. Typically, such substituents contain from 1 to 20 carbon atoms and may be linear, branched, or cyclic. By way of example, the Z₁₀, Z₁₁, Z₁₂, Z₁₃, and Z₁₄ substituents may be selected from methyl, ethyl, n-propyl, cyclopropyl, isopropyl, n-butyl, cyclobutyl, isobutyl, s-butyl, n-pentyl, isopentyl, cyclopentyl, n-hexyl, isohexyl, cyclohexyl, benzyl, phenyl, and napthyl. Where one or more of Z₁₀, Z₁₁, Z₁₂, Z₁₃, and Z₁₄ are substituted hydrocarbyl, for example, they may be independently substituted alkyl, substituted alkenyl, substituted alkynyl, substituted aryl, substituted alkaryl, or substituted aralkyl. Similar to the hydrocarbyl moieties, these substituents may contain 1 to 20 carbon atoms and may be linear, branched, or cyclic; one or more hydrogen atoms of the substituted hydrocarbyl moieties, however, are replaced with a different substituent such as, for example, ═O, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heteroaryl, heterocyclo, and halo (including F, Cl, Br and I), among others, wherein each occurrence of R_{Z} may be hydrocarbyl, substituted hydrocarbyl (e.g., substituted or unsubstituted alkyl, substituted or unsubstituted aryl, or substituted or unsubstituted aralkyl), heteroaryl, or heterocyclo.

Where Z₁ corresponds to the aryl or substituted aryl structure illustrated above and where one or more of Z₁₀, Z₁₁, Z₁₂, Z₁₃, and Z₁₄ are amino, for example, the amino moiety may have the formula: —N(Z_{X})(Z_{Y}) wherein Z_{X} and Z_{Y} are independently hydrogen, hydrocarbyl, substituted hydrocarbyl, heteroaryl, heterocyclo, or an amino protecting group, or Z_{X} and Z_{Y}, together with the nitrogen atom to which they are attached, form a substituted or unsubstituted alicyclic, bicyclic, aryl, heteroaryl, or heterocyclic moiety, typically having 3 to 10 atoms in the ring.

In one particular embodiment, Z₁ has the formula:

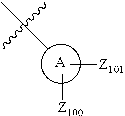

wherein A is phenyl or a five- or six-membered aromatic carbocyclic or heterocyclic ring wherein from one to three carbon atoms may be replaced by a heteroatom selected from N, O, or S, and wherein A is substituted with Z₁₀₀ and Z₁₀₁ through ring carbon atoms or ring heteroatoms, and Z₁₀₀ and Z₁₀₁ are independently hydrogen, hydrocarbyl, substituted hydrocarbyl, heterocyclo, alkoxy, alkenoxy, alkynoxy, aryloxy, heterocyclo(alkoxy), or halo. Where Z₁₀₀ and Z₁₀₁ are hydrocarbyl or substituted hydrocarbyl, for example, they may be substituted or unsubstituted (straight, branched, or cyclic) alkyl, alkenyl, alkynyl, aryl, aralkyl, or arylalkenyl, wherein the substituents for such groups may be, for example, ═O, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heteroaryl, heterocyclo, and halo (including F, Cl, Br and I), among others, wherein each occurrence of R_{Z} may be hydrocarbyl, substituted hydrocarbyl (e.g., substituted or unsubstituted alkyl, substituted or unsubstituted aryl, or substituted or unsubstituted aralkyl), heteroaryl, or heterocyclo. In accordance with these embodiments, for example, Z₁ may be a substituted or unsubstituted thiazole, isoxazole, or furyl moiety. In one particular embodiment, Z₁₀₀ and Z₁₀₁ are selected from hydrogen, alkyl, aryl, arylalkenyl, arylalkoxy, cycloalkenyl, cycloalkyl, halo, heterocyclo, or (heterocyclo)alkoxy. Where Z₁₀₀ and/or Z₁₀₁ are heterocyclo, for example, they may be selected from substituted or unsubstituted morpholino, pyran, tetrahydropyran, piperazinyl, piperidinyl, tetrahydropyridinyl, pyrrolidinyl, 1,4-diazepanyl, and azepinyl.

In another particular embodiment, Z₁ has the structure:

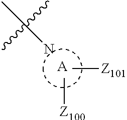

wherein A is a five-, six-, or seven-membered non-aromatic ring containing a nitrogen atom wherein from zero to two carbon atoms are replaced by a heteroatom selected from N, O, or S, and wherein A is substituted with Z₁₀₀ and Z₁₀₁ through ring carbon atoms or ring heteroatoms, and Z₁₀₀ and Z₁₀₁ are independently hydrogen, hydrocarbyl, substituted hydrocarbyl, heterocyclo, alkoxy, alkenoxy, alkynoxy, aryloxy, heterocyclo(alkoxy), or halo. In accordance with these embodiments, for example, Z₁ may be a substituted or unsubstituted piperazine, piperidine, tetrahydropyridine, pyrrolidine, pyrroline, 1,4-diazepane, or azepane moiety. Where Z₁₀₀ and Z₁₀₁ are hydrocarbyl or substituted hydrocarbyl, for example, they may be substituted or unsubstituted (straight, branched, or cyclic) alkyl, alkenyl, alkynyl, aryl, aralkyl, or arylalkenyl, wherein the substituents for such groups may be, for example, ═O, —OH, —OR_{Z}, —COOH, —COOR_{Z}, —CONH₂, —NH₂, —NHR_{Z}, —NR_{Z}, —NO₂, —SH, —SR_{Z}, —SO₂R_{Z}, —SO₂H, —SOR_{Z}, heteroaryl, heterocyclo, and halo (including F, Cl, Br and I), among others, wherein each occurrence of R_{Z} may be hydrocarbyl, substituted hydrocarbyl (e.g., substituted or unsubstituted alkyl, substituted or unsubstituted aryl, or substituted or unsubstituted aralkyl), heteroaryl, or heterocyclo. In one particular embodiment, Z₁₀₀ and Z₁₀₁ are selected from hydrogen, alkyl, aryl, arylalkenyl, arylalkoxy, cycloalkenyl, cycloalkyl, halo, heterocyclo, or (heterocyclo)alkoxy. Where Z₁₀₀ and/or Z₁₀₁ are heterocyclo, for example, they may be selected from substituted or unsubstituted morpholino, pyran, tetrahydropyran, piperazinyl, piperidinyl, tetrahydropyridinyl, pyrrolidinyl, 1,4-diazepanyl, and azepinyl.

As noted above, in certain embodiments, Z₁ is heteroaryl. According to these embodiments, for example, Z₁ may be substituted or unsubstituted furyl, thienyl, pyridyl, oxazolyl, isoxazolyl, imidazolyl, pyridyl, pyrimidyl, purinyl, triazolyl, or thiazolyl. In one particular embodiment, Z₁ is phenyl, substituted phenyl, substituted alkyl, or substituted or unsubstituted furyl, thienyl, pyridyl, pyridinyl, oxazolyl, imidazolyl, pyridyl, pyrimidyl, purinyl, triazolyl, or thiazolyl; more preferably in this embodiment, Z₁ is phenyl, substituted phenyl, pyridinyl, substituted pyridinyl, furyl, or substituted furyl. In these embodiments, the substituents for the substituted groups may correspond to those described above in connection with Z₁₀₀ and Z₁₀₁.

In another embodiment, Z₁ is heterocyclo. In accordance with this embodiment, for example, Z₁ may be substituted or unsubstituted morpholino, pyran, tetrahydropyran, piperazinyl, piperidinyl, tetrahydropyridinyl, pyrrolidinyl, pyrrolinyl, 1,4-diazepanyl, or azepinyl. In these embodiments, the substituents for the substituted groups may correspond to those described above in connection with Z₁₀₀ and Z₁₀₁.

In another embodiment, Z₁ is alkyl or substituted alkyl. In accordance with this embodiment, therefore, Z₁ may be —(CH₂)ₓ—Z₁₀₂ wherein Z₁₀₂ is hydrogen, hydrocarbyl, substituted hydrocarbyl, hydroxyl, protected hydroxyl, heteroaryl, heterocyclo, amino, amido, alkoxy, aryloxy, cyano, nitro, thiol, or an acetal, ketal, ester, ether, or thioether, and x is 1, 2, or 3. Where Z₁₀₂ is amino, for example, Z₁₀₂ may have the formula: —N(Z_{X})(Z_{Y}) wherein Z_{X} and Z_{Y} are independently hydrogen, hydrocarbyl, substituted hydrocarbyl, heteroaryl, heterocyclo, or an amino protecting group, or Z_{X} and Z_{Y}, together with the nitrogen atom to which they are attached, form a substituted or unsubstituted alicyclic, bicyclic, aryl, heteroaryl, or heterocyclic moiety, typically having 3 to 10 atoms in the ring. In one embodiment in which Z₁₀₂ is amino, for example, Z₁₀₂ is a substituted or unsubstituted piperidine, piperazine, or tetrahydroisoquinoline; according to certain embodiments in which Z₁₀₂ is a tetrahydroisoquinoline, the tetrahydroisoquinoline may have the structure:

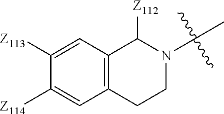

wherein Z₁₁₂, Z₁₁₃, and Z₁₁₄ are independently hydrogen, hydroxyl, hydrocarbyl, substituted hydrocarbyl, alkoxy, alkenoxy, alkynoxy, or aryloxy. In one particular embodiment in which the tetrahydroisoquinoline has the structure shown above, Z₁₁₂, Z₁₁₃, and Z₁₁₄ are independently hydrogen, hydroxyl, alkyl, substituted alkyl, aryl, substituted aryl, alkoxy, or aryloxy.

In combination, among certain of the preferred embodiments are thioacids corresponding to Formula (2) wherein Z₁ is hydrocarbyl, substituted hydrocarbyl, heteroaryl, heterocyclo, or has the formula:

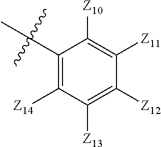

wherein Z₁₀, Z₁₁, Z₁₂, Z₁₃, and Z₁₄ are independently hydrogen, hydrocarbyl, substituted hydrocarbyl, amino, alkoxy, aryl, heteroaryl, heterocyclo, nitro, or trihalomethoxy (e.g., trifluoromethoxy); or Z₁ is —(CH₂)ₓ—Z₁₀₂ wherein Z₁₀₂ is hydrogen, alkyl, substituted alkyl, hydroxyl, protected hydroxyl, heteroaryl, heterocyclo, amino, amido, alkoxy, aryloxy, cyano, nitro, thiol, or an acetal, ketal, ester, ether, or thioether, and x is 1, 2, or 3. Still more preferably in these embodiments, Z₁ is alkyl, substituted alkyl, phenyl, substituted phenyl, or napthyl, each of which may be optionally substituted with one or more amino, alkoxy, nitro, or trihalomethoxy groups, or Z₁ is aminoalkyl, or substituted or unsubstituted thiazolyl, furyl, or isoxazolyl.

In certain embodiments, the thioacids (1) are selected from the group consisting of (TA1), (TA2), (TA3), (TA4), (TA5), (TA6), (TA7), (TA8), (TA9), (TA10), (TA11), (TA12), (TA13), (TA14), and (TA15):

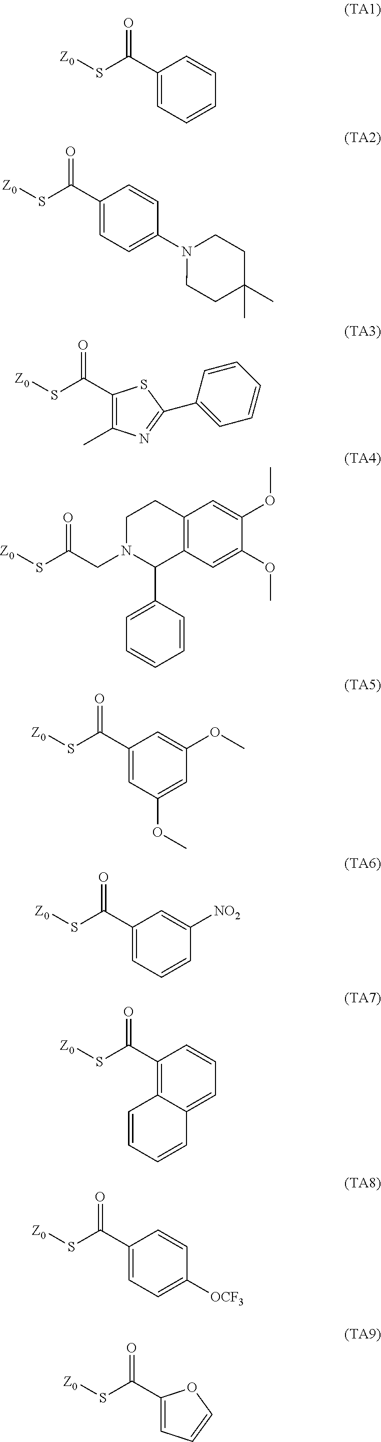

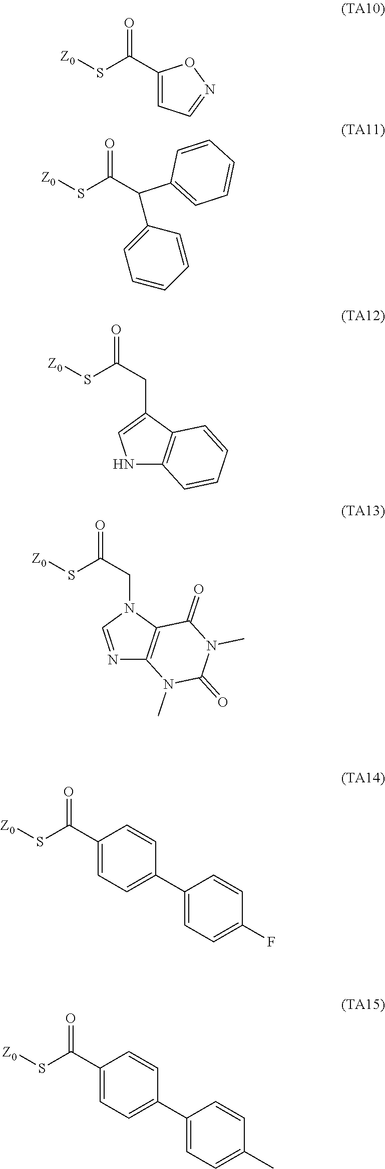

wherein Z₀ in each of (TA1), (TA2), (TA3), (TA4), (TA5), (TA6), (TA7), (TA8), (TA9), (TA10), (TA11), (TA12), (TA13), (TA14), and (TA15) may be hydrogen or a thiol protecting group (e.g., Fmoc).

In one particular embodiment, the thioacid (1) corresponds to one or more of formulae: (TA2), (TA3), (TA4), (TA5), (TA9), and (TA10), wherein Z₀ in each of (TA2), (TA3), (TA4), (TA5), (TA9), and (TA10) may be hydrogen or a thiol protecting group (e.g., Fmoc). In another particular embodiment, the thioacid (1) corresponds to one or more of formulae: (TA2), (TA3), (TA4), (TA5), (TA6), and (TA7), wherein Z₀ in each of (TA2), (TA3), (TA4), (TA5), (TA6), and (TA7) may be hydrogen or a thiol protecting group (e.g., Fmoc). In another particular embodiment, the thioacid (1) corresponds to one or more of formulae: (TA2), (TA3), and (TA8), wherein Z₀ in each of (TA2), (TA3), and (TA8) may be hydrogen or a thiol protecting group (e.g., Fmoc).

In general, the thioacids described above for use in the processes described herein are commercially available or can be prepared according to conventional organic synthesis techniques.

Sulfonyl Azides

The sulfonyl azides for use in reacting with the thioacids corresponding to Formula (1) in the acylsulfonamide-forming processes described herein generally correspond to Formula (2):

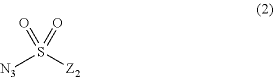

wherein Z₂ is hydrocarbyl, substituted hydrocarbyl, heteroaryl, or heterocyclo. Typically, such hydrocarbyl substituents for Z₂ contain from 1 to 20 carbon atoms and may be linear, branched, or cyclic, and said substituted hydrocarbyl, heteroaryl, and heterocyclo moieties for Z₂ may be substituted with one or more of ═O, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heteroaryl, heterocyclo, and halo (including F, Cl, Br and I), among others, wherein each occurrence of R_{Z} may be hydrocarbyl, substituted hydrocarbyl (e.g., substituted or unsubstituted alkyl, substituted or unsubstituted aryl, or substituted or unsubstituted aralkyl), heteroaryl, or heterocyclo.

In general, although Z₂ is hydrocarbyl, substituted hydrocarbyl, heteroaryl, or heterocyclo, in certain embodiments Z₂ is substituted or unsubstituted alkyl, alkenyl, alkynyl, aryl, alkaryl, or aralkyl. In one particular embodiment, Z₂ is aryl or substituted aryl; thus, for example, Z₂ may have the formula:

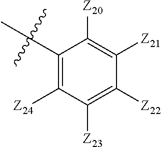

wherein Z₂₀, Z₂₁, Z₂₂, Z₂₃, and Z₂₄ are independently hydrogen, halo, hydrocarbyl, substituted hydrocarbyl, alkoxy, alkenoxy, alkynoxy, aryloxy, nitro, cyano, amino, or amido, or two of Z₂₀, Z₂₁, Z₂₂, Z₂₃, and Z₂₄, together with the carbon atoms to which they are attached, form a fused carbocyclic (e.g., napthyl) or heterocyclic ring. In another particular embodiment, Z₂ is phenyl, substituted phenyl, napthyl, or substituted napthyl.

In one embodiment in which Z₂ corresponds to the aryl or substituted aryl structure illustrated above, Z₂₀, Z₂₁, Z₂₂, Z₂₃, and Z₂₄ are independently alkyl, substituted alkyl, amino, alkoxy, alkenoxy, alkynoxy, or aryloxy. In a particular embodiment, Z₂₀, Z₂₁, Z₂₃, and Z₂₄ are hydrogen and Z₂₃ is alkyl, substituted alkyl, amino, alkoxy, alkenoxy, alkynoxy, or aryloxy.

Where one or more of Z₂₀, Z₂₁, Z₂₂, Z₂₃, and Z₂₄ are substituted alkyl, for example, the alkylene moieties may be further substituted, for example, with ═O, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heteroaryl, heterocyclo, and halo (including F, Cl, Br and I), among others, wherein each occurrence of R_{Z} may be hydrocarbyl, substituted hydrocarbyl (e.g., substituted or unsubstituted alkyl, substituted or unsubstituted aryl, or substituted or unsubstituted aralkyl), heteroaryl, or heterocyclo. In one particular embodiment, Z₂ corresponds to the aryl or substituted aryl structure illustrated above, wherein Z₂₀, Z₂₁, Z₂₃, and Z₂₄ are hydrogen and Z₂₂ is —N(Z₂₂₀)(Z₂₂₁) or —CH₂—N(Z₂₂₀)(Z₂₂₁), wherein Z₂₂₀ and Z₂₂₁ are independently hydrogen, alkyl, substituted alkyl, aryl, substituted aryl, or Z₂₂₀ and Z₂₂₁ together with the nitrogen atom to which they are attached, form a substituted or unsubstituted alicyclic, bicyclic, aryl, heteroaryl, or heterocyclic moiety. In another particular embodiment, Z₂ corresponds to the aryl or substituted aryl structure illustrated above, wherein Z₂₀, Z₂₁, Z₂₃, and Z₂₄ are hydrogen and Z₂₂ is —N(Z₂₂₀)(Z₂₂₁) or —CH₂—N(Z₂₂₀)(Z₂₂₁), wherein Z₂₂₀ and Z₂₂₁ are independently hydrogen, alkyl, substituted alkyl, aryl, substituted aryl, or Z₂₂₀ and Z₂₂₁ together with the nitrogen atom to which they are attached, form a substituted or unsubstituted alicyclic, bicyclic, aryl, heteroaryl, or heterocyclic moiety; more preferably in this embodiment, Z₂ is an N,N-disubstituted (amino)phenyl or (aminomethyl)phenyl. Substituents for the Z₂₂₀ and Z₂₂₁ moieties may be, for example, ═O, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heteroaryl, heterocyclo, and halo (including F, Cl, Br and I), among others, wherein each occurrence of R_{Z} may be hydrocarbyl, substituted hydrocarbyl (e.g., substituted or unsubstituted alkyl, substituted or unsubstituted aryl, or substituted or unsubstituted aralkyl), heteroaryl, or heterocyclo.

In one particular embodiment in which Z₂ corresponds to the aryl or substituted aryl structure illustrated above, Z₂₀, Z₂₁, Z₂₂, Z₂₃, and Z₂₄ are independently alkyl (straight, branched, or cyclic), alkenyl (straight, branched, or cyclic), alkynyl (straight or branched), aryl, alkoxy, arylalkoxy, aryloxy, aryloxyalkoxy, alkylcarbonyloxy, alkylsulfanyl, arylsulfanyl, arylsulfanylalkoxy, cycloalkylalkoxy, cycloalkyloxy, cyano, halo, haloalkyl, haloalkoxy, heterocyclo, (heterocyclo)oxy, nitro, and amino. Where one or more of Z₂₀, Z₂₁, Z₂₂, Z₂₃, and Z₂₄ are amino, the amino moiety may have the formula: —N(Z_{X})(Z_{Y}) wherein Z_{X} and Z_{Y} are independently hydrogen, alkyl, alkenyl, alkoxyalkyl, alkoxycarbonylalkyl, alkylsulfanylalkyl, alkylsulfonylalkyl, aryl, arylalkyl, arylalkylsulfanylalkyl, aryloxyalkyl, arylsulfanylalkyl, arylsulfinylalkyl, arylsulfonylalkyl, carboxyalkyl, cycloalkenyl, cycloalkenylalkyl, cycloalkyl, (cycloalkyl)alkyl, cycloalkylcarbonyl, heterocyclo, (heterocyclo)alkyl, (heterocyclo)sulfanylalkyl, hydroxyalkyl, or a nitrogen protecting group, or Z_{X} and Z_{Y}, together with the nitrogen atom to which they are attached, form a substituted or unsubstituted imidazolyl, morpholinyl, piperazinyl, piperidinyl, pyrrolidinyl, pyrrolyl, thiomorpholinyl, or thiomorpholinyl dioxide moiety.

In another particular embodiment, Z₂ is alkyl or substituted alkyl. In accordance with this embodiment, therefore, Z₂ may be —(CH₂)ₓ—Z₂₀₀ wherein Z₂₀₀ is hydrogen, hydrocarbyl, substituted hydrocarbyl, hydroxyl, protected hydroxyl, heteroaryl, heterocyclo, amino, amido, alkoxy, aryloxy, cyano, nitro, thiol, or an acetal, ketal, ester, ether, or thioether, and x is 1, 2, or 3. In one embodiment, Z₂ is —(CH₂)ₓ—Z₂₀₀ wherein x is 1, 2, or 3 and Z₂₀₀ is —N(Z₂₀₁)(Z₂₀₂), wherein Z₂₀₁ and Z₂₀₂ are independently hydrogen, alkyl, substituted alkyl, aryl, substituted aryl, or Z₂₀₁ and Z₂₀₂, together with the nitrogen atom to which they are attached, for a substituted or unsubstituted alicyclic, bicyclic, aryl, heteroaryl, or heterocyclic moiety.

Alternatively, Z₂ may be heteroaryl. Thus, for example, Z₂ may be substituted or unsubstituted furyl, thienyl, pyrrolyl, oxazolyl, imidazolyl, pyridyl, pyrimidyl, purinyl, triazolyl, or thiazolyl.

In another alternative embodiment, Z₂ is heterocyclo. In accordance with this embodiment, for example, Z₂ may be substituted or unsubstituted morpholino, pyran, tetrahydropyran, piperazinyl, piperidinyl, tetrahydropyridinyl, pyrrolidinyl, pyrrolinyl, 1,4-diazepanyl, or azepinyl. In these embodiments, the substituents for the substituted groups may correspond to those described above in connection with Z₂₀, Z₂₁, Z₂₂, Z₂₃, and Z₂₄.

In one particular embodiment, for example, the sulfonyl azide corresponds to Formula (2A) or (2B):

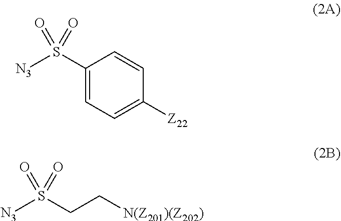

wherein Z₂₂ is hydrogen, halo, alkoxy, alkyl, or substituted alkyl; and

Z₂₀₁ and Z₂₀₂ are independently hydrogen, alkyl, substituted alkyl, aryl, substituted aryl, or Z₂₀₁ and Z₂₀₂ together with the nitrogen atom to which they are attached, form a substituted or unsubstituted alicyclic, bicyclic, aryl, heteroaryl, or heterocyclic moiety. Where one or more of Z₂₂, Z₂₀₁, and Z₂₀₂ are substituted alkyl, for example, the alkyl moieties may be substituted, for example, with ═O, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heteroaryl, heterocyclo, and halo (including F, Cl, Br and I), among others, wherein each occurrence of R_{Z} may be hydrocarbyl, substituted hydrocarbyl (e.g., substituted or unsubstituted alkyl, substituted or unsubstituted aryl, or substituted or unsubstituted aralkyl), heteroaryl, or heterocyclo.

In certain embodiments, the sulfonyl azides (2) are selected from the group consisting of (SZ1), (SZ2), (SZ3), (SZ4), (SZ5), (SZ6), (SZ7), (SZ8), (SZ9), (SZ10), (SZ11), (SZ12), (SZ13), (SZ14), (SZ15), (SZ16), (SZ17), (SZ18), (SZ19), (SZ20), (SZ21), (SZ22), (SZ23), (SZ24), (SZ25), (SZ26), (SZ27), (SZ28), (SZ29), (SZ30), and (SZ31):

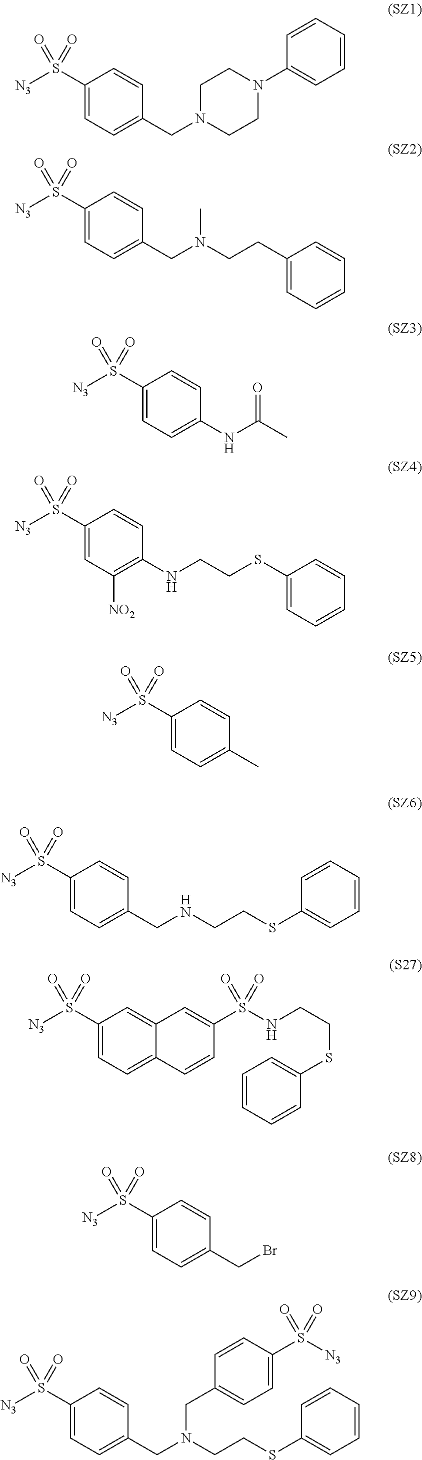

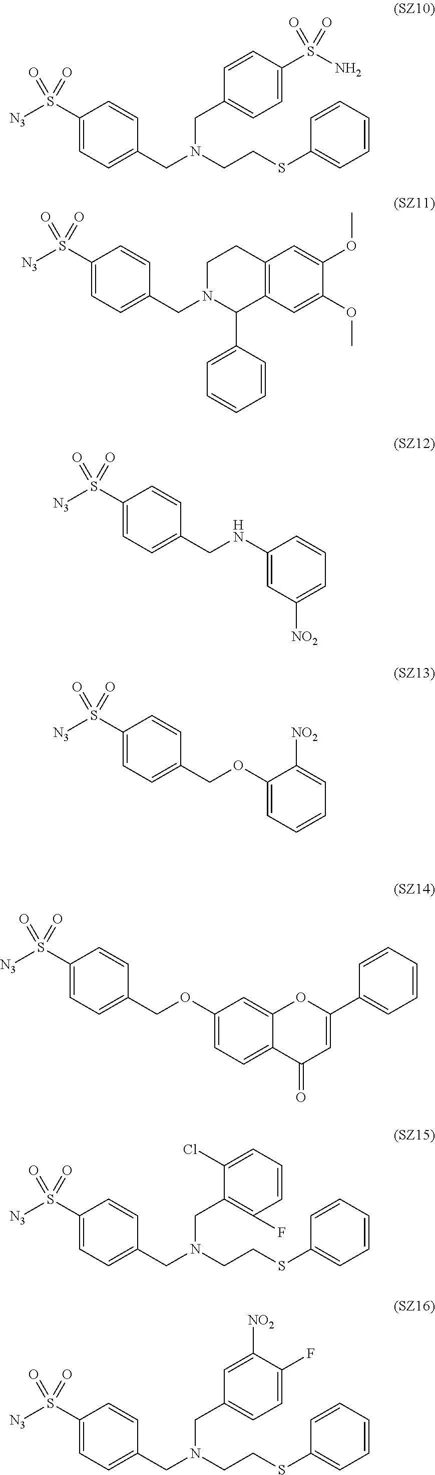

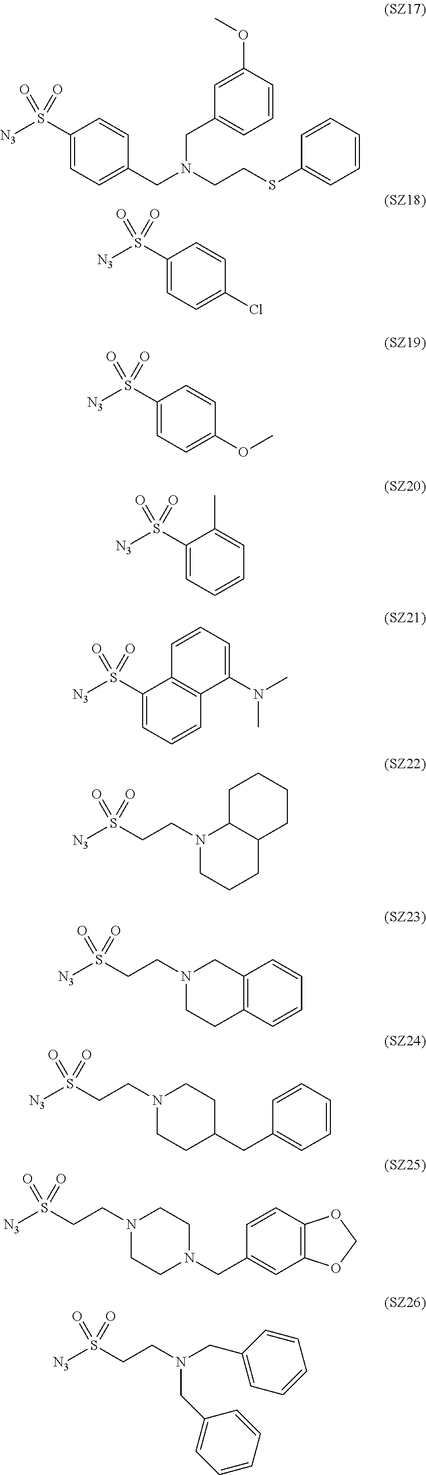

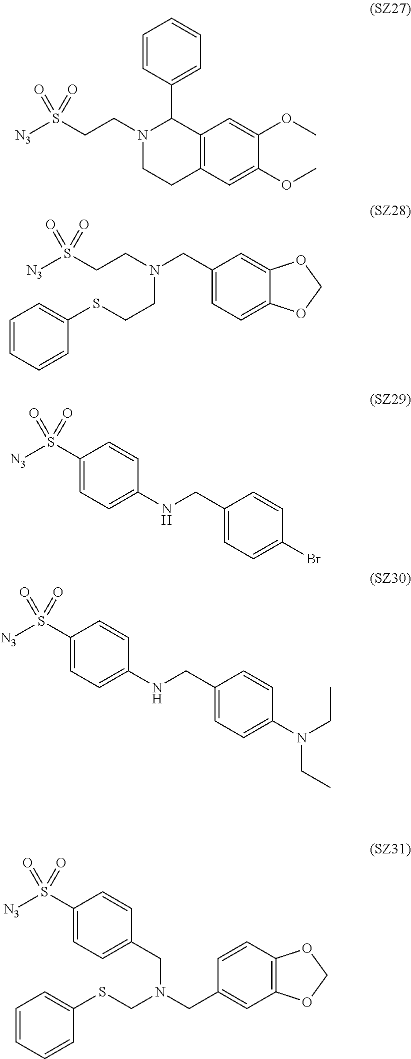

In one particular embodiment, the sulfonyl azide (2) corresponds to one or more of formulae: (SZ9), (SZ10), (SZ11), (SZ15), (SZ16), (SZ17) and (SZ31). In another particular embodiment, the sulfonyl azide (3) corresponds to one or more of formulae: (SZ15), (SZ17), and (SZ31). In general, the sulfonyl azides described above for use in the processes described herein are commercially available or can be prepared according to conventional organic synthesis techniques.

Bcl-2 Family Proteins

The thioacid (1) and the sulfonyl azide (2), or libraries thereof, are reacted in the presence of a biological target. In general, the biological target is a biological molecule involved in one or more biological pathways associated with various diseases and conditions including cancer, diabetes, neurodegenerative diseases, cardiovascular diseases, respiratory diseases, digestive system diseases, infectious diseases, inflammatory diseases, autoimmune diseases, and the like. Likewise, a range of biological pathways may be involved, including cell cycle regulation (e.g., cellular proliferation and apoptosis), angiogenesis, signaling pathways, tumor suppressor pathways, inflammation, oncogenes, and growth factor receptors, among a variety of others.

As noted above, the Bcl-2 family of proteins includes both anti-apoptotic molecules and pro-apoptotic molecules. The anti-apoptotic Bcl-2 family members (e.g., Bcl-2, Bcl-X_{L}, Mcl-1, A1/BFL-1, Boo/Diva, Bcl-w, and Bcl-y) inhibit the release of certain pro-apoptotic factors from mitochondria, whereas pro-apoptotic Bcl-2 family members (e.g., Bak, Bax, Bad, tBid, Harakiri (HRK), Bim, Bcl-Xs, Bmf, Egl-1, Puma, and Noxa) induce the release of mitochondrial apoptogenic molecules into the cytosol. In accordance the process described herein, the thioacid(s) (1) is/are reacted with the sulfonyl azide(s) (2) in the presence of a protein of the Bcl-2 family; thus, in one embodiment the Bcl-2 family protein is an anti-apoptotic Bcl-2 family protein, and in another embodiment the Bcl-2 family protein is a pro-apoptotic Bcl-2 family protein. In some of these embodiments, the Bcl-2 family proteins contemplated include, but are not limited to, Bcl-2, Bcl-X_{L}, Mcl-1, A1/BFL-1, Boo/Diva, Bcl-w, Bcl-y, Bak, Bax, Bad, tBid, Harakiri, Bim, Bcl-Xs, Bmf, Egl-1, Puma, and Noxa. It is also contemplated that two or more Bcl-2 protein family members may be utilized in the reaction. In one particular embodiment, the Bcl-2 family protein is Bcl-X_{L}. In another particular embodiment, the Bcl-2 family protein is Mcl-1. In certain preferred embodiments, the resulting acylsulfonamide (3) has a specificity to Mcl-1 over Bcl-X_{L}.

Acylsulfonamides

The processes described herein generally utilize the biological target molecule (e.g., Bcl-X_{L} or Mcl-1) as the reaction vessel or reaction template to assemble an acylsulfonamide compound having preferential binding to the biological target, from one or more thioacids and one or more sulfonyl azides. Thus, the target-guided synthesis strategy utilizes the biological molecule itself as a template for generating potential ligand inhibitors from the initial building block fragments or scaffolds (i.e., the thioacids and the sulfonyl azides in the library), that are selectively bound to the target biomolecule and then irreversibly linked to each other within the confines of the binding pockets of the target protein. As this approach employs the biological target to assemble its own inhibitors from relatively few starting reagents (which can be combined in thousands or tens of thousands of different ways), rather than requiring tedious synthesis, purification, and screening of thousands of library compounds, it is more efficient than conventional combinatorial chemistry techniques. However, as described in further detail below, certain aspects of combinatorial chemistry can be used in the methods described herein.

The thioacids and the sulfonyl azides generally combine to form an acylsulfonamide. These techniques are capable of producing high-affinity inhibitors by assembling the building block reagents irreversibly inside the binding pockets of a target biomolecule. Subsequent screening of target biomolecule-generated “hits” then establish their binding affinity to and specificity for the target. Once the “hit” compounds are determined, they can be synthesized according to conventional organic chemistry methods such as described below, or extracted from the target protein and purified in trace amounts.

For bivalent molecules that have multiple interactions with the Bcl-2 family protein, the resulting hits are very potent (e.g., high affinity); the bivalent molecules bind to the protein binding site and reach into the substrate pocket. For entropy reasons (e.g., avoidance of the loss of three degrees of rotational and translational freedom), among other things, ligand inhibitors display much higher affinity to their biological targets than the individual components. Thus, even initial compound (e.g., thioacids and sulfonyl azides) fragments with only modest micromolar affinity to individual binding pockets can generate nanomolar inhibitors when coupled together to permit optimal binding interactions with the biological target. Thus, the binding affinity of the building block reagent (i.e., scaffold) or precursor to the Bcl-2 family protein does not need to be in the nanomolar range.

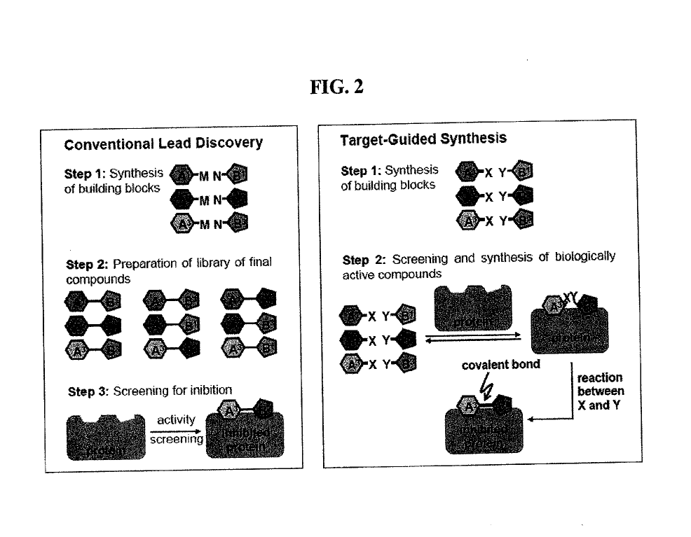

The general approach of in situ ligation chemistry is illustrated in FIG. 2, and ligation chemistry techniques are described, for example, in the following references: Kolb et al., Angew. Chem. Int. Ed. 2001, 40, 2004-2021; Kolb et al., Drug Discovery Today 2003, 8, 1128-1137; Rostovtsev et al., Angew. Chem. Int. Ed. 2002, 41, 2596-2599; Tornoe et al., Journal of Organic Chemistry 2002, 67, 3057-3064; Wang et al., Journal of the American Chemical Society 2003, 125, 3192-3193; Lee et al., Journal of the American Chemical Society 2003, 125, 9588-9589; Lewis et al., Angew. Chem., Int. Ed. 2002, 41, 1053-1057; Manetsch et al., Journal of the American Chemical Society 2004, 126, 12809-12818; Mocharla et al., Angew. Chem. Int. Ed. 2005, 44, 116-120; Whiting et al., Angew. Chem. 2006, 118, 1463-1467; Whiting et al., Angew. Chem. Int. Ed. Engl. 2006, 45, 1435-1439; and Sharpless et al., Expert Opin. Drug Discovery 2006, 1, 525-538.

In particular, the thioacids and sulfonyl azides corresponding to Formula (1) and (2), respectively, undergo an amidation reaction as illustrated in Reaction Scheme (1) (see also, e.g., Shangguan et al. J. Am. Chem. Soc. 2003, 125, 7754-7755). As noted above, the reaction of the thioacid and the sulfonyl azide is templated by the biological target molecule, a Bcl-2 family protein, in situ within its binding pockets. Typically, several thioacids (1) and sulfonyl azides (2) in the form of one or more libraries will be reacted in the presence of the Bcl-2 family protein; the resulting acylsulfonamide(s) (3) which bind(s) to the Bcl-2 family protein will be the compound(s) of interest (e.g., for further synthesis, testing, and analysis).

Thus, the acylsulfonamides which can be prepared in accordance with the process described herein generally correspond to Formula (3):

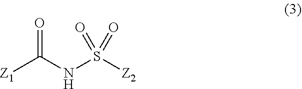

wherein Z₁ and Z₂ are as defined in connection with Formulae (1) and (2).

For instance, in one embodiment, Z₁ is aryl, substituted aryl, or heteroaryl. Thus, in certain embodiments. Z₁ has the formula:

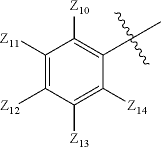

wherein

Z₁₀, Z₁₁, Z₁₂, Z₁₃, and Z₁₄ are independently hydrogen, hydroxyl, protected hydroxyl, halo, hydrocarbyl, substituted hydrocarbyl, heterocyclo, heteroaryl, alkoxy, alkenoxy, alkynoxy, aryloxy, arylalkoxy (heterocyclo)alkoxy, trihaloalkoxy, amino, amido, or cyano, or two of Z₁₀, Z₁₁, Z₁₂, Z₁₃, and Z₁₄, together with the carbon atoms to which they are attached, form a fused carbocyclic (e.g., napthyl) or heterocyclic ring. In one particular embodiment, Z₁₀, Z₁₁, Z₁₂, Z₁₃, and Z₁₄ are independently hydrogen, hydrocarbyl, substituted hydrocarbyl, amino, alkoxy, nitro, or trihalomethoxy.

In an alternative embodiment, Z₁ has the formula:

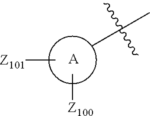

wherein A is phenyl or a five- or six-membered aromatic carbocyclic or heterocyclic ring wherein from one to three carbon atoms may be replaced by a heteroatom selected from N, O, or S, and wherein A is substituted with Z₁₀₀ and Z₁₀₁ through ring carbon atoms or ring heteroatoms, and Z₁₀₀ and Z₁₀₁ are independently hydrogen, hydrocarbyl, substituted hydrocarbyl, heterocyclo, alkoxy, alkenoxy, alkynoxy, aryloxy, heterocyclo(alkoxy), or halo.

In other embodiments, Z₁ is substituted or unsubstituted furyl, thienyl, pyridyl, oxazolyl, isoxazolyl, imidazolyl, pyridyl, pyrimidyl, purinyl, triazolyl, or thiazolyl, or Z₁ is substituted or unsubstituted morpholino, pyran, tetrahydropyran, piperazinyl, piperidinyl, tetrahydropyridinyl, pyrrolidinyl, pyrrolinyl, 1,4-diazepanyl, or azepinyl. In still other embodiments, Z₁ is alkyl or substituted alkyl; here, for example, Z₁ may be —(CH₂)ₓ—Z₁₀₂ wherein Z₁₀₂ is hydrogen, hydrocarbyl, substituted hydrocarbyl, hydroxyl, protected hydroxyl, heteroaryl, heterocyclo, amino, amido, alkoxy, aryloxy, cyano, nitro, thiol, or an acetal, ketal, ester, ether, or thioether, and x is 1, 2, or 3. Substituents for such groups in these embodiments may be selected from the group consisting of ═O, —OH, —OR_{Z}, —COOH, —COOR_{Z}, —CONH₂, —NH₂, —NHR_{Z}, —NR_{Z}, —NO₂, —SH, —SR_{Z}, —SO₂R_{Z}, —SO₂H, —SOR_{Z}, heteroaryl, heterocyclo, and halo (including F, Cl, Br and I), among others, wherein each occurrence of R_{Z} may be hydrocarbyl or substituted hydrocarbyl (e.g., substituted or unsubstituted alkyl, substituted or unsubstituted aryl, or substituted or unsubstituted aralkyl).

Alternatively, Z₁ may be hydrocarbyl, substituted hydrocarbyl, heteroaryl, heterocyclo, or have the formula:

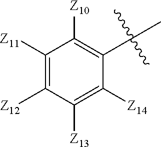

wherein Z₁₀, Z₁₁, Z₁₂, Z₁₃, and Z₁₄ are independently hydrogen, amino, alkoxy, aryl, heteroaryl, heterocyclo, nitro, or trihalomethoxy (e.g., trifluoromethoxy); or Z₁ may be —(CH₂)ₓ—Z₁₀₂ wherein Z₁₀₂ is hydrogen, alkyl, substituted alkyl, hydroxyl, protected hydroxyl, heterocyclo, amino, amido, alkoxy, aryloxy, cyano, nitro, thiol, or an acetal, ketal, ester, ether, or thioether, and x is 1, 2, or 3.

Similarly, in these and other embodiments, Z₂ may be substituted or unsubstituted alkyl, alkenyl, alkynyl, aryl, alkaryl, or aralkyl. Thus, in certain embodiments, for example, Z₂ may have the formula:

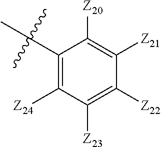

wherein

Z₂₀, Z₂₁, Z₂₂, Z₂₃, and Z₂₄ are independently hydrogen, halo, hydrocarbyl, substituted hydrocarbyl, alkoxy, alkenoxy, alkynoxy, aryloxy, nitro, cyano, amino, or amido, or two of Z₂₀, Z₂₁, Z₂₂, Z₂₃, and Z₂₄, together with the carbon atoms to which they are attached, form a fused carbocyclic or heterocyclic ring. For instance, Z₂₀, Z₂₁, Z₂₂, Z₂₃, and Z₂₄ may independently be alkyl, substituted alkyl, amino, alkoxy, alkenoxy, alkynoxy, or aryloxy. In another embodiment, Z₂ is phenyl, substituted phenyl, napthyl, or substituted napthyl.

In other embodiments, Z₂ is substituted or unsubstituted furyl, thienyl, pyridyl, oxazolyl, isoxazolyl, imidazolyl, pyridyl, pyrimidyl, purinyl, triazolyl, or thiazolyl, or Z₂ is substituted or unsubstituted morpholino, pyran, tetrahydropyran, piperazinyl, piperidinyl, tetrahydropyridinyl, pyrrolidinyl, pyrrolinyl, 1,4-diazepanyl, or azepinyl. In still other embodiments, Z₂ is alkyl or substituted alkyl; here, for example, Z₂ may be —(CH₂)ₓ—Z₂₀₀ wherein Z₂₀₀ is hydrogen, hydroxyl, protected hydroxyl, heterocyclo, amino, amido, alkoxy, aryloxy, cyano, nitro, thiol, or an acetal, ketal, ester, ether, or thioether, and x is 1, 2, or 3.

In combination, Z₁ and Z₂ may each be a substituted or unsubstituted aryl or heteroaryl moiety. In one particular embodiment, Z₁ is an N,N-disubstituted (aminomethyl)phenyl moiety and Z₂ is a para-substituted benzene or napthyl moiety. The substituents for Z₁ and/or Z₂ in this embodiment may be, for example, ═O, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heteroaryl, heterocyclo, and halo (including F, Cl, Br and I), among others, wherein each occurrence of R_{Z} may be hydrocarbyl or substituted hydrocarbyl (e.g., substituted or unsubstituted alkyl, substituted or unsubstituted aryl, or substituted or unsubstituted aralkyl), among other things.

In one particular embodiment, the acylsulfonamide (3) has the formula:

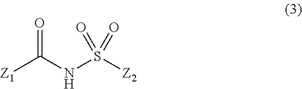

wherein

Z₁ is:

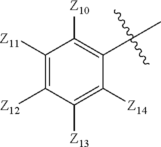

Z₂ is substituted alkyl or:

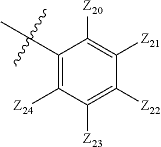

Z₁₁ and Z₁₃ are alkyl, substituted alkyl, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heterocyclo, and halo, among others, wherein each occurrence of R_{Z} is substituted or unsubstituted alkyl, substituted or unsubstituted aryl, or substituted or unsubstituted aralkyl;

Z₂₂ is —N(Z₂₂₀)(Z₂₂₁) or —CH₂—N(Z₂₂₀)(Z₂₂₁), wherein Z₂₂₀ and Z₂₂₁ are independently hydrogen, alkyl, substituted alkyl, aryl, substituted aryl, or Z₂₂₀ and Z₂₂₁ together with the nitrogen atom to which they are attached, form a substituted or unsubstituted alicyclic, bicyclic, aryl, or heterocyclic moiety; and

Z₁₀, Z₁₂, Z₁₄, Z₂₀, Z₂₁, Z₂₃, and Z₂₄ are hydrogen. In one embodiment in which Z₂ is substituted alkyl, Z₂ is —(CH₂)ₓ—Z₂₀₀ wherein x is 1, 2, or 3 and Z₂₀₀ is —N(Z₂₀₁)(Z₂₀₂), wherein Z₂₀₁ and Z₂₀₂ are independently hydrogen, alkyl, substituted alkyl, aryl, substituted aryl, or Z₂₀₁ and Z₂₀₂, together with the nitrogen atom to which they are attached, for a substituted or unsubstituted alicyclic, bicyclic, aryl, heteroaryl, or heterocyclic moiety.

In certain embodiments, the acylsulfonamide (3) is selected from the group consisting of (SZ4TA2), (SZ7TA2), (SZ9TA5), (SZ9TA2), (SZ10TA2), (SZ15TA3), (SZ15TA8), (SZ16TA6), (SZ16TA8), (SZ17TA7), (SZ2TA1), (SZ2TA2), (SZ2TA3), (SZ4TA1), (SZ5TA1), (SZ5TA2), (SZ9TA1), (SZ10TA1), (SZ10TA5), (SZ15TA1), (SZ15TA2), (SZ15TA4), (SZ15TA5), (SZ15TA6), (SZ15TA7), (SZ15TA9, (SZ15TA10), (SZ17TA3), (SZ3TA6), (SZ3TA9), and (SZ9TA7):

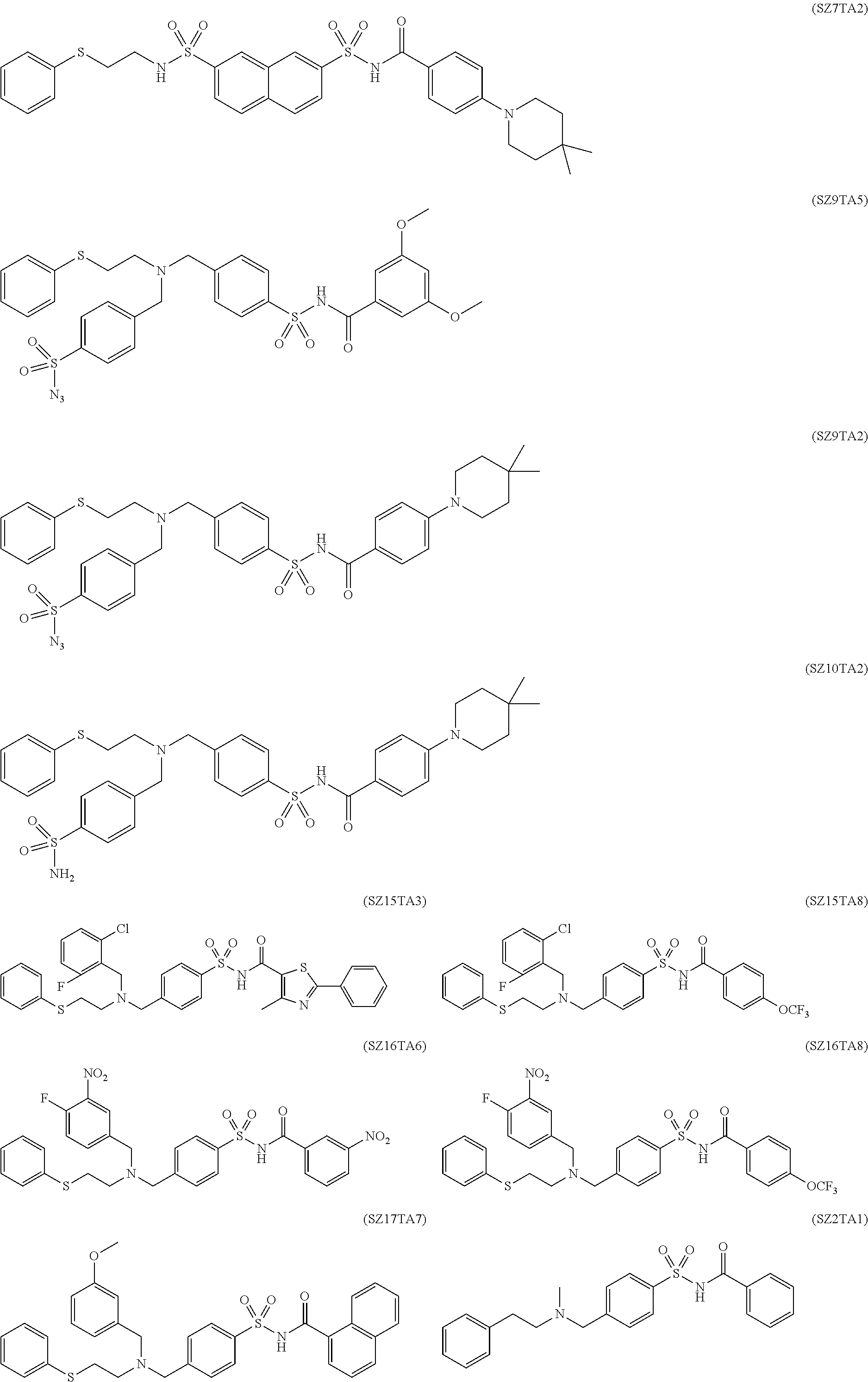

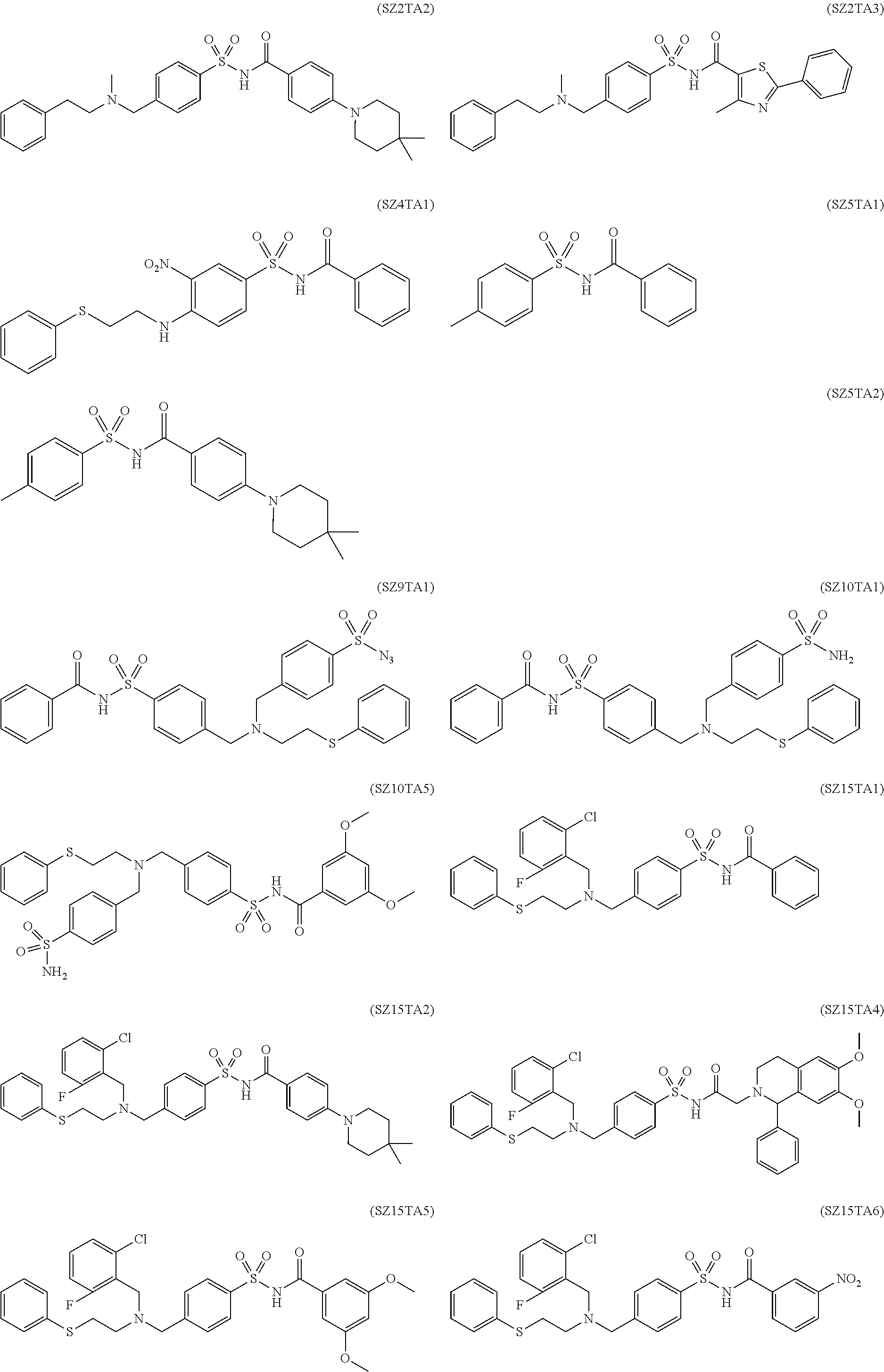

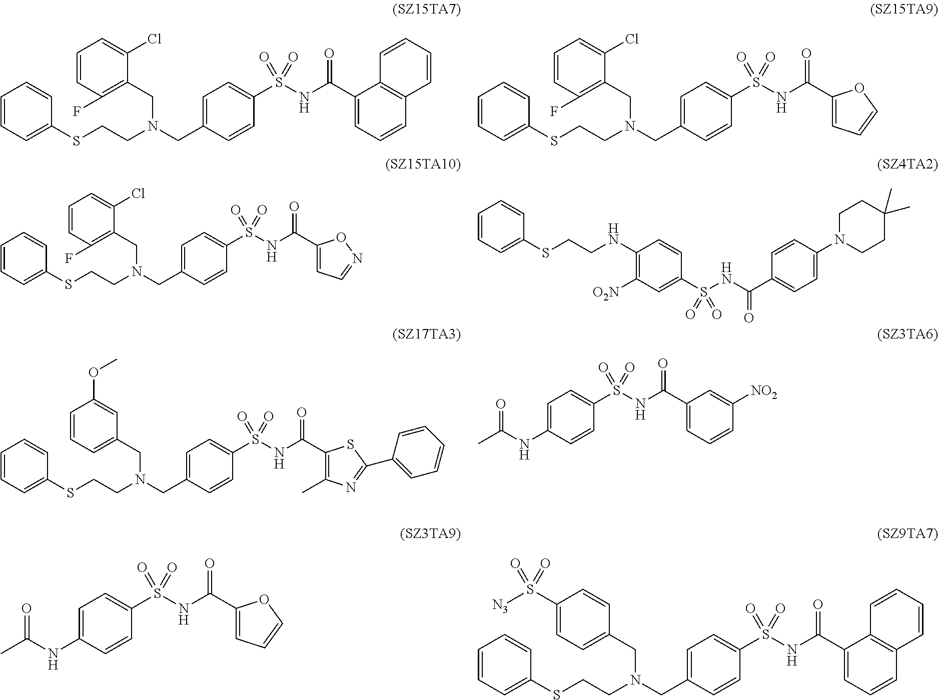

In one particular embodiment, the acylsulfonamide (3) corresponds to one or more of formulae: (SZ4TA2), (SZ7TA2), (SZ9TA5), (SZ9TA2), (SZ10TA2), (SZ15TA3), (SZ15TA8), (SZ16TA6), (SZ16TA8), and (SZ17TA7). In another particular embodiment, the acylsulfonamide (3) corresponds to one or more of formulae: (SZ2TA1), (SZ2TA2), (SZ2TA3), (SZ4TA1), (SZ5TA1), (SZ5TA2), (SZ9TA1), (SZ10TA1), (SZ10TA5), (SZ15TA1), (SZ15TA2), (SZ15TA4), (SZ15TA5), (SZ15TA6), (SZ15TA7), (SZ15TA9, (SZ15TA10), (SZ17TA3), (SZ3TA6), (SZ3TA9), and (SZ9TA7).

In other embodiments, the acylsulfonamide (3) is selected from the group consisting of (SZ1TA3), (SZ2TA4), (SZ2TA10), (SZ3TA1), (SZ3TA7), (SZ3TA8), (SZ6TA2), (SZ6TA7), (SZ9TA3), (SZ11TA3), (SZ11TA4), (SZ11TA5), (SZ11TA8), (SZ11TA9), (SZ16TA7), (SZ17TA4), (SZ17TA8), (SZ21TA7), (SZ23TA5), (SZ30TA7), (SZ31TA2), (SZ31TA3), (SZ31TA5), (SZ31TA6), (SZ31TA7), (SZ31TA8), and (SZ31TA15):

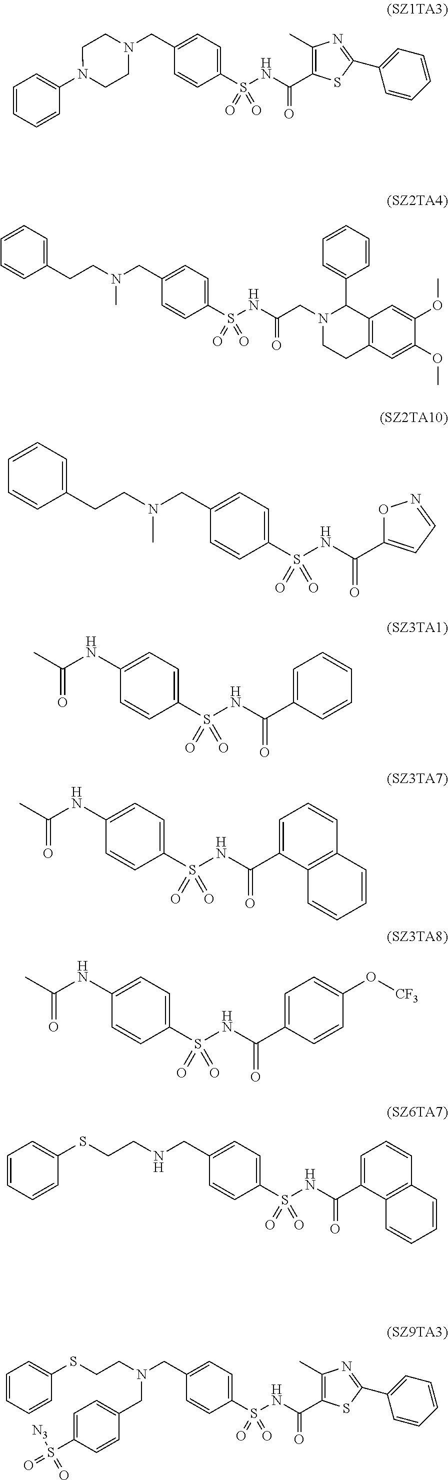

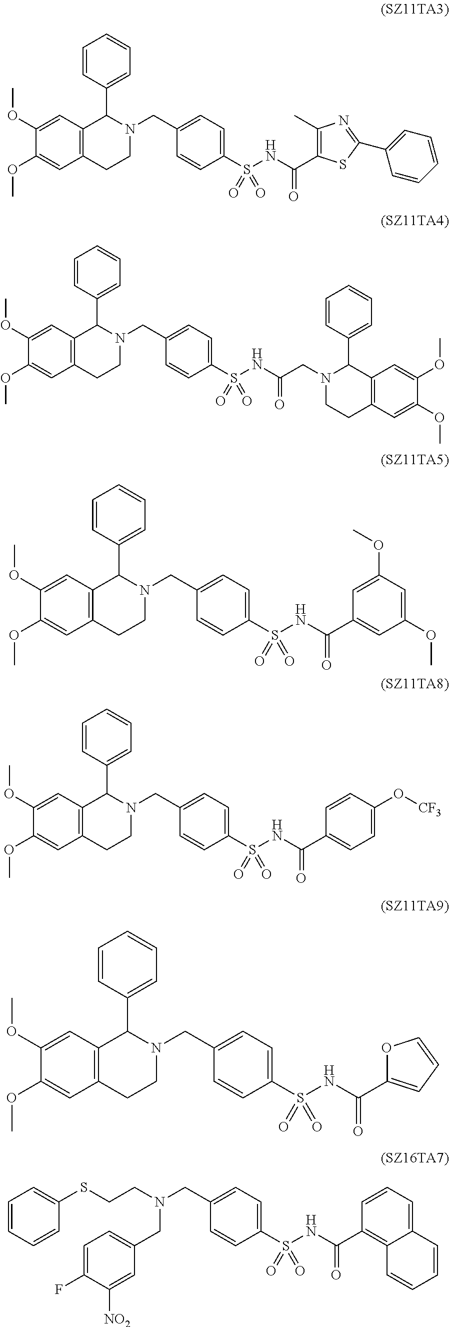 [SMILES: COc1cc2c(cc1OC)C(c1ccccc1)N(Cc1ccc(S(=O)(=O)NC(=O)c3sc(-c4ccccc4)nc3C)cc1)CC2 ; COc1cc2c(cc1OC)C(c1ccccc1)N(CC(=O)NS(=O)(=O)c1ccc(CN3CCc4cc(OC)c(OC)cc4C3c3ccccc3)cc1)CC2 ; COc1cc(OC)cc(C(=O)NS(=O)(=O)c2ccc(CN3CCc4cc(OC)c(OC)cc4C3c3ccccc3)cc2)c1 ; COc1cc2c(cc1OC)C(c1ccccc1)N(Cc1ccc(S(=O)(=O)NC(=O)c3ccc(OC(F)(F)F)cc3)cc1)CC2 ; COc1cc2c(cc1OC)C(c1ccccc1)N(Cc1ccc(S(=O)(=O)NC(=O)c3ccco3)cc1)CC2 ; O=C(NS(=O)(=O)c1ccc(CN(CCSc2ccccc2)Cc2ccc(F)c([N+](=O)[O-])c2)cc1)c1cccc2ccccc12]

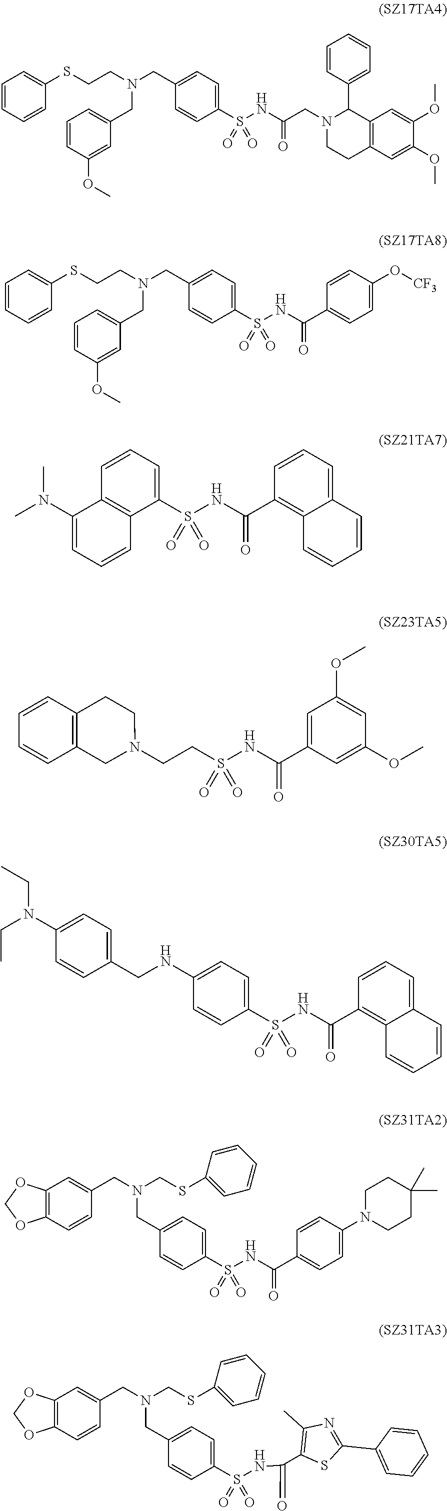

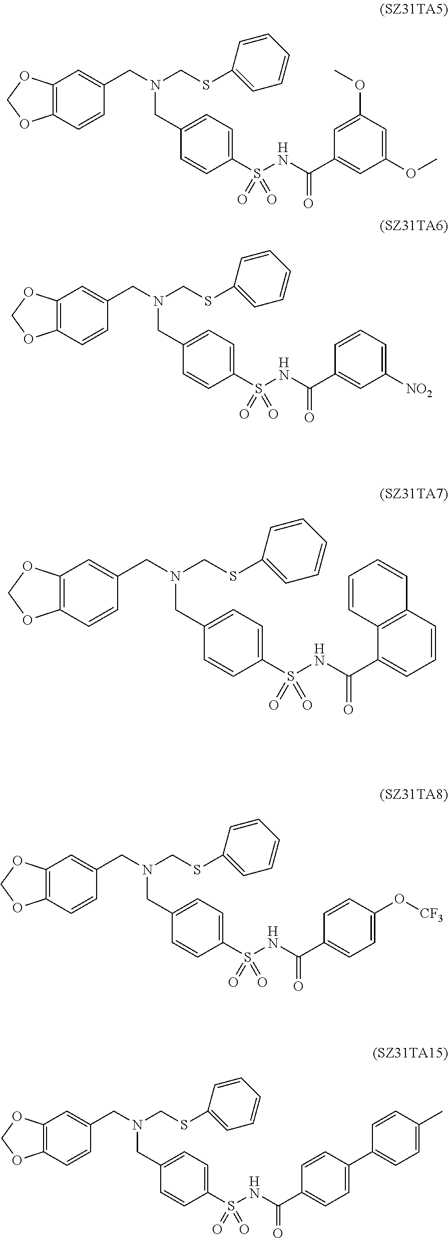 [SMILES: COc1cc(OC)cc(C(=O)NS(=O)(=O)c2ccc(CN(CSc3ccccc3)Cc3ccc4c(c3)OCO4)cc2)c1 ; O=C(NS(=O)(=O)c1ccc(CN(CSc2ccccc2)Cc2ccc3c(c2)OCO3)cc1)c1cccc([N+](=O)[O-])c1 ; O=C(NS(=O)(=O)c1ccc(CN(CSc2ccccc2)Cc2ccc3c(c2)OCO3)cc1)c1cccc2ccccc12 ; O=C(NS(=O)(=O)c1ccc(CN(CSc2ccccc2)Cc2ccc3c(c2)OCO3)cc1)c1ccc(OC(F)(F)F)cc1 ; Cc1ccc(-c2ccc(C(=O)NS(=O)(=O)c3ccc(CN(CSc4ccccc4)Cc4ccc5c(c4)OCO5)cc3)cc2)cc1]

In the interest of space, the structure of each and every acylsulfonamide that can be formed from the various combinations of thioacids and sulfonyl azides described above are not depicted herein. Thus, the generic “SZTA” nomenclature is used herein to refer to an acylsulfonamide compound (i.e., an “SZTA” compound) that was formed as a result of the above-referenced reaction between a particular sulfonyl azide a particular thioacid. By way of example, the acylsulfonamide (SZ31TA2) is the compound that results from the reaction of sulfonyl azide (SZ31) and thioacid (TA2). Thus, for example, in various embodiments the acylsulfonamide can be selected from one or more of the following “SZTA” compounds: (SZ1TA1), (SZ1TA2), (SZ1TA3), (SZ1TA4), (SZ1TA5), (SZ1TA6), (SZ1TA7), (SZ1TA8), (SZ1TA9), (SZ1TA10), (SZ1TA11), (SZ1TA12), (SZ1TA13), (SZ1TA14), (SZ1TA15), (SZ2TA1), (SZ2TA2), (SZ2TA3), (SZ2TA4), (SZ2TA5), (SZ2TA6), (SZ2TA7), (SZ2TA8), (SZ2TA9), (SZ2TA10), (SZ2TA11), (SZ2TA12), (SZ2TA13), (SZ2TA14), (SZ2TA15), (SZ3TA1), (SZ3TA2), (SZ3TA3), (SZ3TA4), (SZ3TA5), (SZ3TA6), (SZ3TA7), (SZ3TA8), (SZ3TA9), (SZ3TA10), (SZ3TA11), (SZ3TA12), (SZ3TA13), (SZ3TA14), (SZ3TA15), (SZ4TA1), (SZ4TA2), (SZ4TA3), (SZ4TA4), (SZ4TA5), (SZ4TA6), (SZ4TA7), (SZ4TA8), (SZ4TA9), (SZ4TA10), (SZ4TA11), (SZ4TA12), (SZ4TA13), (SZ4TA14), (SZ4TA15), (SZ5TA1), (SZ5TA2), (SZ5TA3), (SZ5TA4), (SZ5TA5), (SZ5TA6), (SZ5TA7), (SZ5TA8), (SZ5TA9), (SZ5TA10), (SZ5TA11), (SZ5TA12), (SZ5TA13), (SZ5TA14), (SZ5TA15), (SZ6TA1), (SZ6TA2), (SZ6TA3), (SZ6TA4), (SZ6TA5), (SZ6TA6), (SZ6TA7), (SZ6TA8), (SZ6TA9), (SZ6TA10), (SZ6TA11), (SZ6TA12), (SZ6TA13), (SZ6TA14), (SZ6TA15), (SZ7TA1), (SZ7TA2), (SZ7TA3), (SZ7TA4), (SZ7TA5), (SZ7TA6), (SZ7TA7), (SZ7TA8), (SZ7TA9), (SZ7TA10), (SZ7TA11), (SZ7TA12), (SZ7TA13), (SZ7TA14), (SZ7TA15), (SZ8TA1), (SZ8TA2), (SZ8TA3), (SZ8TA4), (SZ8TA5), (SZ8TA6), (SZ8TA7), (SZ8TA8), (SZ8TA9), (SZ8TA10), (SZ8TA11), (SZ8TA12), (SZ8TA13), (SZ8TA14), (SZ8TA15), (SZ9TA1), (SZ9TA2), (SZ9TA3), (SZ9TA4), (SZ9TA5), (SZ9TA6), (SZ9TA7), (SZ9TA8), (SZ9TA9), (SZ9TA10), (SZ9TA11), (SZ9TA12), (SZ9TA13), (SZ9TA14), (SZ9TA15), (SZ10TA1), (SZ10TA2), (SZ10TA3), (SZ10TA4), (SZ10TA5), (SZ10TA6), (SZ10TA7), (SZ10TA8), (SZ10TA9), (SZ10TA10), (SZ10TA11), (SZ10TA12), (SZ10TA13), (SZ10TA14), (SZ10TA15), (SZ11TA1), (SZ11TA2), (SZ11TA3), (SZ11TA4), (SZ11TA5), (SZ11TA6), (SZ11TA7), (SZ11TA8), (SZ11TA9), (SZ11TA10), (SZ11TA11), (SZ11TA12), (SZ11TA13), (SZ11TA14), (SZ11TA15), (SZ12TA1), (SZ12TA2), (SZ12TA3), (SZ12TA4), (SZ12TA5), (SZ12TA6), (SZ12TA7), (SZ12TA8), (SZ12TA9), (SZ12TA10), (SZ12TA11), (SZ12TA12), (SZ12TA13), (SZ12TA14), (SZ12TA15), (SZ13TA1), (SZ13TA2), (SZ13TA3), (SZ13TA4), (SZ13TA5), (SZ13TA6), (SZ13TA7), (SZ13TA8), (SZ13TA9), (SZ13TA10), (SZ13TA11), (SZ13TA12), (SZ13TA13), (SZ13TA14), (SZ13TA15), (SZ14TA1), (SZ14TA2), (SZ14TA3), (SZ14TA4), (SZ14TA5), (SZ14TA6), (SZ14TA7), (SZ14TA8), (SZ14TA9), (SZ14TA10), (SZ14TA11), (SZ14TA12), (SZ14TA13), (SZ14TA14), (SZ14TA15), (SZ15TA1), (SZ15TA2), (SZ15TA3), (SZ15TA4), (SZ15TA5), (SZ15TA6), (SZ15TA7), (SZ15TA8), (SZ15TA9), (SZ15TA10), (SZ15TA11), (SZ15TA12), (SZ15TA13), (SZ15TA14), (SZ15TA15), (SZ16TA1), (SZ16TA2), (SZ16TA3), (SZ16TA4), (SZ16TA5), (SZ16TA6), (SZ16TA7), (SZ16TA8), (SZ16TA9), (SZ16TA10), (SZ16TA11), (SZ16TA12), (SZ16TA13), (SZ16TA14), (SZ16TA15), (SZ17TA1), (SZ17TA2), (SZ17TA3), (SZ17TA4), (SZ17TA5), (SZ17TA6), (SZ17TA7), (SZ17TA8), (SZ17TA9), (SZ17TA10), (SZ17TA11), (SZ17TA12), (SZ17TA13), (SZ17TA14), (SZ17TA15), (SZ18TA1), (SZ18TA2), (SZ18TA3), (SZ18TA4), (SZ18TA5), (SZ18TA6), (SZ18TA7), (SZ18TA8), (SZ18TA9), (SZ18TA10), (SZ18TA11), (SZ18TA12), (SZ18TA13), (SZ18TA14), (SZ18TA15), (SZ19TA1), (SZ19TA2), (SZ19TA3), (SZ19TA4), (SZ19TA5), (SZ19TA6), (SZ19TA7), (SZ19TA8), (SZ19TA9), (SZ19TA10), (SZ19TA11), (SZ19TA12), (SZ19TA13), (SZ19TA14), (SZ19TA15), (SZ20TA1), (SZ20TA2), (SZ20TA3), (SZ20TA4), (SZ20TA5), (SZ20TA6), (SZ20TA7), (SZ20TA8), (SZ20TA9), (SZ20TA10), (SZ20TA11), (SZ20TA12), (SZ20TA13), (SZ20TA14), (SZ20TA15), (SZ21TA1), (SZ21TA2), (SZ21TA3), (SZ21TA4), (SZ21TA5), (SZ21TA6), (SZ21TA7), (SZ21TA8), (SZ21TA9), (SZ21TA10), (SZ21TA11), (SZ21TA12), (SZ21TA13), (SZ21TA14), (SZ21TA15), (SZ22TA1), (SZ22TA2), (SZ22TA3), (SZ22TA4), (SZ22TA5), (SZ22TA6), (SZ22TA7), (SZ22TA8), (SZ22TA9), (SZ22TA10), (SZ22TA11), (SZ22TA12), (SZ22TA13), (SZ22TA14), (SZ22TA15), (SZ23TA1), (SZ23TA2), (SZ23TA3), (SZ23TA4), (SZ23TA5), (SZ23TA6), (SZ23TA7), (SZ23TA8), (SZ23TA9), (SZ23TA10), (SZ23TA11), (SZ23TA12), (SZ23TA13), (SZ23TA14), (SZ23TA15), (SZ24TA1), (SZ24TA2), (SZ24TA3), (SZ24TA4), (SZ24TA5), (SZ24TA6), (SZ24TA7), (SZ24TA8), (SZ24TA9), (SZ24TA10), (SZ24TA11), (SZ24TA12), (SZ24TA13), (SZ24TA14), (SZ24TA15), (SZ25TA1), (SZ25TA2), (SZ25TA3), (SZ25TA4), (SZ25TA5), (SZ25TA6), (SZ25TA7), (SZ25TA8), (SZ25TA9), (SZ25TA10), (SZ25TA11), (SZ25TA12), (SZ25TA13), (SZ25TA14), (SZ25TA15), (SZ26TA1), (SZ26TA2), (SZ26TA3), (SZ26TA4), (SZ26TA5), (SZ26TA6), (SZ26TA7), (SZ26TA8), (SZ26TA9), (SZ26TA10), (SZ26TA11), (SZ26TA12), (SZ26TA13), (SZ26TA14), (SZ26TA15), (SZ27TA1), (SZ27TA2), (SZ27TA3), (SZ27TA4), (SZ27TA5), (SZ27TA6), (SZ27TA7), (SZ27TA8), (SZ27TA9), (SZ27TA10), (SZ27TA11), (SZ27TA12), (SZ27TA13), (SZ27TA14), (SZ27TA15), (SZ28TA1), (SZ28TA2), (SZ28TA3), (SZ28TA4), (SZ28TA5), (SZ28TA6), (SZ28TA7), (SZ28TA8), (SZ28TA9), (SZ28TA10), (SZ28TA11), (SZ28TA12), (SZ28TA13), (SZ28TA14), (SZ28TA15), (SZ29TA1), (SZ29TA2), (SZ29TA3), (SZ29TA4), (SZ29TA5), (SZ29TA6), (SZ29TA7), (SZ29TA8), (SZ29TA9), (SZ29TA10), (SZ29TA11), (SZ29TA12), (SZ29TA13), (SZ29TA14), (SZ29TA15), (SZ30TA1), (SZ30TA2), (SZ30TA3), (SZ30TA4), (SZ30TA5), (SZ30TA6), (SZ30TA7), (SZ30TA8), (SZ30TA9), (SZ30TA10), (SZ30TA11), (SZ30TA12), (SZ30TA13), (SZ30TA14), (SZ30TA15), (SZ31TA1), (SZ31TA2), (SZ31TA3), (SZ31TA4), (SZ31TA5), (SZ31TA6), (SZ31TA7), (SZ31TA8), (SZ31TA9), (SZ31TA10), (SZ31TA11), (SZ31TA12), (SZ31TA13), (SZ31TA14), or (SZ31TA15), and therapeutically acceptable salts, prodrugs, salts of prodrugs, and metabolites thereof. One aspect of the present disclosure, therefore, is directed to an acylsulfonamide having a structure that corresponds to one of the preceding SZTA compounds. Another aspect of the present disclosure is directed to a pharmaceutical composition comprising one or more of the preceding SZTA compounds and a pharmaceutically acceptable carrier. In one embodiment, the SZTA compound is selected from SZ31TA2, SZ15TA2, and SZ17TA2. In one preferred embodiment, the SZTA compound is SZ31TA2. In another particular embodiment, the SZTA compound differs in at least one moiety or substituent as compared to the generic and specific compounds described in U.S. Patent Application Publication No. US2007/0027135 (hereby incorporated by reference herein).

In one embodiment, the acylsulfonamide has a selectivity index against Bcl-X_{L} and/or Mcl-1 of at least about 15; more preferably at least about 20; still more preferably at least about 25; still more preferably at least about 30; still more preferably at least about 35; still more preferably at least about 40; still more preferably at least about 45; still more preferably at least about 50; still more preferably at least about 55; and still more preferably at least about 60. In certain other embodiments, the selectivity index against Bcl-X_{L} and/or Mcl-1 is greater than 60 (e.g., 70, 80, 90, or greater). In one particular embodiment; the acylsulfonamide has a selectivity index against Bcl-X_{L} of at least about 15; more preferably at least about 20; still more preferably at least about 25; still more preferably at least about 30; still more preferably at least about 35; still more preferably at least about 40; still more preferably at least about 45; still more preferably at least about 50; still more preferably at least about 55; and still more preferably at least about 60. In certain other embodiments, the selectivity index against Bcl-X_{L} is greater than 60 (e.g., 70, 80, 90, or greater). In another particular embodiment; the acylsulfonamide has a selectivity index against Mcl-1 of at least about 15; more preferably at least about 20; still more preferably at least about 25; still more preferably at least about 30; still more preferably at least about 35; still more preferably at least about 40; still more preferably at least about 45; still more preferably at least about 50; still more preferably at least about 55; and still more preferably at least about 60. In certain other embodiments, the selectivity index against Mcl-1 is greater than 60 (e.g., 70, 80, 90, or greater). In these and other embodiments, the acylsulfonamide has a ligand efficiency of at least about 0.15; more preferably about 0.18; still more preferably about 0.21; still more preferably about 0.24; and still more preferably 0.27 or larger.

In combination, in one embodiment the acylsulfonamide has a selectivity index against Bcl-X_{L} and/or Mcl-1 of at least about 15; more preferably at least about 30; still more preferably at least about 45; and still more preferably at least about 60; and a ligand efficiency of at least about 0.15; more preferably about 0.18; still more preferably about 0.21; still more preferably about 0.24. In another embodiment, the acylsulfonamide has a selectivity index against Bcl-X_{L} of at least about 15; more preferably at least about 30; still more preferably at least about 45; and still more preferably at least about 60; and a ligand efficiency of at least about 0.15; more preferably about 0.18; still more preferably about 0.21; still more preferably about 0.24. In another embodiment, the acylsulfonamide has a selectivity index against Mcl-1 of at least about 15; more preferably at least about 30; still more preferably at least about 45; and still more preferably at least about 60; and a ligand efficiency of at least about 0.15; more preferably about 0.18; still more preferably about 0.21; still more preferably about 0.24.

Certain other preferred acylsulfonamides are disclosed in U.S. Pat. No. 6,720,338 to Augeri et al.; U.S. Pat. No. 7,030,115 to Elmore et al.; and U.S. Pat. No. 7,390,799 to Bruncko et al., each of which is hereby incorporated by reference in its entirety.

Generally, the processes described herein are not wholly dependent on the screening of final compounds, prepared through traditional means, but rather allow the Bcl-2 family protein to select and combine building blocks that fit into its binding site to assemble its own inhibitor molecules. For example, with just 2 to 200 building blocks (1 to 100 mono-thioacids and 1 to 100 mono-sulfonyl azides, e.g., in libraries of compounds), one can quickly scan through 1 to 10,000 possible combinations (1×1 to 100×100) without actually having to make and test these compounds via conventional synthesis and analysis. This number becomes even larger, with the same number of building blocks, if one includes di- or tri-thioacids or -sulfonyl azides, thereby providing the target protein with greater flexibility to choose the appropriate building block and functional group at the same time. The screening method is as simple as determining whether or not the product has been formed in a given test mixture by LC/MS, or other suitable instrument. A compound that is formed by the target Bcl-2 family protein likely to be a good and selective binder, due to the multivalent nature of the interaction. Typically, about 1 to 100 thioacids (e.g., 5, 10, 25, 50, or 75) and about 1 to 100 sulfonyl azides (e.g., 5, 10, 25, 50, or 75) are incubated or reacted in the presence of a Bcl-2 family protein. The particular number of thioacids and sulfonyl azides may also depend upon the reaction vessel(s) being employed in the screening method (e.g., 6-, 12-, 24, 36-, 48-, 64-, 96-, 384-, or 1536-well plates). In one embodiment, 1 to 10 thioacids corresponding to Formula (1) and 1 to 17 sulfonyl azides corresponding to Formula (2) are incubated or reacted in the presence of the Bcl-2 family protein. In another embodiment, 1 to 15 thioacids corresponding to Formula (1) and 1 to 31 sulfonyl azides corresponding to Formula (2) are incubated or reacted in the presence of a Bcl-2 family protein. In various embodiments, for example, the Bcl-2 protein is Bcl-X_{L} or Mcl-1. In one preferred embodiment, the Bcl-2 protein is Bcl-X_{L}. In another preferred embodiment, the Bcl-2 protein is Mcl-1.

Additional aspects, for example, involve screening methods for identifying a plurality of molecules that exhibit affinity for the binding site of the target Bcl-2 family protein. A functional group capable of participating in a ligation chemistry reaction, such as an thio or azide group, present on the compounds of Formulae (1) and (2), is also attached to the molecule, optionally via a linker. Individual members of the resulting plurality of molecules are then mixed with the target molecule and individual members of a plurality or library of compounds that may exhibit affinity for a substrate binding site of the protein. The members of the substrate-binding library have been chemically modified to include a ligation chemistry functional group compatible with the functional group of the library of protein-binding molecules. Thus, any pair of thioacid and sulfonyl azide compounds, one from each library, that exhibits affinity for the binding sites of the protein will covalently bond via the acylsulfonamide ligation chemistry functional groups in situ. The screening process can utilize conventional screening equipment known in the art such as multi-well microtiter plates.

A mass spectrometer may be used for sequential, automated data analysis of the screening process. Exemplary spectrometer equipment that can be used include the Agilent MSD 1100 SL system, linear ion trap systems (ThermoFinnigan LTQ), quadrupole ion trap (LCQ), or a quadrupole time-of-flight (QTOF from Waters or Applied Biosystems). Each of these analyzers have very effective HPLC interfaces for LC-MS experiments.

In accordance with one embodiment, using the starting precursor fragment, that may be an anchor molecule, discovery can be performed by designing small, targeted compound libraries (e.g., less than 100 compounds) based on known drugs and/or substrates. These libraries may be screened using traditional binding assays. The anchor molecules may be incubated with the Bcl-2 family protein target and small libraries of complementary ligation chemistry reagents or precursors (e.g., thioacids, if the anchor molecule is a sulfonyl azide, and vice versa). Each reaction mixture may be analyzed by LC/MS to identify products that are formed by the Bcl-2 protein. Hit validation is performed through competition experiments to demonstrate that the compound is indeed formed by the protein, and binding assays may establish the binding affinities of the protein-generated hits.

The thioacids and sulfonyl azides may also include various linker moieties between the Z₁ substituent and the carbonyl carbon, between the thiol moiety and the carbonyl carbon, between the Z₂ substituent and the —S(═O)₂— moiety, or between the azide moiety and the —S(═O)₂— moiety. The nature and the length of the linker between the two reacting groups or precursors may be selected to afford compounds with optimal binding affinities. Therefore, various types of linkers can be attached to the substrate mimics discussed above. This can readily be accomplished through carbon-heteroatom bond-forming reactions, which can involve the azide groups either directly (acylsulfonamide formation) or indirectly (azide reduction, followed by acylation or sulfonylation of the resulting amines), or other synthesis techniques.

Combinatorial Chemistry Approaches

In a combinatorial approach for identifying or optimizing acylsulfonamides and/or the thioacid and sulfonyl azide building blocks from which they are prepared, a large compositional space (e.g., of thioacids, sulfonyl azides, acylsulfonamides, target proteins, buffer(s), or of relative ratios of two or more of the aforementioned) and/or a large reaction condition space (e.g., of temperature, pressure, reaction time, or other parameter(s)) may be rapidly explored by preparing libraries of thioacids, sulfonyl azides, acylsulfonamides, and/or target proteins and then rapidly screening such libraries. The libraries can comprise, for example, the two or more thioacids, two or more sulfonyl azides, and/or two or more target biomolecules (for use in the preparation of acylsulfonamides), or two or more acylsulfonamides resulting from the reactions described above that are varied with respect to such scaffolds, proteins, and reaction conditions.

Combinatorial approaches for screening a library can include an initial, primary screening, in which initial reaction mixtures or reaction product mixtures are rapidly evaluated to provide valuable preliminary data and, optimally, to identify several “hits,” e.g., particular candidate materials having characteristics that meet or exceed certain predetermined metrics (e.g., performance characteristics, desirable properties, unexpected and/or unusual properties, etc., such as binding, inhibition, and so on). Such metrics may be defined, for example, by the characteristics of a known or standard thioacid, sulfonyl azide, target protein, acylsulfonamide, synthetic scheme, or binding parameters. Because local performance maxima may exist in compositional spaces between those evaluated in the primary screening of the first libraries or alternatively, in process-condition spaces different from those considered in the first screening, it may be advantageous to screen more focused libraries (e.g., libraries focused on a smaller range of compositional gradients, or libraries comprising compounds having incrementally smaller structural variations relative to those of the identified hits) and additionally or alternatively, subject the initial hits to variations in process conditions. Hence, a primary screen can be used reiteratively to explore localized and/or optimized compositional space in greater detail. The preparation and evaluation of more focused (thioacid, sulfonyl azide, target protein, or acylsulfonamide) libraries can continue as long as the high-throughput primary screen can meaningfully distinguish between neighboring library compositions or compounds.

Once one or more hits have been satisfactorily identified based on the primary screening, initial scaffold or final product libraries focused around the primary-screen hits can be evaluated with a secondary screen, e.g., a screen designed to provide (and typically verified, based on known materials, to provide) chemical process conditions that relate with a greater degree of confidence to commercially-important processes and conditions than those applied in the primary screen. For example, certain “real-world-modeling” considerations may be incorporated into the secondary screen at the expense of methodology speed (e.g., as measured by sample throughput) compared to a corresponding primary screen. Particular compounds, proteins, reaction conditions, or post-synthesis processing conditions having characteristics that surpass the predetermined metrics for the secondary screen may then be considered to be “leads.” If desired, additional thioacid, sulfonyl azide, acylsulfonamide, or other libraries focused about such lead materials can be screened with additional secondary screens or with tertiary screens. Identified lead thioacids, sulfonyl azides, acylsulfonamides, proteins, and/or reaction conditions may be subsequently developed for commercial applications through traditional bench-scale and/or pilot scale experiments.

While the concept of primary screens and secondary screens as outlined above provides a valuable combinatorial research model for investigating thioacid/sulfonyl azide/acylsulfonamide/Bcl-2 family protein reactions, a secondary screen may not be necessary for certain chemical processes where primary screens provide an adequate level of confidence as to scalability and/or where market conditions warrant a direct development approach. Similarly, where optimization of materials having known properties of interest is desired, it may be appropriate to start with a secondary screen. In general, the systems, devices and methods, and the building block or final compounds described herein may be applied as either a primary or a secondary screen, depending on the specific research program and goals thereof.

According to certain aspects, methods, systems and devices are disclosed that improve the efficiency and/or effectiveness of the steps necessary to characterize a thioacid or sulfonyl azide sample or a plurality of thioacid or sulfonyl azide samples, or an acylsulfonamide sample or a plurality of acylsulfonamide samples (e.g., libraries of initial and final product mixtures comprising the thioacids and sulfonyl azides, and the acylsulfonamides, respectively). In certain preferred embodiments, a property of a plurality of samples or of components thereof can be detected in a characterization system with an average sample-throughput sufficient for an effective combinatorial or TGS research program. The property may be, for example, protein binding, protein inhibition, or other related or unrelated parameter.

Characterizing a (building block and/or final) sample can include (i) preparing the sample (e.g., synthesis or dilution), (ii) injecting the sample into a mobile phase of a flow characterization system (e.g., liquid chromatography system, flow-injection analysis system, or related apparatus), (iii) separating the sample chromatographically, (iv) detecting a property of the sample or of one or more components thereof, and/or (v) correlating the detected property or parameter to a characterizing property or parameter of interest. Various characterization protocols may be employed involving some or all of the aforementioned steps. For example, a property of a thioacid, sulfonyl azide, or resulting acylsulfonamide sample (or libraries thereof) may be detected in a non-flow, static system either with preparation (steps (i) and (iv)) or without preparation (step (iv)). Alternatively, a property of a sample may be detected in a flow characterization system, either with or without sample preparation and either with or without chromatographic separation. In certain characterization protocols involving flow characterization systems without chromatographic analysis or separation, for example, a property of a sample may be detected in a flow-injection analysis system either with preparation (steps (i), (ii), and (iv)) or without preparation (steps (ii) and (iv)). If chromatographic separation of a sample is desired, a property of the sample may be detected in a liquid chromatography system either with preparation (steps (i), (ii), (iii), and (iv)) or without preparation (steps (ii), (iii), and (iv)). While the physically-detected property (e.g., refracted light, absorbed light, scattered light) from two samples being screened could be compared directly, in most cases the detected property is preferably correlated to a characterizing property of interest (e.g., molecular weight, protein binding, inhibition, etc.) (step (v)).

A plurality of samples may be characterized as described above. As a general approach for improving the sample throughput for a plurality of thioacids, sulfonyl azides, acylsulfonamides, or proteins, each of the steps, applicable to a given characterization protocol can be optimized with respect to time and quality of information, both individually and in combination with each other. Additionally or alternatively, each or some of such steps can be effected in a rapid-serial, parallel, serial-parallel or hybrid parallel-serial manner, as understood in accordance with conventional combinatorial chemistry protocols.

The throughput of a plurality of samples through a single step in a characterization process is improved by optimizing the speed of that step, while maintaining, to the extent necessary, the information-quality aspects of that step. In many cases, such as with chromatographic or mass spectroscopic analysis, speed can be gained at the expense of resolution of the separated or analyzed components. Although conventional research norms, developed in the context in which research was rate-limited primarily by the synthesis of samples, may find such an approach less than wholly satisfactory, the degree of rigor can be entirely satisfactory for a primary or a secondary screen of a combinatorial library of samples. For combinatorial research (and as well, for many on-line process control systems), the quality of information should be sufficiently rigorous to provide for scientifically acceptable distinctions between the compounds or process conditions being investigated, and for a secondary screen, to provide for scientifically acceptable correlation (e.g., values or, for some cases, trends) with more rigorous, albeit more laborious and time-consuming traditional characterization approaches.

The throughput of a plurality of samples through a series of steps, where such steps are repeated for the plurality of samples, can also be optimized. In accordance with one approach, one or more steps of the cycle can be compressed relative to traditional approaches or can have upstream or downstream aspects truncated to allow other steps of the same cycle to occur sooner compared to the cycle with traditional approaches. In another approach, the earlier steps of a second cycle can be performed concurrently with the later steps of a first cycle. In a rapid-serial approach for characterizing a sample, for instance, sample preparation for a second sample in a series can be effected while the first sample in the series is being synthesized, detected, and/or analyzed. As another example, a second sample in a series can be injected while the first sample in the series is being synthesized, detected, and/or analyzed.

A characterization protocol for a plurality of samples can involve a single-step process. In a rapid-serial detection approach for a single-step process, the plurality of samples and a single detector are serially positioned in relation to each other for serial detection of the samples. In a parallel detection approach, two or more detectors are employed to detect a property of two or more samples simultaneously. In a direct, non-flow detection protocol, for example, two or more samples and two or more detectors can be positioned in relation to each other to detect a property of the two or more samples simultaneously. In a serial-parallel detection approach, a property of a larger number of samples (e.g., three, four, or more) is detected as follows. First, a property of a subset of the three, four, or more samples (e.g., 2 samples) is detected in parallel for the subset of samples, and then serially thereafter, a property of another subset of four or more samples is detected in parallel.

For characterization protocols involving more than one step (e.g., two or more of steps (i), (ii), (iii), (iv), and (v), above), optimization approaches to effect high-throughput characterization of thioacids, sulfonyl azides, target biomolecules, and resulting acylsulfonamides) can vary. For instance, a plurality of samples can be characterized with a single characterization system (A) in a rapid-serial approach in which each of the plurality of samples (A₁, A₂, A₃ . . . Aₙ) are processed serially through the characterization system (A) with each of the steps ((i), (ii), (iii), (iv), and (v)) effected in series on each of the of samples to produce a serial stream of corresponding characterizing property data (d₁, d₂, d₃ . . . dₙ). This approach benefits from relatively minimal capital investment, and may provide sufficient throughput, particularly when the steps (i), (ii), (iii), (iv), and (v) have been optimized with respect to speed and quality of information. As another example, a plurality of samples can be characterized with two or more characterization systems (A, B, C, D . . . N) in a pure parallel (or for larger libraries, serial-parallel) approach in which the plurality of samples (A₁, A₂, A₃ . . . Aₙ) or a subset thereof are processed through the two or more characterization systems (A, B, C, D . . . ZZ) in parallel, with each individual system effecting each step on one of the samples to produce the characterizing property information (A₁, A₂, A₃ . . . Aₙ; B₁, B₂, B₃ . . . Bₙ; C₁, C₂, C₃ . . . Cₙ, etc.) in parallel. This approach is advantageous with respect to overall throughput, but may be constrained by the required capital investment.

In a hybrid approach, certain of the steps of the characterization process can be effected in parallel, while certain other steps can be effected in series. Preferably, for example, it may be desirable to effect the longer, throughput-limiting steps in parallel for the plurality of samples, while effecting the faster, less limiting steps in series. Such a parallel-series hybrid approach can be exemplified, by parallel sample preparation (step (i)) of a plurality of thioacid, sulfonyl azide, or acylsulfonamide samples (A₁, A₂, A₃ . . . Aₙ), followed by serial injection, chromatographic analysis, detection and correlation (steps (ii), (iii), (iv), and (v)) with a single characterization system (A) to produce a serial stream of corresponding characterizing property information (d₁, d₂, d₃ . . . dₙ). In another exemplary parallel-series hybrid approach, a plurality of thioacid, sulfonyl azide, or acylsulfonamide samples (A₁, A₂, A₃ . . . Aₙ) are prepared, reacted, and injected in series into the mobile phase of four or more characterizing systems (e.g., LC/MS) (A, B, C . . . ZZ), and then detected and correlated in a slightly offset (staggered) parallel manner to produce the characterizing property information (d₁, d₂, d₃ . . . dₙ) in the same staggered-parallel manner. If each of the systems has the same processing rates, then the extent of the parallel offset (or staggering) will be primarily determined by the speed of the serial preparation and reaction. In a variation of the preceding example, where the detection and correlation steps are sufficiently rapid, a plurality of thioacid, sulfonyl azide, or acylsulfonamide samples (A₁, A₂, A₃ . . . Aₙ) could be characterized by serial sample preparation and reaction, staggered-parallel analysis, and then serial correlation, to produce the characterizing property information (d₁, d₂, d₃ . . . dₙ) in series. In this case, the rate of injection into the various separation columns is preferably synchronized with the rate of detection. In general, optimization of individual characterization steps (e.g., steps (i), (ii), (iii), (iv), and (v)) with respect to speed and quality of information can improve sample throughput regardless of whether the overall characterization scheme involves a rapid-serial or parallel aspect (i.e., true parallel, serial-parallel or hybrid parallel-series approaches).

A plurality or library of samples generally comprises 2 or more thioacid, sulfonyl azide, target protein, or acylsulfonamide samples. The individual compounds may be physically or temporally separated from each other, e.g., by residing in different sample containers, by having a membrane or other partitioning material positioned between samples, by being partitioned (e.g., in-line) with an intervening fluid, by being temporally separated in a flow process line (e.g., as sampled for process control purposes), or otherwise, or two, three, or more compound samples may be combined or otherwise reside in the same sample container. In certain embodiments, the plurality (or library) of samples typically comprises 4 or more samples (e.g., 4 or more different thioacid, sulfonyl azide, or acylsulfonamide compounds), while in certain other embodiments, 8 or more samples (e.g., 4 or more different thioacid, sulfonyl azide, or acylsulfonamide compounds). Four samples can be employed, for example, in connection with experiments having one control sample and three samples varying (e.g., with respect to compound, target, or process conditions as discussed above) to be representative of a high, a medium and a low-value of the varied factor, and thereby, to provide some indication as to trends. Four samples may also be a minimum number of samples to effect a serial-parallel characterization approach, as described above (e.g., with two detector/analyzers operating in parallel). Eight samples can provide for additional variations in the explored factor space. Higher numbers of samples and libraries thereof can be investigated, according to the methods described herein, to provide additional insights into larger compositional and/or process space. In some cases, for example, the plurality of samples can be 15 or more samples, 20 or more samples, 40 or more samples, 80 or more samples, or more. Such numbers can be loosely associated with standard configurations of other parallel reactor configurations and/or of standard sample containers (e.g., 96-well microtiter plate-type formats). Moreover, even larger numbers of samples can be characterized according to the methods described herein for larger scale research endeavors. Hence, the number of thioacid, sulfonyl azide, and acylsulfonamide samples prepared and analyzed can be 150 or more, 400 or more, 500 or more, 750 or more, 1,000 or more, 1,500 or more, 2,000 or more, 5,000 or more and 10,000 or more. As such, the number of samples can range from about 2 samples to about 10,000 samples, or more, and preferably from about 8 samples to about 10,000 samples, or more. In some cases, in which processing of samples using typical 96-well microtiter-plate formatting is convenient or otherwise desirable, the number of samples can be 96*N, where N is an integer ranging from about 1 to about 100. For many applications, N can suitably range from 1 to about 20, and in some cases, from 1 to about 7.

The plurality of samples can likewise be a library of samples, e.g., a library of thioacids, a library of sulfonyl azides, and/or a library of acylsulfonamides. A library of samples generally comprises an array of two or more different thioacid, sulfonyl azide, and/or acylsulfonamide samples spatially separated, e.g., on a common substrate, or temporally separated, e.g., in a flow system. Candidate samples (i.e., members) within a library may differ in a definable and typically predefined way, including with regard to chemical structure (i.e., the substituents on the thioacid or sulfonyl azide), processing (e.g., synthesis) history (including the biological target utilized in the target-guided synthesis), mixtures of interacting components, purity, etc. The samples may be spatially separated, for instance, at an exposed surface of the substrate, such that the array of samples are separately addressable for characterization thereof. The two or more different samples can reside in sample containers formed as wells in a surface of the substrate. The number of samples included within the library can generally be the same as the number of samples included within the plurality of samples, as discussed above. In general, however, not all of the samples within a library of samples need to be different samples. When process conditions are to be evaluated, the libraries may contain only one type of sample. Typically, however, for combinatorial research applications, at least two or more, preferably at least four or more, even more preferably eight or more and, in many cases most, and allowably each of the plurality of samples in a given library of samples will be different from each other. Specifically, a different sample can be included within at least about 50%, preferably at least 75%, preferably at least 80%, even more preferably at least 90%, still more preferably at least 95%, yet more preferably at least 98% and most preferably at least 99% of the samples included in the sample library. In some cases, all of the samples in a library of samples will be different from each other.

In general, the substrate can be a structure having a rigid or semi-rigid surface on which or into which the array of samples can be formed or deposited. The substrate can be of any suitable material, and preferably consists essentially of materials that are inert with respect to the samples of interest (including, for example, the thioacid, sulfonyl azide, acylsulfonamide, or the biological target molecule (e.g., the Bcl-2 family protein(s)). Certain materials will, therefore, be less desirably employed as a substrate material for certain reaction process conditions (e.g., high temperatures or high pressures) and/or for certain reaction mechanisms. Stainless steel, silicon, including polycrystalline silicon, single-crystal silicon, sputtered silicon, and silica (SiO₂) in any of its forms (quartz, glass, etc.), for example, may be substrate materials. Other known materials (e.g., silicon nitride, silicon carbide, metal oxides (e.g., alumina), mixed metal oxides, metal halides (e.g., magnesium chloride), minerals, zeolites, and ceramics) may also be suitable for a substrate material in some applications. Organic and inorganic polymers may also be suitably employed in some applications. Exemplary polymeric materials that can be suitable as a substrate material in particular applications include polystyrenes, polyimides such as Kapton™, polypropylene, polytetrafluoroethylene (PTFE) and/or polyether etherketone (PEEK), among others. The substrate material is also preferably selected for suitability in connection with known fabrication techniques. As to form, the sample containers formed in, at or on a substrate can be preferably, but are not necessarily, arranged in a substantially flat, substantially planar surface of the substrate. The sample containers can be formed in a surface of the substrate as dimples, wells, raised regions, trenches, or the like. Non-conventional substrate-based sample containers, such as relatively flat surfaces having surface-modified regions (e.g., selectively wettable regions) can also be employed. The overall size and/or shape of the substrate is not limiting. The size and shape can be chosen, however, to be compatible with commercial availability, existing fabrication techniques, and/or with known or later-developed automation techniques, including automated sampling and automated substrate-handling devices, as well as detection and analysis equipment. The substrate is also preferably sized to be portable by humans. The substrate can be thermally insulated if needed, for example, for high-temperature and/or low-temperature applications. In preferred embodiments, the substrate is designed such that the individually addressable regions of the substrate can act as reaction vessels for preparing the acylsulfonamides from the reaction of the thioacids and the sulfonyl azides in the presence of the biological target (e.g., a Bcl-2 protein) in a product mixture (as well as sample containers for the samples during subsequent characterization thereof). Glass-lined, 96-well, 384-well and 1536-well microtiter-type plates, fabricated from stainless steel, aluminum, composite, polystyrene or other polymers or plastics, may be used as substrates for a library of samples. The choice of an appropriate specific substrate material and/or form for certain applications will be apparent to those of skill in the art in view of the guidance provided herein.

The library of materials can be a combinatorial library of building blocks (e.g., thioacids, sulfonyl azides) or a combinatorial library of product mixtures (e.g., acylsulfonamides). Thioacid libraries can comprise, for example, a variety of thioacids corresponding to Formula (1) to be used in the target-guided synthesis approaches described herein. Similarly, sulfonyl azide libraries can comprise, for example, a variety of sulfonyl azides corresponding to Formula (2) to be used in the target-guided synthesis approaches described herein. Acylsulfonamide libraries can comprise, for example, product mixtures resulting from such reactions of thioacids and sulfonyl azides (including libraries thereof) that are varied with respect to, for example, particular substituent patterns, buffers, biological targets, the relative amounts of components, reaction conditions (e.g., pH, temperature, pressure, reaction time) or any other factor that may affect the reaction. Design variables for reactions are well known in the art. A library of thioacid/sulfonyl azide/acylsulfonamide samples may be prepared in arrays, in parallel reactors or in a serial fashion. In certain embodiments, the libraries can be characterized directly, without being isolated, from the reaction vessel in which the compound(s) was synthesized.

While such methods may be generally preferred for a combinatorial approach to lead compound research, they are to be considered exemplary and non-limiting. As noted above, the particular samples characterized according to the methods and with the apparatus disclosed herein can be from any source, including, but not limited to product mixtures resulting from combinatorial synthesis approaches or from target-guided synthesis approaches.

Pharmaceutical Compositions and Methods for Treatment

Other aspects involve methods for treatment of various conditions and diseases using the compounds described herein. According to methods of treatment, the compounds described herein, and particularly the acylsulfonamides corresponding to Formula (3) can be useful for the prevention of metastases from the tumors described above either when used alone or in combination with radiotherapy and/or other chemotherapeutic treatments conventionally administered to patients for treating cancer. When using the compounds for chemotherapy, for example, the specific therapeutically effective dose level for any particular patient will depend upon factors such as the disorder being treated and the severity of the disorder; the activity of the particular compound used; the specific compound employed; the age, body weight, general health, sex, and diet of the patient; the time of administration; the route of administration; the rate of excretion of the compound employed; the duration of treatment; and drugs used in combination with or coincidentally with the compound used. For example, when used in the treatment of solid tumors, the compounds can be administered with chemotherapeutic agents such as alpha interferon, COMP (cyclophosphamide, vincristine, methotrexate, and prednisone), etoposide, mBACOD (methotrexate, bleomycin, doxorubicin, cyclophosphamide, vincristine, and dexamethasone), PRO-MACE/MOPP (prednisone, methotrexate (w/leucovin rescue), doxorubicin, cyclophosphamide, paclitaxel, docetaxel, etoposide/mechlorethamine, vincristine, prednisone, and procarbazine), vincristine, vinblastine, angioinhibins, TNP-470, pentosan polysulfate, platelet factor 4, angiostatin, LM-609, SU-101, CM-101, Techgalan, thalidomide, SP-PG, and the like. For example, a tumor may be treated conventionally with surgery, radiation or chemotherapy and a compound disclosed herein subsequently administered to extend the dormancy of micrometastases and to stabilize and inhibit the growth of any residual primary tumor.

Additional aspects include compounds which have been described in detail hereinabove or to pharmaceutical compositions which comprise an effective amount of one or more compounds according to the disclosure, optionally in combination with a pharmaceutically acceptable carrier, additive or excipient (described in further detail below).

Dosage and Amount and Time Course of Treatment

The dose or amount of pharmaceutical compositions including the acylsulfonamide compositions described above administered to the mammal should be an effective amount for the intended purpose, i.e., treatment (or prophylaxis) of one or more of the diseases, pathological disorders, and medical conditions noted above. Generally speaking, the effective amount of the composition administered to the mammal can vary according to a variety of factors such as, for example, the age, weight, sex, diet, route of administration, and the medical condition of the mammal. Specifically preferred doses are discussed more fully below. It will be understood, however, that the total daily usage of the compositions described herein will be decided by the attending physician or veterinarian within the scope of sound medical judgment.

The specific therapeutically effective dose level for any particular mammal will depend upon a variety of factors including the disorder being treated and the severity of the disorder; activity of the specific compound(s) employed; the age, body weight, general health, sex and diet of the patient; the time of administration; the route of administration; the rate of excretion of the specific compound(s) employed; the duration of the treatment; drugs used in combination or coincidental with the specific compound(s) employed and like factors well known in the medical and/or veterinary arts. For example, it is well within the skill of the art to start doses of the compound(s) at levels lower than those required to achieve the desired therapeutic effect and to gradually increase the dosage until the desired effect is achieved. If desired, the effective daily doses may be divided into multiple doses for purposes of administration. Consequently, single dose compositions may contain such amounts or submultiples to make up the daily dose.

Administration of the pharmaceutical composition can occur as a single event or over a time course of treatment. For example, one or more of the compositions can be administered hourly (e.g., every hour, every two hours, every three hours, every four hours, every five hours, every six hours, and so on), daily, weekly, bi-weekly, or monthly. For treatment of acute conditions, the time course of treatment may be at least several hours or days. Certain conditions could extend treatment from several days to several weeks. For example, treatment could extend over one week, two weeks, or three weeks. For more chronic conditions, treatment could extend from several weeks to several months, a year or more, or the lifetime of the mammal in need of such treatment. Alternatively, the compositions can be administered hourly, daily, weekly, bi-weekly, or monthly, for a period of several weeks, months, years, or over the lifetime of the mammal as a prophylactic measure.

One or more of the compounds may be utilized in a pharmaceutically acceptable carrier, additive or excipient at a suitable dose ranging from about 0.05 to about 200 mg/kg of body weight per day, preferably within the range of about 0.1 to 100 mg/kg/day, most preferably in the range of 0.25 to 50 mg/kg/day. As noted above, the desired dose may conveniently be presented in a single dose or as divided doses administered at appropriate intervals, for example as two, three, four or more sub-doses per day.

Ideally, the active ingredient should be administered to achieve effective peak plasma concentrations of the active compound within the range of from about 0.05 uM to about 5 uM. This may be achieved, for example, by the intravenous injection of about a 0.05 to 10% solution of the active ingredient, optionally in saline, or orally administered as a bolus containing about 1 mg to about 5 g, preferably about 5 mg to about 500 mg of the active ingredient, depending upon the active compound and its intended target. Desirable blood levels may be maintained by a continuous infusion to preferably provide about 0.01 mg/kg/hour to about 2.0 mg/kg/hour or by intermittent infusions containing about 0.05 mg/kg to about 15 mg/kg of the active ingredient. Oral dosages, where applicable, will depend on the bioavailability of the compositions from the GI tract, as well as the pharmacokinetics of the compositions to be administered. While it is possible that, for use in therapy, one or more compositions of the invention may be administered as the raw chemical, it is preferable to present the active ingredient as a pharmaceutical formulation, presented in combination with a pharmaceutically acceptable carrier, excipient, or additive.

Routes of Administration, Formulations/Pharmaceutical Compositions

As noted above, the above-described compounds may be dispersed in a pharmaceutically acceptable carrier prior to administration to the mammal. The carrier, also known in the art as an excipient, vehicle, auxiliary, adjuvant, or diluent, is typically a substance which is pharmaceutically inert, confers a suitable consistency or form to the composition, and does not diminish the efficacy of the compound. The carrier is generally considered to be “pharmaceutically or pharmacologically acceptable” if it does not produce an unacceptably adverse, allergic or other untoward reaction when administered to a mammal, especially a human.

The selection of a pharmaceutically acceptable carrier will also, in part, be a function of the route of administration. In general, the compositions can be formulated for any route of administration so long as the blood circulation system is available via that route. For example, suitable routes of administration include, but are not limited to, oral, parenteral (e.g., intravenous, intraarterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration.

Pharmaceutically acceptable carriers for use in combination with the acylsulfonamide compounds are well known to those of ordinary skill in the art and are selected based upon a number of factors: the particular compound used, and its concentration, stability and intended bioavailability; the subject, its age, size and general condition; and the route of administration. Suitable nonaqueous, pharmaceutically-acceptable polar solvents include, but are not limited to, alcohols (e.g., α-glycerol formal, β-glycerol formal, 1,3-butyleneglycol, aliphatic or aromatic alcohols having 2 to 30 carbon atoms such as methanol, ethanol, propanol, isopropanol, butanol, t-butanol, hexanol, octanol, amylene hydrate, benzyl alcohol, glycerin (glycerol), glycol, hexylene glycol, tetrahydrofurfuryl alcohol, lauryl alcohol, cetyl alcohol, or stearyl alcohol, fatty acid esters of fatty alcohols such as polyalkylene glycols (e.g., polypropylene glycol, polyethylene glycol), sorbitan, sucrose and cholesterol); amides (e.g., dimethylacetamide (DMA), benzyl benzoate DMA, dimethylformamide, N-(β-hydroxyethyl)-lactamide, N,N-dimethylacetamide amides, 2-pyrrolidinone, 1-methyl-2-pyrrolidinone, or polyvinylpyrrolidone); esters (e.g., 1-methyl-2-pyrrolidinone, 2-pyrrolidinone, acetate esters such as monoacetin, diacetin, and triacetin, aliphatic or aromatic esters such as ethyl caprylate or octanoate, alkyl oleate, benzyl benzoate, benzyl acetate, dimethylsulfoxide (DMSO), esters of glycerin such as mono, di-, or tri-glyceryl citrates or tartrates, ethyl benzoate, ethyl acetate, ethyl carbonate, ethyl lactate, ethyl oleate, fatty acid esters of sorbitan, fatty acid derived PEG esters, glyceryl monostearate, glyceride esters such as mono, di-, or tri-glycerides, fatty acid esters such as isopropyl myristrate, fatty acid derived PEG esters such as PEG-hydroxyoleate and PEG-hydroxystearate, N-methylpyrrolidinone, pluronic 60, polyoxyethylene sorbitol oleic polyesters such as poly(ethoxylated)₃₀₋₆₀ sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate, polyoxyethylene sorbitan esters such as polyoxyethylene-sorbitan monooleate, polyoxyethylene-sorbitan monopalmitate, polyoxyethylene-sorbitan monolaurate, polyoxyethylene-sorbitan monostearate, and Polysorbate® 20, 40, 60 or 80 from ICI Americas, Wilmington, Del., polyvinylpyrrolidone, alkyleneoxy modified fatty acid esters such as polyoxyl 40 hydrogenated castor oil and polyoxyethylated castor oils (e.g., Cremophor® EL solution or Cremophor® RH 40 solution), saccharide fatty acid esters (i.e., the condensation product of a monosaccharide (e.g., pentoses such as ribose, ribulose, arabinose, xylose, lyxose and xylulose, hexoses such as glucose, fructose, galactose, mannose and sorbose, trioses, tetroses, heptoses, and octoses), disaccharide (e.g., sucrose, maltose, lactose and trehalose) or oligosaccharide or mixture thereof with a C₄ to C₂₂ fatty acid(s) (e.g., saturated fatty acids such as caprylic acid, capric acid, lauric acid, myristic acid, palmitic acid and stearic acid, and unsaturated fatty acids such as palmitoleic acid, oleic acid, elaidic acid, erucic acid and linoleic acid)), or steroidal esters); alkyl, aryl, or cyclic ethers having 2 to 30 carbon atoms (e.g., diethyl ether, tetrahydrofuran, dimethyl isosorbide, diethylene glycol monoethyl ether); glycofurol (tetrahydrofurfuryl alcohol polyethylene glycol ether); ketones having 3 to 30 carbon atoms (e.g., acetone, methyl ethyl ketone, methyl isobutyl ketone); aliphatic, cycloaliphatic or aromatic hydrocarbons having 4 to 30 carbon atoms (e.g., benzene, cyclohexane, dichloromethane, dioxolanes, hexane, n-decane, n-dodecane, n-hexane, sulfolane, tetramethylenesulfon, tetramethylenesulfoxide, toluene, dimethylsulfoxide (DMSO), or tetramethylenesulfoxide); oils of mineral, vegetable, animal, essential or synthetic origin (e.g., mineral oils such as aliphatic or wax-based hydrocarbons, aromatic hydrocarbons, mixed aliphatic and aromatic based hydrocarbons, and refined paraffin oil, vegetable oils such as linseed, tung, safflower, soybean, castor, cottonseed, groundnut, rapeseed, coconut, palm, olive, corn, corn germ, sesame, persic and peanut oil and glycerides such as mono-, di- or triglycerides, animal oils such as fish, marine, sperm, cod-liver, haliver, squalene, squalane, and shark liver oil, oleic oils, and polyoxyethylated castor oil); alkyl or aryl halides having 1 to 30 carbon atoms and optionally more than one halogen substituent; methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (Solutol® HS-15, from BASF, Ludwigshafen, Germany); polyoxyethylene glycerol; sodium laurate; sodium oleate; or sorbitan monooleate.

Other pharmaceutically acceptable solvents for use in the invention are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.) (Marcel Dekker, Inc., New York, N.Y., 1995), The Pharmacological Basis of Therapeutics, (Goodman & Gilman, McGraw Hill Publishing), Pharmaceutical Dosage Forms, (H. Lieberman et al., eds.) (Marcel Dekker, Inc., New York, N.Y., 1980), Remington's Pharmaceutical Sciences (A. Gennaro, ed., 19th ed.) (Mack Publishing, Easton, Pa., 1995), The United States Pharmacopeia 24, The National Formulary 19, (National Publishing, Philadelphia, Pa., 2000), and A. J. Spiegel et al., Use of Nonaqueous Solvents in Parenteral Products, Journal of Pharmaceutical Sciences, Vol. 52, No. 10, pp. 917-927 (1963).

Formulations containing the above acylsulfonamide compounds may take the form of solid, semi-solid, lyophilized powder, or liquid dosage forms such as, for instance, aerosols, capsules, creams, emulsions, foams, gels/jellies, lotions, ointments, pastes, powders, soaps, solutions, sprays, suppositories, suspensions, sustained-release formulations, tablets, tinctures, transdermal patches, and the like, preferably in unit dosage forms suitable for simple administration of precise dosages.

Salts and Prodrugs

As noted above, the pharmaceutical compositions may include acylsulfonamide compounds in their salt form. Typically, the salt will be a pharmaceutically acceptable salt; that is, a salt prepared from pharmaceutically acceptable non-toxic acids, including inorganic acids and organic acids. Suitable non-toxic acids include inorganic and organic acids of basic residues such as amines, for example, acetic, benzenesulfonic, benzoic, amphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, barbaric acid, p-toluenesulfonic and the like; and alkali or organic salts of acidic residues such as carboxylic acids, for example, alkali and alkaline earth metal salts derived from the following bases: sodium hydride, sodium hydroxide, potassium hydroxide, calcium hydroxide, aluminum hydroxide, lithium hydroxide, magnesium hydroxide, zinc hydroxide, ammonia, trimethylammonia, triethylammonia, ethylenediamine, lysine, arginine, ornithine, choline, N,N″-dibenzylethylenediamine, chloroprocaine, diethanolamine, procaine, n-benzylphenethylamine, diethylamine, piperazine, tris(hydroxymethyl)-aminomethane, tetramethylammonium hydroxide, and the like. Pharmaceutically acceptable salts of the compounds described herein can be prepared by reacting the free acid or base forms of these compositions with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, each of which is hereby incorporated by reference herein.

In various embodiments, for example, the acylsulfonamides described herein may exist as acid addition salts, basic addition salts or zwitterions. Salts of compounds described herein are typically prepared during their isolation or following their purification. Acid addition salts are those derived from the reaction of the compounds described herein with acid. Accordingly, salts including the acetate, adipate, alginate, bicarbonate, citrate, aspartate, benzoate, benzenesulfonate (besylate), bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate and undecanoate salts of the compounds described herein are intended to be included within the disclosure. Basic addition salts of compounds are those derived from the reaction of the compounds described herein with the bicarbonate, carbonate, hydroxide or phosphate of cations such as lithium, sodium, potassium, calcium and magnesium.

Since prodrugs are known to enhance numerous desirable pharmaceuticals (e.g., solubility, bioavailability, manufacturing), the compound(s) may be delivered in prodrug form. Thus, the present disclosure is intended to cover prodrugs of the compounds (e.g., acylsulfonamides) described above, methods of delivering the same and compositions containing them. Prodrugs generally include any covalently bonded carriers which release an active parent drug in vivo when such prodrug is administered to a mammalian subject. Prodrugs are generally prepared by modifying functional groups present in the compound in such a way that the modifications are cleaved, either in routine manipulation or in vivo, to the parent compound. Prodrugs include compounds wherein a hydroxyl or amino group is bonded to any group that, when the prodrug is administered to a mammalian subject, cleaves to form a free hydroxyl or free amino group, respectively. Examples of prodrugs include, but are not limited to, acetate, formate, and benzoate derivatives of alcohol and amine functional groups in the compounds and conjugates disclosed herein. Prodrugs of the compound are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compositions of the invention. Prodrugs may refer to compounds that are rapidly transformed in vivo to yield the compound(s) above, for example by hydrolysis in blood. A thorough discussion of prodrugs is provided in the following: Design of Prodrugs, H. Bundgaard, ea., Elsevier, 1985; Methods in Enzymology, K. Widder et al, Ed., Academic Press, 42, p. 309-396, 25 1985; A Textbook of Drug Design and Development, Krogsgaard-Larsen and H. Bundgaard, ea., Chapter 5; “Design and Applications of Prodrugs” p. 113-191, 1991; Advanced Drug Delivery Reviews, H. Bundgard, 8, p. 1-38, 1992; Journal of Pharmaceutical Sciences, 77, p. 285, 30 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ea., American Pharmaceutical Association and Pergamon Press, 1987, each of which is hereby incorporated by reference herein.

It will also be understood that metabolites of the compounds described herein, produced by in vitro or in vivo metabolic processes, may also have utility for treating diseases associated with expression of a Bcl-family protein member such as Bcl-X_{L} and/or Mcl-1.

Compounds described herein may also be radiolabeled with a radioactive isotope such as a radioactive isotope of carbon (i.e., ¹³C), hydrogen (i.e., ³H), nitrogen (i.e., ¹⁵N), phosphorus (i.e., ³²P), sulfur (i.e., ³⁵S) or iodide (i.e., ¹²⁵I). Radioactive isotopes may be incorporated into the compounds described herein by reacting the same and a radioactive derivatizing agent or by incorporating a radiolabeled intermediate into their syntheses. The radiolabeled compounds described herein are useful for both prognostic and diagnostic applications as well as for in vivo and in vitro imaging.

Certain precursor compounds which may be metabolized in vitro or in vivo to form compounds having the various formulae described herein (e.g., Formula (3)) may also have utility for treating diseases associated with expression of a Bcl-family protein member such as Bcl-X_{L} and/or Mcl-1.

Additional Pharmaceutical Components

The above-described pharmaceutical compositions including the acylsulfonamides may additionally include one or more pharmaceutically active components. Suitable pharmaceutically active agents that may be included in the compositions include, for instance, anesthetics, antihypertensives, antianxiety agents, anticlotting agents, anticonvulsants, blood glucose-lowering agents, decongestants, antihistamines, antitussives, antineoplastics, beta blockers, anti-inflammatory agents, antipsychotic agents, cognitive enhancers, cholesterol-reducing agents, antiobesity agents, autoimmune disorder agents, anti-impotence agents, antibacterial and antifungal agents, hypnotic agents, anti-Parkinsonism agents, anti-Alzheimer's Disease agents, antibiotics, anti-depressants, and antiviral agents, among others.

The individual components of such combinations may be administered either sequentially or simultaneously in separate or combined pharmaceutical formulations.

Uses

Other aspects of the present disclosure relate to methods of treating, ameliorating, or preventing diseases during which one or more Bcl-family proteins are expressed. The methods generally comprise administering to a patient in need of such treatment a composition comprising an acylsulfonamide described herein and a pharmaceutically acceptable carrier.

In one embodiment, the disease involves abnormal cell growth and/or dysregulated apoptosis, such as cancer, mesothioloma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, ovarian cancer, cervical cancer, colon cancer, rectal cancer, cancer of the anal region, stomach cancer, gastrointestinal (gastric, colorectal, and duodenal), chronic lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular cancer (hepatic and billiary duct), primary or secondary central nervous system tumor, primary or secondary brain tumor, Hodgkin's disease, chronic or acute leukemia, chronic myeloid leukemia, lymphocytic lymphomas, lymphoblastic leukemia, follicular lymphoma, lymphoid malignancies of T-cell or B-cell origin, melanoma, multiple myeloma, oral cancer, ovarian cancer, non-small cell lung cancer, prostate cancer, small cell lung cancer, cancer of the kidney and ureter, renal cell carcinoma, carcinoma of the renal pelvis, neoplasms of the central nervous system, primary central nervous system lymphoma, non Hodgkin's lymphoma, spinal axis tumors, brains stem glioma, pituitary adenoma, adrenocortical cancer, gall bladder cancer, cancer of the spleen, cholangiocarcinoma, fibrosarcoma, neuroblastoma, retinoblasitoma, or a combination thereof.

In another aspect, methods described herein may be used to treat or prevent mesothioloma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, ovarian cancer, cervical cancer, colon cancer, rectal cancer, cancer of the anal region, stomach cancer, gastrointestinal (gastric, colorectal, and duodenal), chronic lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular cancer (hepatic and billiary duct), primary or secondary central nervous system tumor, primary or secondary brain tumor, Hodgkin's disease, chronic or acute leukemia, chronic myeloid leukemia, lymphocytic lymphomas, lymphoblastic leukemia, follicular lymphoma, lymphoid malignancies of T-cell or B-cell origin, melanoma, multiple myeloma, oral cancer, ovarian cancer, non-small cell lung cancer, prostate cancer, small cell lung cancer, cancer of the kidney and ureter, renal cell carcinoma, carcinoma of the renal pelvis, neoplasms of the central nervous system, primary central nervous system lymphoma, non Hodgkin's lymphoma, spinal axis tumors, brains stem glioma, pituitary adenoma, adrenocortical cancer, gall bladder cancer, cancer of the spleen, cholangiocarcinoma, fibrosarcoma, neuroblastoma, retinoblasitoma, or a combination of one or more of the above cancers in a patient, the treatment comprising administering to a patient in need of such treatment a composition comprising an acylsulfonamide described herein and a pharmaceutically acceptable carrier.

Still another aspect is directed to compositions for treating bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer and spleen cancer, said compositions comprising an excipient and a therapeutically effective amount of an acylsulfonamide described herein.

Still another aspect is directed to a method of treating bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer and spleen cancer in a patient, said methods comprising administering to a patient in need of such treatment a therapeutically effective amount of an acylsulfonamide described herein.

Still another aspect is directed to compositions for treating diseases in a patient during which are expressed one or more than one of a Bcl-X_{L} protein or an Mcl-1 protein, said compositions comprising an excipient and a therapeutically effective amount of an acylsulfonamide described herein and a therapeutically effective amount of one additional therapeutic agent or more than one additional therapeutic agent.

Still another aspect is directed to methods of treating diseases in a patient during which is expressed one or more than one of a Bcl-X_{L} protein or an Mcl-1 protein, said methods comprising administering to the patient a therapeutically effective amount of an acylsulfonamide described herein and a therapeutically effective amount of one additional therapeutic agent or more than one additional therapeutic agent.

Still another aspect is directed to compositions for treating mesothioloma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, ovarian cancer, cervical cancer, colon cancer, rectal cancer, cancer of the anal region, stomach cancer, gastrointestinal (gastric, colorectal, and duodenal), chronic lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular cancer (hepatic and billiary duct), primary or secondary central nervous system tumor, primary or secondary brain tumor, Hodgkin's disease, chronic or acute leukemia, chronic myeloid leukemia, lymphocytic lymphomas, lymphoblastic leukemia, follicular lymphoma, lymphoid malignancies of T-cell or B-cell origin, melanoma, multiple myeloma, oral cancer, ovarian cancer, non-small cell lung cancer, prostate cancer, small cell lung cancer, cancer of the kidney and ureter, renal cell carcinoma, carcinoma of the renal pelvis, neoplasms of the central nervous system, primary central nervous system lymphoma, non Hodgkin's lymphoma, spinal axis tumors, brains stem glioma, pituitary adenoma, adrenocortical cancer, gall bladder cancer, cancer of the spleen, cholangiocarcinoma, fibrosarcoma, neuroblastoma, retinoblasitoma, or a combination of one or more of the above cancers, said compositions comprising an excipient and therapeutically effective amount of an acylsulfonamide described herein and one additional therapeutic agent or more than one additional therapeutic agent.

Still another aspect is directed to methods of treating mesothioloma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, ovarian cancer, cervical cancer, colon cancer, rectal cancer, cancer of the anal region, stomach cancer, gastrointestinal (gastric, colorectal, and duodenal), chronic lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular cancer (hepatic and billiary duct), primary or secondary central nervous system tumor, primary or secondary brain tumor, Hodgkin's disease, chronic or acute leukemia, chronic myeloid leukemia, lymphocytic lymphomas, lymphoblastic leukemia, follicular lymphoma, lymphoid malignancies of T-cell or B-cell origin, melanoma, multiple myeloma, oral cancer, ovarian cancer, non-small cell lung cancer, prostate cancer, small cell lung cancer, cancer of the kidney and ureter, renal cell carcinoma, carcinoma of the renal pelvis, neoplasms of the central nervous system, primary central nervous system lymphoma, non Hodgkin's lymphoma, spinal axis tumors, brains stem glioma, pituitary adenoma, adrenocortical cancer, gall bladder cancer, cancer of the spleen, cholangiocarcinoma, fibrosarcoma, neuroblastoma, retinoblasitoma, or a combination of one or more of the above cancers in a patient, said methods comprising administering thereto therapeutically effective amounts of an acylsulfonamide described herein and one additional therapeutic agent or more than one additional therapeutic agent.

Still another aspect is directed to methods of treating mesothioloma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, ovarian cancer, cervical cancer, colon cancer, rectal cancer, cancer of the anal region, stomach cancer, gastrointestinal (gastric, colorectal, and duodenal), chronic lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular cancer (hepatic and billiary duct), primary or secondary central nervous system tumor, primary or secondary brain tumor, Hodgkin's disease, chronic or acute leukemia, chronic myeloid leukemia, lymphocytic lymphomas, lymphoblastic leukemia, follicular lymphoma, lymphoid malignancies of T-cell or B-cell origin, melanoma, multiple myeloma, oral cancer, ovarian cancer, non-small cell lung cancer, prostate cancer, small cell lung cancer, cancer of the kidney and ureter, renal cell carcinoma, carcinoma of the renal pelvis, neoplasms of the central nervous system, primary central nervous system lymphoma, non Hodgkin's lymphoma, spinal axis tumors, brains stem glioma, pituitary adenoma, adrenocortical cancer, gall bladder cancer, cancer of the spleen, cholangiocarcinoma, fibrosarcoma, neuroblastoma, retinoblasitoma, or a combination of one or more of the above cancers in a patient, said methods comprising administering thereto therapeutically effective amounts of an acylsulfonamide described herein and one or more than one of etoposide vincristine CHOP, rituximab, rapamycin, R-CHOP or bortezomib.

### ABBREVIATIONS AND DEFINITIONS

The following definitions and methods are provided to better define the present disclosure and to guide those of ordinary skill in the art in the practice of the present disclosure. Unless otherwise noted, terms are to be understood according to conventional usage by those of ordinary skill in the relevant art.

With regard to stereoisomers, it should be understood that a solid line designation for the bonds in the compounds corresponding to Formulae (1), (2), and (3) (and others herein) for attachment of an substituent group (e.g., Z₁, Z₂, and further substituents on these groups) to a chiral carbon atom of the compound indicates that these groups may lie either below or above the plane of the page (i.e., [symbol] R or [symbol] R). All isomeric forms of the compounds disclosed herein are contemplated, including racemates, racemic mixtures, and individual enantiomers or diastereomers.

Compounds of this invention may contain asymmetrically substituted carbon atoms in the R or S configuration, wherein the terms “R” and “S” are as defined in Pure Appl. Chem. (1976) 45, 13-10. Compounds having asymmetrically substituted carbon atoms with equal amounts of R and S configurations are racemic at those atoms. Atoms having excess of one configuration over the other are assigned the configuration in excess, preferably an excess of about 85%-90%, more preferably an excess of about 95%-99%, and still more preferably an excess greater than about 99%. Accordingly, this invention is meant to embrace racemic mixtures and relative and absolute diastereoisomers of the compounds thereof.

Compounds of this invention may also contain carbon-carbon double bonds or carbon-nitrogen double bonds in the Z or E configuration, in which the term “Z” represents the larger two substituents on the same side of a carbon-carbon or carbon-nitrogen double bond and the term “E” represents the larger two substituents on opposite sides of a carbon-carbon or carbon-nitrogen double bond. The compounds of this invention may also exist as a mixture of “Z” and “E” isomers.

Compounds of this invention may also exist as tautomers or equilibrium mixtures thereof wherein a proton of a compound shifts from one atom to another. Examples of tautomers include, but are not limited to, keto-enol, phenol-keto, oxime-nitroso, nitro-aci, imine-enamine and the like.

The terms “acetal” and “ketal,” as used herein alone or as part of another group, denote the moieties represented by the following formulae, respectively:

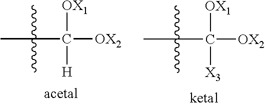

wherein X₁ and X₂ are independently hydrocarbyl, substituted hydrocarbyl, heterocyclo, or heteroaryl, and X₃ is hydrocarbyl or substituted hydrocarbyl, as defined in connection with such terms, and the wavy lines represent the attachment point of the acetal or ketal moiety to another moiety or compound.

The term “acyl,” as used herein alone or as part of another group, denotes the moiety formed by removal of the hydroxyl group from the group —COOH of an organic carboxylic acid, e.g., X₄C(O)—, wherein X₄ is X¹, X¹O—, X¹X²N—, or X¹S—, X¹ is hydrocarbyl, heterosubstituted hydrocarbyl, or heterocyclo, and X² is hydrogen, hydrocarbyl or substituted hydrocarbyl. Exemplary acyl moieties include acetyl, propionyl, benzoyl, pyridinylcarbonyl, and the like.

The term “acyloxy,” as used herein alone or as part of another group, denotes an acyl group as described above bonded through an oxygen linkage (—O—), e.g., X₄C(O)O— wherein X₄ is as defined in connection with the term “acyl.”

The term “alkanol,” as used herein alone or as part of another group, denotes an alkyl radical having 1 to 10 carbon atoms, which is substituted by one, two or three, or more, hydroxyl group(s). Examples of alkanols include methanol, ethanol, n-propan-2-ol, n-propan-3-ol, isopropanol, i-butanol, and the like.

The term “alkanoyl,” as used herein, represents an alkyl group attached to the parent molecular moiety through a carbonyl group. The alkanoyl groups of this invention can be optionally substituted with one or two groups independently selected from the group consisting of hydroxyl and amino.

The term “alkanoylalkyl,” as used herein, represents an alkanoyl group attached to the parent molecular moiety through an alkyl group.

The term “alkoxy,” as used herein alone or as part of another group, denotes an —OX₅ radical, wherein X₅ is as defined in connection with the term “alkyl.” Exemplary alkoxy moieties include methoxy, ethoxy, propoxy, or 2-propoxy, n-, iso-, or tert-butoxy, and the like.

The term “alkenoxy,” as used herein alone or as part of another group, denotes an —OX₆ radical, wherein X₆ is as defined in connection with the term “alkenyl.” Exemplary alkenoxy moieties include ethenoxy, propenoxy, butenoxy, hexenoxy, and the like.

The term “alkynoxy,” as used herein alone or as part of another group, denotes an —OX₇ radical, wherein X₇ is as defined in connection with the term “alkynyl.” Exemplary alkynoxy moieties include ethynoxy, propynoxy, butynoxy, hexynoxy, and the like.

The term “alkoxyalkanoyl,” as used herein, represents an alkoxy group attached to the parent molecular moiety through an alkanoyl group.

The term “alkoxyalkoxy,” as used herein, represents an alkoxy group attached to the parent molecular moiety through another alkoxy group.

The term “alkoxyalkoxyalkyl,” as used herein, represents an alkoxyalkoxy group attached to the parent molecular moiety through an alkyl group.

The term “alkoxyalkoxycarbonyl,” as used herein, represents an alkoxyalkoxy group attached to the parent molecular moiety through a carbonyl group.

The term “alkoxyalkyl,” as used herein, represents an alkoxy group attached to the parent molecular moiety through an alkyl group.

The term “alkoxycarbonyl,” as used herein, represents an alkoxy group attached to the parent molecular moiety through a carbonyl group.

The term “alkoxycarbonylalkyl,” as used herein, represents an alkoxycarbonyl group attached to the parent molecular moiety through an alkyl group.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkylamino,” as used herein, represents —N(X₈)₂, wherein X₈ is alkyl.

The term “alkylaminoalkyl,” as used herein, represents an alkylamino group attached to the parent molecular moiety through an alkyl group.

The term “alkylaminocarbonyl,” as used herein, represents an alkylamino group attached to the parent molecular moiety through a carbonyl group.

The term “alkylaminocarbonylalkyl,” as used herein, represents an alkylaminocarbonyl group attached to the parent molecular moiety through an alkyl group.

The term “alkylidene,” as used herein, represents an alkyl group attached to the parent molecular moiety through a carbon-carbon double bond.

The term “alkylsulfanyl,” as used herein, represents an alkyl group attached to the parent molecular moiety through a sulfur atom.

The term “alkylsulfanylalkyl,” as used herein, represents an alkylsulfanyl group attached to the parent molecular moiety through an alkyl group.

The term “alkylsulfonyl,” as used herein, represents an alkyl group attached to the parent molecular moiety through a sulfonyl group.

The term “alkylsulfonylalkyl,” as used herein, represents an alkylsulfonyl group attached to the parent molecular moiety through an alkyl group.

The term “alkylene,” as used herein alone or as part of another group, denotes a linear saturated divalent hydrocarbon radical of one to eight carbon atoms or a branched saturated divalent hydrocarbon radical of three to six carbon atoms unless otherwise stated. Exemplary alkylene moieties include methylene, ethylene, propylene, 1-methylpropylene, 2-methylpropylene, butylene, pentylene, and the like. Unless otherwise indicated, one or more hydrogen atoms of the alkylene moieties can be replaced and substituted with one or more of ═O, —OH, —OR_{Z}, —COOH, —COOR_{Z}, —CONH₂, —NH₂, —NHR_{Z}, —NR_{Z}R_{Z}, —NO₂, —SH, —SR_{Z}, —SO₂R_{Z}, —SO₂H, —SOR_{Z}, heterocyclo, and halo (including F, Cl, Br and I), among others, wherein each occurrence of R_{Z} may be hydrocarbyl or substituted hydrocarbyl (e.g., substituted or unsubstituted alkyl, substituted or unsubstituted aryl, or substituted or unsubstituted aralkyl.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

Unless otherwise indicated, the terms “amine” or “amino,” as used herein alone or as part of another group, represents a group of formula —N(X₉)(X₁₀), wherein X₉ and X₁₀ are independently hydrogen, hydrocarbyl, substituted hydrocarbyl, heteroaryl, or heterocyclo, or X₈ and X₉ taken together form a substituted or unsubstituted alicyclic, aryl, or heterocyclic moiety, each as defined in connection with such term, typically having from 3 to 8 atoms in the ring. “Substituted amine,” for example, refers to a group of formula —N(X₉)(X₁₀), wherein at least one of X₉ and X₁₀ are other than hydrogen. “Unsubstituted amine,” for example, refers to a group of formula —N(X₉)(X₁₀), wherein X₉ and X₁₀ are both hydrogen.

By way of example, X₉ and X₁₀ may be independently selected from hydrogen, alkanoyl, alkenyl, alkoxyalkyl, alkoxyalkoxyalkyl, alkoxycarbonyl, alkyl, alkylaminoalkyl, alkylaminocarbonylalkyl, aryl, arylalkyl, cycloalkyl, (cycloalkyl)alkyl, cycloalkylcarbonyl, haloalkanoyl, haloalkyl, (heterocyclo)alkyl, heterocyclocarbonyl, hydroxyalkyl, an amino protecting group, —C(NH)NH₂, and —C(O)N(X₉)(X₁₀), wherein X₉ and X₁₀ are as previously defined; wherein the aryl; the aryl part of the arylalkyl; the cycloalkyl; the cycloalkyl part of the (cycloalkyl)alkyl and the cycloalkylcarbonyl; and the heterocycle part of the (heterocycle)alkyl and the heterocyclocarbonyl can be optionally substituted with one, two, three, four, or five substituents independently selected from the group consisting of alkanoyl, alkoxy, alkyl, cyano, halo, haloalkoxy, haloalkyl, hydroxyl, and nitro.

The term “aminoalkanoyl,” as used herein, represents an amino group attached to the parent molecular moiety through an alkanoyl group.

The term “aminoalkyl,” as used herein, represents an amino group attached to the parent molecular moiety through an alkyl group.

The term “aminocarbonyl,” as used herein, represents an amino group attached to the parent molecular moiety through a carbonyl group.

The term “aminocarbonylalkyl,” as used herein, represents an aminocarbonyl group attached to the parent molecular moiety through an alkyl group.

The term “aminosulfonyl,” as used herein, represents an amino group attached to the parent molecular moiety through a sulfonyl group.

Unless otherwise indicated, the terms “amido” or “amide,” as used herein alone or as part of another group, represents a group of formula —CON(X₉)(X₁₀), wherein X₉ and X₁₀ are as defined in connection with the terms “amine” or “amino.” In general, “amido” or “amide” groups may be either substituted or unsubstituted. “Substituted amide,” for example, refers to a group of formula —CON(X₉)(X₁₀), wherein at least one of X₉ and X₁₀ are other than hydrogen. “Unsubstituted amido,” for example, refers to a group of formula —CON(X₉)(X₁₀), wherein X₉ and X₁₀ are both hydrogen.

The terms “amino protecting group,” “protected amino,” or “Pr” as used herein denote moieties that block reaction at the protected amino group while being easily removed under conditions that are sufficiently mild so as not to disturb other substituents of the various compounds. Common N-protecting groups comprise benzyl and acyl groups such as acetyl, benzoyl, 2-bromoacetyl, 4-bromobenzoyl, tert-butylacetyl, carboxaldehyde, 2-chloroacetyl, 4-chlorobenzoyl, a-chlorobutyryl, 4-nitrobenzoyl, o-nitrophenoxyacetyl, phthalyl, pivaloyl, propionyl, trichloroacetyl, and trifluoroacetyl; sulfonyl groups such as benzenesulfonyl, and p-toluenesulfonyl; carbamate forming groups such as benzyloxycarbonyl, benzyloxycarbonyl (Cbz), tert-butyloxycarbonyl (Boc), p-chlorobenzyloxycarbonyl, p-methoxybenzyloxycarbonyl, allyloxycarbonyl, fluorenylmethoxycarbonyl (Fmoc), and the like. A variety of protecting groups for the amino group and the synthesis thereof may be found in “Protective Groups in Organic Synthesis” by T. W. Greene and P. G. M. Wuts, John Wiley & Sons, 1999.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. For example, the term “aryl,” may represent a phenyl group or a bicyclic or tricyclic fused ring system wherein one or more of the fused rings is a phenyl group. Bicyclic fused ring systems are exemplified by a phenyl group fused to a cycloalkyl group as defined herein, a cycloalkenyl group as defined herein, or another phenyl group. Tricyclic fused ring systems are exemplified by a bicyclic fused ring system fused to a cycloalkyl group as defined herein, a cycloalkenyl group as defined herein, or another phenyl group. Representative examples of aryl include, but are not limited to, anthracenyl, azulenyl, fluorenyl, indanyl, indenyl, naphthyl, phenyl, and tetrahydronaphthyl. Aryl groups having an unsaturated or partially saturated ring fused to an aromatic ring can be attached through the saturated or the unsaturated part of the group. The aryl groups of this invention can be optionally substituted with one, two, three, four, or five substituents independently selected from the group consisting of alkanoyl, alkenyl, alkoxy, alkoxyalkanoyl, alkoxyalkyl, alkoxycarbonyl, alkoxycarbonylalkyl, alkyl, alkynyl, amino, aminoalkyl, aminocarbonyl, aminocarbonylalkyl, aminosulfonyl, aryl, aryloxy, arylsulfanyl, carbonyloxy, cyano, halo, haloalkoxy, haloalkyl, heterocycle, (heterocycle)alkyl, heterocyclecarbonylalkenyl, heterocyclecarbonylalkyl, hydroxy, hydroxyalkyl, nitro, oxo, and —C(NH)NH₂, wherein the aryl; the aryl part of the aryloxy and the arylsulfanyl; the heterocycle; and the heterocycle part of the (heterocycle)alkyl, the heterocyclecarbonylalkenyl, and the heterocyclecarbonylalkyl can be further optionally substituted with one, two, or three substituents independently selected from the group consisting of alkoxyalkanoyl, alkoxycarbonyl, alkyl, alkylsulfonyl, aminocarbonyl, aminosulfonyl, cyano, halo, haloalkoxy, haloalkyl, hydroxy, nitro, oxo, and —C(NH)NH₂. In addition, the heterocycle and the heterocycle part of the (heterocycle)alkyl, the heterocyclecarbonylalkenyl, and the heterocyclecarbonylalkyl can be further optionally substituted with an additional aryl group, wherein the aryl can be optionally substituted with one, two, or three substituents independently selected from the group consisting of alkoxy, alkyl, cyano, halo, hydroxy, and nitro.

The term “arylalkenyl,” as used herein, represents an aryl group attached to the parent molecular moiety through an alkenyl group.

The term “arylalkoxy,” as used herein, represents an aryl group attached to the parent molecular moiety through an alkoxy group.

The term “arylalkoxyalkanoyl,” as used herein, represents an arylalkoxy group attached to the parent molecular moiety through an alkanoyl group.

The term “arylalkoxycarbonyl,” as used herein, represents an arylalkoxy group attached to the parent molecular moiety through a carbonyl group.

The term “arylalkylsulfanyl,” as used herein, represents an arylalkyl group attached to the parent molecular moiety through a sulfur atom.

The term “arylalkylsulfanylalkyl,” as used herein, represents an arylalkylsulfanyl group attached to the parent molecular moiety through an alkyl group.

The term “arylalkylsulfonyl,” as used herein, represents an arylalkyl group attached to the parent molecular moiety through a sulfonyl group.

The term “arylcarbonyl,” as used herein, represents an aryl group attached to the parent molecular moiety through a carbonyl group.

The term “aryloxy,” as used herein, represents an aryl group attached to the parent molecular moiety through an oxygen atom.

The term “aryloxyalkoxy,” as used herein, represents an aryloxy group attached to the parent molecular moiety through an alkoxy group.

The term “aryloxyalkyl,” as used herein, represents an aryloxy group attached to the parent molecular moiety through an alkyl group.

The term “arylsulfanyl,” as used herein, represents an aryl group attached to the parent molecular moiety through a sulfur atom.

The term “arylsulfanylalkoxy,” as used herein, represents an arylsulfanyl group attached to the parent molecular moiety through an alkoxy group.

The term “arylsulfanylalkyl,” as used herein, represents an arylsulfanyl group attached to the parent molecular moiety through an alkyl group. The alkyl part of the arylsulfanylalkyl can be optionally substituted with one or two substituents independently selected from the group consisting of alkoxy, alkoxycarbonyl, amino, aminocarbonyl, arylalkoxy, azido, carboxy, cycloalkyl, halo, heterocycle, (heterocycle)alkoxy, (heterocycle)carbonyl, and hydroxy.

The term “arylsulfinyl,” as used herein, represents an aryl group attached to the parent molecular moiety through a sulfinyl group.

The term “arylsulfinylalkyl,” as used herein, represents an arylsulfinyl group attached to the parent molecular moiety through an alkyl group. The alkyl part of the arylsulfinylalkyl can be optionally substituted with one or two amino groups.

The term “arylsulfonyl,” as used herein, represents an aryl group attached to the parent molecular moiety through a sulfonyl group.

The term “arylsulfonylalkyl,” as used herein, represents an arylsulfonyl group attached to the parent molecular moiety through an alkyl group. The alkyl part of the arylsulfonylalkyl can be optionally substituted with one or two amino groups.

The term “arylene”, as used herein alone or part of another group refers to a divalent aryl radical of one to twelve carbon atoms. Non-limiting examples of “arylene” include phenylene, pyridinylene, pyrimidinylene and thiophenylene.

The terms “aralkyl,” “arylalkyl,” or “alkylene aryl,” as used herein alone or as part of another group, denotes an -(alkylene)-X₁₁ radical, wherein X₁₁ is as defined in connection with the term “aryl.” Non-limiting examples of “aralkyl” or “alkylene aryl” moieties include benzyl, —(CH₂)ₙ-phenyl where n is 2 to 6, or —CH-(phenyl)₂.

The terms “alkaryl” or “alkylaryl,” as used herein alone or as part of another group, denotes an -(arylene)-X₁₁ radical, wherein X₁₁ is as defined in connection with the term “alkyl.”

The term “azido,” as used herein, represents a —N₃ moiety.

The term “carbocyclic,” as used herein alone or as part of another group, denotes a ring wherein the atoms forming the ring backbone are selected from only carbon atoms. The carbocyclic rings may be optionally substituted, fully saturated or unsaturated, monocyclic or bicyclic, aromatic or nonaromatic, and generally include 3 to 20 carbon atoms.

The term “carbonyl,” as used herein, represents a —C(O)— moiety.

The term “carbonyloxy,” as used herein, represents an alkanoyl group attached to the parent molecular moiety through an oxygen atom.

The term “carboxy,” as used herein, represents a —CO₂H moiety.

The term “carboxyalkyl,” as used herein, represents a carboxy group attached to the parent molecular moiety through an alkyl group.

The term “cyano,” as used herein alone or as part of another group, denotes a group of formula —CN.

The term “cyanoalkyl,” as used herein, represents a cyano group attached to the parent molecular moiety through an alkyl group.

The term “cycloalkyl,” as used herein alone or as part of another group, denotes a cyclic saturated monovalent bridged or non-bridged hydrocarbon radical of three to twelve carbon atoms. Exemplary cycloalkyl moieties include cyclopropyl, cyclobutyl, cyclopentyl, cyclohexyl, or adamantyl. By way of example, the term “cycloalkyl” may represent a saturated ring system having three to twelve carbon atoms and one to three rings. Examples of cycloalkyl groups include cyclopropyl, cyclopentyl, bicyclo(3.1.1)heptyl, adamantyl, and the like. The cycloalkyl groups of this invention can be optionally substituted with one, two, three, four, or five substituents independently selected from the group consisting of alkoxy, alkoxycarbonyl, alkyl, aminoalkyl, arylalkoxy, aryloxy, arylsulfanyl, halo, haloalkoxy, haloalkyl, and hydroxy, wherein the aryl part of the arylalkoxy, the aryloxy, and the arylsulfanyl can be further optionally substituted with one, two, or three substituents independently selected from the group consisting of alkoxy, alkyl, halo, haloalkoxy, haloalkyl, and hydroxy.

The term “cycloalkylalkoxy,” as used herein, represents a cycloalkyl group attached to the parent molecular moiety through an alkoxy group.

The term “(cycloalkyl)alkyl,” as used herein, represents a cycloalkyl group attached to the parent molecular moiety through an alkyl group.

The term “cycloalkylcarbonyl,” as used herein, represents a cycloalkyl group attached to the parent molecular moiety through a carbonyl group.

The term “cycloalkyloxy,” as used herein, represents a cycloalkyl group attached to the parent molecular moiety through an oxygen atom.

The term “cycloalkenyl,” as used herein, represents a non-aromatic ring system having three to ten carbon atoms and one to three rings, wherein each five-membered ring has one double bond, each six-membered ring has one or two double bonds, each seven- and eight-membered ring has one to three double bonds, and each nine- to ten-membered ring has one to four double bonds. Examples of cycloalkenyl groups include cyclohexenyl, octahydronaphthalenyl, norbornylenyl, and the like. The cycloalkenyl groups of this invention can be optionally substituted with one, two, three, four, or five substituents independently selected from the group consisting of alkoxy, alkoxycarbonyl, alkyl, aminoalkyl, arylalkoxy, aryloxy, arylsulfanyl, halo, haloalkoxy, haloalkyl, and hydroxy, wherein the aryl part of the arylalkoxy, the aryloxy, and the arylsulfanyl can be further optionally substituted with one, two, or three substituents independently selected from the group consisting of alkoxy, alkyl, halo, haloalkoxy, haloalkyl, and hydroxy.

The term “cycloalkenylalkyl,” as used herein, represents a cycloalkenyl group attached to the parent molecular moiety through an alkyl group.

The term “ester,” as used herein alone or as part of another group, denotes a group of formula —COOX₁₂ wherein X₁₂ is alkyl or aryl, each as defined in connection with such term.

The term “ether,” as used herein alone or as part of another group, includes compounds or moieties which contain an oxygen atom bonded to two carbon atoms. For example, ether includes “alkoxyalkyl” which refers to an alkyl, alkenyl, or alkynyl group substituted with an alkoxy group.

The term “formyl,” as used herein, represents a —CHO moiety.

The term “formylalkyl,” as used herein, represents a formyl group attached to the parent molecular moiety through an alkyl group.

The terms “halide,” “halogen” or “halo” as used herein alone or as part of another group refer to chlorine, bromine, fluorine, and iodine.

The term “haloalkyl,” as used herein, represents an alkyl group substituted by one, two, three, or four halogen atoms.

The term “haloalkanoyl,” as used herein, represents a haloalkyl group attached to the parent molecular moiety through a carbonyl group.

The term “haloalkoxy,” as used herein, represents a haloalkyl group attached to the parent molecular moiety through an oxygen atom.

The term “heteroatom” shall mean atoms other than carbon and hydrogen.

The terms “heteroaralkyl” and “alkylene heteroaryl,” as used herein alone or as part of another group, denotes an -(alkylene)-X₁₃ radical, wherein X₁₃ is as defined in connection with the term “heteroaryl.” Non-limiting examples of “heteroaralkyl” or “alkylene heteroaryl” moieties include —(CH₂)ₙ-indolyl where n is 1 to 6.

The term “heteroalkylene,” as used herein, represents a divalent group of two to eight atoms derived from a saturated straight or branched chain containing one or two heteroatoms independently selected from the group consisting of nitrogen, oxygen, and sulfur, wherein the remaining atoms are carbon. The heteroalkylene groups of the present invention can be attached to the parent molecular moiety through the carbon atoms or the heteroatoms in the chain.

The term “heteroalkenylene,” as used herein, represents a divalent group of three to eight atoms derived from a straight or branched chain containing at least one carbon-carbon double bond that contains one or two heteroatoms independently selected from the group consisting of nitrogen, oxygen, and sulfur, wherein the remaining atoms are carbon. The heteroalkenylene groups of the present invention can be attached to the parent molecular moiety through the carbon atoms or the heteroatoms in the chain.

The term “heterocyclo” or “heterocycle,” as used herein, represents a monocyclic, bicyclic, or tricyclic ring system wherein one or more rings is a four-, five-, six-, or seven-membered ring containing one, two, or three heteroatoms independently selected from the group consisting of nitrogen, oxygen, and sulfur. Monocyclic ring systems are exemplified by any 3- or 4-membered ring containing a heteroatom independently selected from the group consisting of oxygen, nitrogen and sulfur; or a 5-, 6- or 7-membered ring containing one, two or three heteroatoms wherein the heteroatoms are independently selected from the group consisting of nitrogen, oxygen and sulfur. The 3- and 4-membered rings have no double bonds, the 5-membered ring has from 0-2 double bonds and the 6- and 7-membered rings have from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, a cycloalkenyl group, as defined herein, or another monocyclic heterocycle ring system. Representative examples of bicyclic ring system include but are not limited to, benzimidazole, benzothiazole, benzothiophene, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, pyranopyridine, quinoline, quinolizine, quinoxaline, quinazoline, tetrahydroisoquinoline, tetrahydroquinoline, thiopyranopyridine, and the like. Tricyclic rings systems are exemplified by any of the above bicyclic ring systems fused to an aryl group as defined herein, a cycloalkyl group as defined herein, a cycloalkenyl group as defined herein, or another monocyclic heterocycle ring system. Representative examples of tricyclic ring systems include, but are not limited to, acridine, carbazole, carboline, dibenzofuran, dibenzothiophene, naphthofuran, naphthothiophene, oxanthrene, phenazine, phenoxathiin, phenoxazine, phenothiazine, thianthrene, thioxanthene, xanthene, and the like. Heterocycle groups can be attached to the parent molecular moiety through a carbon atom or a nitrogen atom in the group.

The heterocyclo groups of the present invention can be optionally substituted with one, two, three, four, or five substituents independently selected from the group consisting of alkanoyl, alkanoylalkyl, alkenyl, alkoxy, alkoxyalkoxycarbonyl, alkoxyalkyl, alkoxycarbonyl, alkoxycarbonylalkyl, alkyl, alkylsulfanylalkyl, alkynyl, amino, aminoalkanoyl, aminoalkyl, aminocarbonyl, aminocarbonylalkyl, aminosulfonyl, aryl, arylalkoxyalkanoyl, arylalkoxycarbonyl, arylalkyl, arylalkylsulfonyl, arylcarbonyl, aryloxy, arylsulfanyl, arylsulfanylalkyl, arylsulfonyl, carbonyloxy, carboxy, cyano, cyanoalkyl, cycloalkyl, (cycloalkyl)alkyl, cycloalkylcarbonyl, formyl, formylalkyl, halo, haloalkoxy, haloalkyl, heterocycle, (heterocycle)alkyl, (heterocycle)alkylidene, heterocyclecarbonyl, heterocyclecarbonylalkyl, hydroxy, hydroxyalkyl, nitro, oxo, spirocycle, spiroheterocycle, and —C(NH)NH₂; wherein the aryl; the aryl part of the arylalkylsulfonyl, the arylcarbonyl, the aryloxy, the arylalkoxyalkanoyl, the arylalkoxycarbonyl, the arylalkyl, the arylsulfanyl, the arylsulfanylalkyl, and the arylsulfonyl; the heterocycle; and the heterocycle part of the (heterocycle)alkyl, the (heterocycle)alkylidene, the heterocyclecarbonyl, and the heterocyclecarbonylalkyl can be further optionally substituted with one, two, three, four, or five substituents independently selected from the group consisting of alkanoyl, alkoxy, alkoxyalkoxycarbonyl, alkoxycarbonyl, alkyl, halo, haloalkoxy, haloalkyl, hydroxy, hydroxyalkyl, and nitro.

The term “(heterocyclo)alkoxy,” as used herein, represents a heterocyclo group attached to the parent molecular moiety through an alkoxy group.

The term “(heterocyclo)alkyl,” as used herein, represents a heterocyclo group attached to the parent molecular moiety through an alkyl group.

The term “(heterocyclo)alkylidene,” as used herein, represents a heterocyclo group attached to the parent molecular moiety through an alkylidene group.

The term “heterocyclocarbonyl,” as used herein, represents a heterocyclo group attached to the parent molecular moiety through a carbonyl group.

The term “heterocyclocarbonylalkenyl,” as used herein, represents a heterocyclecarbonyl group attached to the parent molecular moiety through an alkenyl group.

The term “heterocyclocarbonylalkyl,” as used herein, represents a heterocyclocarbonyl group attached to the parent molecular moiety through an alkyl group.

The term “(heterocyclo)oxy,” as used herein, represents a heterocyclo group attached to the parent molecular moiety through an oxygen atom.

The term “(heterocyclo)sulfanyl,” as used herein, represents a heterocyclo group attached to the parent molecular moiety through a sulfur atom.

The term “(heterocyclo)sulfanylalkyl,” as used herein, represents a heterocyclosulfanyl group attached to the parent molecular moiety through an alkyl group.

The term “heteroaromatic” or “heteroaryl” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “hydroxy” or “hydroxyl,” as used herein alone or as part of another group, denotes a group of formula —OH.

The term “hydroxyalkyl,” as used herein, represents a hydroxy group attached to the parent molecular moiety through an alkyl group.

The term “hydroxyl protecting group,” as used herein alone or as part of another group, denote a group capable of protecting a free hydroxyl group (“protected hydroxyl”) which, subsequent to the reaction for which protection is employed, may be removed without disturbing the remainder of the molecule. Exemplary hydroxyl protecting groups include ethers (e.g., allyl, triphenylmethyl (trityl or Tr), benzyl, p-methoxybenzyl (PMB), p-methoxyphenyl (PMP)), acetals (e.g., methoxymethyl (MOM), β-methoxyethoxymethyl (MEM), tetrahydropyranyl (THP), ethoxy ethyl (EE), methylthiomethyl (MTM), 2-methoxy-2-propyl (MOP), 2-trimethylsilylethoxymethyl (SEM)), esters (e.g., benzoate (Bz), allyl carbonate, 2,2,2-trichloroethyl carbonate (Troc), 2-trimethylsilylethyl carbonate), silyl ethers (e.g., trimethylsilyl (TMS), triethylsilyl (TES), triisopropylsilyl (TIPS), triphenylsilyl (TPS), t-butyldimethylsilyl (TBDMS), t-butyldiphenylsilyl (TBDPS) and the like. A variety of protecting groups for the hydroxyl group and the synthesis thereof may be found in “Protective Groups in Organic Synthesis” by T. W. Greene and P. G. M. Wuts, John Wiley & Sons, 1999.

The term “keto,” as used herein alone or as part of another group, denotes a double bonded oxygen moiety (i.e., ═O).

The term “nitro,” as used herein alone or as part of another group, denotes a group of formula —NO₂.

The term “oxo,” as used herein, represents a (═O) moiety.

The term “spirocycle,” as used herein, represents an alkyl diradical of two to eight atoms, each end of which is attached to the same carbon atom of the parent molecular moiety.

The term “spiroheterocycle,” as used herein, represents a heteroalkylene diradical, each end of which is attached to the same carbon atom of the parent molecular moiety. Examples of spiroheterocycles include dioxolanyl, tetrahydrofuranyl, pyrrolidinyl, and the like.

The term “sulfinyl,” as used herein, represents a —S(═O)— moiety.

The term “sulfonyl,” as used herein, represents —S(═O)₂— moiety

Unless otherwise indicated, the “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxyl, protected hydroxyl, keto, acyl, acyloxy, nitro, amino, amido, nitro, cyano, thiol, ketals, acetals, esters, ethers, and thioethers.

The term “thioester,” as used herein alone or as part of another group, denotes a group of formula —C(O)—S—X₁₄, wherein X₁₄ is alkyl or aryl as defined in connection with such term.

The term “thioether,” as used herein alone or as part of another group, denotes compounds and moieties that contain a sulfur atom bonded to two different carbon or hetero atoms (i.e., —S—), and also includes compounds and moieties containing two sulfur atoms bonded to each other, each of which is also bonded to a carbon or hetero atom (i.e., dithioethers (—S—S—)). Examples of thioethers include, but are not limited to, alkylthioalkyls, alkylthioalkenyls, and alkylthioalkynyls. The term “alkylthioalkyls” includes compounds with an alkyl, alkenyl, or alkynyl group bonded to a sulfur atom that is bonded to an alkyl group. Similarly, the term “alkylthioalkenyls” and alkylthioalkynyls” refer to compounds or moieties where an alkyl, alkenyl, or alkynyl group is bonded to a sulfur atom that is covalently bonded to an alkynyl group.

The term “thiol,” as used herein alone or as part of another group, denotes a group of formula —SH. The term “thiol protecting group,” as used herein alone or as part of another group, denote a group capable of protecting a free thiol group (“protected thiol”) which, subsequent to the reaction for which protection is employed, may be removed without disturbing the remainder of the molecule. Suitable thiol protecting groups include, but are not limited to ethers, esters such as —C(O)CH₃, and the like. Other suitable thiol protecting groups include trityl (Trt), allyloxycarbonyl (Alloc), 1-(4,4-dimethyl-2,6-dioxocyclohex-1-ylidene)ethyl (Dde), acetamidomethyl (Acm), t-butyl (tBu), and fluorenylmethyl oxycarbonyl (Fmoc). A variety of protecting groups for the thiol group and the synthesis thereof may be found in “Protective Groups in Organic Synthesis” by T. W. Greene and P. G. M. Wuts, John Wiley & Sons, 1999.

Having described the invention in detail, it will be apparent that modifications and variations are possible without departing the scope of the invention defined in the appended claims. Furthermore, it should be appreciated that all examples in the present disclosure are provided as non-limiting examples.

In general, all reactions were run under an atmosphere of nitrogen unless otherwise indicated. Prior to use of solvents in reactions, they were purified by passing the degassed solvents through a column of activated alumina and transferred by an oven-dried syringe or cannula. Thin layer chromatography was performed on Merck TLC plates (silica gel 60 F₂₅₄). ¹H-NMR and ¹³C-NMR were recorded on a Varian Inova 400 (400 MHz) or a Bruker Avance DPX-250 (250 MHz) instrument. The HRMS data were measured on an Agilent 1100 Series MSD/TOF with electrospray ionization. LC/MS data were measured on an Agilent 1100 LC/MSD-VL with electrospray ionization. Sulfonyl azide (SZ8) prepared as reported procedure.

### Example 1

### Preparation of Building Blocks

1.1 Sulfonylazide (SZ8)

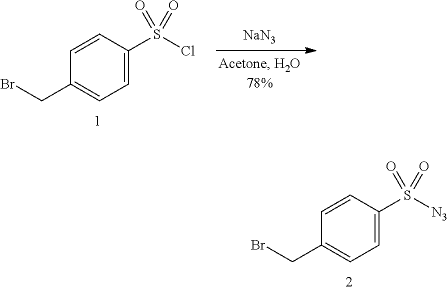 [SMILES: O=S(=O)(Cl)c1ccc(CBr)cc1 ; [N-]=[N+]=NS(=O)(=O)c1ccc(CBr)cc1 ; "NaN3"]

A saturated solution of sodium azide (1.2 g, 18.5 mmol) in water was added slowly to a saturated solution of 1 (5 g, 18.5 mmol) in acetone at room temperature. The mixture was stirred at room temperature for 3 hours. Ethyl acetate (50 mL) and saturated aqueous potassium carbonate solution (50 mL) were added. After extraction with ethyl acetate (50 mL×3), the combined organic phases were dried over anhydrous sodium sulfate and concentrated. The product 2 (SZ8) (4.0 g, 78%) was obtained by flash chromatography (hexanes:EtOAc=24:1). Rf=0.4 (hexanes:EtOAc=8:1). ¹H-NMR (400 MHz, CDCl₃) δ: 7.91 (d, J=8.3 Hz, 2H), 7.60 (d, J=8.3 Hz, 2H), 4.50 (s, 2H) ppm. ¹³C-NMR (100 MHz, CDCl₃) δ: 145.2, 138.4, 130.4, 128.2, 31.1 ppm. HRMS (ESI⁺) for [M+NH₄]⁺; calculated: 292.97024. found: 292.96949 (error m/z=−2.54 ppm).

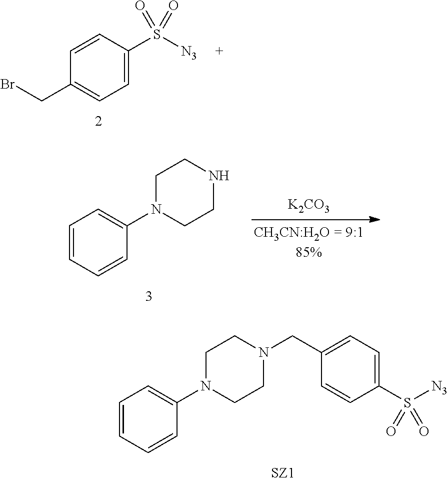

A mixture of (SZ8) (100 mg, 0.36 mmol), 3 (60 mg, 0.36 mmol) and potassium carbonate (100 mg, 0.72 mmol) in acetonitrile and water (9:1) was stirred at room temperature for 12 hours. The reaction mixture was then mixed with ethyl acetate (20 mL) and water (20 mL), and extracted with ethyl acetate (20 mL×3). The combined organic phases were dried over anhydrous sodium sulfate and concentrated. Product (SZ1) (110 mg, 85%) was obtained by flash chromatography (hexane:EtOAc=12:1). Rf=0.45 (hexanes:EtOAc=4:1). ¹H-NMR (400 MHz, CDCl₃) δ: 7.91 (d, J=8.2 Hz, 2H), 7.62 (d, J=8.2 Hz, 2H), 7.26-7.24 (m, 2H), 6.94-6.84 (m, 3H), 3.66 (s, 2H), 3.23-3.20 (m, 4H), 2.64-2.61 (m, 4H) ppm. ¹³C-NMR (100 MHz, CDCl₃) δ: 151.2, 146.5, 137.1, 129.9, 129.1, 127.5, 119.8, 116.1, 62.2, 53.2, 49.1 ppm. HRMS (ESI⁺) for [M+H]⁺; calculated: 358.13322. found: 358.13320 (error m/z=−0.07 ppm).

1.2 Sulfonylazide (SZ2)

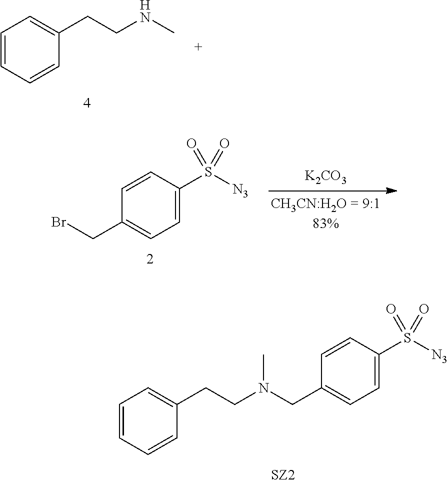

The mixture of (SZ8) (100 mg, 0.36 mmol), 4 (50 mg, 0.36 mmol) and potassium carbonate (100 mg, 0.72 mmol) in acetonitrile and water (9:1), was stirred at room temperature for 12 hours. After mixed with ethyl acetate (20 mL) and water (20 mL), the system was extracted by ethyl acetate (20 mL×3). The combined organic phase was dried by anhydrous sodium sulfate and concentrated. Product (SZ2) (100 mg, 83%) was obtained by flash chromatography (hexane:EtOAc=14:1; Rf=0.5 in hexane:EtOAc=4:1). ¹H-NMR (400 MHz, CDCl₃) δ: 7.84 (d, J=8.2 Hz, 2H), 7.48 (d, J=8.1 Hz, 2H), 7.30-7.16 (m, 5H), 3.63 (s, 2H), 2.82 (t, J=8.0 Hz, 2H), 2.66 (t, J=8.0 Hz, 2H), 2.30 (s, 3H) ppm. ¹³C-NMR (100 MHz, CDCl₃) δ: 147.7, 140.2, 136.7, 129.7, 128.7, 128.3, 127.4, 126.1, 61.6, 59.2, 42.2, 33.9 ppm. HRMS (ESI⁺) for [M+H]⁺; calculated: 331.12232. found: 331.12269 (error m/z=1.11 ppm).

1.3 Sulfonylazide (SZ3)

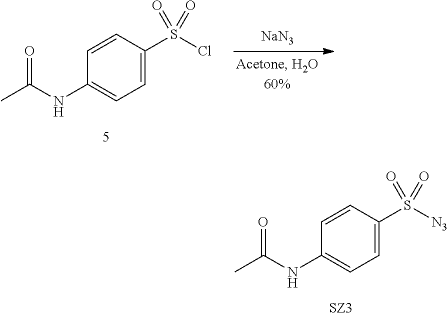 [SMILES: CC(=O)Nc1ccc(S(=O)(=O)Cl)cc1 ; CC(=O)Nc1ccc(S(=O)(=O)N=[N+]=[N-])cc1 ; "NaN3"]

(SZ3) was prepared starting from 5 using the procedure described for the preparation of 2 with 60% yield (hexane:EtOAc=2:1; Rf=0.25 in hexane:EtOAc=1:1). ¹H-NMR (400 MHz, CDCl₃) δ: 8.10 (s, 1H), 7.84 (d, J=8.2 Hz, 2H), 7.74 (d, J=8.2 Hz, 2H), 2.21 (s, 3H) ppm. ¹³C-NMR (100 MHz, CDCl₃) δ: 169.0, 143.8, 132.2, 128.7, 119.4, 24.5 ppm. HRMS (ESI⁺) for [M+NH₄]⁺; calculated: 258.06554. found: 258.06476 (error m/z=−3.02 ppm).

1.4 Sulfonylazide (SZ4)

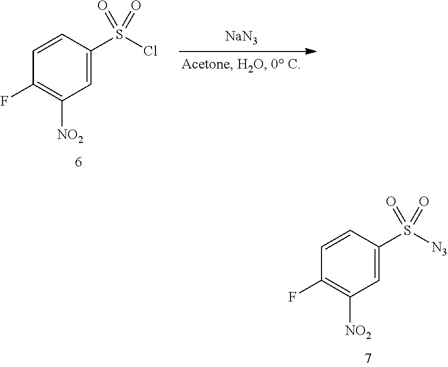 [SMILES: O=[N+]([O-])c1cc(S(=O)(=O)Cl)ccc1F ; [N-]=[N+]=NS(=O)(=O)c1ccc(F)c([N+](=O)[O-])c1 ; "NaN3"]

A saturated solution of sodium azide (280 mg, 4.30 mmol) in water was added slowly to a saturated solution of 6 (see Wendt et al., J. Med. Chem. 2006, 49, 1165-1181) (500 mg, 2.1 mmol) in acetone at 0° C. The mixture was stirred at 0° C. for 3 hours. Ethyl acetate (20 mL) and saturated aqueous potassium carbonate solution (20 mL) were added to the mixture and after extraction with ethyl acetate (20 mL×3), the combined organic phases were dried over anhydrous sodium sulfate and concentrated. Product 7 was used without further purification in the next reaction.

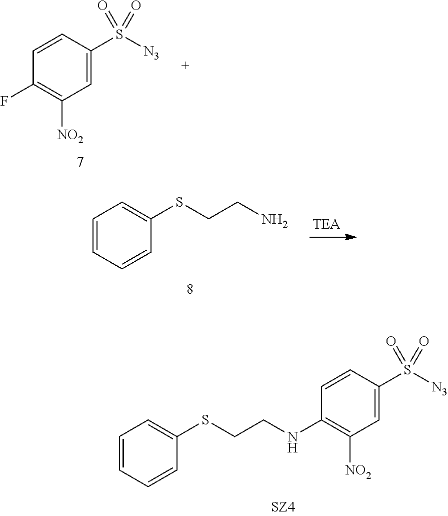 [SMILES: [N-]=[N+]=NS(=O)(=O)c1ccc(F)c([N+](=O)[O-])c1 ; NCCSc1ccccc1 ; [N-]=[N+]=NS(=O)(=O)c1ccc(NCCSc2ccccc2)c([N+](=O)[O-])c1]

A mixture of compounds 7 (see Wendt et al., supra) (280 mg, more than 0.91 mmol) and 8 (224 mg, 0.91 mmol) in triethylamine (5 mL) was stirred overnight at room temperature. To the reaction mixture was added silica (600 mg) and the solvent was removed under reduced pressure. Product (SZ4) (260 mg, 68.5% over two steps) was obtained by flash chromatography (hexane:EtOAc=8:1-2:1; Rf=0.4 in hexane:EtOAc=2:1). ¹H-NMR (400 MHz, CDCl₃) δ: 8.77 (d, J=2.4 Hz, 1H), 8.71 (d, J=2.4 Hz, 1H), 7.79 (dd, J=9.2, 2.4 Hz, 1H), 7.39 (dd, J=8.4, 1.2 Hz, 2H), 7.30-7.24 (m, 3H), 6.85 (d, J=9.2 Hz, 1H), 3.60 (dd, J=9.0, 6.4 Hz, 2H), 3.22 (t, J=6.8 Hz, 2H) ppm. ¹³C-NMR (100 MHz, CDCl₃) δ: 148.1, 134.2, 133.6, 131.3, 129.8, 129.5, 128.5, 127.7, 124.5, 115.0, 42.3, 33.3 ppm. HRMS (ESI⁺) for [M+H]⁺; calculated: 380.04817. found: 380.04795 (error m/z=−0.59 ppm).

1.5 Sulfonylazide (SZ5)

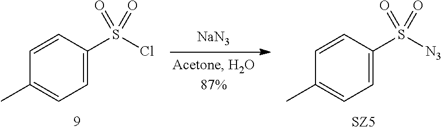

The known (SZ5) (Waser et al., J. Am. Chem. Soc. 2006, 128(35), 11693-11712) was prepared starting from 9 using the procedure described for the preparation of 2 in 87% yield. (DCM:MeOH=60:1; Rf=0.4 in DCM:MeOH=20:1) ¹H-NMR (250 MHz, CDCl₃) δ: 7.84 (d, J=8.4 Hz, 2H), 7.41 (d, J=8.1 Hz, 2H), 2.48 (s, 3H) ppm.

1.6 Sulfonylazide (SZ6)

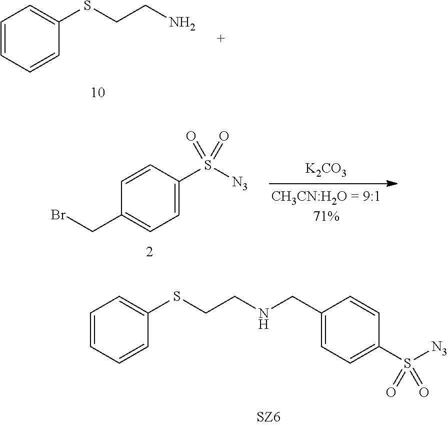

The mixture of (SZ8) (100 mg, 0.36 mmol), 10 (55 mg, 0.36 mmol) and potassium carbonate (100 mg, 0.72 mmol) in acetonitrile and water (9:1), was stirred at room temperature for 12 hours. The reaction mixture was then mixed with ethyl acetate (20 mL) and water (20 mL), and extracted by ethyl acetate (20 mL×3). The combined organic phases were dried over anhydrous sodium sulfate and concentrated. Product (SZ6) (89 mg, 71%) was obtained by flash chromatography (hexane:EtOAc=3:1). Rf=0.45 (hexanes:EtOAc=1:1). ¹H-NMR (400 MHz, CDCl₃) δ: 7.86 (d, J=8.4 Hz, 2H), 7.53 (d, J=8.4 Hz, 2H), 7.33-7.18 (m, 5H), 3.88 (s, 3H), 3.08 (t, J=6.0 Hz, 2H), 2.83 (t, J=6.0 Hz, 2H) ppm. ¹³C-NMR (100 MHz, CDCl₃) δ: 148.2, 136.8, 135.5, 129.8, 129.0, 127.6, 126.4, 52.6, 47.5, 34.2 ppm. HRMS (ESI⁺) for [M+H]⁺; calculated: 349.07874. found: 349.07937 (error m/z=1.79 ppm).

1.7 Sulfonyl Azide (SZ7)

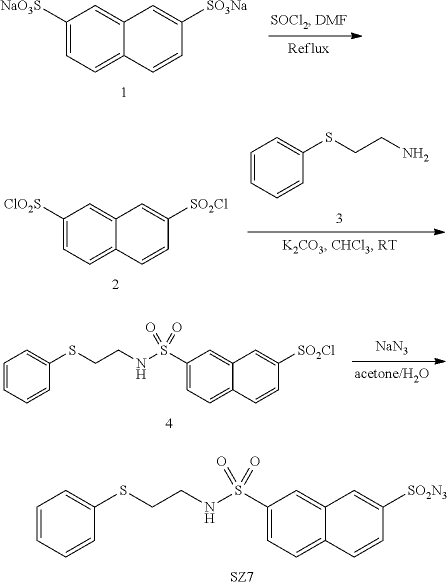

The mixture of compound 1 (664 mg, 2 mmol), SOCl₂ (4 mL), and DMF (0.1 mL) were refluxed for 2 h. Cold water (15 mL) was added, the mixture was extracted with DCM (4×15 mL), and the combined organic extracts were dried over Na₂SO₄. A quick filtration through a pad of silica gel, evaporation, and vacuum-drying gave the crude product 2 according to similar procedure (see Paruch et al., J. Org. Chem.; 2000; 65, 8774-8782). And compound 2 was used directly for next step.

A solution of compounds 2 and 3 (155 mg, 1 mmol) and potassium carbonate (200 mg, 1.44 mmol) in CHCl₃, was stirred at room temperature for 12 hours. The reaction mixture was then concentrated, mixed with ethyl acetate (20 mL) and water (20 mL), and extracted by ethyl acetate (20 mL×3). The combined organic phases were dried over anhydrous sodium sulfate and concentrated. The obtained crude product 4 was dissolved in acetone and added a solution of sodium azide (70 mg, 1 mmol) in water dropwise at 0° C. The mixture was stirred at 0° C. for 3 hours. Ethyl acetate (20 mL) and saturated aqueous potassium carbonate solution (20 mL) were added to the mixture and after extraction with ethyl acetate (20 mL×3), the combined organic phases were dried over anhydrous sodium sulfate and concentrated. Product (SZ7) (315 mg, 70%) was obtained by flash chromatography (hexane:EtOAc=4:1; Rf=0.6 in hexane:EtOAc=1:1).

1.8 Sulfonyl Azide (SZ9)

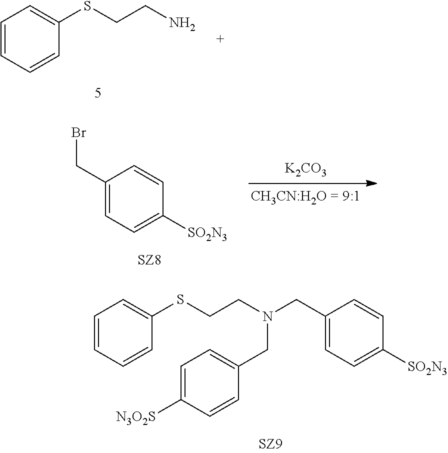

The mixture of 5 (276 mg, 1 mmol), (SZ8) (77 mg, 0.5 mmol) and potassium carbonate (200 mg, 1.44 mmol) in acetonitrile and water (9:1), was stirred at room temperature for 12 hours. After mixed with ethyl acetate (20 mL) and water (20 mL), the system was extracted by ethyl acetate (20 mL×3). The combined organic phase was dried by anhydrous sodium sulfate and concentrated. Product (SZ9) (154 mg, 60%) was obtained by flash chromatography (hexane:EtOAc=6:1; Rf=0.2 in hexane:EtOAc=4:1). ¹H-NMR (400 MHz, CDCl₃) δ: 7.87 (d, J=8.3 Hz, 4H), 7.57 (d, J=8.3 Hz, 4H), 7.24-7.15 (m, 5H), 3.72 (s, 4H), 3.07 (t, J=7.2 Hz, 2H), 2.76 (t, J=7.1 Hz, 2H) ppm. ¹³C-NMR (100 MHz, CDCl₃) δ: 146.6, 137.4, 129.6, 129.2, 129.0, 127.9, 127.6, 126.3, 57.9, 52.7, 31.5 ppm. HRMS (ESI⁺) for [M+H]⁺; calculated: 544.08899. found: 544.08874 (error m/z=−0.46 ppm).

1.9 Sulfonyl Azide (SZ10)

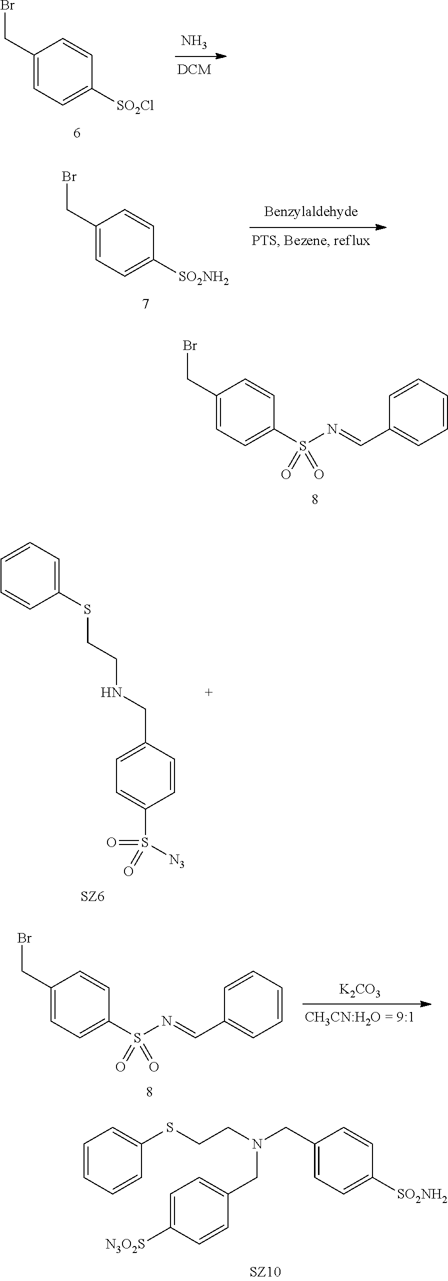

The solution of commercially available compound 6 (1 g, 3.74 mmol) in DCM was bubbled by ammonia gas at 0° C. for 10 min. After mixed with DCM (20 mL) and water (20 mL), the system was extracted by DCM (20 mL×3). The combined organic phase was dried by anhydrous sodium sulfate and concentrated. Product 7 (900 mg, 96%) was obtained by flash chromatography (hexane:EtOAc=3:1; Rf=0.5 in hexane:EtOAc=1:1). ¹H-NMR (400 MHz, DMSO-d6) δ: 7.81 (d, J=8.4 Hz, 2H), 7.63 (d, J=8.4 Hz, 2H), 7.39 (s, 2H), 4.76 (s, 2H) ppm. ¹³C-NMR (100 MHz, DMSO-d6) δ: 143.8, 141.9, 129.8, 126.0, 32.9 ppm.

The compound 7 (900 mg, 3.6 mmol), benzylaldehyde (381 mg, 3.6 mmol) and para-methylbenzylsulfonic acid (10 mg) in benzene had been refluxed for 10 h with Dean-stack condenser. The system was cooled down and extracted by ethyl acetate (20 mL×3). The combined organic phase was dried by anhydrous sodium sulfate, concentrated, and gave the product 8, which was used for next step directly. The mixture of 8, the known (SZ6) (1.25 g, 3.6 mmol) and potassium carbonate (1.0 g, 7.2 mmol) in acetonitrile and water (9:1), was refluxing for 24 hours. After cooled down and mixed with ethyl acetate (20 mL) and water (20 mL), the system was extracted by ethyl acetate (20 mL×3). The combined organic phase was dried by anhydrous sodium sulfate and concentrated. The interesting thing here is the hydrolyzation of the imine happened smoothly under this basic condition. And product (SZ10) (930 mg, 50%) was obtained by flash chromatography (hexane:EtOAc=2:1; Rf=0.2 in hexane:EtOAc=2:1). ¹H-NMR (400 MHz, CDCl₃) δ: 7.83 (d, J=8.4 Hz, 4H), 7.55 (d, J=8.4 Hz, 2H), 7.47 (d, J=8.4 Hz, 2H), 7.24-7.14 (m, 5H), 5.00 (bs, 2H), 3.68 (s, 2H), 3.67 (s, 2H), 3.06 (t, J=6.8 Hz, 2H), 2.75 (t, J=6.8 Hz, 2H) ppm. ¹³C-NMR (100 MHz, CDCl₃) δ: 147.0, 144.1, 140.9, 137.1, 135.8, 129.6, 129.2, 129.1, 128.9, 127.5, 126.5, 126.2, 58.1, 58.0, 53.0, 31.6 ppm. HRMS (ESI⁺) for [M+H]⁺; calculated: 518.09849. found: 518.09993 (error m/z=2.76 ppm).

1.10 Sulfonyl Azide (SZ11)

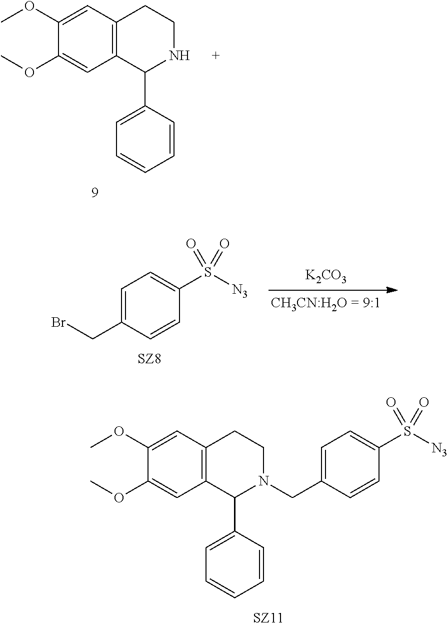

(SZ11) was prepared through the procedure described for the preparation of (SZ9) in 87% yield by using (SZ8) and known compound 9, which is prepared according to the reported method. ¹H-NMR (400 MHz, CDCl₃) δ: 7.82 (d, J=8.4 Hz, 2H), 7.53 (d, J=8.0 Hz, 2H), 7.36-7.22 (m, 5H), 6.62 (s, 1H), 6.20 (s, 1H), 4.57 (s, 1H), 3.83 (d, J=14.8 Hz, 1H), 3.80 (s, 3H), 3.56 (s, 3H), 3.39 (d, J=14.8 Hz, 1H), 3.00-2.98 (m, 2H), 2.71 (d, J=15.2 Hz, 1H), 2.52 (d, J=15.2 Hz, 1H) ppm. ¹³C-NMR (100 MHz, CDCl₃) δ: 147.8, 147.2, 146.8, 143.4, 136.3, 129.5, 129.2, 129.1, 128.1, 127.2, 127.0, 126.2, 111.4, 110.6, 68.1, 57.9, 55.4, 47.3, 28.2 ppm. HRMS (ESI⁺) for [M+H]⁺; calculated: 465.15965. found: 465.15970 (error m/z=0.1 ppm).

1.11 Sulfonyl Azide (SZ12)

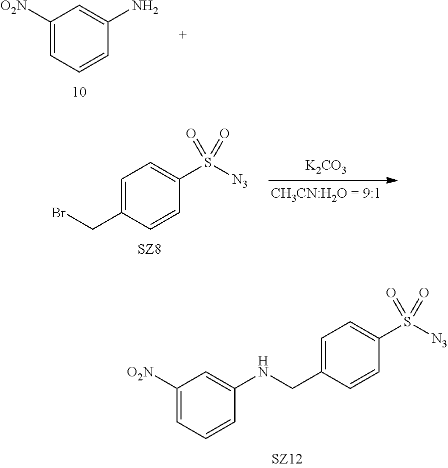

(SZ12) was prepared starting from 10 and (SZ8) using the procedure described for the preparation of (SZ9) in 67% yield. ¹H-NMR (400 MHz, CDCl₃) δ: 7.90 (d, J=6.8 Hz, 2H), 7.59-7.25 (m, 5H), 6.84 (d, J=7.6 Hz, 1H), 4.53 (s, 2H) ppm. ¹³C-NMR (100 MHz, CDCl₃) δ: 148.0, 146.2, 130.0, 128.3, 128.1, 128.0, 127.6, 118.7, 112.8, 106.7, 47.3 ppm. HRMS (ESI⁺) for [M+K]⁺; calculated: 372.01633. found: 372.01449 (error m/z=−4.94 ppm).

1.12 Sulfonyl Azide (SZ13)

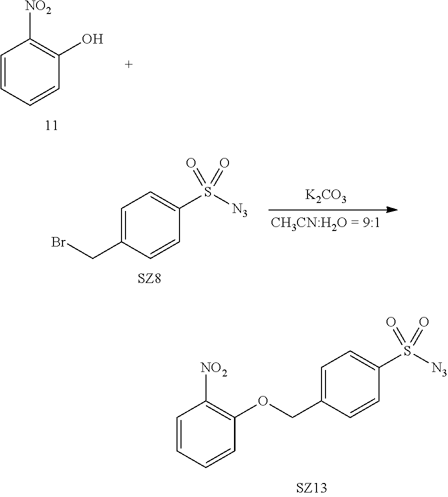

(SZ13) was prepared starting from 11 and (SZ8) using the procedure described for the preparation of (SZ9) in 40% yield. ¹H-NMR (400 MHz, CDCl₃) δ: 7.89-7.84 (m, 3H), 7.61-7.33 (m, 5H), 4.48 (s, 2H) ppm. ¹³C-NMR (100 MHz, CDCl₃) δ: 143.2, 141.1, 134.3, 134.0, 129.0, 128.5, 127.7, 125.8, 125.1, 53.6 ppm. HRMS (ESI⁺) for [M+NH₄]⁺; calculated: 352.0716. found: 352.0719 (error m/z=0.8 ppm).

1.13 Sulfonyl Azide (SZ14)

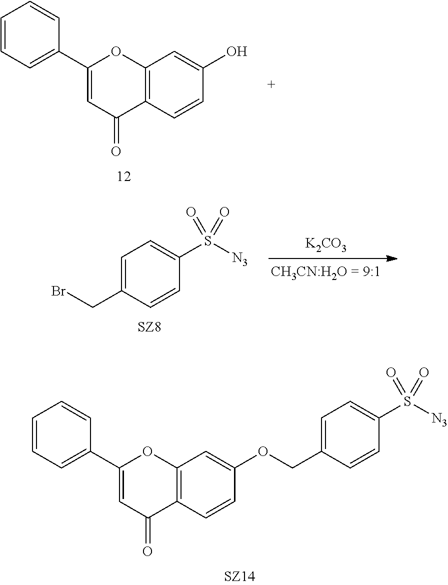

(SZ14) was prepared starting from 12 and (SZ8) using the procedure described for the preparation of (SZ9) in 45% yield. ¹H-NMR (400 MHz, CDCl₃) δ: 8.08 (dd, J=8.8, 3.6 Hz, 1H), 7.87-7.82 (m, 4H), 7.50-7.48 (m, 5H), 7.36 (d, J=2.0 Hz, 1H), 6.91 (dd, J=8.8, 2.0 Hz, 1H), 6.75 (d, J=4.0 Hz, 1H) 4.47 (s, 2H) ppm. ¹³C-NMR (100 MHz, CDCl₃) δ: 177.1, 163.8, 156.3, 152.7, 142.8, 134.4, 131.8, 131.0, 129.0, 128.9, 128.6, 127.4, 126.2, 122.6, 119.4, 112.0, 107.6, 53.6 ppm. HRMS (ESI⁺) for [M+H]⁺; calculated: 434.08052. found: 434.07955 (error m/z=−2.24 ppm).

1.14 Sulfonyl Azide (SZ15)

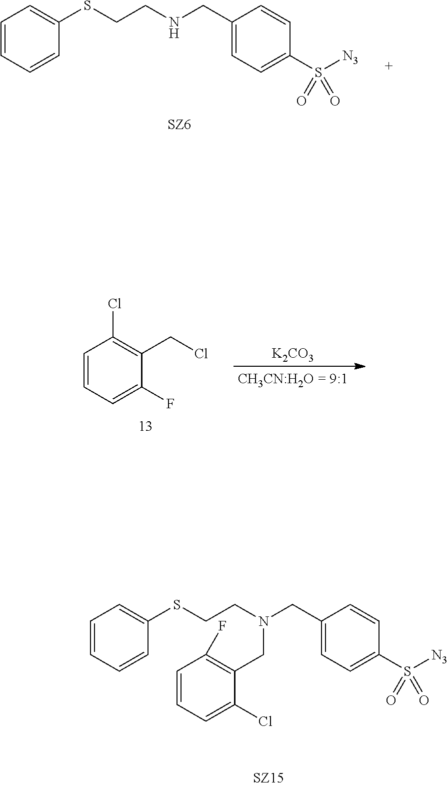

(SZ15) was prepared starting from 13 and known (SZ6) using the procedure described for the preparation of (SZ9) in 54% yield. ¹H-NMR (400 MHz, CDCl₃) δ: 7.78 (d, J=8.0 Hz, 2H), 7.51 (d, J=8.0 Hz, 2H), 7.18-7.11 (m, 7H), 6.94-6.90 (m, 1H), 3.85 (s, 2H), 3.73 (s, 2H), 3.05 (t, J=7.6 Hz, 2H), 2.77 (t, J=7.6 Hz, 2H) ppm. ¹³C-NMR (100 MHz, CDCl₃) δ: 161.9 (d, ¹J_{CF}=247 Hz), 147.8, 136.6, 136.3, 135.8, 129.5, 129.4, 128.9 (d, ²J_{CF}=26 Hz), 127.1, 126.0, 125.5, 124.1 (d, ³J_{CF}=16.8 Hz), 113.9 (d, ²J_{CF}=23.3 Hz), 57.8, 53.6, 49.4, 31.1 ppm.

1.15 Sulfonyl Azide (SZ16)

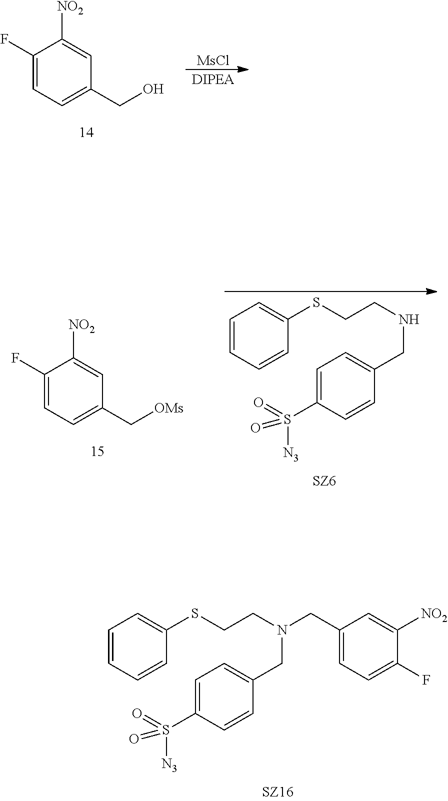 [SMILES: "MsCl" ; O=[N+]([O-])c1cc(CO)ccc1F ; CS(=O)(=O)OCc1ccc(F)c([N+](=O)[O-])c1 ; [N-]=[N+]=NS(=O)(=O)c1ccc(CNCCSc2ccccc2)cc1 ; [N-]=[N+]=NS(=O)(=O)c1ccc(CN(CCSc2ccccc2)Cc2ccc(F)c([N+](=O)[O-])c2)cc1]

(SZ16) was prepared by two steps. First, the mixture of 14 (570 mg, 3.3 mmol) and mesyl chloride (0.45 ml, 5.2 mmol) in N,N-Diisopropylethylamine (10 ml) was stirred for 3 hours and then (SZ6) (1.15 g, 3.3 mmol) was added. After stirring for another 3 hours, 30 ml ethyl acetate and 30 ml CuSO₄ aqueous solution were added. The aqueous phase was extracted twice with 30 ml ethyl acetate and the combined organic phase is dried over Na₂SO₄ and concentrated down. (SZ16) (310 mg, 19% over two steps) was obtained by flash chromatography (hexane:EtOAc=6:1; Rf=0.3 in hexane:EtOAc=2:1). ¹H-NMR (400 MHz, CDCl₃) δ: 8.01 (d, J=6.0 Hz, 1H), C 7.58-7.56 (m, 3H), 7.23-7.14 (m, 6H), 3.70 (s, 2H), 3.63 (s, 2H), 3.05 (t, J=7.3 Hz, 2H), 2.74 (t, J=7.6 Hz, 2H) ppm. ¹³C-NMR (100 MHz, CDCl₃) δ: 154.6 (¹J_{CF}=263.5 Hz), 146.6, 137.3, 135.7 (²J_{CF}=24.4 Hz), 135.3 (³J_{CF}=8.4 Hz), 129.6, 129.2, 128.9, 127.6, 126.3, 125.8, 118.4 (²J_{CF}=20.6 Hz) ppm. HRMS (ESI⁺) for [M+H]⁺; calculated: 502.10135. found: 502.09963 (error m/z=−3.42 ppm).

1.16 Sulfonyl Azide (SZ17)

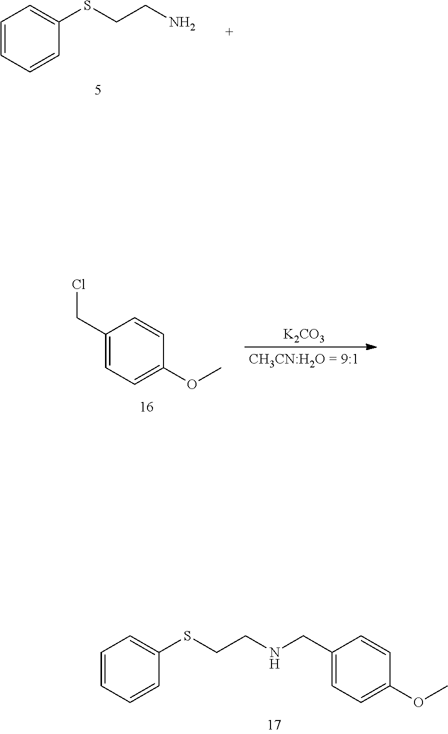

(SZ17) was prepared by two steps. First, the mixture of 5 (500 mg, 3.3 mmol), 16 (5.20 mmol) and potassium carbonate (1.0 g, 7.2 mmol) in acetonitrile and water (9:1, 20 mL), was stirred at room temperature for 12 hours. After mixed with ethyl acetate (20 mL) and water (20 mL), the system was extracted by ethyl acetate (20 mL×3). The combined organic phase was dried by anhydrous sodium sulfate and concentrated. Intermediate 17 (330 mg, 37%) was obtained by flash chromatography (hexane:EtOAc=1:1-1:3; Rf=0.2 in hexane:EtOAc=1:1). ¹H-NMR (400 MHz, CDCl₃) δ: 7.37-7.16 (m, 6H), 6.91-6.89 (m, 2H), 6.81 (d, J=8.4 Hz, 1H), 3.7-3.77 (m, 5H), 3.09 (t, J=6.4 Hz, 2H), 2.87 (t, J=6.0 Hz, 2H), 1.72 (s, 1H) ppm. ¹³C-NMR (100 MHz, CDCl₃) δ: 159.5, 141.6, 135.6, 129.3, 129.1, 128.6, 125.8, 120.1, 113.2, 112.2, 54.8, 53.1, 47.3, 33.9 ppm.

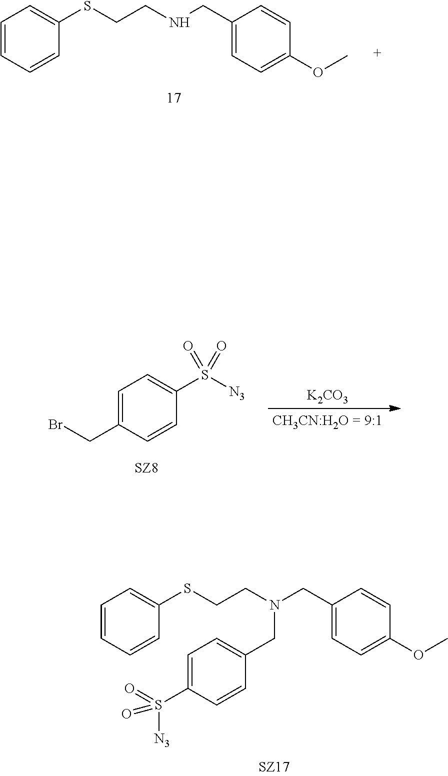

(SZ17) was prepared starting from 17 and known (SZ8) using the procedure described for the preparation of (SZ9) in 54% yield. ¹H-NMR (400 MHz, CDCl₃) δ: 7.86 (d, J=8.0 Hz, 2H), 7.60 (d, J=8.0 Hz, 2H), 7.27-7.13 (m, 6H), 6.99-6.95 (m, 2H), 6.81 (dd, J=8.0, 2.0 Hz, 1H), 3.81 (s, 3H), 3.69 (s, 2H), 3.63 (s, 2H), 3.07 (t, J=7.2 Hz, 2H), 2.76 (t, J=7.2 Hz, 2H) ppm. ¹³C-NMR (100 MHz, CDCl₃) δ: 159.5, 147.6, 140.1, 136.6, 136.0, 129.4, 129.2, 128.7, 128.5, 127.7, 127.2, 125.7, 120.8, 114.2, 112.4, 58.2, 57.5, 54.9, 52.6, 31.1 ppm.

1.17 Thio Acid (TA2)

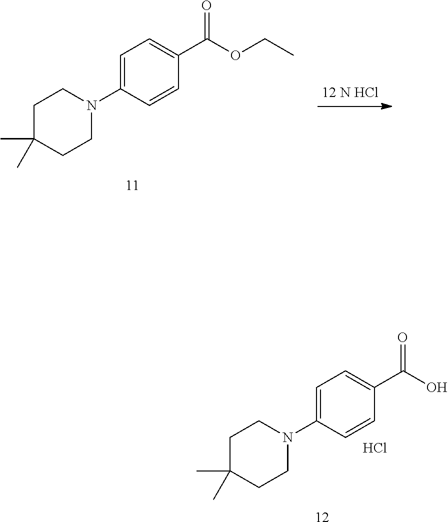

A mixture of known 11 (Kobayashi et al., Synthesis 1985, 6-7, 671-2) (6.0 g, 23 mmol) and 12 N HCl (40 mL) was kept at refluxing temperature overnight. The reaction mixture was slowly cooled down and white crystals precipitated. The flask was then cooled to 0° C. for 10 minutes and the mixture was quickly filtrated. The crystals were washed with dichloromethane yielding the known product 12 (4.5 g, 73.2%). ¹H-NMR (250 MHz, CDCl₃) δ: 7.94 (d, J=6.2 Hz, 2H), 7.67 (bs, 2H), 3.43 (bs, 4H), 1.69 (bs, 4H), 1.01 (s, 6H) ppm. HRMS (ESI⁺) for [M+H]⁺; calculated: 234.14866. found: 234.14797 (error m/z=−3.79 ppm).

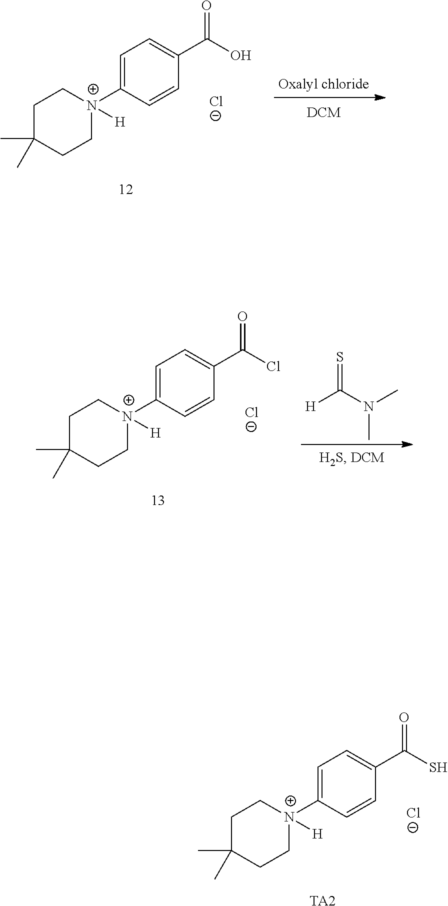

Oxalyl chloride (740 mg, 0.5 mL, 5.83 mmol) was added dropwise into a solution of 12 (500 mg, 1.86 mmol) in dichloromethane at 0° C. The mixture was then stirred at room temperature for 8 hours. Then, dimethylthioformamide (0.6 mL, 6.7 mmol) was added to the above solution, and hydrogen sulfide was passed through the reaction mixture for 10 minutes at a moderate rate. The course of the reaction was monitored by TLC and once compound 13 completely disappeared, the addition of hydrogen sulfide was stopped. An excess of hexanes was added until a yellow powder precipitated. The powder was collected by filtration and product (TA2) (400 mg, 75% yield) was obtained by quick flash chromatography (DCM:MeOH=30:1). Rf=0.65 (DCM:MeOH=18:1) in the absence of light. ¹H-NMR (400 MHz, CDCl₃) δ: 7.75 (d, J=8.4 Hz, 2H), 6.80 (d, J=8.4 Hz, 2H), 4.32 (bs, 1H), 3.36-3.33 (m, 4H), 1.48-1.45 (m, 4H), 0.98 (s, 6H) ppm. ¹³C-NMR (100 MHz, CDCl₃) δ: 187.7, 154.6, 130.1, 125.2, 112.7, 43.8, 37.8, 28.6, 27.7 ppm. HRMS (ESI⁺) for [M+H]⁺; calculated: 250.12601. found: 250.12593 (error m/z=−0.33 ppm).

1.18 Thio Acid (TA3)

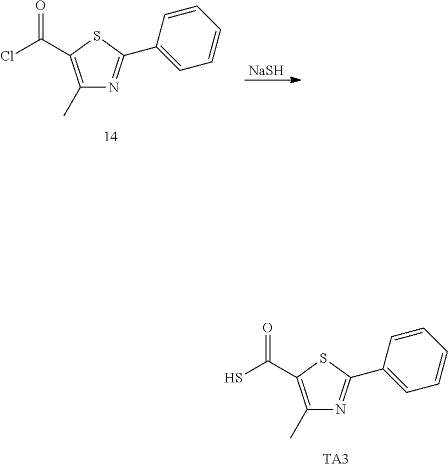

Compound 14 (300 mg, 1.26 mmol) was added slowly to a saturated solution of sodium hydrogensulfide (211 mg, 3.76 mmol) in water at room temperature. The mixture was stirred at room temperature for 3 hours, ethyl acetate (50 mL) was then added followed by saturated aqueous potassium carbonate solution (50 mL). After extraction by ethyl acetate (50 mL×3), the combined organic phases were dried over anhydrous sodium sulfate and concentrated. The product (TA3) (200 mg, 67%) was obtained by quick flash chromatography (hexanes:EtOAc=1:1). Rf=0.25 in hexane:EtOAc=1:1) in the absence of light. ¹H-NMR (400 MHz, CD₃OD) δ: 6.36 (bs, 2H), 5.90 (bs, 3H), 1.76 (bs, 1H), 1.12 (s, 3H) ppm. ¹³C-NMR (100 MHz, CD₃OD) δ: 203.6, 168.8, 152.1, 142.4, 134.6, 131.5, 130.1, 127.4, 17.6 ppm. HRMS (ESI⁺) for [M+H]⁺; calculated: 236.01983. found: 236.01982 (error m/z=−0.04 ppm).

1.19 Thio Acid (TA4)

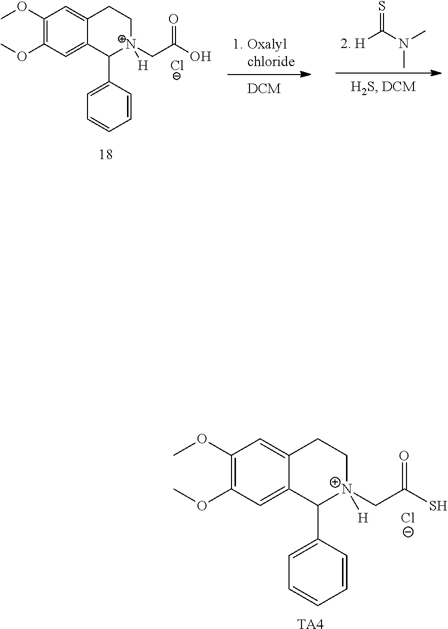

The synthesis of (TA4) was accomplished via the same procedure as described for (TA3). ¹H-NMR (400 MHz, CDCl₃) δ: 7.35-7.15 (m, 5H), 6.66 (s, 1H), 6.25 (s, 1H), 5.53 (s, 1H), 3.85 (s, 3H), 3.81 (bs, 2H), 3.65 (s, 3H), 3.43-3.30 (m, 2H), 3.22-3.00 (m, 2H) ppm. ¹³C-NMR (100 MHz, CDCl₃) δ: 149.7, 148.8, 136.5, 130.6, 130.1, 129.5, 129.4, 123.6, 111.3, 111.0, 66.3, 63.6, 56.2, 56.0, 45.4, 45.4 ppm. HRMS (ESI⁺) for [M]⁺; calculated: 344.13149. found: 344.13149 (error m/z=−0.02 ppm).

1.20 Thio Acid (TA5)

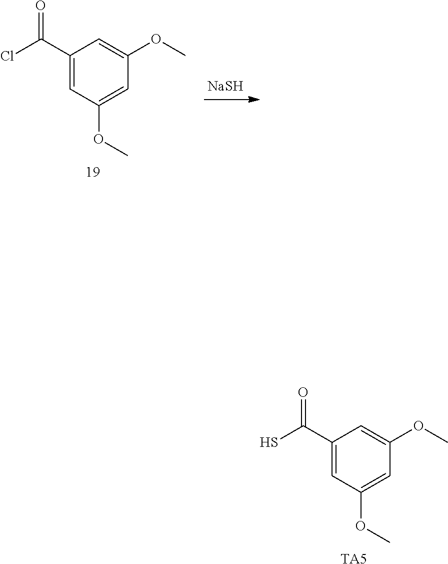

The synthesis of (TA5) was accomplished via the same procedure as described for (TA2). ¹H-NMR (400 MHz, CDCl₃) δ: 7.08 (s, 1H), 7.07 (s, 1H), 6.72 (s, 1H), 3.88 (s, 3H), 3.88 (s, 3H) ppm. ¹³C-NMR (100 MHz, CDCl₃) δ: 190.0, 160.7, 138.4, 106.1, 105.6, 55.5 ppm. HRMS (ESI⁺) for [M]⁺; calculated: 344.13149. found: 344.13149 (error m/z=−0.02 ppm).

1.21 Thio Acid (TA6)

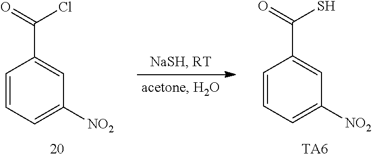

To a solution of NaSH (90 mg, 1.6 mmol) in water (1 ml) was added dropwise a solution of acid chloride in acetone (6 ml). The resulting mixture was stirred for 3 h. The solvent was removed under reduced pressure and resulting crude was basified using 10% NaOH solution (pH=12). The solution was slowly acidified using 2N HCl solution (pH=1). Corresponding thio acid (TA6) crashed out and was filtered, washed with deionized water and dried under vacuum to obtain pale yellow crystals of (TA6). ¹H-NMR (400 MHz, CDCl₃) δ: 8.72-8.71 (m, 1H), 8.46-8.43 (m, 1H), 8.2 (d, J=7.6 Hz, 1H), 7.68 (t, J=8 Hz, 1H) ppm. ¹³C-NMR (100 MHz, CDCl₃) δ: 188.2, 137.9, 133.4, 130.3, 128.3, 122.9 ppm.

1.22 Thio Acid (TA7)

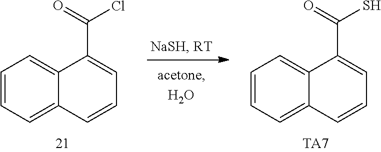

The synthesis of (TA7) was accomplished via the same procedure as described for (TA6). ¹H-NMR (400 MHz, CDCl₃) δ: 8.59-8.52 (m, 1H), 8.34 (d, J=7.2 Hz, 1H), 8.08 (m, 1H), 7.88 (m, 1H), 7.57 (m, 3H) ppm. ¹³C-NMR (100 MHz, CDCl₃) δ: 188.1, 134.3, 133.4, 129.5, 129.0, 128.8, 128.65, 127.3, 127.0, 125.4, 124.7 ppm.

1.23 Other Thioacids

Other thioacids described herein were prepared, in general, in accordance with the methods described above in Examples 1.1 to 1.22 and conventional organic synthesis techniques.

### Example 2

### Reaction/Incubation Procedures and LC/MS Measurements

2.1 General Procedure for Incubations of Bcl-X_{L} with Reactive Fragments

In a 96-well plate, one thio acid building block (1 μL of a 2 mM solution in methanol) and one sulfonyl azide building block (1 μL of a 2 mM solution in methanol) were added to a solution of Bcl-X_{L} (98 μL of a 2 μM Bcl-X_{L} solution in buffer (58 mM Na₂HPO₄, 17 mM NaH₂PO₄, 68 mM NaCl, 1 mM NaN₃, pH=7.40)). The 96-well plate was sealed and incubated at 38.5° C. for six hours. The incubation samples were then subjected to liquid chromatography combined with mass spectrometry analysis in the selected ion mode (LC-MS-SIM, Zorbax SB-C18 preceded by a Phenomenex C18 guard column, electrospray ionization and mass spectroscopic detection in the positive selected ion mode, tuned to the expected molecular mass of the product). The TGS hit compound was identified by the mass and retention time. As a control, identical building block combinations were incubated in buffer without Bcl-X_{L} and subjected to LC/MS-SIM analysis. Comparison of the LC-MS-SIM chromatograms of these control incubations with the chromatograms of the Bcl-X_{L} containing incubations allows to determine whether the protein is templating the corresponding amidation reactions or not. For the Bcl-X_{L} containing incubation sample showing acylsulfonamide formation, a second control has been undertaken. Synthetically prepared acylsulfonamide was subjected to LC/MS-SIM analysis and the retention time was compared with the retention time identified in the Bcl-X_{L} containing incubation.

The gradient used for LC/MS-SIM is shown below:

| Time | B * | Flowrate |
|---|---|---|
| 0 | 10% | 0.7 mL/min |
| 4 | 20% | 0.7 mL/min |
| 12 | 100% | 0.7 mL/min |
| 13 | 100% | 0.7 mL/min |
| 13.01 | 100% | 1.5 mL/min |
| 15.00 | 100% | 1.5 mL/min |
| 15.50 | 20% | 1.5 mL/min |
| 16.50 | 20% | 1.0 mL/min |
| 16.51 | 20% | 0 mL/min |

* eluant A: H₂O (0.05% TFA); eluant B: CH₃CN 0.05% TFA)

2.2 Incubations at Various Bcl-X_{L} Concentrations

Different concentrations of Bcl-X_{L} were explored to determine the ideal protein concentrations for the incubations with building blocks (SZ4) and (TA2). The minimal protein concentration for obtaining a good ratio between templated and non-templated reactions is 2 μM. Incubations at higher concentrations give only slightly better ratios between templated and non-templated reactions. Hence, we determined 2 μM Bcl-X_{L} to be the most economical with regard to the protein consumption.

2.3 Comparison Between Incubations of SZ4 and TA2 Measured by the LC/MS-SIM and the LC/MS-Scan Mode

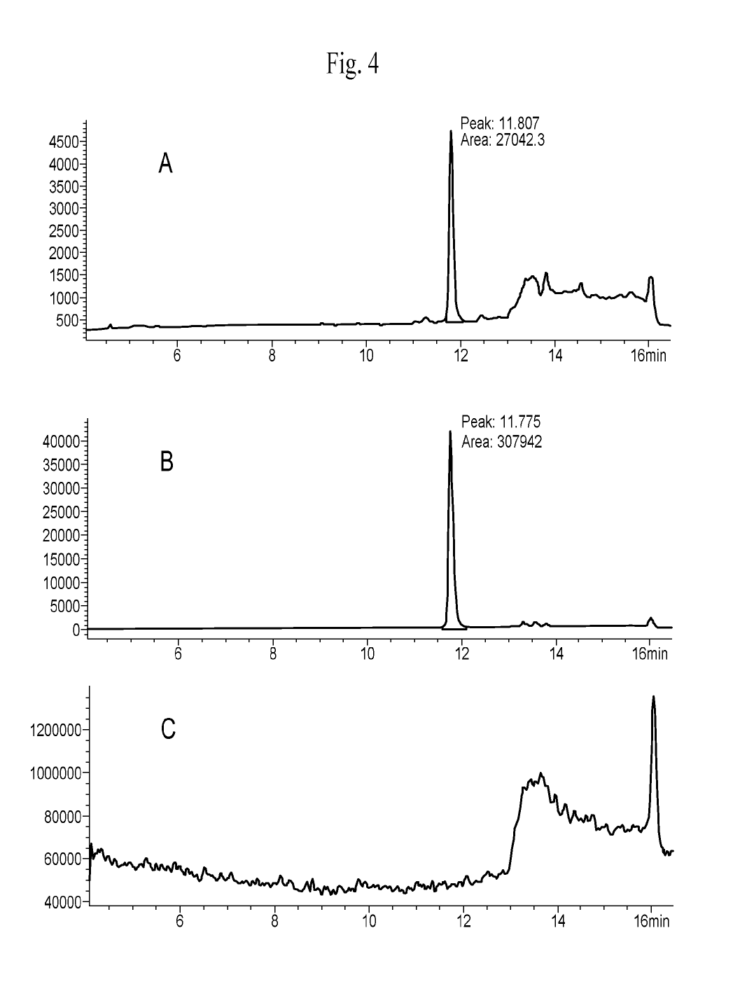

The sensitivity of the LC/MS can be significantly increased by utilizing the MS instrument in the selected ion monitoring (LC/MS-SIM). The advantage of LC/MS-SIM over LC/MS-Scan for kinetic TGS has been previously reported (Manetsch et al., J. Am. Chem. Soc. 2004; 126, 12809-12818). See FIG. 4.

2.4 TGS Screening Criteria and Two Examples of TGS Incubation Samples Failing at Templating the Formation of Acylsulfonamides

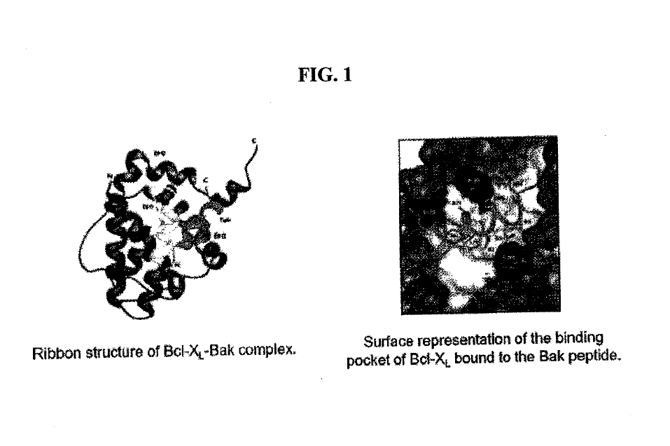

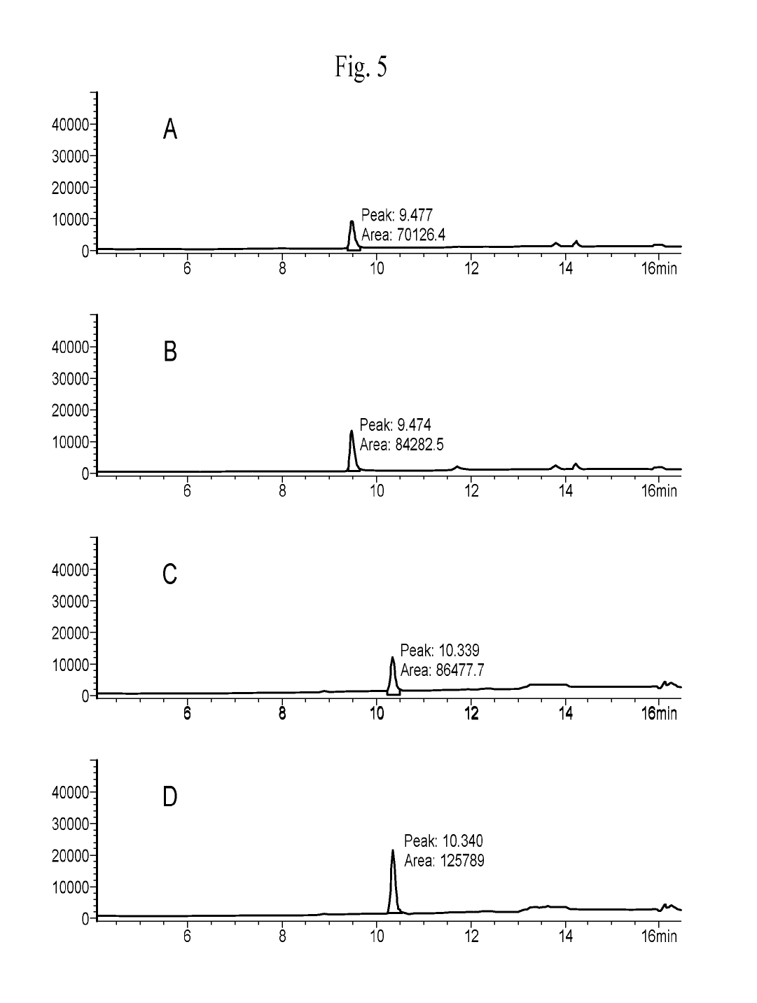

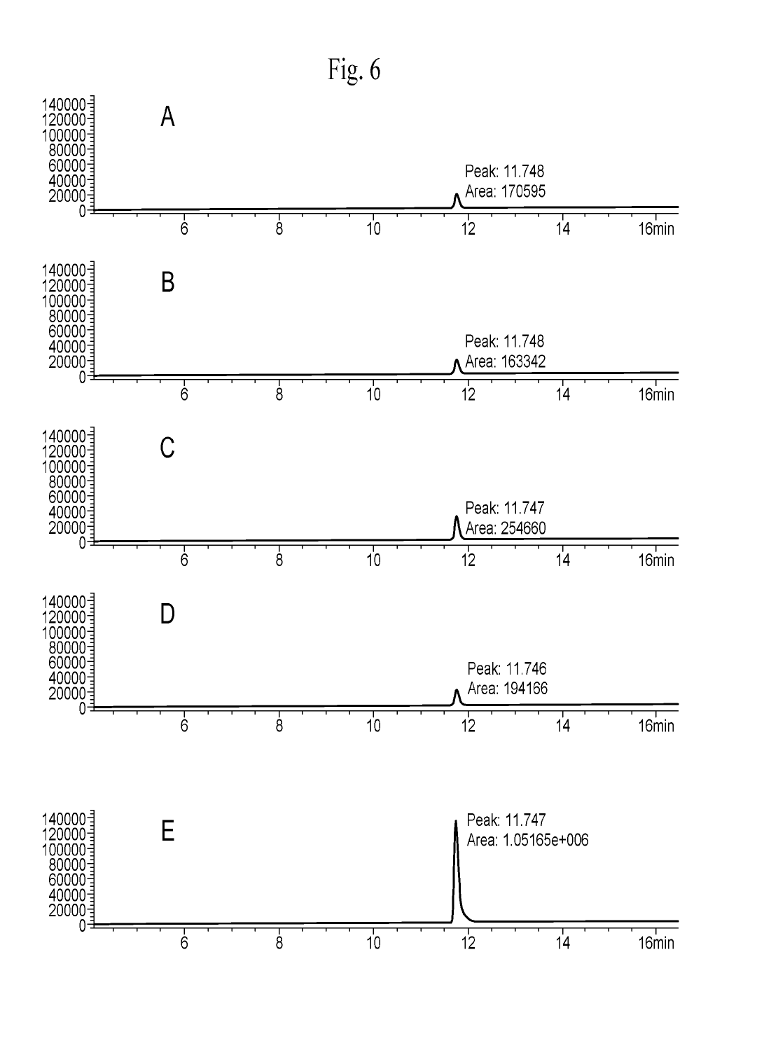

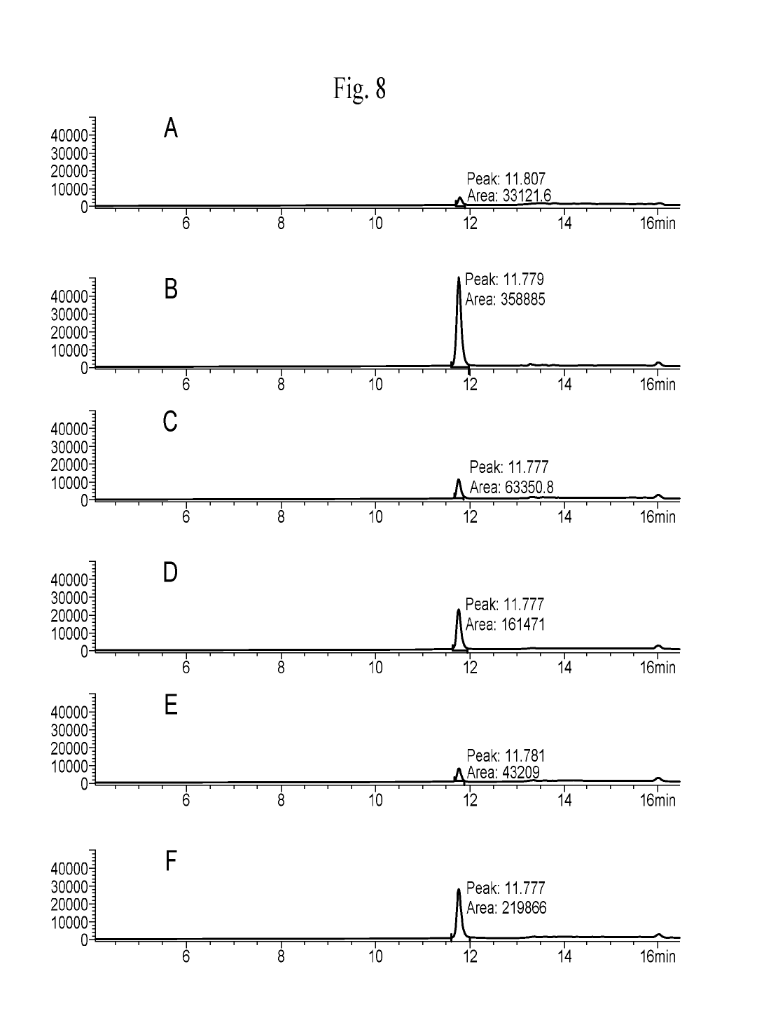

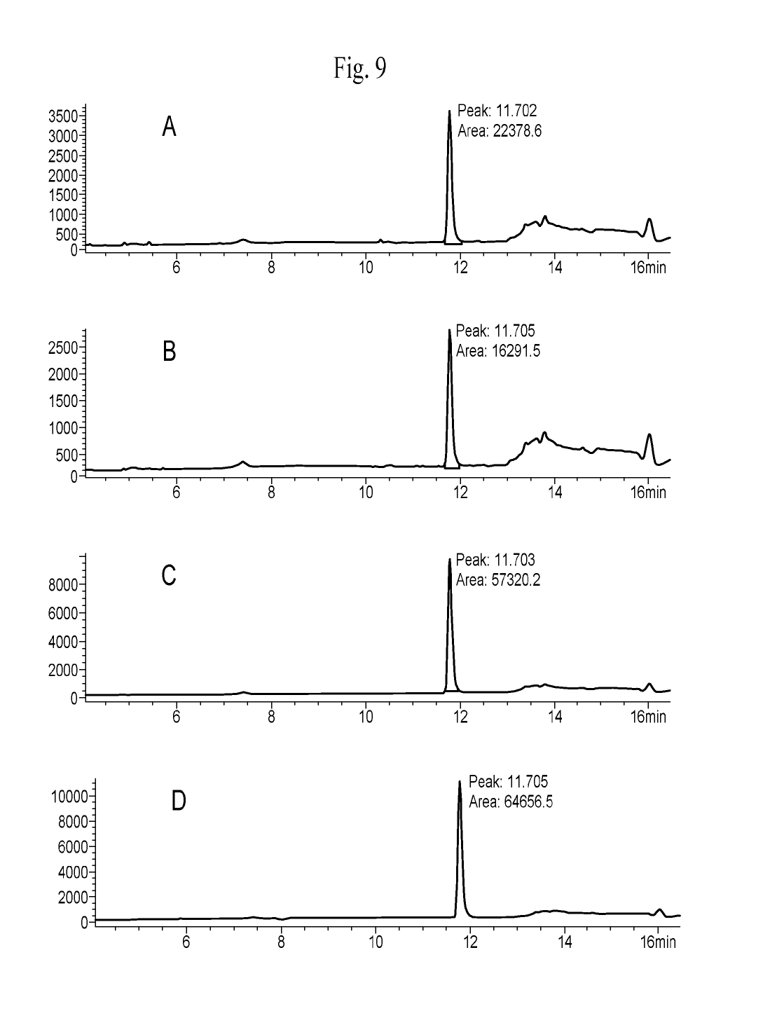

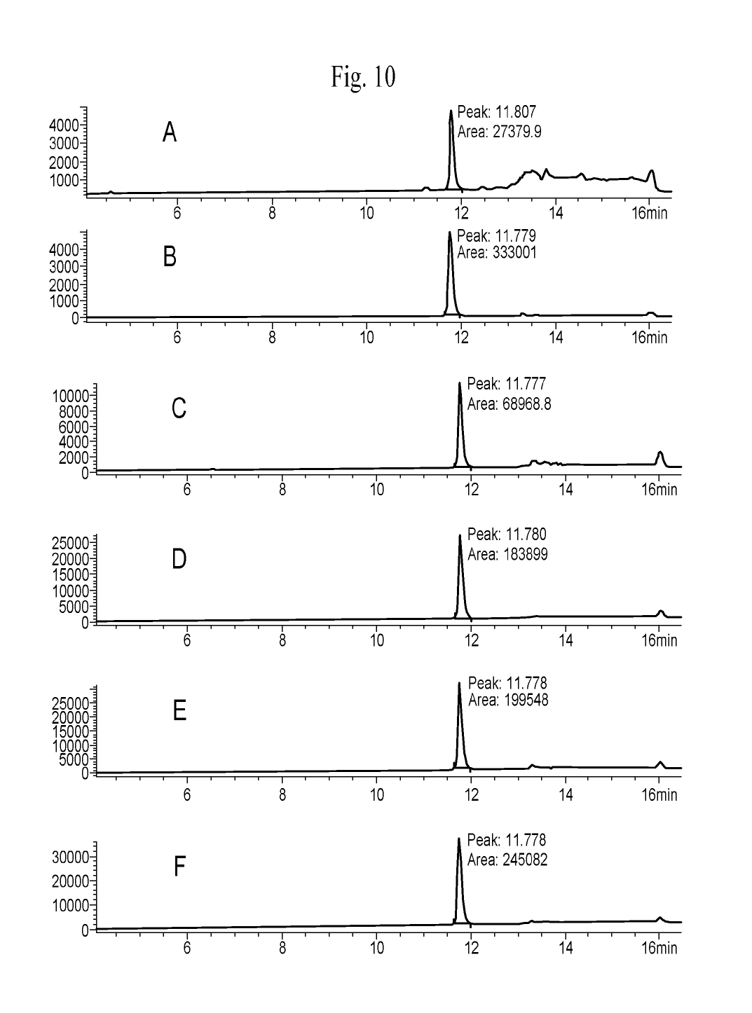

Examples of incubation samples failing at templating the formation of acylsulfonamides are depicted in FIG. 5. To determine whether a building block combination is a TGS hit or not, the ratio between the peak areas of the Bcl-X_{L}-templated reaction over the peak area of the incubation without Bcl-X_{L} is calculated. If this ratio is greater than 4, we consider this particular combination to be a TGS hit combination. Further control incubation experiments (see FIG. 1 in the communication, FIG. 6, FIG. 8, FIG. 9, and FIG. 10) are performed to fully validate this particular TGS hit.

2.5 Incubations of (SZ4) and (TA2) with Bovine Erythrocyte Carbonic Anhydrase II, Concanavalin A and Mouse Acetylcholinesterase.

The building blocks (SZ4) and (TA2) were incubated with proteins (2 M) bovine erythrocyte carbonic anhydrase II (bCAII), concanavalin A (ConA) and mouse acetylcholinesterase (mAChE) respectively to test whether these proteins can also template the formation of acylsulfonamide (SZ4TA2). Incubations at 37° C. for 24 hours failed at yielding pronounced amounts of (SZ4TA2).

2.6 Suppressing Bcl-X_{L}-templated Incubations with Bak BH3 Peptide

Additional control experiments have been performed to test whether the Bcl-X_{L}-templated reaction occurs at the BH3 binding site on Bcl-X_{L}. Reactive building blocks (SZ4) and (TA2) were incubated with Bcl-X_{L} and pro-apoptotic Bak BH3 peptide. Bak is one of the natural Bcl-X_{L} ligands and theoretically competes with the reactive building blocks for binding on Bcl-X_{L} during the incubations. The influence of Bak BH3 peptide on the Bcl-X_{L}-templated reaction was studied at different ratios of Bak BH3 peptide and Bcl-X_{L} (FIG. 10). At 20 μM Bak BH3 peptide, the templated reaction is significantly suppressed compared to the Bcl-X_{L} incubation without Bak BH3.

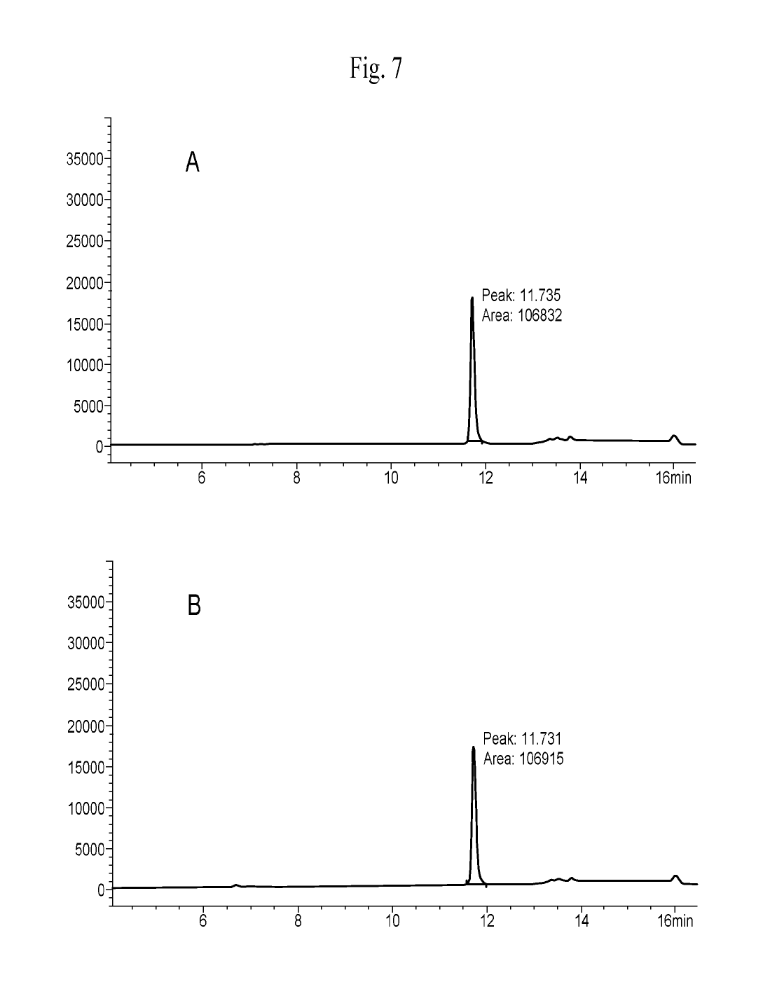

As an additional control experiment, the incubation of (SZ4) and (TA2) with Bak BH3 peptide was carried out for 24 hours (FIG. 7) demonstrating that Bak BH3 can not template the formation of (SZ4TA2).

The sequence of the herein utilized Bak BH3 peptide is the following:

BakBH3 (wt): CMGQVGRQLAIIGDDINRRYDS

2.7 Suppressing Bcl-X_{L}-Templated Incubations with Bim, Mutant Bim and Mutant Bak.

Additional control experiments have been performed with Bim, mutant Bim, and mutant Bak. Mutant Bim and mutant Bak are known to bind with lower affinity towards Bcl-X_{L} compared to wildtype Bak BH3 and Bim peptides. Therefore, the Bcl-X_{L} incubations containing mutant Bak BH3 and mutant Bim display an increased amount of acylsulfonamide (SZ4TA2) compared to the Bcl-X_{L} incubations containing wild type Bak BH3 and Bim peptides. The sequences of the various peptides are shown below:

BakBH3 (wt): CMGQVGRQLAIIGDDINRRYDS
(see Figure 7 and Figure 10)
BakBH3 (mt): CMGQVGRQAAIIGADINRRYDS
BimBH3 (wt): CEIWIAQELRRIGDEFNAYYAR
BimBH3 (mt): CEIWIAQEARRIGAEFNAYYAR

2.8 Bcl-X_{L} Incubations Containing More than Two Reactive Building Blocks.

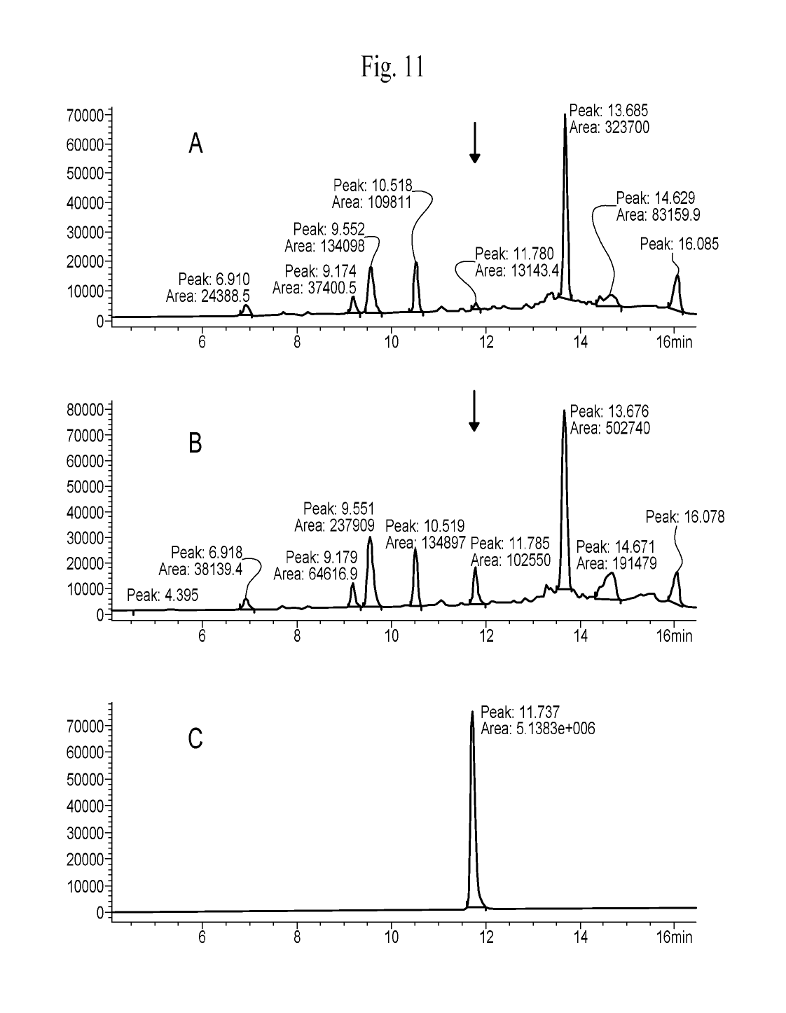

Initial experiments have been performed to investigate whether our TGS screening via the amidation reaction can be performed with incubations containing more than two complimentary reactive building blocks. Previously, this has been shown to be applicable for standard in situ ligation chemistry approaches (Manetsch et al., supra; Krasinski et al., J. Am. Chem. Soc. 2005, 127, 6686-6692). Our study revealed that the incubation sample containing 1 thioacid and 6 sulfonylazides (FIG. 11 with (TA2), (SZ1)-(SZ6)) can give the same results as multiple incubations containing only two complimentary reacting building blocks. Attempts to run an incubation containing simultaneously 3 thioacids (TA1)-(TA3) and 6 azides (SZ1)-(SZ6) failed with 2 μM as well as 10 μM Bcl-X_{L} concentrations.

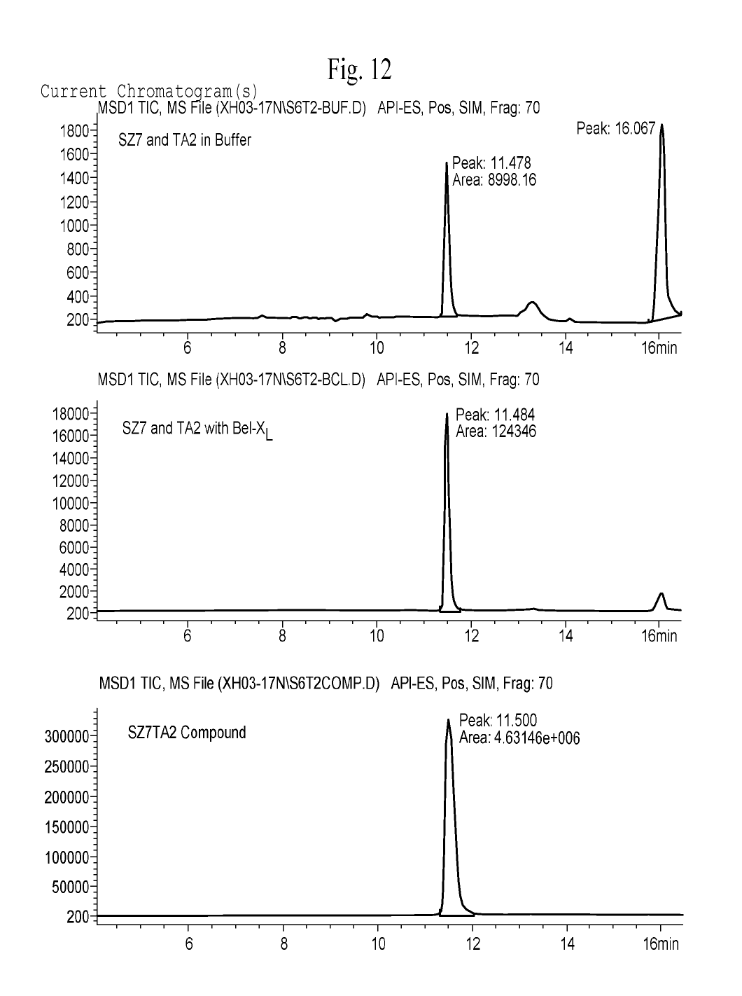

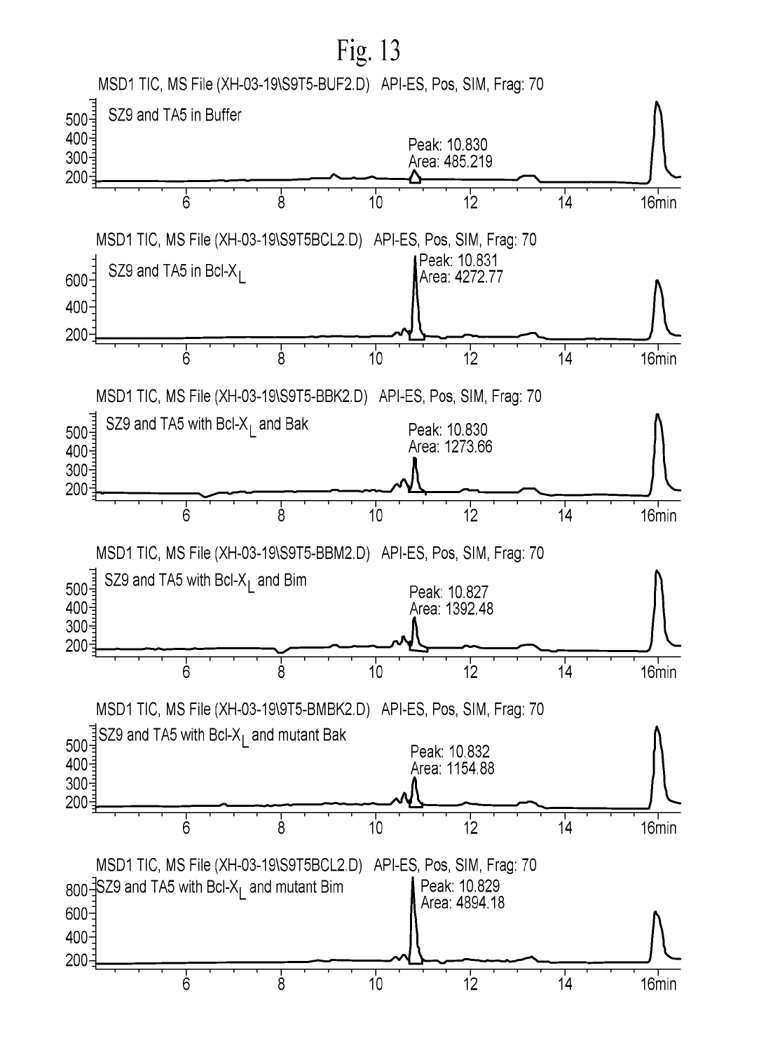

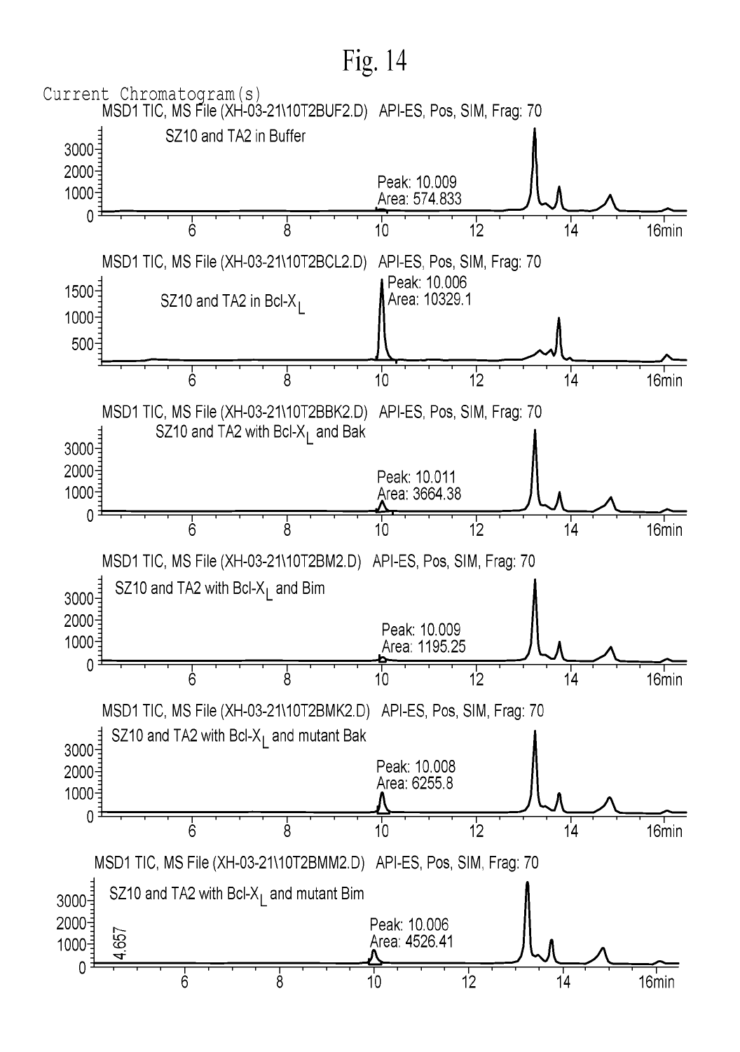

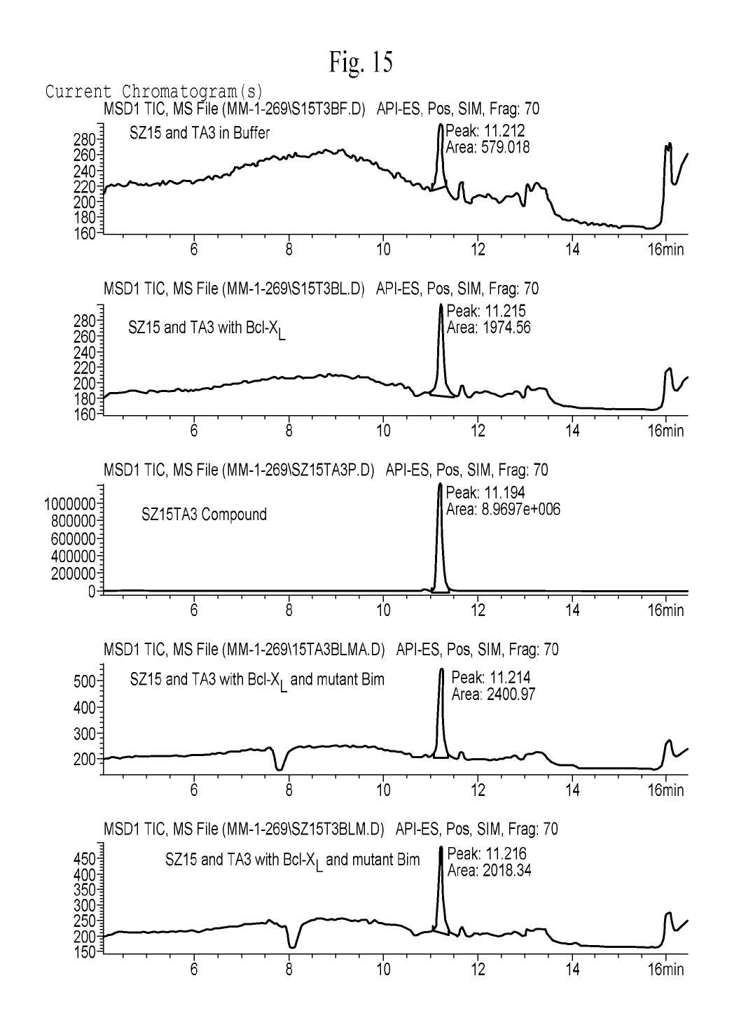

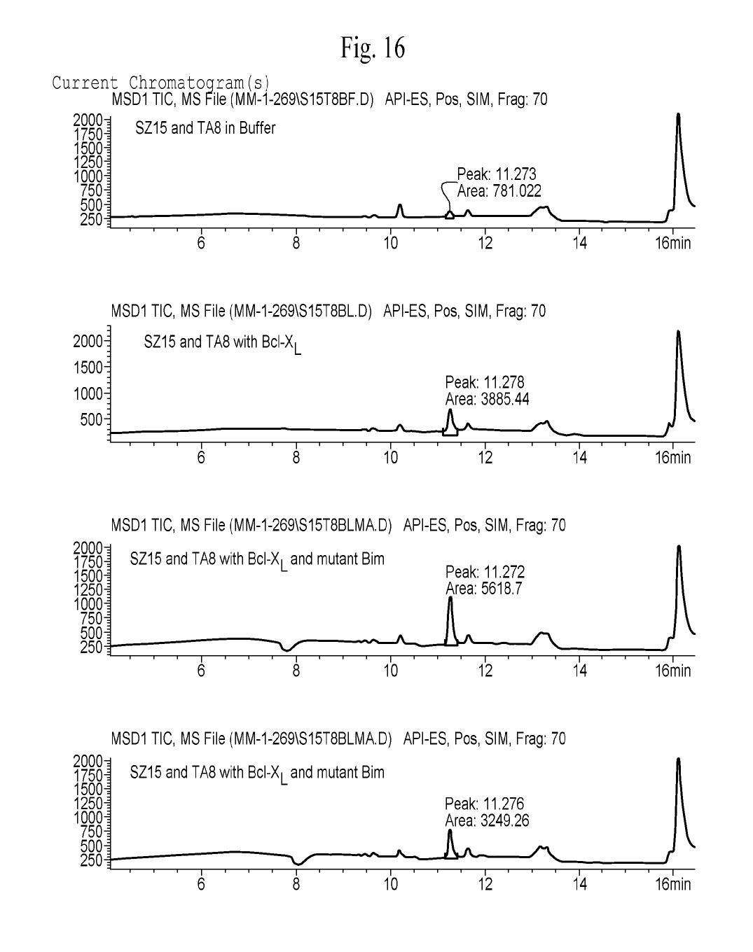

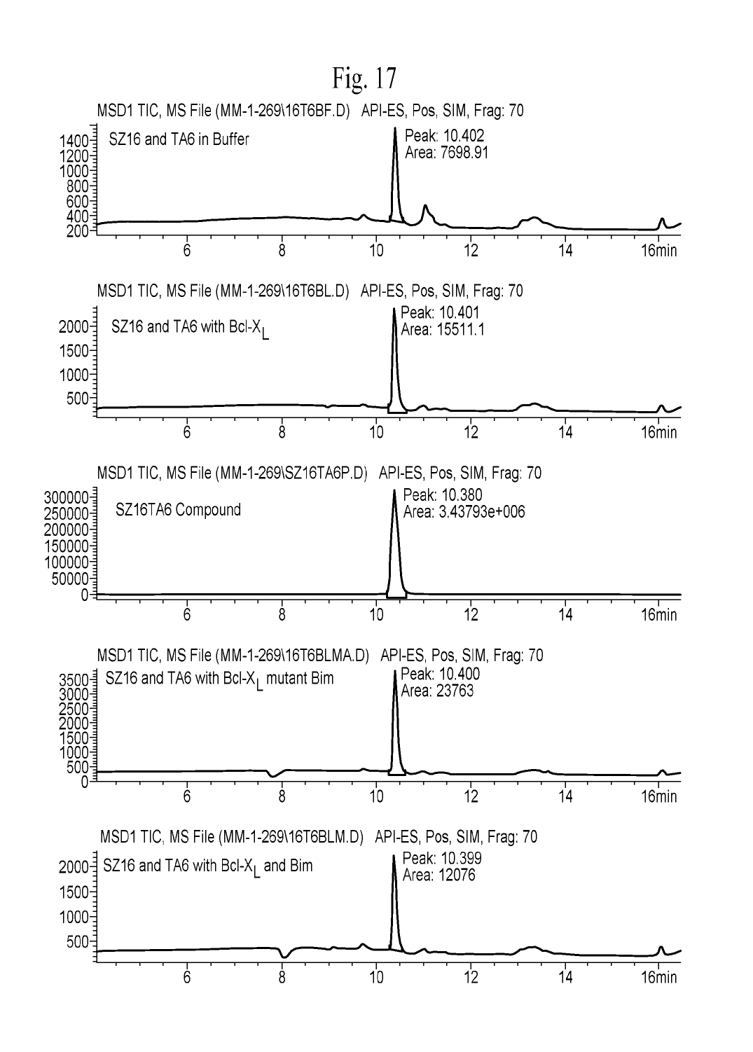

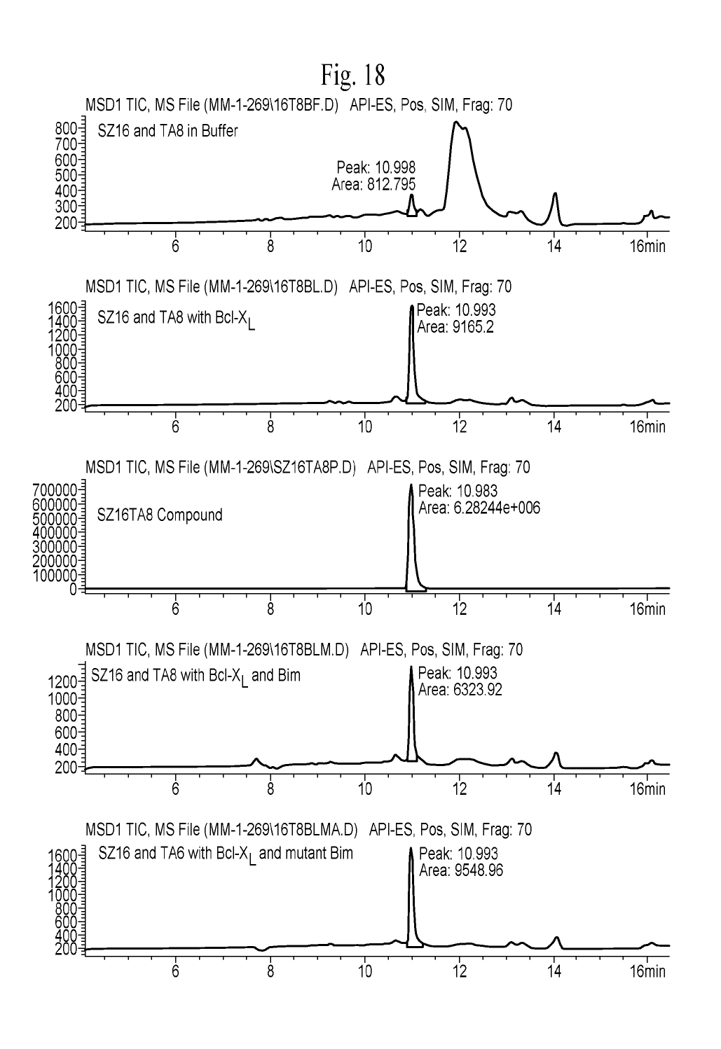

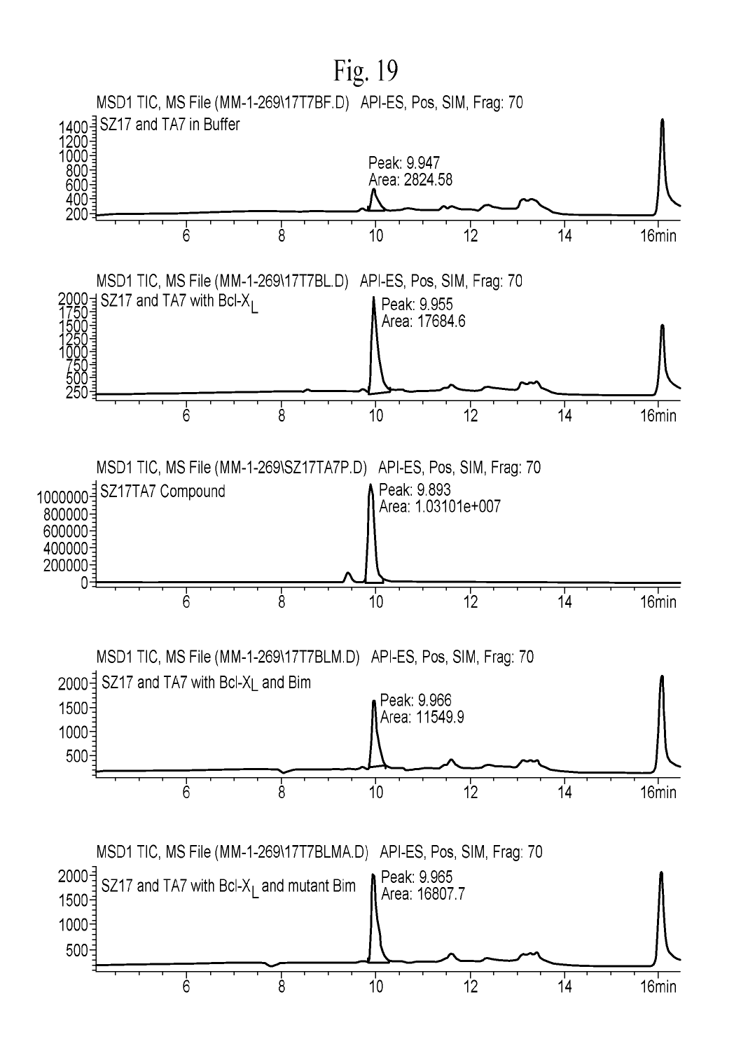

Other positive incubations and their LC/MS-SIM measurements can be seen in the Figures. The concentration of active building blocks, proteins and peptides are same as described above. The incubation reaction details are also as same as above. See FIGS. 12-19.

IC50 values for selected acylsulfonamides:

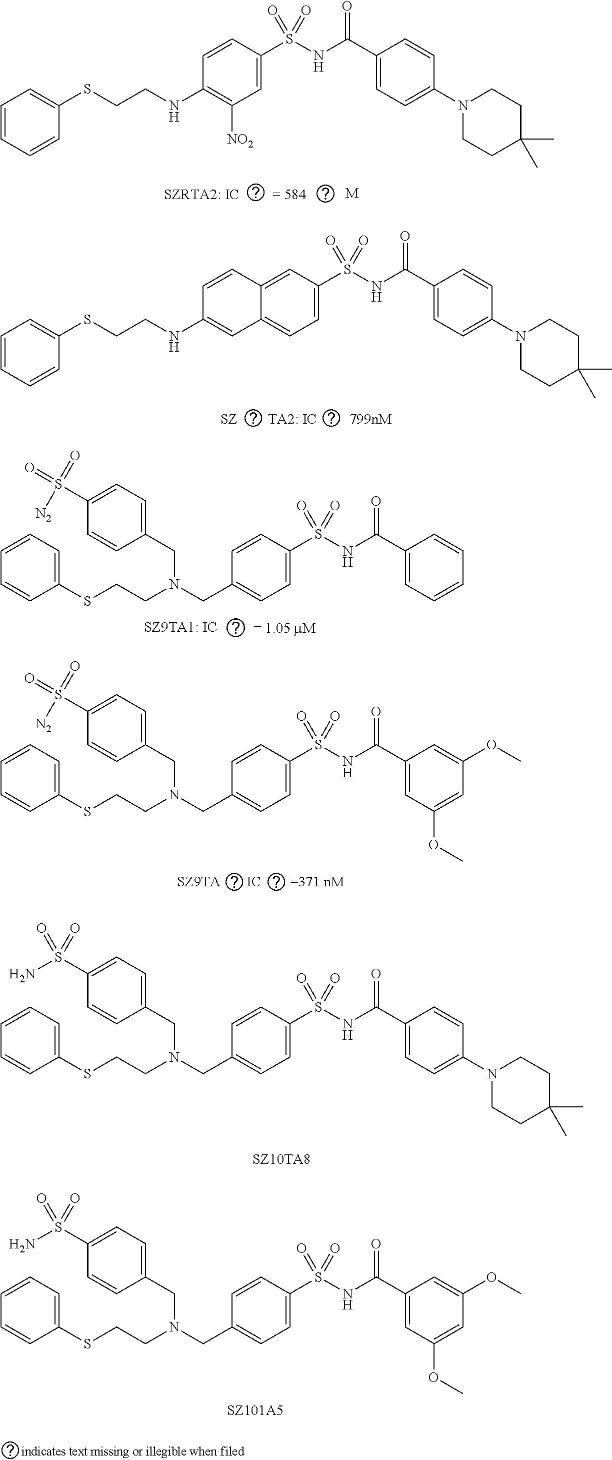

### Example 3

### Synthesis of a Cylsulfonamides

3.1 Acylsulfonamide (SZ7TA2)

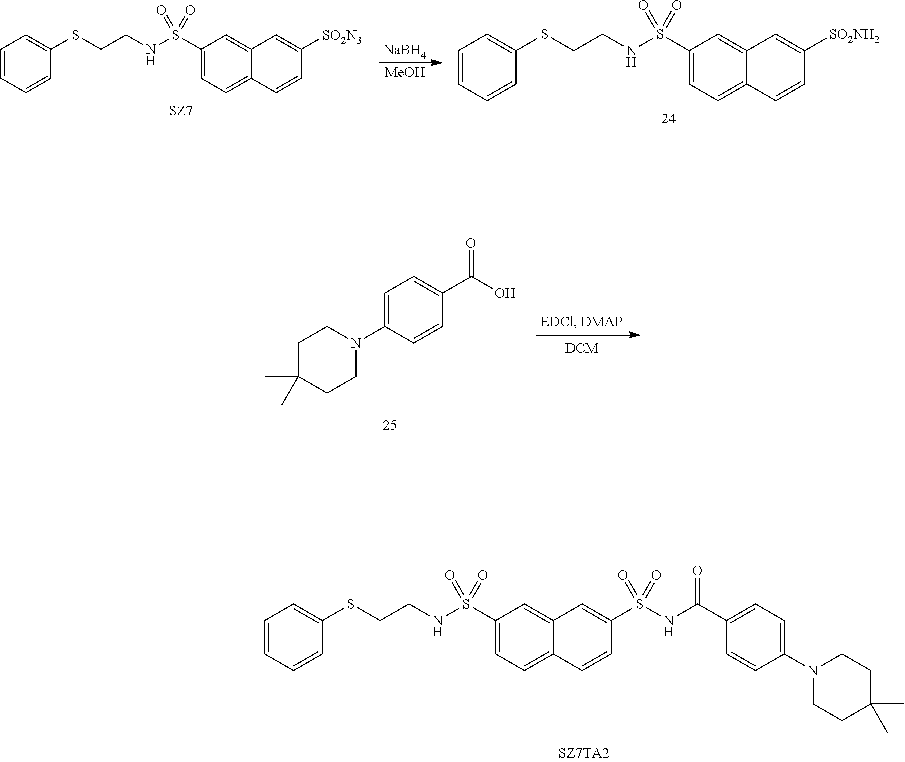

Sodium boron hydride (60 mg, 1.5 mmol) was added slowly to the solution of (SZ7) (450 mg, 1 mmol) in Methanol. The system was stirred for 30 min and removed all the solvent. Intermediate 24 was obtained by flash chromatography and used for next step directly. The solution of 24, 25 (1 mmol), EDCI (2 mmol) and DMAP (0.2 mmol) in DCM was stirred for 12 hours at room temperature, and the system was extracted by ethyl acetate (20 mL×3). The combined organic phase was dried by anhydrous sodium sulfate and concentrated. And product (SZ7TA2) (102 mg, 16%) was obtained by flash chromatography (hexane:EtOAc=1:1; Rf=0.2 in hexane:EtOAc=1:1). ¹H-NMR (400 MHz, CDCl₃) δ: 8.74 (s, 1H), 8.43 (s, 1H), 8.24 (d, J=8.8 Hz, 1H), 7.95-7.89 (m, 3H), 7.67 (d, J=8.4 Hz, 2H), 7.14 (d, J=6.4 Hz, 2H), 7.07 (d, J=6.8 Hz, 2H), 6.72 (d, J=8.8 Hz, 2H), 5.46 (d, J=65.2 Hz, 1H), 3.26-3.25 (m, 4H), 3.13 (d, J=6.0 Hz, 2H), 2.96 (d, J=6.4 Hz, 2H), 1.40-1.39 (m, 4H), 0.93 (s, 6H) ppm. ¹³C-NMR (100 MHz, CDCl₃) δ: 164.1, 154.4, 138.4, 137.9, 136.5, 133.6, 131.2, 130.9, 130.2, 130.0, 129.5, 129.4, 129.0, 126.9, 126.2, 125.4, 117.9, 113.0, 43.7, 41.7, 37.8, 33.9, 28.6, 27.7 ppm. HRMS (ESI⁺) for [M+H]⁺; calculated: 638.18116. found: 638.18097 (error m/z=−0.29 ppm).

3.2 Acylsulfonamide (SZ9TA5)

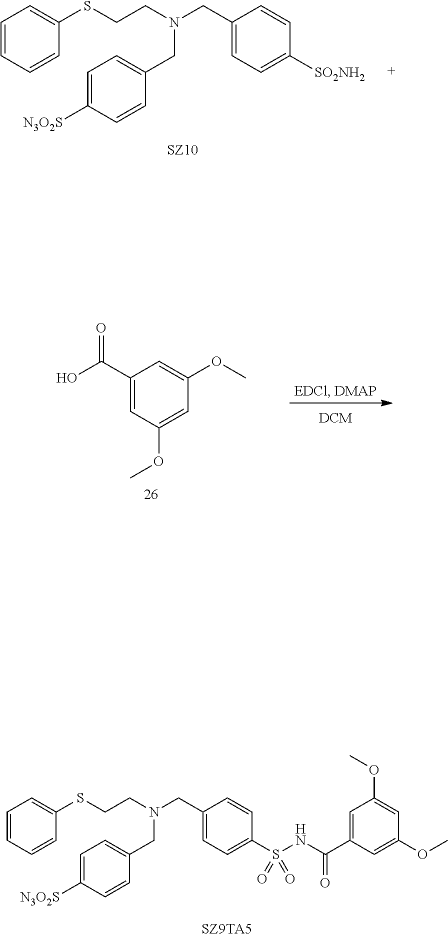

The solution of (SZ10) (1 mmol), 26 (1 mmol), EDCI (2 mmol) and DMAP (0.2 mmol) in DCM was stirred for 12 hours at room temperature, and the system was extracted by ethyl acetate (20 mL×3). The combined organic phase was dried by anhydrous sodium sulfate and concentrated. And product (SZ9TA5) (0.5 mmol, 50%) was obtained by flash chromatography (hexane:EtOAc=1:1; Rf=0.2 in hexane:EtOAc=1:1). ¹H-NMR (400 MHz, CDCl₃) δ: 7.97 (d, J=7.2 Hz, 2H), 7.78 (d, J=8.0 Hz, 2H), 7.46 (d, J=7.6 Hz, 2H), 7.28-6.99 (m, 10H), 6.42 (s, 1H), 3.64-3.56 (m, 10H), 2.98-2.94 (m, 2H), 2.63 (bs, 2H) ppm. ¹³C-NMR (100 MHz, CDCl₃) δ: 160.7, 147.3, 137.2, 136.0, 129.8, 129.2, 129.0, 127.7, 126.4, 106.6, 105.3, 58.1, 57.9, 55.6, 52.9, 31.5 ppm. HRMS (ESI⁺) for [M+H]⁺; calculated: 682.14584. found: 682.14395 (error m/z=−2.77 ppm).

3.3 Acylsulfonamide (SZ10TA2) (and Acylsulfonamide (SZ9TA2))

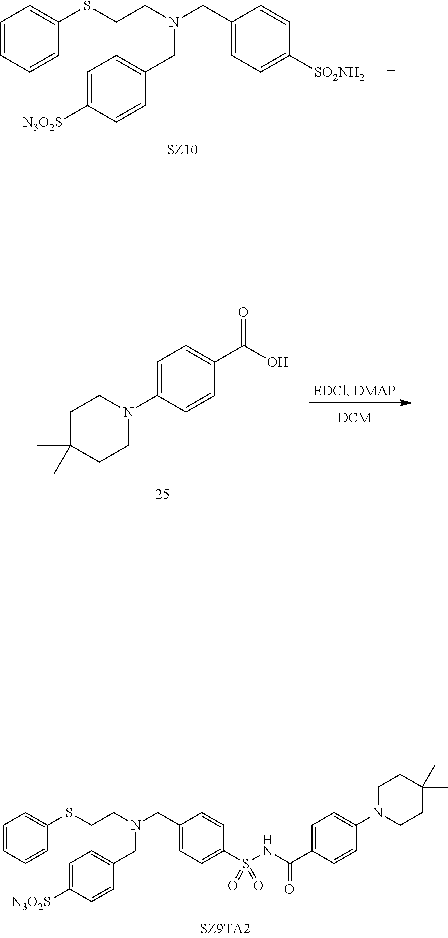

(SZ9TA2) was prepared starting from (SZ10) and known 25 using the procedure described for the preparation of (SZ9TA5) in 40% yield. ¹H-NMR (400 MHz, CDCl₃) δ: 8.09 (d, J=8.4 Hz, 2H), 7.85 (d, J=8.0 Hz, 2H), 7.63 (d, J=8.6 Hz, 2H), 7.56 (d, J=8.4 Hz, 2H), 7.51 (d, J=8.0 Hz, 2H), 7.24-7.16 (m, 7H), 6.86 (d, J=9.2 Hz, 2H), 3.77 (d, J=4.8 Hz, 4H), 3.32 (t, J=4.8 Hz, 4H), 3.06 (t, J=7.2 Hz, 2H), 2.79 (t, J=7.2 Hz, 2H), 1.47 (t, J=5.6 Hz, 4H), 0.98 (s, 6H) ppm. ¹³C-NMR (100 MHz, CDCl₃) δ: 154.1, 147.0, 137.0, 135.7, 130.0, 129.5, 129.0, 128.9, 128.7, 128.3, 127.5, 126.1, 113.1, 57.9, 57.7, 52.7, 43.9, 37.8, 31.3, 28.5, 27.7, 27.6 ppm. HRMS (ESI⁺) for [M+H]⁺; calculated: 733.22951. found: 733.22965 (error m/z=0.19 ppm).

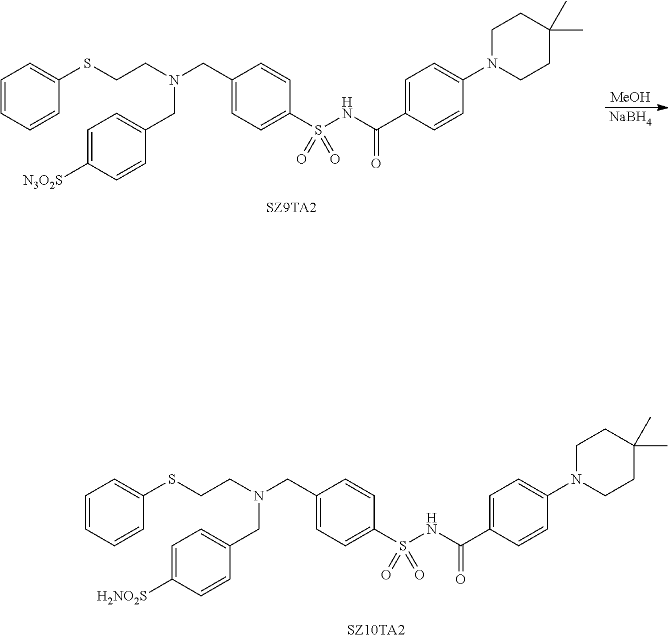

(SZ10TA2) was prepared starting from (SZ9TA2) using the procedure described for the preparation of compounds 24 in 86% yield. ¹H-NMR (400 MHz, CDCl₃) δ: 7.92 (d, J=7.2 Hz, 2H), 7.85 (d, J=7.2 Hz, 2H), 7.79 (d, J=7.2 Hz, 2H), 7.46 (d, J=7.6 Hz, 2H), 7.37 (d, J=7.6 Hz, 2H), 7.14-7.02 (m, 5H), 6.81 (d, J=8.0 Hz, 2H), 3.57 (d, J=10.8 Hz, 4H), 3.34-3.30 (m, 4H), 3.00 (t, J=7.2 Hz, 2H), 2.62 (t, J=7.2 Hz, 2H), 1.44 (t, J=5.2 Hz, 4H), 0.95 (s, 6H) ppm. ¹³C-NMR (100 MHz, CDCl₃) δ: 155.3, 145.6, 144.6, 143.6, 137.5, 130.4, 130.0, 129.8, 129.7, 128.3, 127.2, 126.9, 114.8, 62.9, 58.9, 54.0, 45.8, 39.2, 29.5, 28.3 ppm. HRMS (ESI⁺) for [M+H]⁺; calculated: 707.23901. found: 707.23934 (error m/z=0.46 ppm).

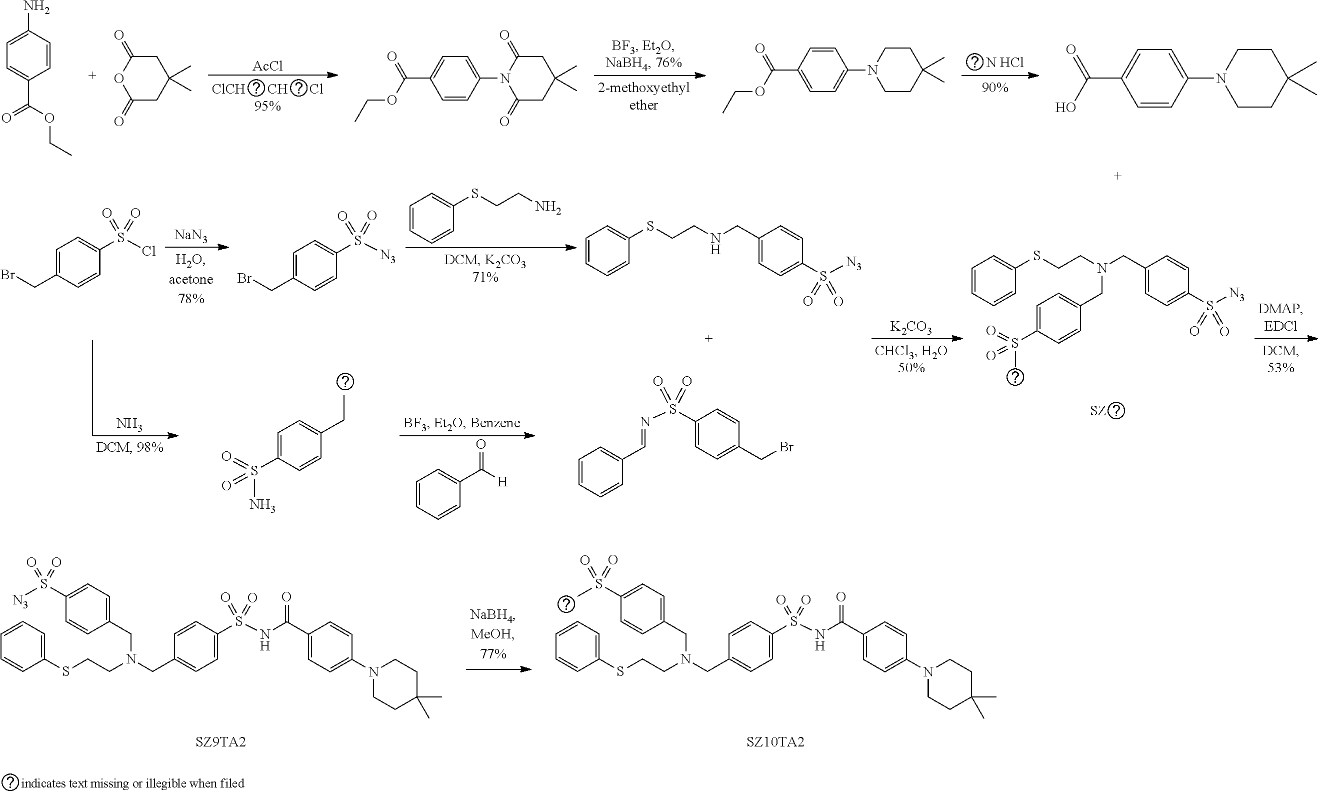

3.4 Acylsulfonamide (SZ15TA3)

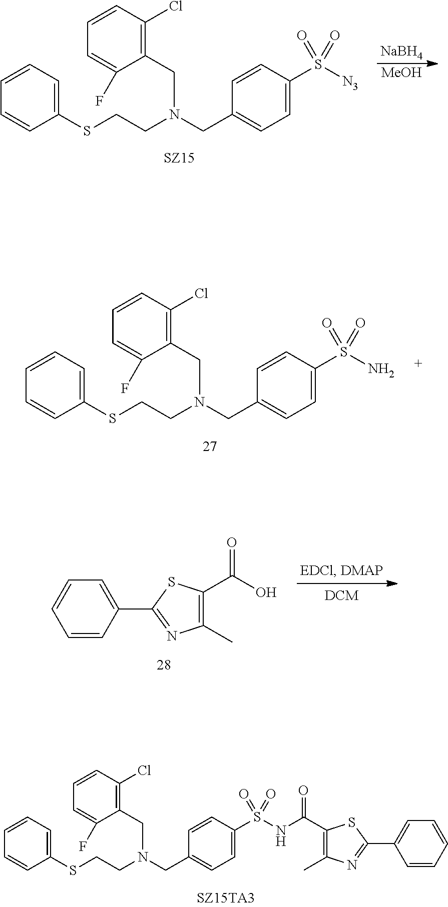 [SMILES: "MeOH" ; [N-]=[N+]=NS(=O)(=O)c1ccc(CN(CCSc2ccccc2)Cc2c(F)cccc2Cl)cc1 ; "NaBH4" ; NS(=O)(=O)c1ccc(CN(CCSc2ccccc2)Cc2c(F)cccc2Cl)cc1 ; Cc1nc(-c2ccccc2)sc1C(=O)O ; Cc1nc(-c2ccccc2)sc1C(=O)NS(=O)(=O)c1ccc(CN(CCSc2ccccc2)Cc2c(F)cccc2Cl)cc1]

Sodium boron hydride (60 mg, 1.5 mmol) was added slowly to the solution of (SZ15) (1 mmol) in Methanol. The system was stirred for 30 min and removed all the solvent. Intermediate 27 was obtained by flash chromatography and used for next step directly. The solution of 27, 28 (1 mmol), EDCI (2 mmol) and DMAP (0.2 mmol) in DCM was stirred for 12 hours at room temperature, and the system was extracted by ethyl acetate (20 mL×3). The combined organic phase was dried by anhydrous sodium sulfate and concentrated. And product (SZ15TA3) (0.5 mmol, 50%) was obtained by purification on preparative HPLC. ¹H-NMR (400 MHz, CDCl₃) δ: 9.95 (s, 2H), 7.99 (d, J=7.5 Hz, 2H), 7.77 (d, J=7.5 Hz, 2H), 7.53 (d, J=7.5 Hz, 2H), 7.35-7.12 (m, 10H), 6.92 (t, J=7.5 Hz, 1H), 4.23 (bs, 4H), 3.14-3.12 (m, 4H), 2.57 (s, 3H) ppm. ¹³C-NMR (100 MHz, CDCl₃) δ: 169.8, 160.3 (d, ¹J_{CF}=150 Hz), 160.2, 139.9, 136.9 (d, ²J_{CF}=12.5 Hz), 132.8, 132.5 (d, ³J_{CF}=10 Hz), 131.9, 131.7, 131.0, 130.8, 129.4, 129.2, 129.2, 127.6, 127.0, 126.3, 122.0, 117.1, 116.8, 114.9 (d, ²J_{CF}=21.5 Hz), 57.5, 52.7, 48.9, 28.8, 17.7 ppm. HRMS (ESI⁺) for [M+H]⁺; calculated: 666.11164. found: 666.10968 (error m/z=−2.94 ppm).

3.5 Acylsulfonamide (SZ15TA8)

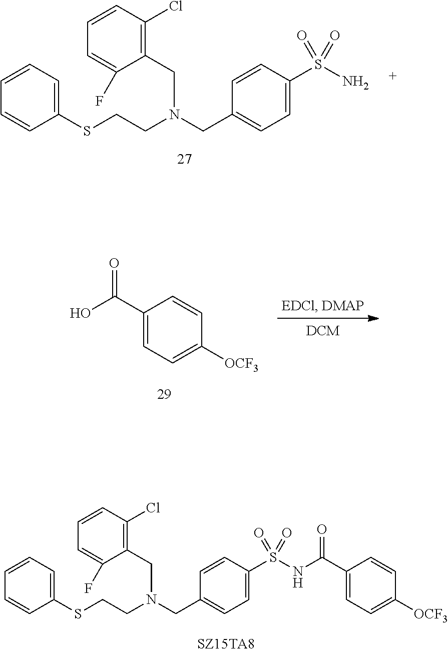 [SMILES: NS(=O)(=O)c1ccc(CN(CCSc2ccccc2)Cc2c(F)cccc2Cl)cc1 ; O=C(O)c1ccc(OC(F)(F)F)cc1 ; O=C(NS(=O)(=O)c1ccc(CN(CCSc2ccccc2)Cc2c(F)cccc2Cl)cc1)c1ccc(OC(F)(F)F)cc1]

The solution of 27, 29 (1 mmol), EDCI (2 mmol) and DMAP (0.2 mmol) in DCM was stirred for 12 hours at room temperature, and the system was extracted by ethyl acetate (20 mL×3). The combined organic phase was dried by anhydrous sodium sulfate and concentrated. And product (SZ15TA8) (0.82 mmol, 82%) was obtained by purification on preparative HPLC. ¹H-NMR (400 MHz, CDCl₃) δ: 10.9 (bs, 1H), 8.04 (d, J=8.0 Hz, 2H), 7.90 (d, J=8.4 Hz, 2H), 7.61 (d, J=8.0 Hz, 2H), 7.23-7.09 (m, 9H), 6.93 (t, J=8.4 Hz, 1H), 4.27 (s, 2H), 4.25 (s, 2H), 3.23-3.16 (m, 4H) ppm. ¹³C-NMR (100 MHz, CDCl₃) δ: 164.1, 162.6 (d, ¹J_{CF}=250 Hz), 161.2 (d, ²J_{CF}=37 Hz), 152.7, 139.6, 137.6, 136.6, 133.0, 132.1 (d, ³J_{CF}=9.5 Hz), 130.8, 130.3, 129.4, 129.2, 129.0, 127.3, 126.1, 121.4, 120.3, 118.8, 117.5, 117.4, 114.6 (d, ²J_{CF}=22.6 Hz), 57.4, 52.7, 48.8, 28.7 ppm. HRMS (ESI⁺) for [M+H]⁺; calculated: 653.09532. found: 653.09349 (error m/z=−2.79 ppm).

3.6 Acylsulfonamide (SZ16TA6)

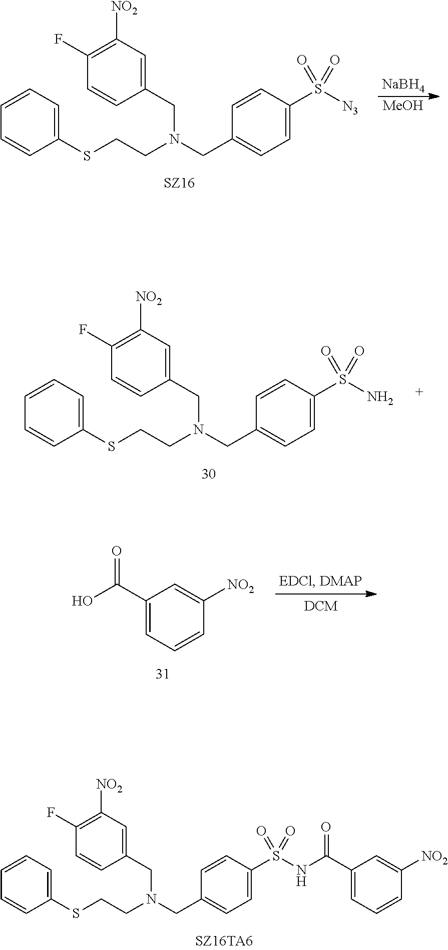

Compound 30 was prepared starting from (SZ16) using the procedure described for the preparation of compounds 27, and (SZ16TA6) was prepared starting from 30 and 31 using the procedure described for the preparation of compounds (SZ15TA8) in 46% yield. ¹H-NMR (400 MHz, CD₃CN) δ: 8.58 (s, 1H), 8.36 (d, J=8.0 Hz, 1H), 8.15 (d, J=7.6 Hz, 1H), 8.10 (d, J=6.8 Hz, 1H), 8.02 (d, J=7.6 Hz, 2H), 7.69-7.65 (m, 2H), 7.62 (d, J=8.0 Hz, 2H), 7.28 (t, J=10 Hz, 1H), 7.17-7.14 (m, 4H), 7.09 (dd J=8.0, 4.0 Hz, 1H), 3.97 (s, 2H), 3.93 (s, 2H), 3.15 (t, J=7.2 Hz, 2H), 2.86 (t, J=7.2 Hz, 2H) ppm. ¹³C-NMR (100 MHz, CD₃CN) δ: 163.8, 155.1 (d, ¹J_{CF}=261 Hz), 148.4, 142.3, 138.9, 137.4 (d, ³J_{CF}=9.1 Hz), 135.1, 134.5, 133.3 (d, ²J_{CF}=21.4 Hz), 130.4, 129.3, 129.2, 128.7, 127.8, 127.4, 126.5, 123.4, 118.7 (d, ²J_{CF}=21.3 Hz), 117.6, 57.2, 56.5, 52.2, 29.2 ppm.

3.7 Acylsulfonamide (SZ16TA8)

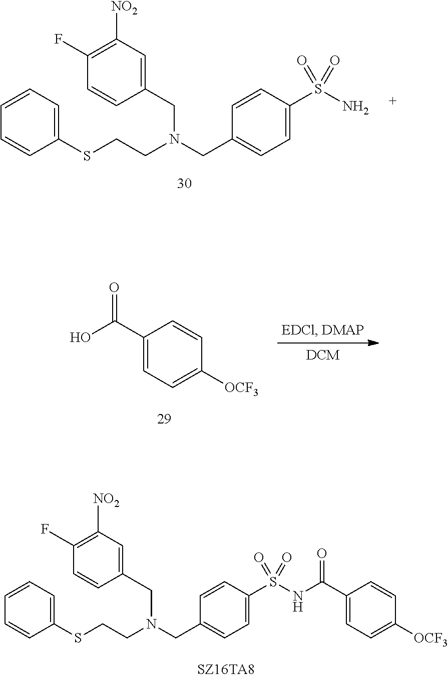 [SMILES: NS(=O)(=O)c1ccc(CN(CCSc2ccccc2)Cc2ccc(F)c([N+](=O)[O-])c2)cc1 ; O=C(O)c1ccc(OC(F)(F)F)cc1 ; O=C(NS(=O)(=O)c1ccc(CN(CCSc2ccccc2)Cc2ccc(F)c([N+](=O)[O-])c2)cc1)c1ccc(OC(F)(F)F)cc1]

(SZ16TA8) was prepared starting from 30 and 29 using the procedure described for the preparation of compounds (SZ15TA8) in 36% yield. ¹H-NMR (400 MHz, CD₃CN) δ: 8.18 (dd, J=6.8, 1.6 Hz, 1H), 8.04 (d, J=8.0 Hz, 2H), 7.90 (d, J=8.8 Hz, 2H), 7.75 (dd, J=4.4, 2.4 Hz, 1H), 7.64 (d, J=8.4 Hz, 2H), 7.37 (d, J=8.8 Hz, 2H), 7.34-7.31 (m, 1H), 7.20-7.15 (m, 4H), 4.20 (s, 2H), 4.17 (s, 2H), 3.23-3.19 (m, 2H), 3.03-2.99 (m, 2H), 2.62 (t, J=7.2 Hz, 2H), 1.44 (t, J=5.2 Hz, 4H), 0.95 (s, 6H) ppm. ¹³C-NMR (100 MHz, CD₃CN) δ: 164.3, 155.6 (d, ¹J_{CF}=263 Hz), 152.6, 139.9, 138.8, 138.3 (d, ³J_{CF}=10 Hz), 134.0, 131.2, 130.9, 130.5, 130.0, 129.4, 128.9, 128.5, 127.1, 120.9, 119.1 (d, ²J_{CF}=21.4 Hz), 57.0, 56.4, 51.7, 28.3 ppm.

3.8 Acylsulfonamide (SZ17TA7)

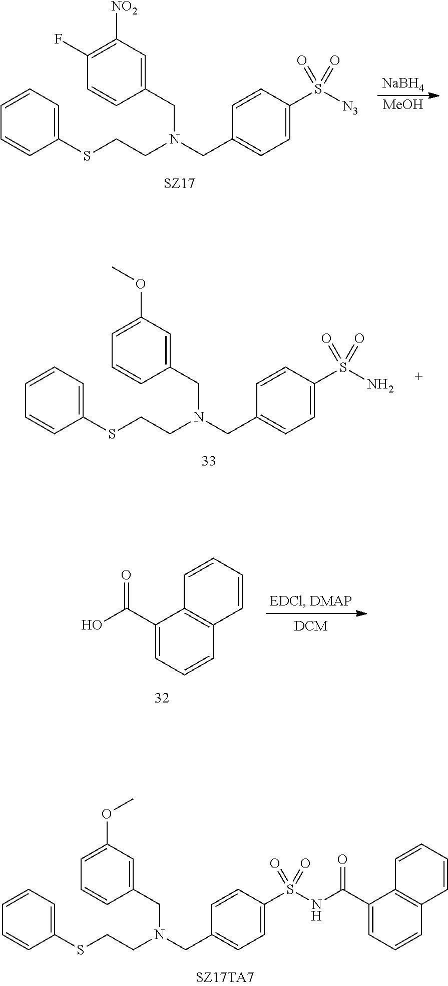

Compound 33 was prepared starting from (SZ17) using the procedure described for the preparation of compounds 27, and (SZ17TA7) was prepared starting from 33 and 32 using the procedure described for the preparation of compounds (SZ15TA8) in 36% yield. HRMS (ESI⁺) for [M+H₂O]⁺; calculated: 614.19037. found: 614.18830 (error m/z=−3.36 ppm).

3.9 Other Sulfonyl Azides

Other sulfonyl azides described herein were prepared, in general, in accordance with the methods described above in Examples 3.1 to 3.8 and conventional organic synthesis techniques.

### Example 4

### Preparation of Other Compounds

4.1 Acylsulfonamide (SZ2TA1)

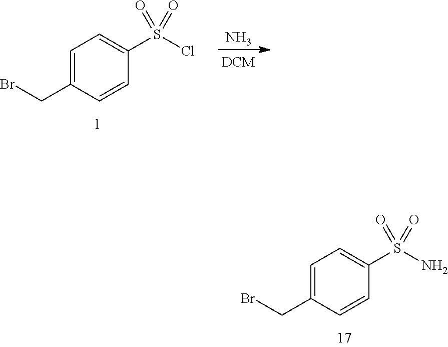

Ammonia gas was passed through a solution of compound 1 (1 g, 3.7 mmol) in DCM (100 mL) at 0° C. for 10 minutes. Brine (20 mL) was added. The separated organic phase was dried over anhydrous sodium sulfate and concentrated. The product 17 (900 mg, 96.8%) was isolated by flash chromatography (hexanes:EtOAc=2:1). ¹H-NMR (250 MHz, Acetone-d6) δ: 7.91 (d, J=10.0 Hz, 2H), 7.67 (d, J=10.0 Hz, 2H), 6.63 (bs, 2H), 4.74 (s, 2H) ppm.

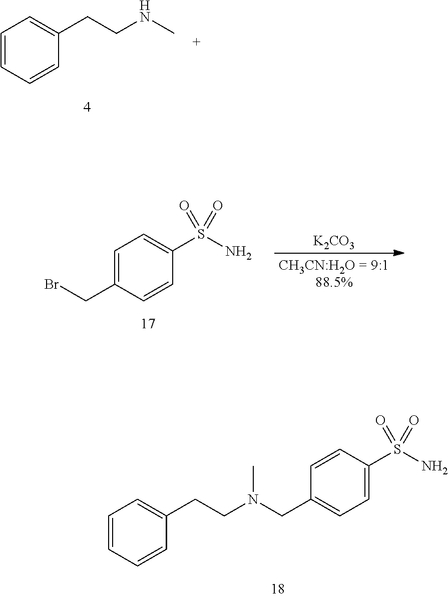

A mixture of 17 (100 mg, 0.40 mmol), 4 (54.3 mg, 0.40 mmol) and potassium carbonate (100 mg, 0.72 mmol) in acetonitrile and water (9:1) was stirred at room temperature for 12 hours. To this mixture, ethyl acetate (20 mL) and water (20 mL) were added, and the resulting biphasic mixture was extracted by ethyl acetate (20 mL×3). The combined organic phases were dried over anhydrous sodium sulfate and concentrated. Product 18 (108 mg, 88.5%) was obtained by flash chromatography (hexane:EtOAc=1:1; Rf=0.2 in hexane:EtOAc=1:1). ¹H-NMR (250 MHz, CDCl₃) δ: 7.73 (d, J=10.0 Hz, 2H), 7.30 (d, J=10.0 Hz, 2H), 7.22-7.07 (m, 5H), 5.18 (bs, 2H), 3.51 (s, 2H), 2.76-2.70 (m, 2H), 2.60-2.54 (m, 2H), 2.19 (s, 3H) ppm. ¹³C-NMR (62.5 MHz, CDCl₃) δ: 144.6, 140.6, 140.2, 129.4, 128.7, 128.4, 126.4, 126.1, 61.6, 59.1, 42.2, 33.8 ppm. HRMS (ESI⁺) for [M+H]⁺; calculated: 305.1324. found: 305.1325. (error m/z=0.3 ppm).

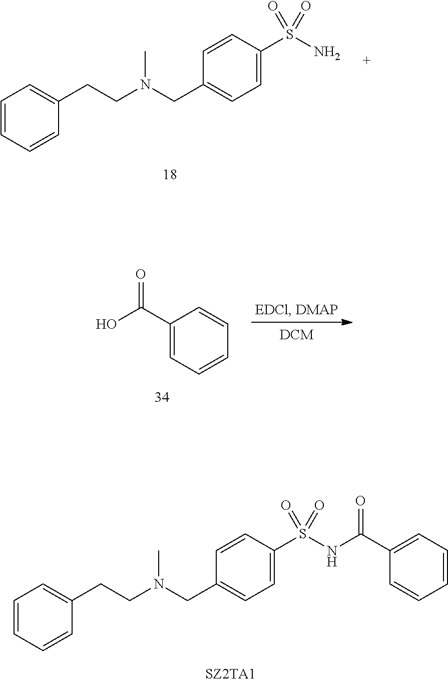 [SMILES: CN(CCc1ccccc1)Cc1ccc(S(N)(=O)=O)cc1 ; O=C(O)c1ccccc1 ; CN(CCc1ccccc1)Cc1ccc(S(=O)(=O)NC(=O)c2ccccc2)cc1]

A mixture of 34 (31 mg, 0.25 mmol), 18 (76 mg, 0.25 mmol), EDCI (60 mg, 0.314 mmol) and DMAP (8 mg, 0.065 mmol) in dichloromethane (10 mL) was stirred for 19 h. Product (SZ2TA1) (87 mg, 85%) was isolated after flash chromatography (DCM:MeOH=18:1). Rf=0.68 (DCM:MeOH=4:1). ¹H-NMR (400 MHz, DMSO-d6) δ: 7.86 (d, J=7.6 Hz, 4H), 7.48 (d, J=8 Hz, 2H), 7.37-7.19 (m, 8H), 4.16 (bs, 2H), 3.09-3.07 (m, 2H), 2.94-2.90 (m, 2H), 2.58 (s, 3H) ppm. ¹³C-NMR (100 MHz, DMSO-d6) δ: 169.7, 146.2, 138.4, 131.2, 130.6, 129.9, 129.4, 129.2, 129.0, 128.3, 128.0, 127.3, 59.5, 57.4, 31.1 ppm. HRMS (ESI⁺) for [M+H]⁺; calculated: 409.1586. found: 409.1582 (error m/z=−0.9 ppm).

4.2 Acylsulfonamide (SZ2TA2)

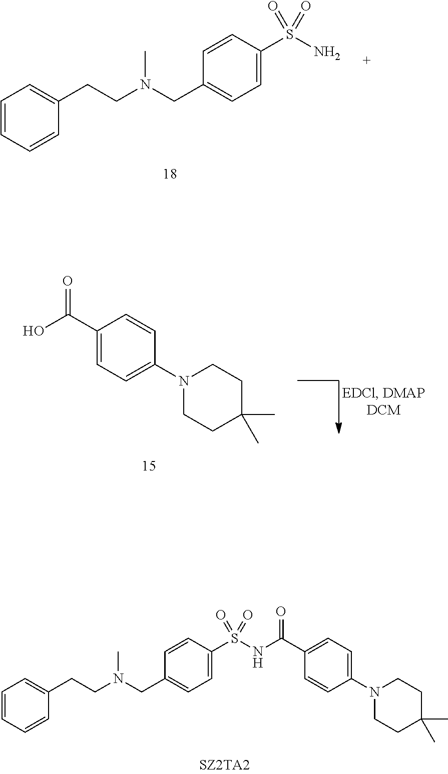 [SMILES: CN(CCc1ccccc1)Cc1ccc(S(N)(=O)=O)cc1 ; CC1(C)CCN(c2ccc(C(=O)O)cc2)CC1 ; CN(CCc1ccccc1)Cc1ccc(S(=O)(=O)NC(=O)c2ccc(N3CCC(C)(C)CC3)cc2)cc1]

A solution of 15 (58 mg, 0.25 mmol), 18 (76 mg, 0.25 mmol), EDCI (60 mg, 0.314 mmol) and DMAP (8 mg, 0.065 mmol) in dichloromethane (10 mL) was stirred for 19 hours. Product (SZ2TA2) (42 mg, 32.5%) was isolated after flash chromatography (DCM:MeOH=24:1). Rf=0.6 (DCM:MeOH=6:1). ¹H-NMR (400 MHz, DMSO-d6) δ: 7.85 (d, J=8 Hz, 2H), 7.69 (d, J=8.8 Hz, 2H), 7.44 (d, J=8 Hz, 2H), 7.24-7.16 (m, 6H), 6.84 (d, J=8.8 Hz, 2H), 3.80 (bs, 2H), 3.27-3.24 (m, 4H), 3.14 (s, 2H), 2.79 (bs, 4H), 2.33 (s, 3H), 1.35-1.32 (m, 2H), 0.91 (s, 6H) ppm. ¹³C-NMR (100 MHz, DMSO-d6) δ: 166.9, 153.9, 139.9, 130.7, 129.9, 129.3, 129.0, 128.1, 126.8, 126.3, 113.5, 60.5, 58.5, 44.5, 41.7, 38.1, 32.6, 29.1, 28.3, 28.2 ppm. HRMS (ESI⁺) for [M+H]⁺; calculated: 520.2634. found: 520.2627 (error m/z=1.3 ppm).

4.3 Acylsulfonamide (SZ2TA3)

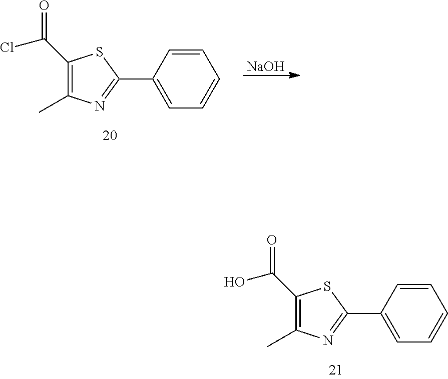

Compound 20 (500 mg, 2.1 mmol) was added into 1M NaOH and then stirred overnight. The resulting mixture was treated with 2N HCl. Product 21 (310 mg, 67%) can be filtered out and dried. The crude product was used for next step directly.

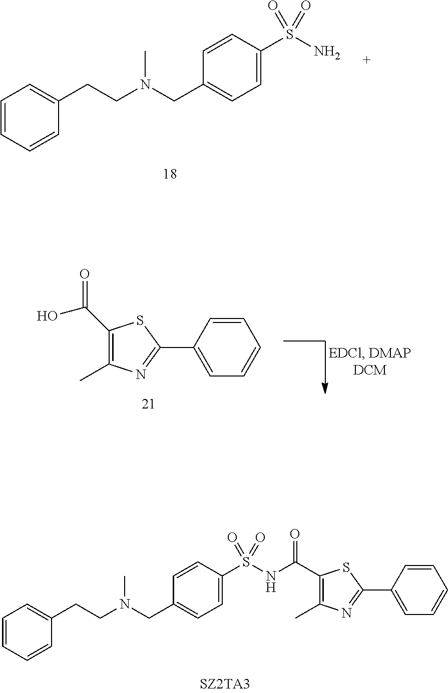 [SMILES: CN(CCc1ccccc1)Cc1ccc(S(N)(=O)=O)cc1 ; Cc1nc(-c2ccccc2)sc1C(=O)O ; Cc1nc(-c2ccccc2)sc1C(=O)NS(=O)(=O)c1ccc(CN(C)CCc2ccccc2)cc1]

A solution of 21 (45 mg, 0.205 mmol), 18 (61 mg, 0.205 mmol), EDCI (60 mg, 0.314 mmol) and DMAP (8 mg, 0.065 mmol) in dichloromethane (10 mL) was stirred for 24 h. Product (SZ2TA3) (58 mg, 55%) was isolated by preparative HPLC. (DCM:MeOH=36:1). Rf=0.55 (DCM:MeOH=8:1). ¹H-NMR (250 MHz, Acetone-d6) δ: 8.03 (d, J=8 Hz, 2H), 7.85-7.78 (m, 4H), 7.39-7.36 (m, 3H), 7.14-7.11 (m, 5H), 4.52 (bs, 2H), 3.36-3.32 (m, 2H), 3.12-3.09 (m, 2H), 2.84 (s, 3H), 2.48 (s, 3H) pm. ¹³C-NMR (100 MHz, CDCl₃) δ: 169.7, 161.2, 159.8, 140.9, 135.1, 134.4, 131.8, 131.6, 131.5, 129.2, 129.1, 129.0, 128.6, 127.5, 126.9, 122.4, 59.3, 57.5, 39.6, 30.5, 17.5 ppm. HRMS (ESI⁺) for [M+H]⁺; calculated: 506.1572. found: 506.1565 (error m/z=−1.4 ppm).

4.4 Acylsulfonamide (SZ4TA1)

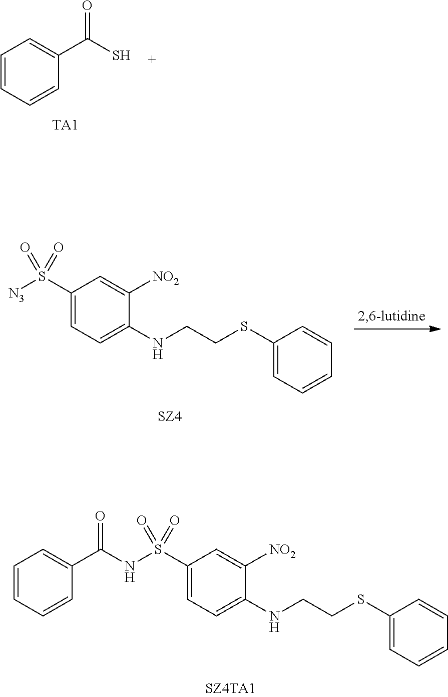

A solution of (TA1) (50 mg, 0.36 mmol), (SZ4) (136 mg, 0.36 mmol) and 2,6-lutidine (40 mg, 0.37 mmol) in chloroform (10 mL) was stirred at 70° C. for 16 h. Chloroform (30 mL) was added to the reaction and the resulting mixture was washed sequentially with saturated copper sulfate aqueous solution (50 mL) and brine (20 mL). The organic phase was then dried over anhydrous sodium sulfate and concentrated. Product (SZ4TA1) (89 mg, 54%) was isolated after flash chromatography (DCM:MeOH=26:1). Rf=0.58 (DCM:MeOH=8:1). ¹H-NMR (400 MHz, CDCl₃) δ: 8.93 (s, 1H), 8.87 (d, J=1.2 Hz, 1H), 8.68 (s, 1H), 8.13 (d, J=8.4 Hz, 1H), 7.76 (d, J=7.6 Hz, 2H), 7.56 (t, J=7.2 Hz, 1H), 7.45-7.37 (m, 4H), 7.29-7.22 (m, 4H), 6.81 (d, J=8.8 Hz, 1H), 3.58-3.54 (m, 2H), 3.20-3.17 (m, 2H) ppm. ¹³C-NMR (100 MHz, CDCl₃) δ: 164.3, 147.6, 135.5, 133.7, 131.2, 130.9, 129.3, 129.0, 127.7, 127.4, 124.5, 113.7, 42.1, 33.2 ppm. HRMS (ESI⁺) for [M+H]⁺; calculated: 458.08389. found: 458.08350 (error m/z=−0.84 ppm).

4.5 Acylsulfonamide (SZ5TA1)

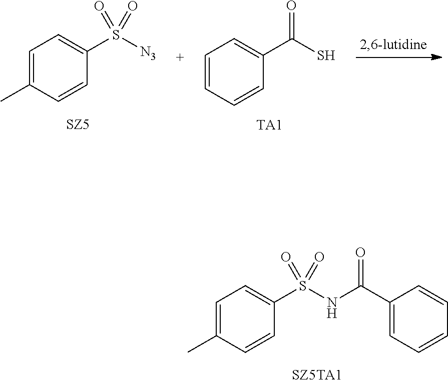

A solution of (TA1) (50 mg, 0.36 mmol), (SZ5) (71 mg, 0.36 mmol) and 2,6-lutidine (40 mg, 0.37 mmol) in chloroform (10 mL) was stirred at 70° C. for 16 h. Chloroform (30 mL) was added to the reaction and the resulting mixture was washed sequentially with saturated copper sulfate aqueous solution (50 mL) and brine (20 mL). The organic phase was then dried over anhydrous sodium sulfate and concentrated. Product (SZ5TA1) (67 mg, 68%) was isolated after flash chromatography (DCM:MeOH=38:1). Rf=0.7 (DCM:MeOH=10:1). ¹H-NMR (250 MHz, CDCl₃) δ: 9.62 (s, 1H), 7.95 (d, J=8 Hz, 2H), 7.75 (d, J=8.0 Hz, 2H), 7.43 (t, J=7.2 Hz, 1H), 7.32-7.22 (m, 4H), 2.32 (s, 3H) ppm. ¹³C-NMR (100 MHz, CDCl₃) δ: 164.5, 145.1, 135.4, 133.4, 129.5, 128.7, 128.5, 127.9, 127.8, 21.6 ppm. HRMS (ESI⁺) for [M+H]⁺; calculated: 276.0694. found: 276.0696 (error m/z=−0.7 ppm).

4.6 Acylsulfonamide (SZ5TA2)

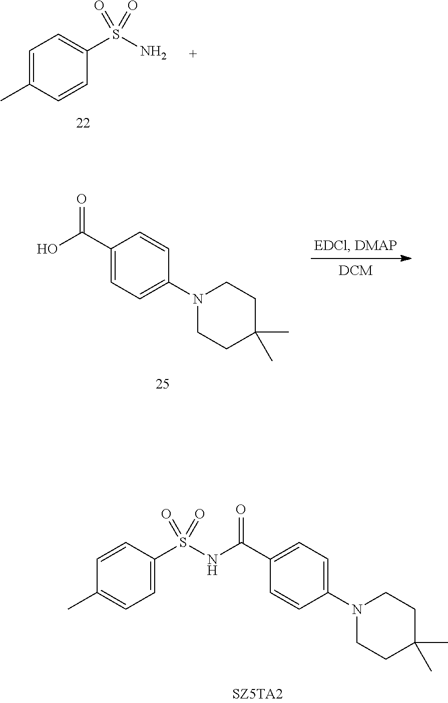

A solution of 15 (58 mg, 0.25 mmol), 22 (43 mg, 0.25 mmol), EDCI (60 mg, 0.314 mmol) and DMAP (8 mg, 0.065 mmol) in dichloromethane (10 mL) was stirred for 30 h. Product (SZ5TA2) (68 mg, 70%) was isolated after flash chromatography (DCM:MeOH=16:1). Rf=0.6 (DCM:MeOH=8:1). ¹H-NMR (400 MHz, CDCl₃) δ: 8.70 (bs, 1H), 8.00 (d, J=8 Hz, 2H), 7.63 (d, J=8.4 Hz, 2H), 7.30 (d, J=8 Hz, 2H), 6.82 (d, J=7.6 Hz, 2H), 3.30 (t, J=6 Hz, 4H), 2.40 (s, 3H), 1.46 (bs, 4H), 0.97 (s, 6H) ppm. ¹³C-NMR (100 MHz, DMSO-d6) δ: 165.1, 154.4, 144.5, 137.9, 130.9, 130.0, 128.3, 119.2, 113.4, 43.9, 38.0, 29.2, 28.3, 21.9 ppm. HRMS (ESI⁺) for [M+H]⁺; calculated: 387.1742. found: 387.1742 (error m/z=−1.8 ppm).

4.7 Acylsulfonamide (SZ9TA1)

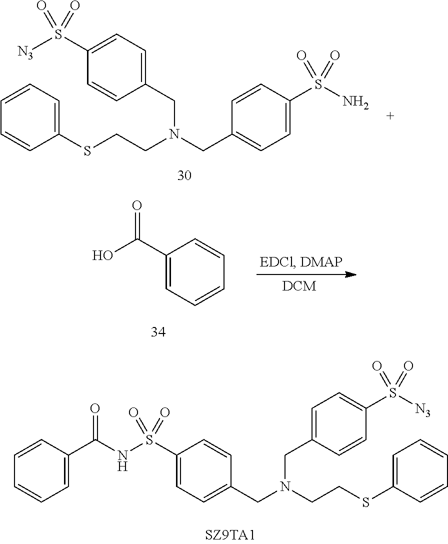 [SMILES: [N-]=[N+]=NS(=O)(=O)c1ccc(CN(CCSc2ccccc2)Cc2ccc(S(N)(=O)=O)cc2)cc1 ; O=C(O)c1ccccc1 ; [N-]=[N+]=NS(=O)(=O)c1ccc(CN(CCSc2ccccc2)Cc2ccc(S(=O)(=O)NC(=O)c3ccccc3)cc2)cc1]

(SZ9TA1) was prepared starting from 30 and 34 using the procedure described for the preparation of compounds (SZ15TA8) in 37% yield. ¹H-NMR (400 MHz, CDCl₃) δ: 8.03 (d, J=8.4 Hz, 2H), 7.78 (d, J=8.4 Hz, 2H), 7.71 (d, J=7.6 Hz, 2H), 7.51-7.46 (m, 4H), 7.41-7.33 (m, 3H), 7.19-7.04 (m, 5H), 3.61 (s, 4H), 2.99 (t, J=7.2 Hz, 2H), 2.68 (t, J=7.2 Hz, 2H) ppm. ¹³C-NMR (100 MHz, CD₃CN) δ: 170.0, 164.3, 146.9, 145.6, 137.4, 137.2, 135.7, 133.5, 131.2, 130.1, 129.5, 129.1, 128.9, 128.8, 128.4, 127.7, 127.6, 126.3, 58.0, 57.9, 52.8, 31.5 ppm. HRMS (ESI⁺) for [M+H]⁺; calculated: 622.12471. found: 622.12402 (error m/z=−1.10 ppm).

4.8 Acylsulfonamide (SZ10TA1)

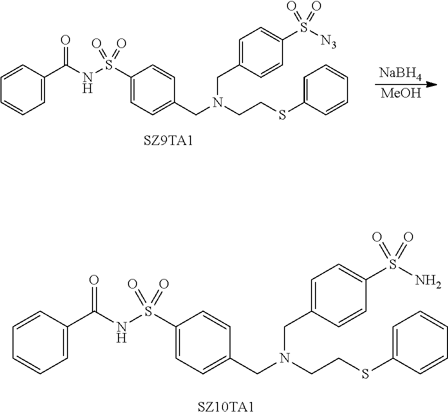

Sodium boron hydride (60 mg, 1.5 mmol) was added slowly to the solution of (SZ9TA1) (1 mmol) in Methanol. The system was stirred for 30 min and removed all the solvent. (SZ10TA1) was obtained by flash chromatography (hexane:EtOAc=1:1; Rf=0.15 in hexane:EtOAc=1:1). ¹H-NMR (400 MHz, CD₃OD) δ: 8.04 (d, J=8.0 Hz, 2H), 7.92 (d, J=7.6 Hz, 2H), 7.79 (d, J=7.6 Hz, 2H), 7.44-7.27 (m, 7H), 7.12-6.97 (m, 5H), 3.55 (s, 2H), 3.49 (s, 2H), 2.96 (t, J=7.2 Hz, 2H), 2.57 (t, J=7.2 Hz, 2H) ppm. ¹³C-NMR (100 MHz, CD₃OD) δ: 173.4, 144.3, 143.6, 142.4, 137.0, 136.3, 131.5, 129.2, 129.0, 128.8, 128.7, 128.6, 127.7, 126.5, 126.0, 125.7, 57.7, 52.7, 30.7 ppm. HRMS (ESI⁺) for [M+H]⁺; calculated: 596.13421. found: 596.13388 (error m/z=−0.55 ppm).

4.9 Acylsulfonamide (SZ10TA5)

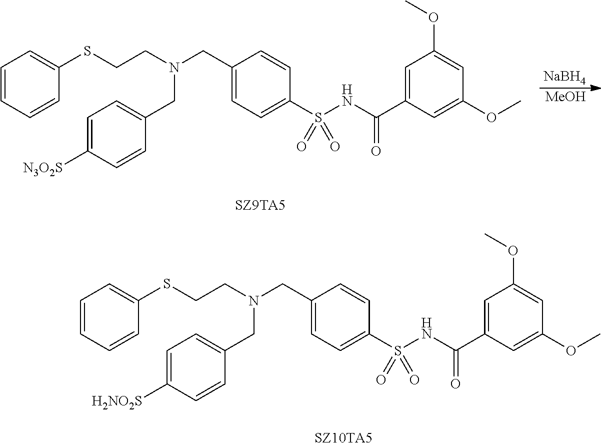

(SZ10TA5) was prepared starting from (SZ9TA5) using the procedure described for the preparation of compounds (SZ10TA1) in 91% yield. ¹H-NMR (400 MHz, CDCl₃) δ: 7.96 (d, J=8.0 Hz, 2H), 7.80 (d, J=8.04 Hz, 2H), 7.50-7.44 (m, 4H), 7.15-7.01 (m, 7H), 6.57 (d, J=1.6 Hz, 1H), 3.71 (s, 6H), 3.60 (s, 2H), 3.58 (s, 2H), 3.31-3.30 (m, 2H), 2.64-2.60 (m, 2H) ppm. ¹³C-NMR (100 MHz, CD₃CN) δ: 171.0, 162.0, 145.7, 145.2, 143.5, 141.6, 138.0, 137.4, 130.6, 130.4, 129.9, 129.8, 128.7, 127.1, 126.9, 107.3, 105.6, 58.8, 56.1, 55.9, 53.9, 31.8 ppm. HRMS (ESI⁺) for [M+H]⁺; calculated: 656.15534. found: 656.15466 (error m/z=−1.03 ppm).

4.10 Acylsulfonamide (SZ15TA1)

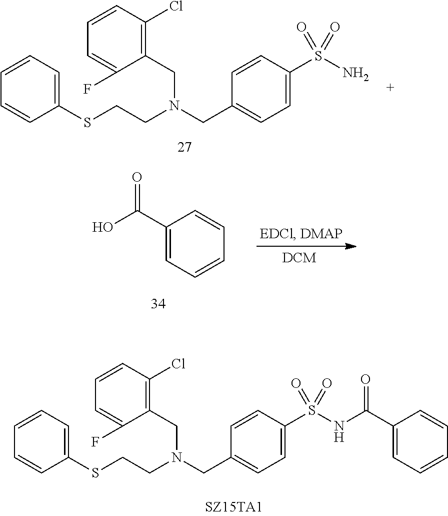 [SMILES: NS(=O)(=O)c1ccc(CN(CCSc2ccccc2)Cc2c(F)cccc2Cl)cc1 ; O=C(O)c1ccccc1 ; O=C(NS(=O)(=O)c1ccc(CN(CCSc2ccccc2)Cc2c(F)cccc2Cl)cc1)c1ccccc1]

(SZ15TA1) was prepared starting from 27 and 34 using the procedure described for the preparation of (SZ15TA3) in 61% yield. ¹H-NMR (400 MHz, CD₃CN) δ: 8.07 (d, J=8.4 Hz, 2H), 7.82 (d, J=7.6 Hz, 2H), 7.21 (d, J=8.0 Hz, 2H), 7.61 (t, J=7.2 Hz, 1H), 7.47 (t, J=7.6 Hz, 2H), 7.38-7.34 (m, 1H), 7.26-7.12 (m, 6H), 7.07 (t, J=8.8 Hz, 1H), 4.40 (s, 2H), 4.32 (s, 2H), 3.35-3.31 (m, 2H), 3.25-3.21 (m, 2H) ppm. ¹³C-NMR (100 MHz, CD₃CN) δ: 165.9, 162.7 (d, ¹J_{CF}=249 Hz), 160.5, 160.1, 140.9, 137.7, 136.9, 134.1, 133.7, 133.4, 133.3 (d, ³J_{CF}=10 Hz), 132.0, 131.9, 130.6, 129.9, 129.3, 128.9, 127.8, 126.7, 115.4 (d, ²J_{CF}=22.5 Hz), 57.8, 53.4, 49.4, 28.3 ppm.

4.11 Acylsulfonamide (SZ15TA2)

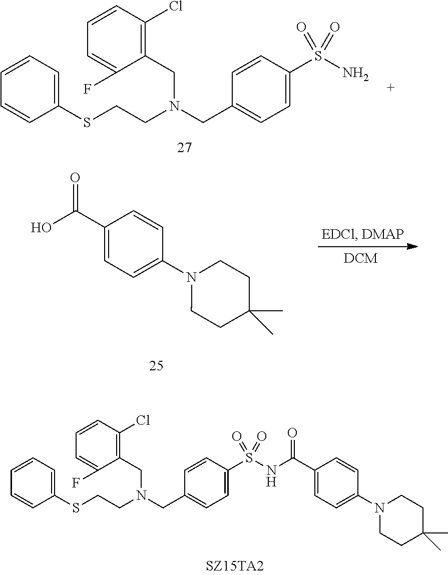 [SMILES: NS(=O)(=O)c1ccc(CN(CCSc2ccccc2)Cc2c(F)cccc2Cl)cc1 ; CC1(C)CCN(c2ccc(C(=O)O)cc2)CC1 ; CC1(C)CCN(c2ccc(C(=O)NS(=O)(=O)c3ccc(CN(CCSc4ccccc4)Cc4c(F)cccc4Cl)cc3)cc2)CC1]

(SZ15TA2) was prepared starting from 27 and 25 using the procedure described for the preparation of (SZ15TA3) in 48.4% yield. ¹H-NMR (400 MHz, CDCl₃) δ: 11.13 (bs, 1H), 7.96 (d, J=7.6 Hz, 2H), 7.79 (d, J=8.0 Hz, 2H), 7.56 (d, J=8.0 Hz, 2H), 7.27-7.12 (m, 10H), 6.93 (t, J=8.8 Hz, 1H), 4.27 (s, 2H), 4.24 (s, 2H), 3.35 (bs, 4H), 3.19 (bs, 2H), 3.15 (bs, 2H), 1.61 (bs, 4H), 0.99 (s, 6H) ppm. ¹³C-NMR (100 MHz, CDCl₃) δ: 164.2, 162.1 (d, ¹J_{CF}=241 Hz), 161.3, 160.8, 149.9, 140.0, 137.0, 136.7, 132.9, 132.3 (d, ³J_{CF}=9.5 Hz), 130.8, 130.5, 130.3, 129.2, 129.0, 127.4, 126.1, 125.8, 117.4, 114.7 (d, ²J_{CF}=22.6 Hz), 57.4, 52.8, 48.8, 48.5, 36.6, 28.6, 27.8, 27.4 ppm.

4.12 Acylsulfonamide (SZ15TA4)

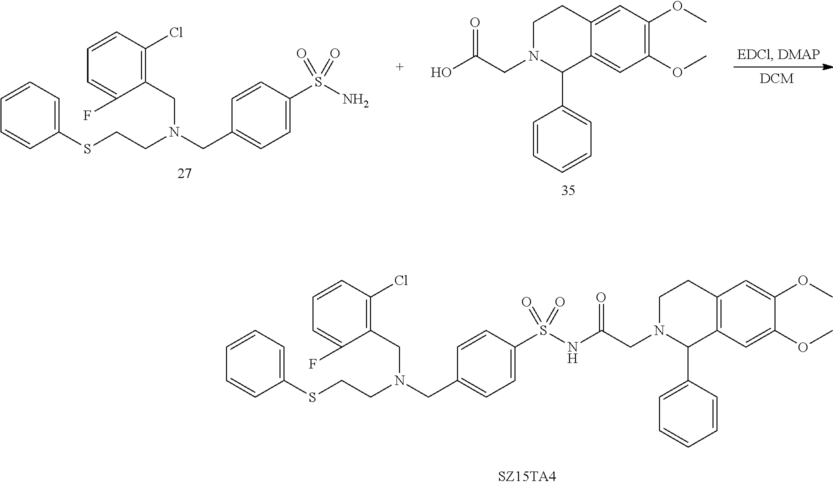 [SMILES: NS(=O)(=O)c1ccc(CN(CCSc2ccccc2)Cc2c(F)cccc2Cl)cc1 ; COc1cc2c(cc1OC)C(c1ccccc1)N(CC(=O)O)CC2 ; COc1cc2c(cc1OC)C(c1ccccc1)N(CC(=O)NS(=O)(=O)c1ccc(CN(CCSc3ccccc3)Cc3c(F)cccc3Cl)cc1)CC2]

(SZ15TA4) was prepared starting from 27 and 35 using the procedure described for the preparation of (SZ15TA3) in 48.4% yield. ¹H-NMR (400 MHz, CDCl₃) δ: 8.56 (bs, 1H), 7.94 (d, J=8.0 Hz, 2H), 7.59 (d, J=8.0 Hz, 2H), 7.35-7.30 (m, 4H), 7.24-7.18 (m, 10H), 6.98 (t, J=8.4 Hz, 1H), 6.65 (s, 1H), 6.18 (s, 1H), 5.70 (s, 1H), 4.28 (bs, 4H), 4.09 (d, J=16.4 Hz, 1H), 3.89 (d, J=16.8 Hz, 1H), 3.83 (s, 3H), 3.57 (s, 3H), 3.45 (bs, 2H), 3.19-3.02 (m, 6H) ppm. ¹³C-NMR (100 MHz, CDCl₃) δ: 165.0, 162.4 (d, ¹J_{CF}=251 Hz), 161.5, 149.8, 148.9, 139.8, 137.4, 137.0, 134.6, 133.2, 132.5 (d, ³J_{CF}=9.5 Hz), 131.3, 130.9, 130.7, 130.6, 129.5, 129.0, 127.7, 126.4, 122.9, 120.8, 117.5, 117.3, 115.0 (d, ²J_{CF}=22.6 Hz), 111.0 (d, ²J_{CF}=14.9 Hz), 66.4, 57.8, 56.1, 56.0, 54.0, 52.9, 49.1, 46.0, 29.0, 23.7 ppm. HRMS (ESI⁺) for [M+H]⁺; calculated: 774.22330. found: 774.22223 (error m/z=−1.37 ppm).

4.13 Acylsulfonamide (SZ15TA5)

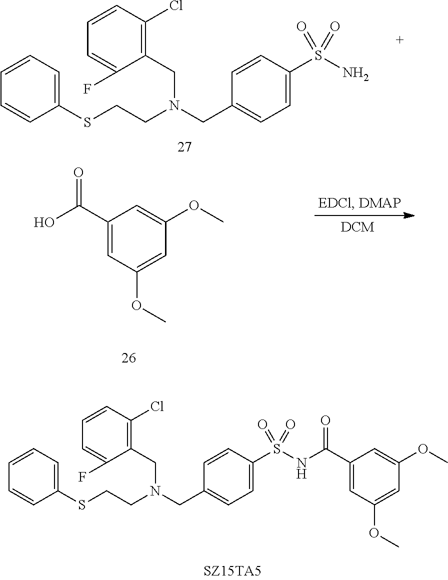 [SMILES: NS(=O)(=O)c1ccc(CN(CCSc2ccccc2)Cc2c(F)cccc2Cl)cc1 ; COc1cc(OC)cc(C(=O)O)c1 ; COc1cc(OC)cc(C(=O)NS(=O)(=O)c2ccc(CN(CCSc3ccccc3)Cc3c(F)cccc3Cl)cc2)c1]

(SZ15TA5) was prepared starting from 27 and 26 using the procedure described for the preparation of (SZ15TA3) in 44.2% yield. ¹H-NMR (400 MHz, CDCl₃) δ: 8.84 (bs, 1H), 8.04 (d, J=7.2 Hz, 2H), 7.60 (d, J=7.6 Hz, 2H), 7.28 (dd, J=14.0, 7.2 Hz, 1H), 7.16 (bs, 6H), 6.97 (t, J=8.4 Hz, 1H), 6.91 (s, 2H), 6.55 (s, 1H), 4.36 (s, 1H), 4.33 (s, 1H), 3.70 (s, 6H), 3.22 (bs, 4H) ppm. ¹³C-NMR (100 MHz, CDCl₃) δ: 165.1, 162.5 (d, ¹J_{CF}=226 Hz), 161.1, 140.3, 136.9, 136.2, 133.1, 133.0, 132.7, 131.3, 131.1, 129.6, 127.9, 126.5, 116.4 (d, ²J_{CF}=16.8 Hz), 115.1 (d, ²J_{CF}=22.5 Hz), 106.3, 106.0, 57.7, 55.8, 52.9, 49.1, 28.7 ppm. HRMS (ESI⁺) for [M+H]⁺; calculated: 629.13415. found: 629.13427 (error m/z=0.20 ppm).

4.14 Acylsulfonamide (SZ15TA6)

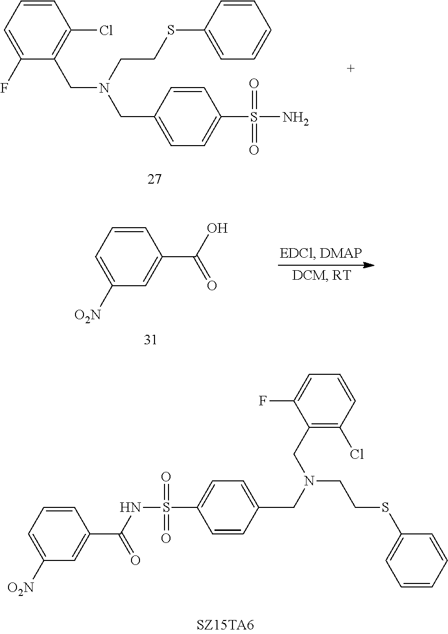 [SMILES: NS(=O)(=O)c1ccc(CN(CCSc2ccccc2)Cc2c(F)cccc2Cl)cc1 ; O=C(O)c1cccc([N+](=O)[O-])c1 ; O=C(NS(=O)(=O)c1ccc(CN(CCSc2ccccc2)Cc2c(F)cccc2Cl)cc1)c1cccc([N+](=O)[O-])c1]

(SZ15TA6) was prepared starting from 27 and 31 using the procedure described for the preparation of (SZ15TA3) in 60.7% yield ¹H-NMR (400 MHz, CDCl₃) δ: 8.63 (s, 1H), 8.24 (d, J=8 Hz, 2H), 8.17 (d, J=7.6 Hz, 2H), 8.05 (d, J=7 Hz, 3H), 7.64 (d, J=7 Hz, 2H), 7.54 (t, J=7 Hz, 1H), 7.28-7.23 (m, 2H), 7.14 (s, 6H), 7.02-6.93 (m, 2H), 4.36 (s, 2H), 4.32 (s, 2H), 3.24-3.21 (m, 4H) ppm. ¹³C-NMR (100 MHz, CDCl₃) δ: 163.5, 162.3 (d, ¹J_{CF}=208 Hz), 160.8, 148.0, 139.8, 136.6, 134.1, 133.0, 132.6, 131.0, 130.5, 129.9, 129.3, 127.5, 126.2, 123.4, 116.6 (d, ²J_{CF}=18 Hz), 114.8 (d, ²J_{CF}=22 Hz), 57.5, 52.6, 48.8, 28.5 ppm. HRMS (ESI⁺) for [M+Na]⁺; calculated: 636.0800. found: 636.0804 (error m/z=0.53 ppm).

4.15 Acylsulfonamide (SZ15TA7)

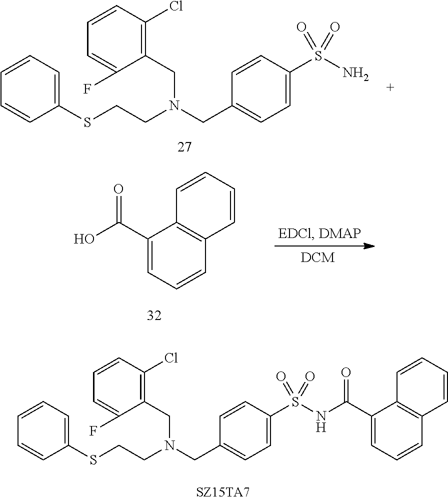 [SMILES: NS(=O)(=O)c1ccc(CN(CCSc2ccccc2)Cc2c(F)cccc2Cl)cc1 ; O=C(O)c1cccc2ccccc12 ; O=C(NS(=O)(=O)c1ccc(CN(CCSc2ccccc2)Cc2c(F)cccc2Cl)cc1)c1cccc2ccccc12]

(SZ15TA7) was prepared starting from 27 and 32 using the procedure described for the preparation of (SZ15TA3) in 48.4% yield. ¹H-NMR (400 MHz, CD₃OD) δ: 8.11 (d, J=8.4 Hz, 2H), 7.98 (d, J=8.4 Hz, 1H), 7.95 (d, J=8.8 Hz, 1H), 7.85 (d, J=8.4 Hz, 1H), 7.68-7.64 (m, 3H), 7.47 (d, J=8.0 Hz, 1H), 7.43 (d, J=9.2 Hz, 1H), 7.39-7.28 (m, 2H), 7.23-7.10 (m, 6H), 7.02 (t, J=8.8 Hz, 1H), 4.28 (s, 2H), 4.21 (s, 2H), 3.24-3.21 (m, 2H), 3.12-3.09 (m, 2H) ppm. ¹³C-NMR (100 MHz, CD₃OD) δ: 168.1, 162.3 (d, ¹J_{CF}=249.6 Hz), 140.2, 136.5, 133.9, 132.3, 132.0, 130.9, 130.6, 130.5, 130.0, 129.2, 128.7, 127.3, 127.1, 126.9, 126.6, 126.0, 124.4 (d, ³J_{CF}=10.7 Hz), 114.5 (d, ²J_{CF}=22.9 Hz), 57.4, 53.3, 48.5, 28.7 ppm.

4.16 Acylsulfonamide (SZ15TA9)

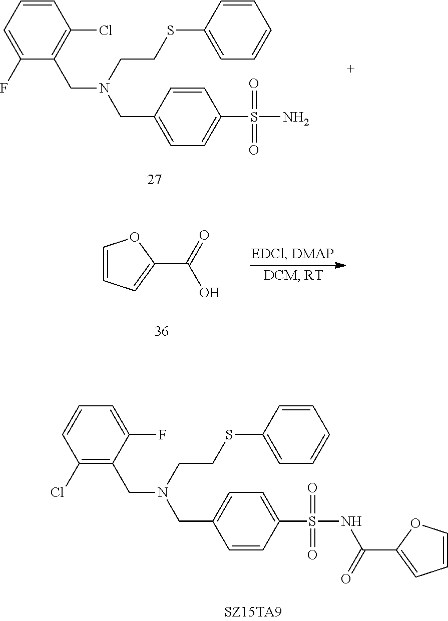 [SMILES: NS(=O)(=O)c1ccc(CN(CCSc2ccccc2)Cc2c(F)cccc2Cl)cc1 ; O=C(O)c1ccco1 ; O=C(NS(=O)(=O)c1ccc(CN(CCSc2ccccc2)Cc2c(F)cccc2Cl)cc1)c1ccco1]

(SZ15TA9) was prepared starting from 27 and 36 using the procedure described for the preparation of (SZ15TA9) in 91.6% yield. ¹H-NMR (400 MHz, CDCl₃) δ: 10.32 (s, 1H), 8.03 (d, J=8 Hz, 2H), 7.64 (d, J=8 Hz, 2H), 7.28-7.22 (m, 3H), 7.14-7.12 (m, 7H), 6.94 (t, J=9 Hz, 1H), 4.45 (s, 2H), 4.37 (s, 2H), 3.27 (s, 4H) ppm. ¹³C-NMR (100 MHz, CDCl₃) δ: 162.0 (d, ¹J_{CF}=251 Hz), 160.9, 160.5, 155.1, 146.6, 144.7, 140.2, 136.5, 135.3, 132.9 (d, ³J_{CF}=9.6 Hz), 132.0, 131.3, 130.7, 129.2, 129.1, 127.7, 126.2, 118.5, 117.1, 115.5 (d, ²J_{CF}=17 Hz), 114.8 (d, ²J_{CF}=22.6 Hz), 114.3, 112.8, 57.4, 52.6, 48.8, 28.1 ppm. HRMS (ESI⁺) for [M+H]⁺; calculated: 559.0923. found: 559.0915 (error m/z=−1.31 ppm).

4.17 Acylsulfonamide (SZ15TA10)

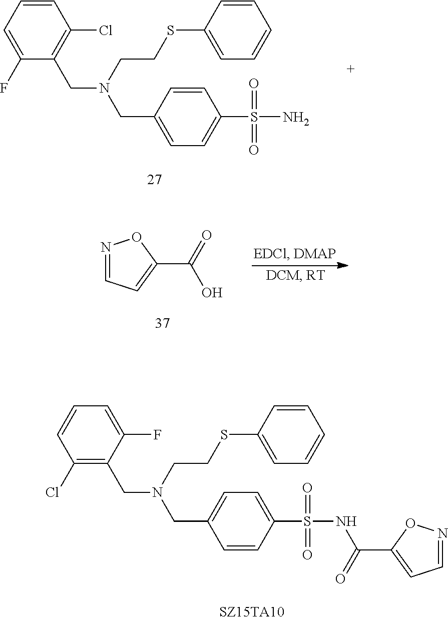 [SMILES: NS(=O)(=O)c1ccc(CN(CCSc2ccccc2)Cc2c(F)cccc2Cl)cc1 ; O=C(O)c1ccno1 ; O=C(NS(=O)(=O)c1ccc(CN(CCSc2ccccc2)Cc2c(F)cccc2Cl)cc1)c1ccno1]

(SZ15TA10) was prepared starting from 27 and 37 using the procedure described for the preparation of (SZ15TA9) in 91.6% yield. ¹H-NMR (400 MHz, CDCl₃) δ: 8.30 (s, 1H), 7.98 (d, J=8 Hz, 2H), 7.83 (bs, 1H), 7.54 (d, J=8 Hz, 2H), 7.18-7.10 (m, 7H), 6.99 (s, 1H), 6.91 (t, J=8.4 Hz, 1H), 4.06 (s, 2H), 4.02 (s, 2H), 3.13-3.11 (m, 2H), 2.97-2.95 (m, 2H) ppm. ¹³C-NMR (100 MHz, CDCl₃) δ: 162.2 (d, ¹J_{CF}=250 Hz), 160.3, 153.4, 151.2, 141.9, 137.9, 136.4, 134.2, 130.9 (d, ³J_{CF}=9.6 Hz), 129.9, 129.7, 129.0, 128.7, 126.6, 125.8, 120.3 (d, ²J_{CF}=17.6 Hz), 114.3 (d, ²J_{CF}=23 Hz), 108.7, 57.5, 52.9, 49.0, 29.6 ppm. HRMS (ESI⁺) for [M+H]⁺; calculated: 560.0875. found: 560.0873 (error m/z=−0.34 ppm).

4.18 Acylsulfonamide (SZ17TA3)

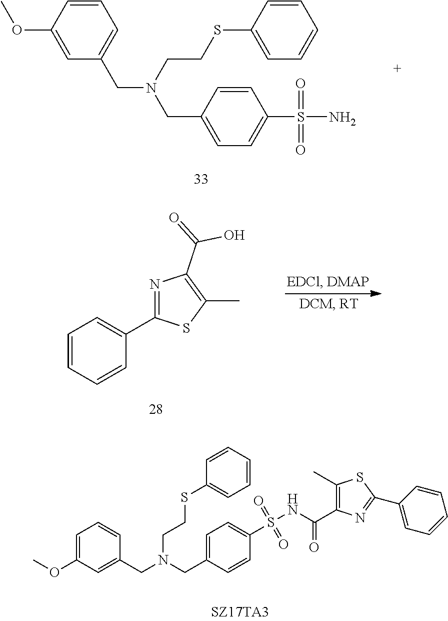 [SMILES: COc1cccc(CN(CCSc2ccccc2)Cc2ccc(S(N)(=O)=O)cc2)c1 ; Cc1sc(-c2ccccc2)nc1C(=O)O ; COc1cccc(CN(CCSc2ccccc2)Cc2ccc(S(=O)(=O)NC(=O)c3nc(-c4ccccc4)sc3C)cc2)c1]

(SZ17TA3) was prepared starting from 33 and 28 using the procedure described for the preparation of (SZ15TA9) in 79.5% yield. ¹H-NMR (400 MHz, CDCl₃) δ: 9.81 (s, 1H), 8.05 (d, J=8 Hz, 2H), 7.79 (d, J=7.2 Hz, 2H), 7.53 (d, J=7.6 Hz, 2H), 7.42-7.33 (m, 3H), 7.21-15 (m, 6H), 6.95 (s, 1H), 6.87 (d, J=8 Hz, 1H), 6.81 (d, J=7.2 Hz, 1H), 4.29 (s, 2H), 4.19 (s, 2H), 3.7 (s, 3H), 3.11-3.10 (m, 4H), 2.60 (s, 3H) ppm.

4.19 Acylsulfonamide (SZ3TA6)

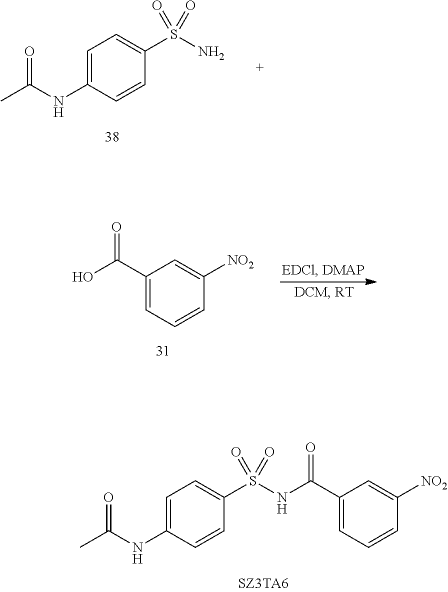 [SMILES: CC(=O)Nc1ccc(S(N)(=O)=O)cc1 ; O=C(O)c1cccc([N+](=O)[O-])c1 ; CC(=O)Nc1ccc(S(=O)(=O)NC(=O)c2cccc([N+](=O)[O-])c2)cc1]

(SZ3TA6) was prepared starting from 38 and 31 using the procedure described for the preparation of (SZ15TA9) in 45.5% yield. ¹H-NMR (250 MHz, acetone-d₆) δ: 8.61 (s, 1H), 8.31 (d, J=7.5 Hz, 1H), 8.22 (d, J=10 Hz, 1H), 7.9 (d, J=7.5 Hz, 2H), 7.75-7.64 (m, 3H), 1.99 (s, 3H) ppm. ¹³C-NMR (60 MHz, acetone-d₆) δ: 169.7, 149.2, 145.1, 135.2, 134.1, 131.1, 130.5, 128.0, 123.9, 119.1, 24.3 ppm.

4.20 Acylsulfonamide (SZ3TA9)

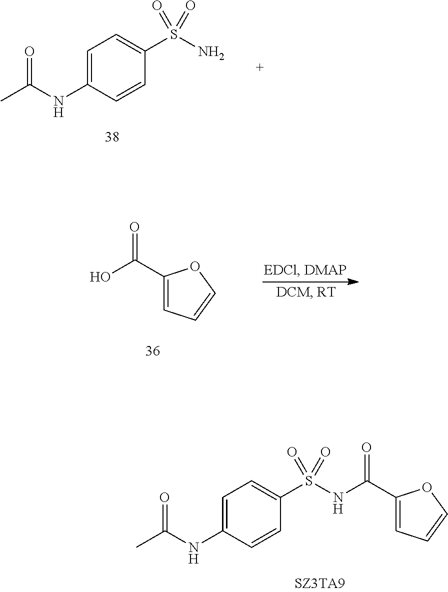 [SMILES: CC(=O)Nc1ccc(S(N)(=O)=O)cc1 ; O=C(O)c1ccco1 ; CC(=O)Nc1ccc(S(=O)(=O)NC(=O)c2ccco2)cc1]

(SZ3TA9) was prepared starting from 38 and 36 using the procedure described for the preparation of (SZ15TA9) in 35.5% yield. ¹H-NMR (400 MHz, CD₃OD) δ: 7.97 (d, J=8.8 Hz, 2H), 7.75 (d, J=8.8 Hz, 2H), 7.70 (d, J=1.2 Hz, 1H), 7.25 (d, J=1.2 Hz, 1H), 6.58 (dd, J=3.2, 1.6 Hz, 1H), 2.13 (s, 3H) ppm. ¹³C-NMR (100 MHz, CD₃OD) δ: 170.9, 156.4, 147.1, 145.6, 143.9, 133.8, 129.4, 118.9, 117.8, 112.4, 22.9 ppm. HRMS (ESI⁺) for [M+H]⁺; calculated: 309.05397. found: 309.05467 (error m/z=2.28 ppm).

4.21 Acylsulfonamide (SZ9TA7)

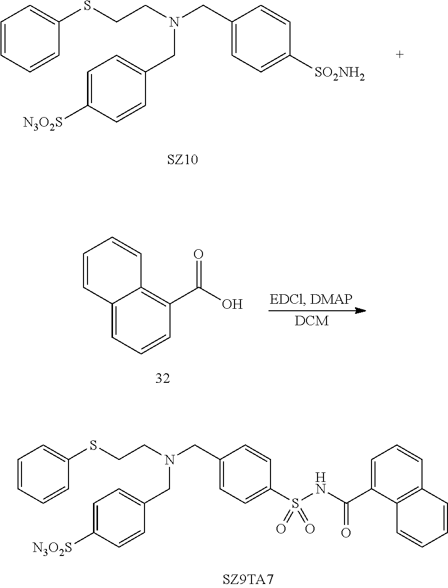

(SZ9TA7) was prepared starting from (SZ10) and 32 using the procedure described for the preparation of (SZ15TA9) in 69.3% yield. ¹H-NMR (400 MHz, CDCl₃) δ: 8.44 (s, 1H), 7.96 (d, J=8.0 Hz, 2H), 7.73-7.71 (m, 3H), 7.57-7.55 (m, 3H), 7.35 (d, J=8.0 Hz, 2H), 7.15-6.95 (m, 10H), 3.37 (s, 2H), 3.31 (s, 2H), 2.86 (t, J=6.4 Hz, 2H), 2.50 (bs, 2H) ppm. ¹³C-NMR (100 MHz, CDCl₃) δ: 147.0, 143.5, 140.0, 136.8, 135.8, 133.4, 131.4, 130.4, 129.5, 128.9, 128.8, 128.7, 128.0, 127.7, 127.4, 127.1, 126.9, 126.1, 125.7, 124.4, 57.6, 57.4, 52.5, 31.2 ppm. HRMS (ESI⁺) for [M+H]⁺; calculated: 309.05397. found: 309.05467 (error m/z=2.28 ppm).

4.22 Other Acylsulfonamides

Other acylsulfonamides described herein were prepared, in general, in accordance with the methods described above in Examples 4.1 to 4.22 and conventional organic synthesis techniques.

### Example 5

### Further Studies

Protein-protein interactions are central to many biological processes and hence represent a large and important class of targets for human therapeutics.^{1,2} Recent discovery of a variety of low-molecular-weight compounds that interfere with biologically relevant protein-protein complexes has fostered the identification and validation of new therapy strategies for a variety of diseases.³ Nevertheless, disrupting or modulating protein-protein interactions (PPIs) with low-molecular-weight compounds is extremely difficult due to the lack of deep binding pockets on protein surfaces. The adaptive nature of binding sites on protein surfaces creates additional challenges for lead compound design. Furthermore, because PPIs occur over a large surface area, it is difficult to identify potent and specific Protein-Protein Interaction Modulators (PPIMs) by conventional high-throughput screening (HTS) of small molecule libraries. Therefore, there is an urgent need to find simple, yet highly reliable and less cumbersome approaches to PPIM discovery. The fragment-based PPIM development strategy, in which screening and synthesis are combined in the same step, directs the medicinal chemist to focus synthetic resources solely on biologically active compounds at the earliest possible stages of PPIM development. The long-term goal of our research is to attack prevalent problems of conventional lead discovery and optimization approaches by establishing mass spectrometry-based (MS) tools to rapidly identify high-quality hits and to develop them into valuable leads.

The proteins of the anti-apoptotic Bcl-2 family (hereinafter referred to as Bcl), namely Bcl-2, Bcl-X_{L}, and Mcl-1, are among the protein-protein interaction targets which have been validated for cancer therapy.^{4,5} Bcl proteins are central regulators of programmed cell death. They are overexpressed in many cancers and contribute to tumor initiation, progression, and resistance to therapy.⁶ The conserved BH3 domain within the pro-apoptotic Bcl-2 family proteins has been shown to be critical for their cell death-inducing function and binding to anti-apoptotic Bcl proteins. Several efforts to develop low-molecular-weight compounds that mimic the BH3 domain of pro-apoptotic Bcl proteins have been reported in recent years. While a small number of compounds with potent anti-Bcl-2 and anti-Bcl-X_{L} activity have been discovered, only a few compounds are known to interact with Mcl-1.⁷ However, accumulating evidence suggests that it is advantageous to develop compounds selectively targeting Mcl-1, Bcl-2 or Bcl-X_{L} to induce apoptosis in most types of cancer.^{8,9} The herein presented disclosure utilizes a kinetic target-guided synthesis, PPIM development strategy to generate selective and potent PPIMs of the Bcl proteins, particularly Mcl-1.

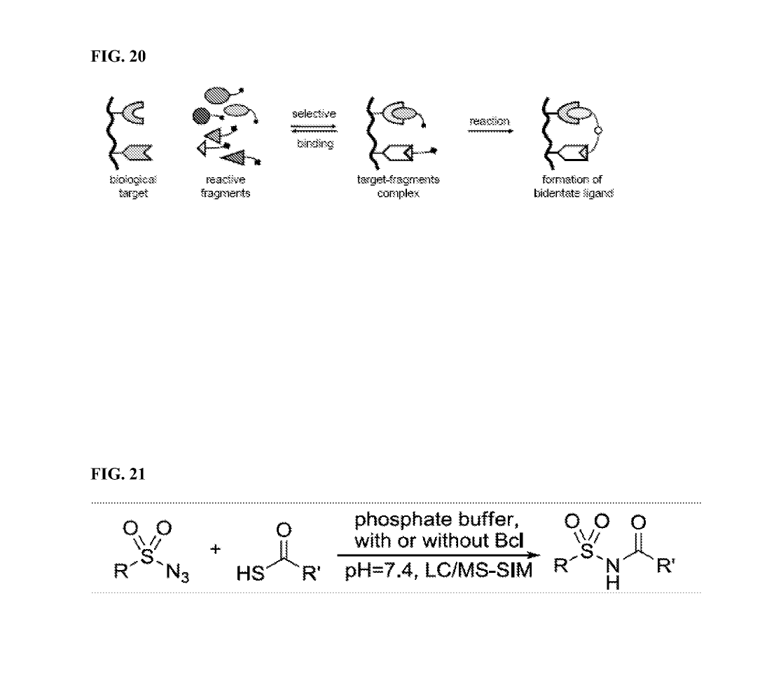

In the last decade, kinetic Target-Guided Synthesis (TGS) has been developed in which the biological target is actively engaged in the assembly of its own inhibitory bidentate ligand from a pool of smaller reactive fragments (FIG. 20).¹⁰⁻¹² To date, kinetic TGS has exclusively been applied to enzymatic targets and these TGS applications have been successful because of a unique combination of (a) the slow nature of the chemical reaction combining the two fragments into a single molecule and (b) the use of reactive fragments showing moderate to high affinity towards binding pockets of the enzyme. Compared to kinetic TGS screening of enzymes, however, the discovery of PPIMs is more challenging and thus requires major improvements over the existing kinetic TGS approaches. It is believed that the studies described herein will overcome the challenges associated with the application of kinetic TGS to PPIMs. These are:

(1) Study of irreversible, bio-orthogonal reactions in kinetic TGS approaches targeting Bcl-X_{L}. A repertoire of bio-orthogonal reactions will be established, which under buffered conditions and in the presence of Bcl-X_{L} covalently link two scaffolds into a single molecule with enhanced inhibitory properties.

(2) Implementation of MS-based affinity screening against Bcl proteins and incorporation of hit fragments in kinetic TGS approaches targeting Mcl-1 selectively over Bcl-2 and Bcl-X_{L}. A MS-based screening of a large fragment library will identify small fragments binding selectively to one of the three Bcl proteins. Subsequent evolution of fragment hits via kinetic TGS screenings will lead to the identification of high-quality bidentate ligands, which will be assessed for activity in cell-free assays.

(3) Hit-to-Lead progression of kinetic TGS hit compounds and assessment of biological activity in cell-free and cellular assays. Kinetic TGS hits will be developed into leads by a combination of kinetic TGS, conventional synthetic chemistry, physicochemical profiling, and molecular modeling with the objective to obtain selective PPIMs and to test whether kinetic TGS can be utilized in hit-to-lead progression. Furthermore, a physicochemical property and biological activity decision matrix will be implemented to only progress PPIMs further with acceptable inhibitory and physicochemical profiles.

### Example 6

### Research Strategy

A. Significance

High levels of the anti-apoptotic Bcl-2 family proteins (Bcl-2, Bcl-XL, Bcl-w, Mcl-1, and A1/BfI-1) are associated with the pathogenesis of cancer and resistance to therapy.^{13,14} A recent analysis of somatic copy-number alterations (SCNAs) showed that two anti-apoptotic Bcl-2 family genes (Bcl-X_{L} and Mcl-1) undergo frequent somatic amplifications in multiple cancers and cancer cells carrying Bcl-X_{L} and Mcl-1 amplifications are dependent on the expression of these genes for survival.¹⁵ Thus, the anti-apoptotic Bcl-2 family proteins are very attractive targets for the development of anticancer agents.

Over the last few years, several small-molecule Bcl-2 inhibitors have been synthesized and some of these molecules have entered clinical trials.^{16,17} Although Bcl-2 and Bcl-X_{L} have been the primary focus for the design of small-molecule inhibitors, recent studies have demonstrated that Mcl-1 also plays an important role for cancer cell survival and that Bcl-2, Bcl-X_{L} and Mcl-1 must be simultaneously neutralized for apoptosis induction in many types of cancer cells.¹⁸ Obatoclax (GX15-070) is a pan-Bcl-2 inhibitor¹⁹ but seems to have additional targets besides anti-apoptotic Bcl-2 family proteins and thus may lead to unpredicted, non-mechanism based toxicity.²⁰ Clearly, the more specific the inhibitor for individual Bcl-2 family members, the less non-mechanism based toxicity may be expected. To date, the most potent and selective small-molecule Bcl-2 inhibitors are ABT-737 and its orally active analog ABT-263, which only inhibit Bcl-2, Bcl-X_{L} and Bcl-w but do not target Mcl-1 or A1.²¹ Thus, these agents generally lack efficacy in tumors with elevated Mcl-1 or A1 and in many instances this resistance can be overcome by down-regulation of Mcl-1.²¹⁻²⁷ For example, knockdown of Mcl-1 or overexpression of Noxa, a BH3-only protein that selectively binds to and inhibits Mcl-1, sensitizes MCF-7 breast cancer cells to ABT-737.²³ Similarly, we have demonstrated that suppression of Mcl-1 expression allows ABT-737 to promote anoikis (detachment-induced apoptosis) in MDA-MB-231 breast cancer cells.²⁸ Most recently, it was shown in a non-Hodgkin's lymphoma model that acquired resistance was developed after the cells were exposed to ABT-737 for three weeks and transcriptional upregulation of Mcl-1 was detected,²⁹ suggesting a treatment regime combining ABT-737 with an Mcl-1-specific inhibitor may be necessary in order to overcome the resistance against ABT-737. Thus, there is an urgent need to develop Mcl-1 inhibitors for the treatment of ABT-737 resistant, Mcl-1-dependent human cancers.

Generally, cell-permeable small modulators of PPIs have been considered to be desirable tools.^{1,2} Nevertheless, reliable yet straightforward techniques or approaches for the development of potent and effective PPIMs are currently unavailable. Over the past 15 years, a variety of fragment-based lead discovery approaches have been developed and successfully applied for the development of potent PPIMs.³⁰⁻³² These approaches are commonly based on the detection of fragments binding to the target protein followed by the study of their binding to the protein target at atomic level resolution using X-ray crystallography or NMR spectroscopy. The initial hits are further optimized via fragment growing, in which fragments are extended into identified binding sites step-by-step, or via fragment linking, in which fragments identified to bind to adjacent binding sites are covalently linked together.³²⁻³⁵ Even though fragment-based discovery strategies have been very successful for the development of PPIMs, they are mainly limited by two constraints. Detection and quantification of fragment binding requires specially designed methodology due to the weak binding typically observed for fragments. Furthermore, the optimization of fragments into potent and selective compounds is not straightforward and not rapidly achievable, even though structural information is available.^{36,37} For example, though good quality NMR structures were available, the well-known development of Bcl-X_{L} PPIMs by Abbott^{7,38} required several design iterations and the preparation and testing of more than a thousand compounds³⁹ in order to yield ABT-737. Furthermore, of the very first design consisting of 21 different compounds containing the structural motifs of the initial fragments identified by NMR, most compounds bound to Bcl-X_{L} with a dissociation constant greater than 10 μM.⁴⁰

Recently, fragment-based discovery strategies have been reported which involve the protein target directly to select and assemble its own inhibitory compounds from pools of reactive fragments. These approaches, also termed in situ click chemistry or kinetic TGS approaches,^{41,42} were conceptually described in detail in the 1980s⁴³ and are still relatively unexplored compared to dynamic combinatorial chemistry. Thus far kinetic TGS has mainly been applied to the identification of potent enzyme inhibitors, nevertheless kinetic TGS offers an attractive approach to PPIM lead discovery because it allows the protein to select and combine building blocks that fit best into its binding sites, thus assembling larger compounds.^{41,42} The screening method can be as simple as determining whether or not the PPIM product has been formed in a given test mixture. Additionally, if one considers a protein target to be an ensemble of interconverting conformers, it is easy to imagine this protein undergoing dynamic motion to repeatedly expose unique structural elements vulnerable to strong binding by the right inhibitor. Unfortunately, such short-lived targets of opportunity cannot be ‘seen’ or easily discovered with present techniques. We speculate that kinetic TGS also has the potential to intercept and stabilize short-lived conformations of the protein target by the compounds assembled within the protein target. We hypothesize that the kinetic TGS approach to lead discovery will help medicinal chemists by providing a dynamic search method for tracking and intercepting the dynamic protein targets.

B. Innovation

Kinetic TGS shows great promise in fragment-based lead discovery because it combines the synthesis and screening of libraries of low-molecular-weight compounds into one single step. Our approach is to probe the protein's synthetic prowess with sets of reaction-capable building blocks that react only when held together properly. The real power of our PPIM discovery and optimization approach is derived entirely from the simple tactic of leaving the last step of the synthesis to the protein, thereby avoiding time- and resource-consuming syntheses of compounds that would not show biological activity in follow-up confirmatory studies. Furthermore, in contrast to conventional approaches, accumulated experimental evidence suggests that our PPIM screening reduces the number of false positives, cutting down the number of screening hits to be validated in confirmatory assays. The main objective of this proposal with Bcl proteins is to provide a general test case for the kinetic TGS discovery approach to generate selective hits targeting the proteins of the Bcl-2 family, which will be further progressed into valuable leads. We believe that the herein proposed PPIM discovery strategy for Bcl proteins is general and easily implementable to lead development initiatives targeting other PPIs such as MDM2/p53, IAP/caspase, Rb/Raf, and others.^{2,44,45}

C. Approach

C.1 Preliminary Results

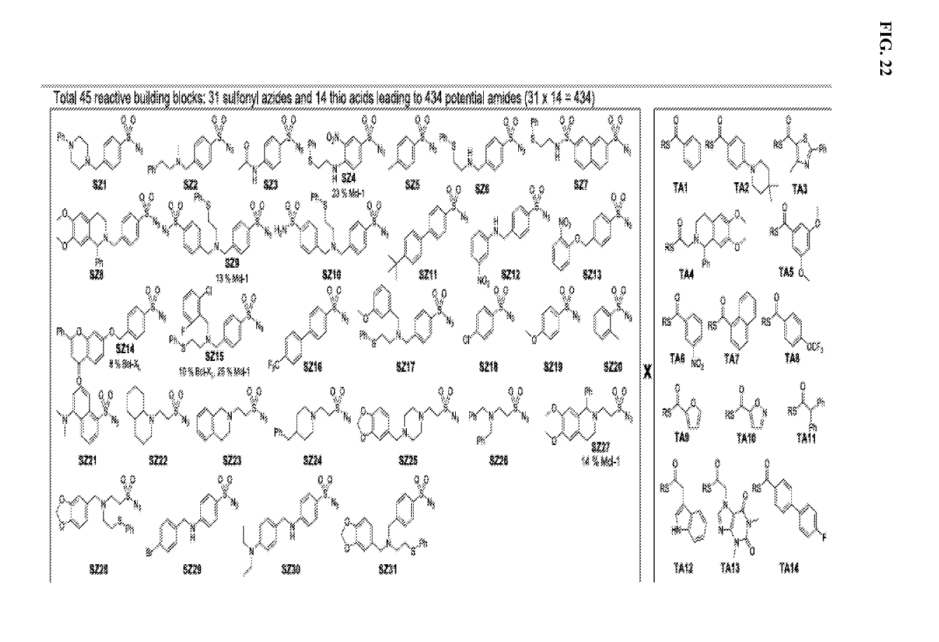

C.1.1 Proof-of-Concept Targeting Bcl-X_{L} Initial Proof-of-Concept Study⁴⁶: Williams and coworkers recently developed a reaction in which alkyl azides or sulfonyl azides react with thio acids to give corresponding amides (FIG. 21).^{47,48} This particular amidation reaction is high yielding at room temperature under dilute conditions and is effective in both organic and aqueous solvents. In a proof-of-concept study, we demonstrated that Bcl-X_{L} assembles its own PPIM from thio acids TA1-TA3 and sulfonyl azides SZ1-SZ6 (FIG. 22).⁴⁶ Analysis of each incubation sample by liquid chromatography combined with mass spectrometry detection in the selected ion mode (LC/MS-SIM)⁴⁹ showed that of all possible products, only compound SZ4TA2, which was originally developed by Abbott Laboratories, was detected.⁴⁶ Control incubations with wildtype (WT) and mutant versions of pro-apoptotic Bim BH3 peptide were conducted to assess whether the hit combination assembles at the targeted binding sites of Bcl-X_{L} or randomly elsewhere on the protein surface. WT BH3 peptides outcompete the reactive fragments for binding at the BH3 binding site of Bcl-X_{L} and thus suppress the Bcl-X_{L}-templated assembly of SZ4TA2, whereas the mutant BH3 peptides do not block the binding site of Bcl-X_{L}, and thus permit the assembly of SZ4TA2.⁴⁶

Proof-of-Concept with an Extended Fragment Library:

Our initial success motivated us to investigate whether kinetic TGS would also be successful in generating hit compounds which have not been previously reported. The thio acid and sulfonyl azide sublibraries have been extended by random compounds and by reactive fragments designed to be structurally related to ABT-737. Eighty one binary mixtures containing a single thio acid (TA1-TA9) and a single sulfonyl azide (SZ1-SZ9) were incubated with the target protein Bcl-X_{L} for 6 hours at 38° C. (FIG. 22). In parallel, identical binary fragment mixtures were incubated in buffer without Bcl-X_{L}. Similar to in situ click chemistry,41,50 all incubations were directly subjected to HPLC analysis with acylsulfonamide product detection by LC/MS-SIM.⁴⁹ Comparison of the LC/MS-SIM traces of identical fragment combinations with or without protein Bcl-X_{L} led to the identification of the previously reported fragment combination SZ4TA2⁴⁶ and three new combinations SZ7TA2, SZ9TA1 and SZ9TA5 with increased amounts of acylsulfonamide products in the incubations containing Bcl-X_{L}.

Control Incubations:

Next, the assembly of the new products SZ7TA2, SZ9TA1 and SZ9TA5 was confirmed via the aforementioned control incubations with Bcl-X_{L} and BH3 peptides. Furthermore, experiments were designed in which mutated Bcl-X_{L} proteins were incubated with reactive fragments. We reasoned that changes of the Bcl-X_{L} binding site directly affect the binding of hit reactive fragments to the protein which in turn alters the rate of the protein-templated reactions. The purpose of these mutant Bcl-X_{L} proteins was to expand the repertoire of controls with Bak BH3 and Bim BH3 peptides with complementary experiments indicating whether the TGS reaction occurs (a) with the help of the target protein Bcl-X_{L} and (b) specifically at the binding sites of interest. The known mutant of Bcl-X_{L}, in which Phe131 and Asp133 have been substituted by alanines, has been prepared since it fails to interact with either Bak or Bim BH3 peptides.⁵¹ In addition, a second mutant Bcl-X_{L} has been prepared, in which Arg139 has been replaced by alanine Arg 139 has been identified to be a key residue that interacts with ABT737 and analogues thereof.^{∂}Incubations of the two mutants with building blocks SZ4 and TA2 were undertaken first. In comparison to the incubation with WT Bcl-X_{L}, a reduction of the templation activity by approximately 60-80% has been observed in both mutant Bcl-X_{L}-templated reactions (Table 1).

TABLE 1
Incubations of SZ4 and TA2 with WT Bcl-X_{L}
and corresponding control incubations with Bak or Bim
BH3 peptides or mutated Bcl-X_{L}.

| Entry | Experiment | PA | % Signal |
|---|---|---|---|
| 1 | Buffer alone | 26,794 | 7.4 |
| 2 | WT Bcl-X_{L} | 363,187 | 100.0 |
| 3 | WT Bcl-X_{L} and WT Bak | 59,437 | 16.3 |
| 4 | WT Bcl-X_{L} and mutant Bak | 181,156 | 49.8 |
| 5 | WT Bcl-X_{L} and WT Bim | 51,773 | 14.3 |
| 6 | WT Bcl-X_{L} and mutant Bim | 217,813 | 59.9 |
| 7 | F131A, D133A Bcl-X_{L} | 157,059 | 43.2 |
| 8 | R139A Bcl-X_{L} | 95,154 | 26.2 |

PA = peak area

This observation can possibly be explained by closer examination of the reported NMR-structure (PDB#ID: 1YSI) of Bcl-X_{L} complexed with an acylsulfonamide, which is structurally related to TGS product SZ4TA2.⁷ Comparison of the location of Phe131 and Asp133 with the position of the acylsulfonamide in the Bcl-X_{L} binding sites reveals that the residues Phe131 and Asp133, although important for the binding to Bak and Bim BH3 peptides, are not in direct contact with the acylsulfonamide, while Arg139 appears to be closer to the acylsulfonamide. Thus, as expected, ^{R139A}Bcl-X_{L} displays a more significant suppression of the templated reaction compared to ^{F131A,D133A}Bcl-XL. For the confirmatory studies of the remaining 3 hit compounds SZ7TA2, SZ9TA1, and SZ9TA5, only ^{R139A}Bcl-X_{L} protein was utilized. The ^{R139A}Bcl-X_{L}-templated assembly of the hit combinations failed suggesting that SZ7TA2, SZ9TA1 and SZ9TA5 are true positives.

Activity of Kinetic TGS Hits:

To assess whether the kinetic TGS hits are more potent than acylsulfonamides, which were not identified in the kinetic TGS screening, hit compounds SZ7TA2, SZ9TA1 and SZ9TA5 and 33 additional acylsulfonamides were synthesized. All compounds were tested at a 50 μM concentration for PPI disruption in a fluorescence polarization assay (FP; see also Section C.4.1). The total number of acylsulfonamides tested corresponds to 45.7% of the 81 member library. Strikingly, the 4 kinetic TGS hits were the most potent compounds tested, disrupting the Bcl-X_{L}/BH3 interaction by 60% or more, while the randomly selected acylsulfonamides demonstrated an average activity of 15% (Table 2).

TABLE 2
Percentage Inhibition at 50 μM acylsulfonamide concentrations.
A total of 37 acylsulfonamides were tested for inhibition
at 50 μM concentration. Of the 37 compounds tested, the
four most potent compounds were identified by kinetic TGS.

| Frag- | | | | | | | | | |
|---|---|---|---|---|---|---|---|---|---|
| ments | SZ1 | SZ2 | SZ3 | SZ4 | SZ5 | SZ6 | SZ7 | SZ8 | SZ9 |
| TA1 | nd | 2 | 0 | 14 | 29 | nd | nd | 19 | 80 |
| TA2 | nd | 9 | nd | 100 | 28 | 26 | 76 | nd | 38 |
| TA3 | 6 | 7 | nd | nd | nd | nd | nd | 30 | 22 |
| TA4 | nd | 25 | nd | nd | nd | nd | nd | 8 | nd |
| TA5 | 5 | nd | nd | nd | 0 | nd | 15 | 11 | 60 |
| TA6 | 4 | nd | 0 | nd | 0 | nd | 20 | nd | 46 |
| TA7 | nd | nd | 0 | nd | nd | nd | 47 | 30 | nd |
| TA8 | 3 | nd | 0 | nd | nd | nd | nd | 38 | nd |
| TA9 | nd | nd | 0 | nd | 1 | nd | nd | 24 | nd |

ND = not determined

Only 4 of the 33 randomly selected acylsulfonamides demonstrated moderate activity between 35% and 45% inhibition. The kinetic TGS hits were subjected to dose response studies to obtain IC₅₀s. As reported by Abbott, SZ4TA2 was observed to have an IC₅₀ in the nM range against Bcl-X_{L} (FIG. 23).³⁸ The other 3 hits showed IC₅₀s in the low range. These results demonstrate that kinetic TGS identified the more active members of a library of potential compounds arising from the reactive fragments SZ1-SZ9 and TA1-TA9. This outcome is consistent with previously reported kinetic TGS studies, in which the enzyme carbonic anhydrase II preferably accelerates the formation of the better inhibitory compounds from a pool of reactive fragments.^{52,53} Other kinetic TGS examples using in situ click chemistry also suggest that the triazoles generated in the protein-templated reactions are the more potent inhibitors.^{49,53-59}

C.1.6 Kinetic TGS Screening against Mcl-1: The building blocks of the acylsulfonamide library (FIG. 23) have been screened in all possible combinations against Mcl-1. The initial kinetic TGS screening focused on fragments (SZ1-SZ30 and TA1-TA14), which were designed randomly. Following the optimized kinetic TGS screening protocol, incubations containing combinations of the sulfonyl azides SZ9, SZ15 or SZ17 with thio acids TA3, TA7, or TA8 were identified to form significant amounts of corresponding acylsulfonamides in the Mcl-1 templated reactions (Table 4). Furthermore, synthesized hit combinations containing especially the SZ15 or SZ17 moiety were shown to generate acylsulfonamides with moderate IC50s and respectable ligand efficiencies between 0.16-0.18. This observation led to the design of benzodioxolane SZ31, a slightly larger and more electron rich variant of SZ15 and SZ17, which produced additional kinetic TGS hits displaying slightly improved PPI inhibition (Table 4). These results are encouraging since fragment design directly guided by hits identified in the kinetic TGS screening appears to be capable of guiding medicinal chemists in the right direction.

C.1.7 Physicochemical Properties and Biological Activity in Cell-Free and Cellular Assays:

SAR and PPIM Activity:

Acylsulfonamides have been tested for their ability to disrupt the Bcl-X_{L}/BH3 and Mcl-1/BH3 interactions in the FP assays. We synthesized all acylsulfonamides (Table 4, entries 4-16), which have thus far been identified in the kinetic TGS screening against Mcl-1. In addition, a selection of structurally related compounds has been prepared to test whether these compounds have similar biological activity to the kinetic TGS hits (entries 1-3). Similar to our screening against Bcl-X_{L} (Table 2), the kinetic TGS hits are more potent than randomly selected acylsulfonamides. The SZ15, SZ17 or SZ31 acylsulfonamide series with their bis-benzylic tertiary amines are the most active compounds with IC₅₀s in the single digit μM range against Mcl-1. Compound SZ31TA2 is the most potent Mcl-1 PPIM, while SZ15TA1, approximately 10-fold less active than SZ31TA2, exhibits the best ligand efficiency. Furthermore, the majority of the compounds are approximately 3- to 7-fold less potent against Bcl-X_{L}. SZ31TA8 and SZ31TA2 are the most selective compounds with calculated selectivity indices of 19 and ≧61, respectively. Comparison of the structural features of the TA moieties with the Bcl-X_{L} inhibition constants suggests that the selectivity increases as the aromatic ring of the TA moiety is substituted with a rigid and/or lipophilic residue (TA2, TA3, TA7, or TA8 versus TA1, TA5, or TA6). To prove our hypothesis, acylsulfonamide SK-2-271 containing a biphenyl was synthesized and tested against Mcl-1 and Bcl-X_{L}. Though SK-2-271 was moderately potent against Mcl-1, its IC₅₀ against Bcl-X_{L} is greater than 200 μM rendering a selectivity index ≧15. In summary, to the best of our knowledge, SZ31TA2 with its selectivity index greater than 60 and a ligand efficiency of 0.159 is among the most selective Mcl-1 PPIM reported thus far. The identification of selective compounds is among the most important and challenging issues in drug discovery. Though SZ31TA2 with an IC₅₀ of 3.3 μM is only moderately active, its selectivity renders it a superb candidate for hit-to-lead optimization.

Biological Activity in Cellular Systems:

Selected SZ15- and SZ17-containing compounds have been studied in a cell viability assay at 100 μM concentration. The synthesized acylsulfonamides have been tested for their ability to induce apoptosis by inhibiting Bcl-X_{L} or Mcl-1 in K562 human leukemia cells stably expressing Bcl-X_{L} and Bim or Mcl-1 and Bim at a 1:1 ratio (Section C.4.1). Because the survival of these cells is dependent on the balanced association of Bim with Bcl-X_{L} or Mcl-1, the disruption of Bim association with Bcl-X_{L} or Mcl-1 by PPIMs will induce apoptosis and reduce cell viability. All tested compounds displayed moderate activity with a preference for Mcl-1-expressing cell lines reproducing a similar selectivity to the one observed in the cell-free assay.

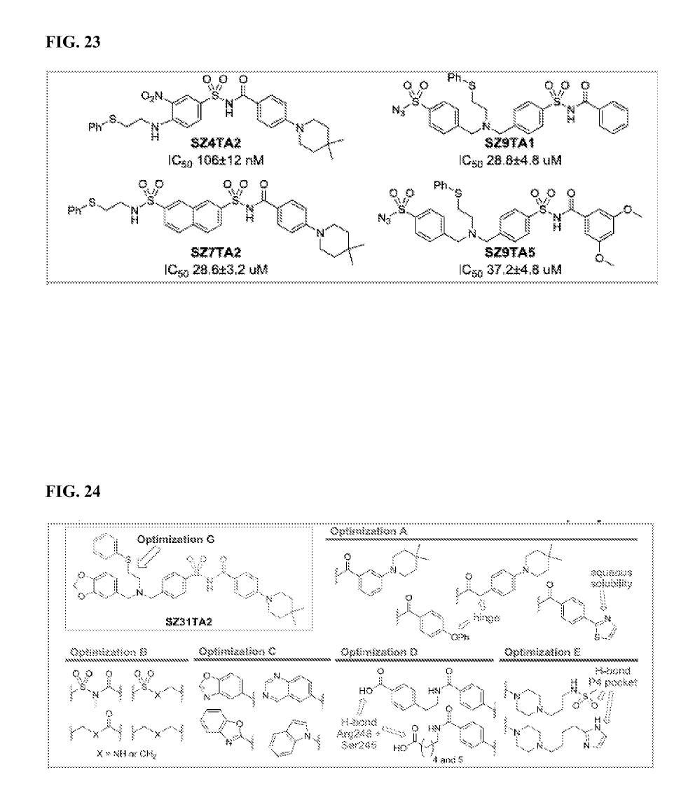

C.4.1 Hit Optimization to Improve Potency and Selectivity: Kinetic TGS hit compounds will be further optimized using a combination of conventional medicinal chemistry, kinetic TGS and molecular modeling. SZ15-, SZ17- and SZ31-containing molecules are considered to be interesting candidates for hit-to-lead optimization (Section C.1.7). A selected number of typical optimization efforts focusing solely on SZ31TA2 are described below (FIG. 24). Nevertheless, similar optimizations will be performed with the best disubstituted 2-propanol compounds or with other kinetic TGS hits, which will be identified in the course of this project. Besides improving potency and selectivity, optimization efforts will also aim at improving physicochemical properties. For SZ31TA2, the immediate medicinal chemistry plans entail:

Optimization A:

Current results suggest that the structure of the TA2 moiety influences the selectivity. TA2, TA3 and TA8 variants will be prepared with the objective to augment the ligand efficiency against Mcl-1 while improving the selectivity. The prepared fragments will be submitted for kinetic TGS against the hit fragments SZ15, SZ17 and SZ31. Hit combinations will be synthesized and tested for biological activity.

Optimization B:

Molecular modeling suggests that the acylsulfonamide linker is hydrogen bonding to Arg263 of Mcl-1. Structural modifications will probe the acylsulfonamide as a hydrogen bond donor or hydrogen bond acceptor. This optimization will identify the optimal structural motif linking the TA2 and the SZ31 together.

Optimization C:

The benezodioxolane moiety in SZ31TA2 will be replaced by other moieties with the objective to improve the potency, selectivity and/or aqueous solubility. The use of heterocycles such as the oxazole or quinazoline will also increase the aqueous solubility.

Optimization D:

Molecular modeling identified Arg248 and Ser245 to be potential residues to interact with SZ31TA2. Compounds with an acidic moiety extending from the thiophenyl ring will be synthesized and tested. A selection of potential acidic extensions is shown.

Optimization E:

Molecular modeling also identified potential hydrogen bond donors and acceptors in the P4 pocket. Replacement of the piperidine of SZ31TA2 by a piperazine enables to rapidly derivatize the compound with a hydrogen bond donor and/or acceptor. A selection of potential modifications is shown.

Optimization F:

The lengths of the various linkers connecting the peripheral aryl groups to the tertiary amine in SZ31 will be varied (not shown in FIG. 24).

### REFERENCES

1. Arkin, M. R.; Wells, J. A. Small-molecule inhibitors of protein-protein interactions: Progressing towards the dream. Nature Reviews Drug Discovery 2004, 3, 301-317.
2. Berg, T. Modulation of protein-protein interactions with small organic molecules. Angewandte Chemie-International Edition 2003, 42, 2462-2481.
3. Arkin, M. Protein-protein interactions and cancer: small molecules going in for the kill. Current Opinion in Chemical Biology 2005, 9, 317-324.
4. Verdine, G. L.; Walensky, L. D. The Challenge of Drugging Undruggable Targets in Cancer: Lessons Learned from Targeting BCI-2 Family Members. Clinical Cancer Research 2007, 13, 7264-7270.
5. Danial, N. N.; Korsmeyer, S. J. Cell death: Critical control points. Cell 2004, 116, 205-219.
6. Kirkin, V.; Joos, S.; Zornig, M. The role of BcI-2 family members in tumorigenesis. Biochimica Et Biophysica Acta-Molecular Cell Research 2004, 1644, 229-249.
7. Oltersdorf, T.; Elmore, S. W.; Shoemaker, A. R.; Armstrong, R. C.; Augeri, D. J.; Belli, B. A.; Bruncko, M.; Deckwerth, T. L.; Dinges, J.; Hajduk, P. J.; Joseph, M. K.; Kitada, S.; Korsmeyer, S. J.; Kunzer, A. R.; Letai, A.; Li, C.; Mitten, M. J.; Nettesheim, D. G.; Ng, S.; Nimmer, P. M.; O'Connor, J. M.; Oleksijew, A.; Petros, A. M.; Reed, J. C.; Shen, W.; Tahir, S. K.; Thompson, C. B.; Tomaselli, K. J.; Wang, B. L.; Wendt, M. D.; Zhang, H. C.; Fesik, S. W.; Rosenberg, S. H. An inhibitor of BcI-2 family proteins induces regression of solid tumours. Nature 2005, 435, 677-681.
8. Tse, C.; Shoemaker, A. R.; Adickes, J.; Anderson, M. G.; Chen, J.; Jin, S.; Johnson, E. F.; Marsh, K. C.; Mitten, M. J.; Nimmer, P.; Roberts, L.; Tahir, S. K.; Xiao, Y.; Yang, X.; Zhang, H.; Fesik, S.; Rosenberg, S. H.; Elmore, S. W. ABT-263: A Potent and Orally Bioavailable BcI-2 Family Inhibitor. Cancer Research 2008, 68, 3421-3428.
9. Shoemaker, A. R.; Mitten, M. J.; Adickes, J.; Ackler, S.; Refici, M.; Ferguson, D.; Oleksijew, A.; O'Connor, J. M.; Wang, B.; Frost, D. J.; Bauch, J.; Marsh, K.; Tahir, S. K.; Yang, X.; Tse, C.; Fesik, S. W.; Rosenberg, S. H.; Elmore, S. W. Activity of the BcI-2 Family Inhibitor ABT-263 in a Panel of Small Cell Lung Cancer Xenograft Models. Clinical Cancer Research 2008, 14, 3268-3277.
10. Shuker, S. B.; Hajduk, P. J.; Meadows, R. P.; Fesik, S. W. Discovering high-affinity ligands for proteins: SAR by NMR. Science 1996, 274, 1531-1534.
11. Huth, J. R.; Sun, C. H. Utility of NMR in lead optimization: Fragment-based approaches. Combinatorial Chemistry & High Throughput Screening 2002, 5, 631-643.
12. Vaj da, S.; Guarnieri, F. Characterization of protein-ligand interaction sites using experimental and computational methods. Current Opinion in Drug Discovery & Development 2006, 9, 354-362.
13. Reed, J. C.; Miyashita, T.; Takayama, S.; Wang, H. G.; Sato, T.; Krajewski, S.; Aime-Sempe, C.; Bodrug, S.; Kitada, S.; Hanada, M. BCI-2 family proteins: regulators of cell death involved in the pathogenesis of cancer and resistance to therapy. J Cell Biochem 1996, 60, 23-32.
14. Reed, J. C. BcI-2 family proteins: strategies for overcoming chemoresistance in cancer. Adv Pharmacol 1997, 41, 501-32.
15. Beroukhim, R.; Mermel, C. H.; Porter, D.; Wei, G.; Raychaudhuri, S.; Donovan, J.; Barretina, J.; Boehm, J. S.; Dobson, J.; Urashima, M.; Mc Henry, K. T.; Pinchback, R. M.; Ligon, A. H.; Cho, Y. J.; Haery, L.; Greulich, H.; Reich, M.; Winckler, W.; Lawrence, M. S.; Weir, B. A.; Tanaka, K. E.; Chiang, D. Y.; Bass, A. J.; Loo, A.; Hoffman, C.; Prensner, J.; Liefeld, T.; Gao, Q.; Yecies, D.; Signoretti, S.; Maher, E.; Kaye, F. J.; Sasaki, H.; Tepper, J. E.; Fletcher, J. A.; Tabernero, J.; Baselga, J.; Tsao, M. S.; Demichelis, F.; Rubin, M. A.; Janne, P. A.; Daly, M. J.; Nucera, C.; Levine, R. L.; Ebert, B. L.; Gabriel, S.; Rustgi, A. K.; Antonescu, C. R.; Ladanyi, M.; Letai, A.; Garraway, L. A.; Loda, M.; Beer, D. G.; True, L. D.; Okamoto, A.; Pomeroy, S. L.; Singer, S.; Golub, T. R.; Lander, E. S.; Getz, G.; Sellers, W. R.; Meyerson, M. The landscape of somatic copy-number alteration across human cancers. Nature 2010, 463, 899-905.
16. Yip, K. W.; Reed, J. C. BcI-2 family proteins and cancer. Oncogene 2008, 27, 6398-6406.
17. Vogler, M.; Dinsdale, D.; Dyer, M. J.; Cohen, G. M. BcI-2 inhibitors: small molecules with a big impact on cancer therapy. Cell Death Differ 2009, 16, 360-367.
18. Willis, S. N.; Chen, L.; Dewson, G.; Wei, A.; Naik, E.; Fletcher, J. I.; Adams, J. M.; Huang, D. C. Proapoptotic Bak is sequestered by McI-1 and BcI-xL, but not BcI-2, until displaced by BH3-only proteins. Genes Dev 2005, 19, 1294-1305.
19. Nguyen, M.; Marcellus, R. C.; Roulston, A.; Watson, M.; Serfass, L.; Murthy Madiraju, S. R.; Goulet, D.; Viallet, J.; Belec, L; Billot, X.; Acoca, S.; Purisima, E.; Wiegmans, A.; Cluse, L.; Johnstone, R. W.; Beauparlant, P.; Shore, G. C. Small molecule obatoclax (GX15-070) antagonizes MCL-1 and overcomes MCL-1-mediated resistance to apoptosis. Proc Natl Acad Sci USA 2007, 104, 19512-19517.
20. Konopleva, M.; Watt, J.; Contractor, R.; Tsao, T.; Harris, D.; Estrov, Z.; Bornmann, W.; Kantarjian, H.; Viallet, J.; Samudio, I.; Andreeff, M. Mechanisms of antileukemic activity of the novel BcI-2 homology domain-3 mimetic GX15-070 (obatoclax). Cancer Res 2008, 68, 3413-3420.
21. Tse, C.; Shoemaker, A. R.; Adickes, J.; Anderson, M. G.; Chen, J.; Jin, S.; Johnson, E. F.; Marsh, K. C.; Mitten, M. J.; Nimmer, P.; Roberts, L.; Tahir, S. K.; Xiao, Y.; Yang, X.; Zhang, H.; Fesik, S.; Rosenberg, S. H.; Elmore, S. W. ABT-263: a potent and orally bioavailable BcI-2 family inhibitor. Cancer Res 2008, 68, 3421-3428.
22. Oltersdorf, T.; Elmore, S. W.; Shoemaker, A. R.; Armstrong, R. C.; Augeri, D. J.; Belli, B. A.; Bruncko, M.; Deckwerth, T. L.; Dinges, J.; Hajduk, P. J.; Joseph, M. K.; Kitada, S.; Korsmeyer, S. J.; Kunzer, A. R.; Letai, A.; Li, C.; Mitten, M. J.; Nettesheim, D. G.; Ng, S.; Nimmer, P. M.; O'Connor, J. M.; Oleksijew, A.; Petros, A. M.; Reed, J. C.; Shen, W.; Tahir, S. K.; Thompson, C. B.; Tomaselli, K. J.; Wang, B.; Wendt, M. D.; Zhang, H.; Fesik, S. W.; Rosenberg, S. H. An inhibitor of BcI-2 family proteins induces regression of solid tumours. Nature 2005, 435, 677-681.
23. van Delft, M. F.; Wei, A. H.; Mason, K. D.; Vandenberg, C. J.; Chen, L.; Czabotar, P. E.; Willis, S. N.; Scott, C. L.; Day, C. L.; Cory, S.; Adams, J. M.; Roberts, A. W.; Huang, D. C. The BH3 mimetic ABT-737 targets selective BcI-2 proteins and efficiently induces apoptosis via Bak/Bax if McI-1 is neutralized. Cancer Cell 2006, 10, 389-399.
24. Chen, S.; Dai, Y.; Harada, H.; Dent, P.; Grant, S. McI-1 down-regulation potentiates ABT-737 lethality by cooperatively inducing Bak activation and Bax translocation. Cancer Res 2007, 67, 782-791.
25. Konopleva, M.; Contractor, R.; Tsao, T.; Samudio, I.; Ruvolo, P. P.; Kitada, S.; Deng, X.; Zhai, D.; Shi, Y. X.; Sneed, T.; Verhaegen, M.; Soengas, M.; Ruvolo, V. R.; McQueen, T.; Schober, W. D.; Watt, J. C.; Jiffar, T.; Ling, X.; Marini, F. C.; Harris, D.; Dietrich, M.; Estrov, Z.; McCubrey, J.; May, W. S.; Reed, J. C.; Andreeff, M. Mechanisms of apoptosis sensitivity and resistance to the BH3 mimetic ABT-737 in acute myeloid leukemia. Cancer Cell 2006, 10, 375-388.
26. Lin, X.; Morgan-Lappe, S.; Huang, X.; Li, L.; Zakula, D. M.; Vernetti, L. A.; Fesik, S. W.; Shen, Y. ‘Seed’ analysis of off-target siRNAs reveals an essential role of McI-1 in resistance to the small-molecule BcI-2/BcI-X_{L} inhibitor ABT-737. Oncogene 2007, 26, 3972-3979.
27. Tahir, S. K.; Yang, X.; Anderson, M. G.; Morgan-Lappe, S. E.; Sarthy, A. V.; Chen, J.; Warner, R. B.; Ng, S. C.; Fesik, S. W.; Elmore, S. W.; Rosenberg, S. H.; Tse, C. Influence of BcI-2 family members on the cellular response of small-cell lung cancer cell lines to ABT-737. Cancer Res 2007, 67, 1176-1183.
28. Woods, N. T.; Yamaguchi, H.; Lee, F. Y.; Bhalla, K. N.; Wang, H. G. Anoikis, initiated by McI-1 degradation and Bim induction, is deregulated during oncogenesis. Cancer Res 2007, 67, 10744-10752.
29. Yecies, D.; Carlson, N. E.; Deng, J.; Letai, A. Acquired resistance to ABT-737 in lymphoma cells that upregulate of MCL-1 and BFL-1. Blood, blood-2009-07-233304.
30. Albert, J. S.; Blomberg, N.; Breeze, A. L.; Brown, A. J. H.; Burrows, J. N.; Edwards, P. D.; Folmer, R. H. A.; Geschwindner, S.; Griffen, E. J.; Kenny, P. W.; Nowak, T.; Olsson, L. L.; Sanganee, H.; Shapiro, A. B. An integrated approach to fragment-based lead generation: Philosophy, strategy and case studies from AstraZeneca's drug discovery programmes. Current Topics in Medicinal Chemistry 2007, 7, 1600-1629.
31. Erlanson, D. A. Fragment-based lead discovery: a chemical update. Current Opinion in Biotechnology 2006, 17, 643-652.
32. Can, R. A. E.; Congreve, M.; Murray, C. W.; Rees, D. C. Fragment-based lead discovery: leads by design. Drug Discovery Today 2005, 10, 987-992.
33. Poulsen, S. A.; Bornaghi, L. F. Fragment-based drug discovery of carbonic anhydrase II inhibitors by dynamic combinatorial chemistry utilizing alkene cross metathesis. Bioorganic & Medicinal Chemistry 2006, 14, 3275-3284.
34. Schulz, M. N.; Hubbard, R. E. Recent progress in fragment-based lead discovery. Current Opinion in Pharmacology 2009, 9, 615-621.
35. Congreve, M.; Chessari, G.; Tisi, D.; Woodhead, A. J. Recent developments in fragment-based drug discovery. Journal of Medicinal Chemistry 2008, 51, 3661-3680.
36. Schulz Michele, N.; Hubbard Roderick, E. Recent progress in fragment-based lead discovery. Current opinion in pharmacology 2009, 9, 615-621.
37. Murray, C. W.; Rees, D. C. The rise of fragment-based drug discovery. Nature Chemistry 2009, 1, 187192.
38. Wendt, M. D.; Shen, W.; Kunzer, A.; McClellan, W. J.; Bruncko, M.; Oost, T. K.; Ding, H.; Joseph, M. K.; Zhang, H. C.; Nimmer, P. M.; Ng, S. C.; Shoemaker, A. R.; Petros, A. M.; Oleksijew, A.; Marsh, K.; Bauch, J.; Oltersdorf, T.; Belli, B. A.; Martineau, D.; Fesik, S. W.; Rosenberg, S. H.; Elmore, S. W. Discovery and structure-activity relationship of antagonists of B-cell lymphoma 2 family proteins with chemopotentiation activity in vitro and in vivo. Journal of Medicinal Chemistry 2006, 49, 1165-1181.
39. Hajduk, P. J. Fragment-Based Drug Design: How Big Is Too Big? Journal of Medicinal Chemistry 2006, 49, 6972-6976.
40. Petros, A. M.; Dinges, J.; Augeri, D. J.; Baumeister, S. A.; Betebenner, D. A.; Bures, M. G.; Elmore, S. W.; Hajduk, P. J.; Joseph, M. K.; Landis, S. K.; Nettesheim, D. G.; Rosenberg, S. H.; Shen, W.; Thomas, S.; Wang, X. L.; Zanze, I.; Zhang, H. C.; Fesik, S. W. Discovery of a potent inhibitor of the antiapoptotic protein BcI-x(L) from NMR and parallel synthesis. Journal of Medicinal Chemistry 2006, 49, 656-663.
41. Sharpless, K. B.; Manetsch, R. In situ click chemistry: a powerful means for lead discovery. Expert Opin. Drug Discovery 2006, 1, 525-538.
42. Hu, X. D.; Manetsch, R. Kinetic target-guided synthesis. Chemical Society Reviews 2010, 39, 1316-1324.
43. Jencks, W. P. On the Attribution and Additivity of Binding-Energies. Proceedings of the National Academy of Sciences of the United States of America-Biological Sciences 1981, 78, 4046-4050.
44. Wells, J. A.; McClendon, C. L. Reaching for high-hanging fruit in drug discovery at protein-protein interfaces. Nature (London, United Kingdom) 2007, 450, 1001-1009.
45. Bohacek, R. S.; McMartin, C.; Guida, W. C. The art and practice of structure-based drug design: a molecular modeling perspective. Medicinal Research Reviews 1996, 16, 3-50.
46. Hu, X.; Sun, J.; Wang, H.-G.; Manetsch, R. BcI-X_{L}-Templated Assembly of Its Own Protein-Protein Interaction Modulator from Fragments Decorated with Thio Acids and Sulfonyl Azides. Journal of the American Chemical Society 2008, 130, 13820-13821.
47. Shangguan, N.; Katukojvala, S.; Greenburg, R.; Williams, L. J. The reaction of thio acids with azides: A new mechanism and new synthetic applications. Journal of the American Chemical Society 2003, 125, 7754-7755.
48. Kolakowski, R. V.; Shangguan, N.; Sauers, R. R.; Williams, L. J. Mechanism of thio acid/azide amidation. Journal of the American Chemical Society 2006, 128, 5695-5702.
49. Manetsch, R.; Krasinski, A.; Radic, Z.; Raushel, J.; Taylor, P.; Sharpless, K. B.; Kolb, H. C. In situ click chemistry: Enzyme inhibitors made to their own specifications. Journal of the American Chemical Society 2004, 126, 12809-12818.
50. Mamidyala, S. K.; Finn, M. G. In situ click chemistry: probing the binding landscapes of biological molecules. Chemical Society Reviews 39, 1252-1261.
51. Yamaguchi, H.; Wang, H.-G. BcI-X_{L} protects BimEL-induced Bax conformational change and cytochrome c release independent of interacting with Bax or BimEL. Journal of Biological Chemistry 2002, 277, 4160441612.
52. Nguyen, R.; Huc, I. Using an enzyme's active site to template inhibitors. Angewandte Chemie-International Edition 2001, 40, 1774-1776.
53. Mocharla, V. P.; Colasson, B.; Lee, L. V.; Roper, S.; Sharpless, K. B.; Wong, C. H.; Kolb, H. C. In situ click chemistry: Enzyme-generated inhibitors of carbonic anhydrase II. Angewandte Chemie-International Edition 2005, 44, 116-120.
54. Krasinski, A.; Radic, Z.; Manetsch, R.; Raushel, J.; Taylor, P.; Sharpless, K. B.; Kolb, H. C. In situ selection of lead compounds by click chemistry: Target-guided optimization of acetylcholinesterase inhibitors. Journal of the American Chemical Society 2005, 127, 6686-6692.
55. Whiting, M.; Muldoon, J.; Lin, Y. C.; Silverman, S. M.; Lindstrom, W.; Olson, A. J.; Kolb, H. C.; Finn, M. G.; Sharpless, K. B.; Elder, J. H.; Fokin, V. V. Inhibitors of HIV-1 protease by using in situ click chemistry. Angewandte Chemie-International Edition 2006, 45, 1435-1439.
56. Wang, J.; Sui, G.; Mocharla, V. P.; Lin, R. J.; Phelps, M. E.; Kolb, H. C.; Tseng, H.-R. Integrated microfluidics for parallel screening of an in situ click chemistry library. Angewandte Chemie, International Edition 2006, 45, 5276-5281.
57. Hirose, T.; Sunazuka, T.; Sugawara, A.; Endo, A.; Iguchi, K.; Yamamoto, T.; Ui, H.; Shiomi, K.; Watanabe, T.; Sharpless, K. B.; Omura, S. Chitinase inhibitors: extraction of the active framework from natural argifin and use of in situ click chemistry. Journal of Antibiotics 2009, 62, 277-282.
58. Agnew, H. D.; Rohde, R. D.; Millward, S. W.; Nag, A.; Yeo, W.-S.; Hein, J. E.; Pitram, S. M.; Tariq, A. A.; Burns, V. M.; Krom, R. J.; Fokin, V. V.; Sharpless, K. B.; Heath, J. R. Iterative In Situ Click Chemistry Creates Antibody-like Protein-Capture Agents. Angewandte Chemie, International Edition 2009, 48, 49444948, 54944/1-84944/29.
59. Wang, Y.; Lin, W.-Y.; Liu, K.; Lin, R. J.; Selke, M.; Kolb, H. C.; Zhang, N.; Zhao, X.-Z.; Phelps, M. E.; Shen, C. K. F.; Faull, K. F.; Tseng, H.-R. An integrated microfluidic device for large-scale in situ click chemistry screening. Lab on a Chip 2009, 9, 2281-2285.
60. Crich, D.; Sasaki, K. Reaction of Thioacids with Isocyanates and Isothiocyanates: A Convenient Amide Ligation Process. Organic Letters 2009, 11, 3514-3517.
61. Greasley, S. E.; Marsilje, T. H.; Cai, H.; Baker, S.; Benkovic, S. J.; Boger, D. L.; Wilson, I. A. Unexpected formation of an epoxide-derived multisubstrate adduct inhibitor on the active site of GAR transformylase. Biochemistry 2001, 40, 13538-13547.
62. Park, C.-M.; Bruncko, M.; Adickes, J.; Bauch, J.; Ding, H.; Kunzer, A.; Marsh, K. C.; Nimmer, P.; Shoemaker, A. R.; Song, X.; Tahir, S. K.; Tse, C.; Wang, X.; Wendt, M. D.; Yang, X.; Zhang, H.; Fesik, S. W.; Rosenberg, S. H.; Elmore, S. W. Discovery of an Orally Bioavailable Small Molecule Inhibitor of Prosurvival B-Cell Lymphoma 2 Proteins. Journal of Medicinal Chemistry 2008, 51, 6902-6915.
63. Bemis, G. W.; Murcko, M. A. The properties of known drugs. 1. Molecular frameworks. Journal of medicinal chemistry 1996, 39, 2887-93.
64. Bemis, G. W.; Murcko, M. A. Properties of Known Drugs. 2. Side Chains. Journal of Medicinal Chemistry 1999, 42, 5095-5099.
65. Ghose, A. K.; Viswanadhan, V. N.; Wendoloski, J. J. A Knowledge-Based Approach in Designing Combinatorial or Medicinal Chemistry Libraries for Drug Discovery. 1. A Qualitative and Quantitative Characterization of Known Drug Databases. Journal of Combinatorial Chemistry 1999, 1, 55-68.
66. Cancilla, M. T.; Erlanson, D. A. Tethering: fragment-based drug discovery by mass spectrometry. Methods and Principles in Medicinal Chemistry 2007, 36, 305-320.
67. Di, L.; Kerns, E. H. Stability Challenges in Drug Discovery. Chem. Biodiversity 2009, 6, 1875-1886.
68. Kerns, E. H.; Di, L. Utility of mass spectrometry for pharmaceutical profiling applications. Curr. Drug Metab. 2006, 7, 457-466.
69. Di, L.; Kerns, E. H.; Hong, Y.; Chen, H. Development and application of high throughput plasma stability assay for drug discovery. Int. J. Pharm. 2005, 297, 110-119.
70. Lombardo, F.; Shalaeva, M. Y.; Tupper, K. A.; Gao, F. ElogD(oct): a tool for lipophilicity determination in drug discovery. 2. Basic and neutral corn pounds. J. Med. Chem. 2001, 44, 2490-2497.
71. Valko, K.; Nunhuck, S.; Bevan, C.; Abraham Michael, H.; Reynolds Derek, P. Fast gradient HPLC method to determine compounds binding to human serum albumin. Relationships with octanol/water and immobilized artificial membrane lipophilicity. J Pharm Sci 2003, 92, 2236-2248.
72. Lipinski, C. A.; Lombardo, F.; Dominy, B. W.; Feeney, P. J. Experimental and computational approaches to estimate solubility and permeability in drug discovery and development settings. Advanced Drug Delivery Reviews 1997, 23, 3-25.
73. Lipinski, C. A.; Lombardo, F.; Dominy, B. W.; Feeney, P. J. Experimental and computational approaches to estimate solubility and permeability in drug discovery and development settings. Advanced Drug Delivery Reviews 2001, 46, 3-26.
74. Friesner, R. A.; Banks, J. L.; Murphy, R. B.; Halgren, T. A.; Klicic, J. J.; Mainz, D. T.; Repasky, M. P.; Knoll, E. H.; Shelley, M.; Perry, J. K.; Shaw, D. E.; Francis, P.; Shenkin, P. S. Glide: A new approach for rapid, accurate docking and scoring. 1. Method and assessment of docking accuracy. Journal of Medicinal Chemistry 2004, 47, 1739-1749.
75. Halgren, T. A.; Murphy, R. B.; Friesner, R. A.; Beard, H. S.; Frye, L. L.; Pollard, W. T.; Banks, J. L. Glide: A new approach for rapid, accurate docking and scoring. 2. Enrichment factors in database screening. Journal of Medicinal Chemistry 2004, 47, 1750-1759.
76. Czabotar, P. E.; Lee, E. F.; van Delft, M. F.; Day, C. L.; Smith, B. J.; Huang, D. C. S.; Fairlie, W. D.; Hinds, M. G.; Colman, P. M. Structural insights into the degradation of McI-1 induced by BH3 domains. Proceedings of the National Academy of Sciences of the United States of America 2007, 104, 6217-6222.
77. Berman, H. M.; Westbrook, J.; Feng, Z.; Gilliland, G.; Bhat, T. N.; Weissig, H.; Shindyalov, I. N.; Bourne, P. E. The Protein Data Bank. Nucleic Acids Research 2000, 28, 235-242.
78. Lee, E. F.; Sadowsky, J. D.; Smith, B. J.; Czabotar, P. E.; Peterson-Kaufman, K. J.; Colman, P. M.; Gellman, S. H.; Fairlie, W. D. High-Resolution Structural Characterization of a Helical alpha/beta-Peptide Foldamer Bound to the Anti-Apoptotic Protein BcI-x(L). Angewandte Chemie-International Edition 2009, 48, 4318-4322.
79. Swayze, E. E.; Jefferson, E. A.; Sannes-Lowery, K. A.; Blyn, L. B.; Risen, L. M.; Arakawa, S.; Osgood, S. A.; Hofstadler, S. A.; Griffey, R. H. SAR by MS: A Ligand Based Technique for Drug Lead Discovery Against Structured RNA Targets. Journal of Medicinal Chemistry 2002, 45, 3816-3819.
80. Muckenschnabel, I.; Falchetto, R.; Mayr, L. M.; Filipuzzi, I. SpeedScreen: label-free liquid chromatography-mass spectrometry-based high-throughput screening for the discovery of orphan protein ligands. Analytical Biochemistry 2004, 324, 241-249.
81. Carrillo, N.; Davalos, E. A.; Russak, J. A.; Bode, J. W. Iterative, aqueous synthesis of beta(3)-oligopeptides without coupling reagents. Journal of the American Chemical Society 2006, 128, 1452-1453.
82. Bode, J. W.; Fox, R. M.; Baucom, K. D. Chemoselective amide ligations by decarboxylative condensations of N-alkylhydroxylamines and alpha-ketoacids. Angewandte Chemie-International Edition 2006, 45, 1248-1252.
83. Ryadnov, M. G.; Woofson, D. N. Self-assembled templates for polypeptide synthesis. Journal of the American Chemical Society 2007, 129, 14074-14081.
84. Ryadnov, M. G.; Woolfson, D. N. Self-assembled templates for polypeptide synthesis. J. Am. Chem. Soc. FIELD Full Journal Title: Journal of the American Chemical Society 2007, 129, 14074-14081.
85. Sasaki, T.; Eguchi, S.; Yamaguchi, M.; Esaki, T. Synthesis of Adamantane Derivatives 0.52. 1,3-Dipolar Cycloaddition Reactions of 1-Azidoadamantane—Reactivity, Regioselectivity, and C-13 Nuclear Magnetic-Resonance Spectra of 141-Adamantyl)-Delta-2-1,2,3-Triazolines and 1-(1-Adamantyl-Delta-2-1h-1,2,3-Triazoles. Journal of Organic Chemistry 1981, 46, 1800-1804.
86. Roque, D. R.; Neill, J. L.; Antoon, J. W.; Stevens, E. P. Synthesis of 1,2,3-triazoles by cycloadditions of azides with enol ethers. Synthesis-Stuttgart 2005, 2497-2502.
87. Park, C.-M.; Oie, T.; Petros, A. M.; Zhang, H.; Nimmer, P. M.; Henry, R. F.; Elmore, S. W. Design, Synthesis, and Computational Studies of Inhibitors of BcI-X_{L}. J. Am. Chem. Soc. 2006, 128, 16206-16212.
88. Pellecchia, M.; Reed, J. C. Inhibition of anti-apoptotic BcI-2 family proteins by natural polyphenols: New avenues for cancer chemoprevention and chemotherapy. Current Pharmaceutical Design 2004, 10, 13871398.
89. Yin, H.; Lee, G. I.; Sedey, K. A.; Rodriguez, J. M.; Wang, H. G.; Sebti, S. M.; Hamilton, A. D. Terephthalamide derivatives as mimetics of helical peptides: Disruption of the BcI-x(L)/Bak interaction. Journal of the American Chemical Society 2005, 127, 5463-5468.
90. Yin, H.; Lee, G. I.; Sedey, K. A.; Kutzki, 0.; Park, H. S.; Omer, B. P.; Ernst, J. T.; Wang, H. G.; Sebti, S. M.; Hamilton, A. D. Terphenyl-based bak BH3 alpha-helical proteomimetics as low-molecular-weight antagonists of BcI-X-L. Journal of the American Chemical Society 2005, 127, 10191-10196.
91. Kutzki, O.; Park, H. S.; Ernst, J. T.; Orner, B. P.; Yin, H.; Hamilton, A. D. Development of a potent BcI-x(L) antagonist based on alpha-helix mimicry. Journal of the American Chemical Society 2002, 124, 1183811839.
92. Blom, K. F.; Larsen, B. S.; McEwen, C. N. Determining affinity-selected ligands and estimating binding affinities by online size exclusion chromatography liquid chromatography-mass spectrometry. Journal of Combinatorial Chemistry 1999, 1, 82-90.
93. Annis, D. A.; Nazef, N.; Chuang, C. C.; Scott, M. P.; Nash, H. M. A general technique to rank protein-ligand binding affinities and determine allosteric versus direct binding site competition in compound mixtures. Journal of the American Chemical Society 2004, 126, 15495-15503.
94. Annis, D. A.; Athanasopoulos, J.; Curran, P. J.; Felsch, J. S.; Kalghatgi, K.; Lee, W. H.; Nash, H. M.; Orminati, J. P. A.; Rosner, K. E.; Shipps, G. W.; Thaddupathy, G. R. A.; Tyler, A. N.; Vilenchik, L.; Wagner, C. R.; Wintner, E. A. An affinity selection-mass spectrometry method for the identification of small molecule ligands from self-encoded combinatorial libraries—Discovery of a novel antagonist of E-coli dihydrofolate reductase. International Journal of Mass Spectrometry 2004, 238, 77-83.
95. Hu, X.; Sun, J.; Wang, H. G.; Manetsch, R. BcI-X_{L}-templated assembly of its own protein-protein interaction modulator from fragments decorated with thio acids and sulfonyl azides. J Am Chem Soc 2008, 130, 13820-13821.
96. Tang, G.; Yang, C. Y.; Nikolovska-Coleska, Z.; Guo, J.; Qiu, S.; Wang, R.; Gao, W.; Wang, G.; Stuckey, J.; Krajewski, K.; Jiang, S.; Roller, P. P.; Wang, S. Pyrogallol-based molecules as potent inhibitors of the antiapoptotic BcI-2 proteins. J Med Chem 2007, 50, 1723-1726.
97. Czabotar, P. E.; Lee, E. F.; van Delft, M. F.; Day, C. L.; Smith, B. J.; Huang, D. C.; Fairlie, W. D.; Hinds, M. G.; Colman, P. M. Structural insights into the degradation of McI-1 induced by BH3 domains. Proc Natl Acad Sci USA 2007, 104, 6217-6222.
98. Takahashi, Y.; Karbowski, M.; Yamaguchi, H.; Kazi, A.; Wu, J.; Sebti, S. M.; Youle, R. J.; Wang, H. G. Loss of Bif-1 suppresses Bax/Bak conformational change and mitochondrial apoptosis. Mol Cell Biol 2005, 25, 9369-9382.
99. Yamaguchi, H.; Wang, H. G. BcI-X_{L} protects BimEL-induced Bax conformational change and cytochrome C release independent of interacting with Bax or BimEL. J Biol Chem 2002, 277, 41604-41612.
100. Balani, S. K.; Miwa, G. T.; Gan, L. S.; Wu, J. T.; Lee, F. W. Strategy of utilizing in vitro and in vivo ADME tools for lead optimization and drug candidate selection. Curr Top Med Chem 2005, 5, 1033-1038.
101. Kutzki, O.; Park, H. S.; Ernst, J. T.; Orner, B. P.; Yin, H.; Hamilton, A. D. Development of a potent BcI-x(L) antagonist based on alpha-helix mimicry. J Am Chem Soc 2002, 124, 11838-11839.
102. Yin, H.; Lee, G. I.; Sedey, K. A.; Kutzki, 0.; Park, H. S.; Orner, B. P.; Ernst, J. T.; Wang, H. G.; Sebti, S. M.; Hamilton, A. D. Terphenyl-Based Bak BH3 alpha-helical proteomimetics as low-molecular-weight antagonists of BcI-xL. J Am Chem Soc 2005, 127, 10191-10196.
103. Kellenberger, E.; Rodrigo, J.; Muller, P.; Rognan, D. Comparative evaluation of eight docking tools for docking and virtual screening accuracy. Proteins 2004, 57, 225-242.

APPENDIX

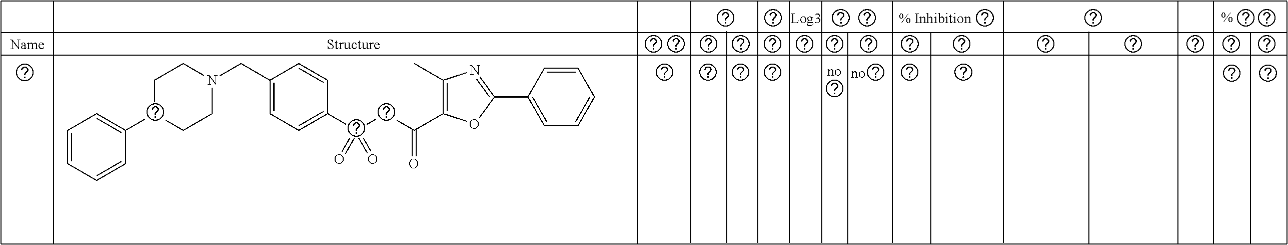

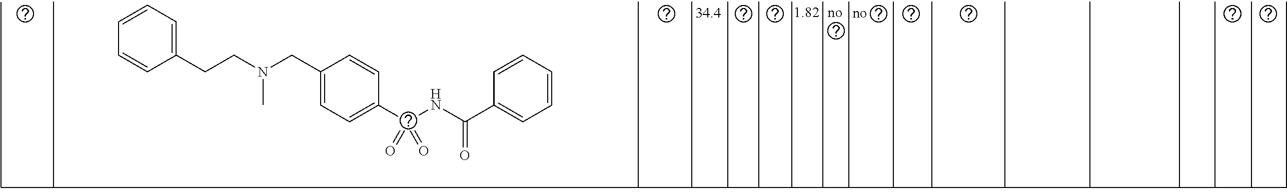

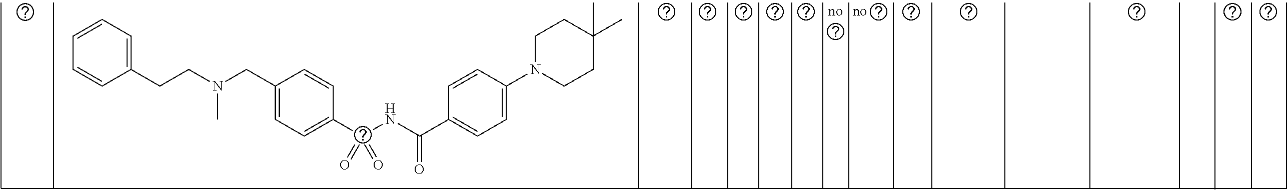

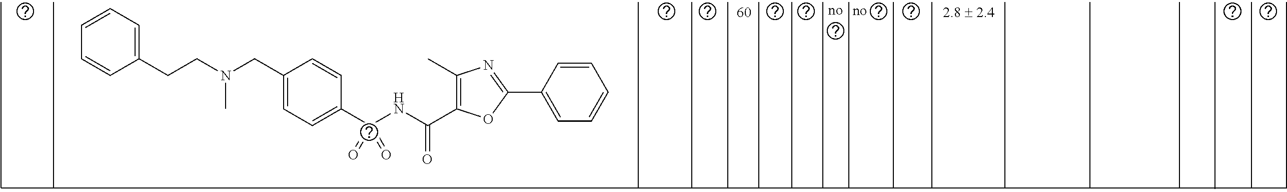

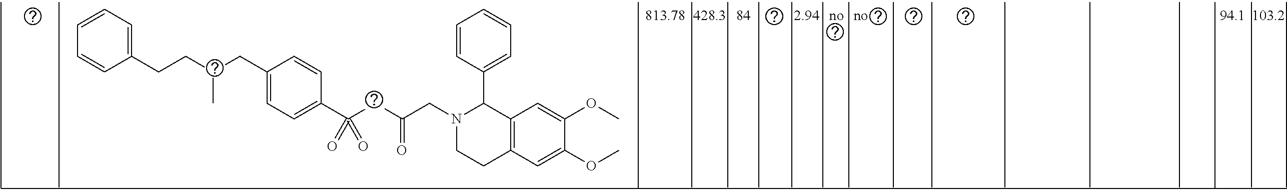

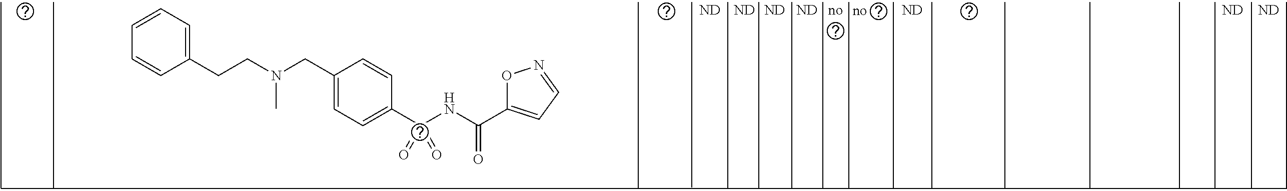

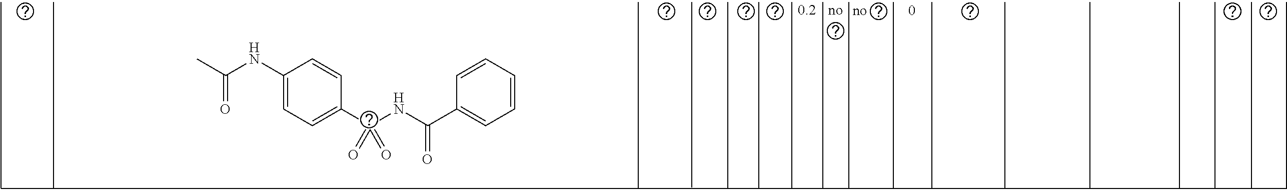

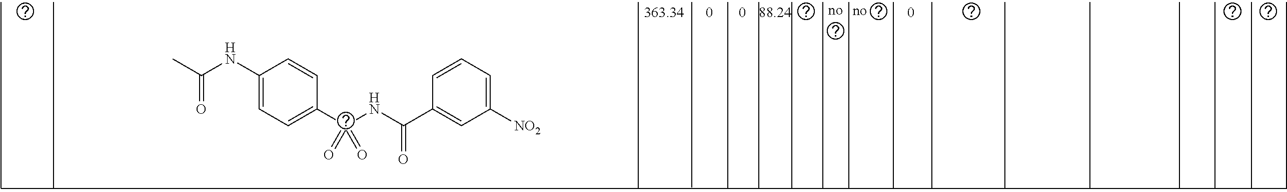

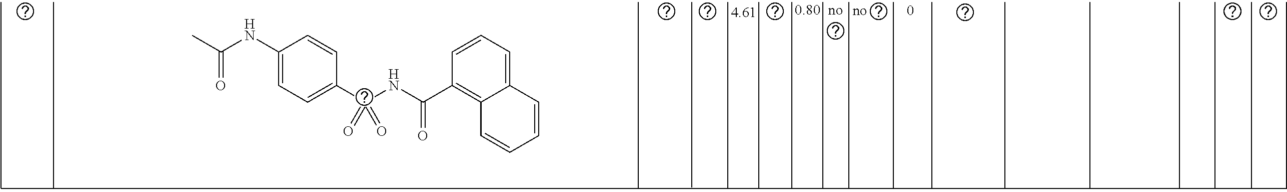

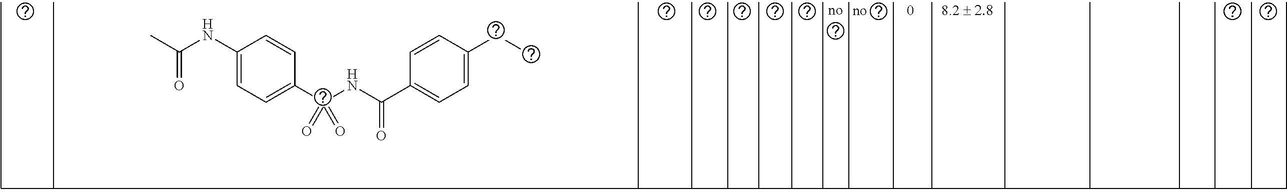

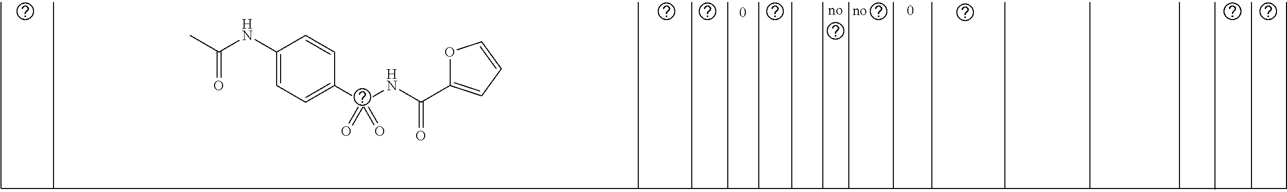

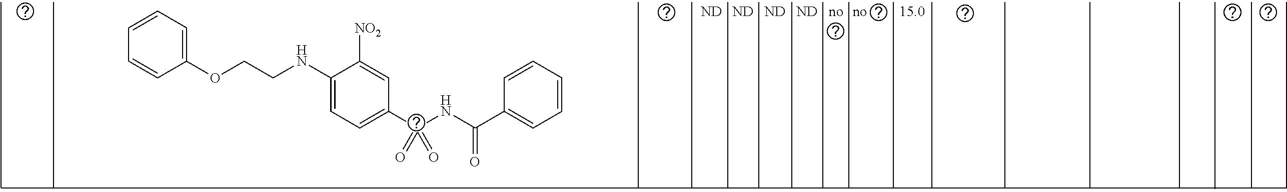

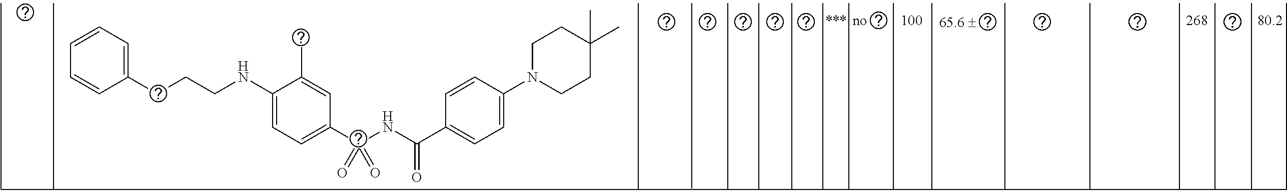

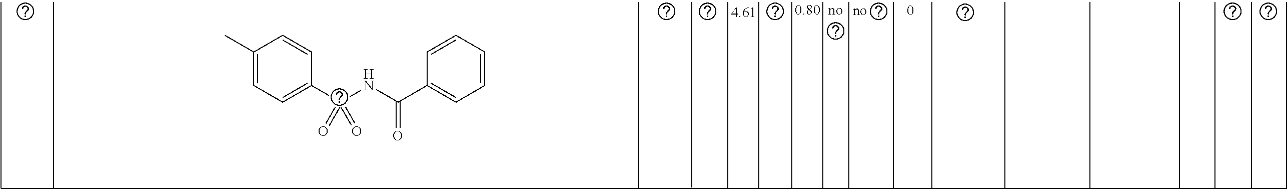

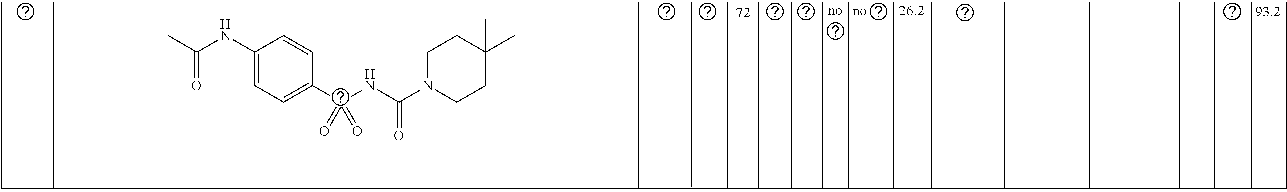

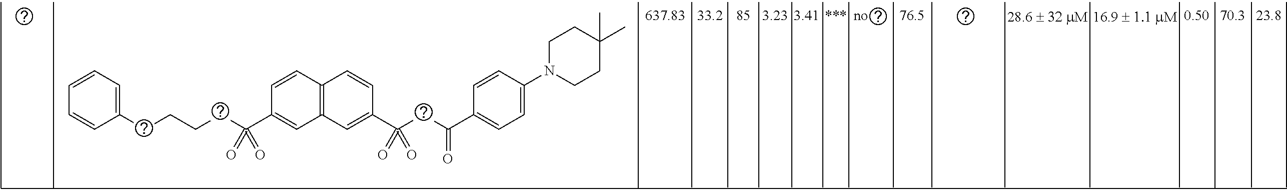

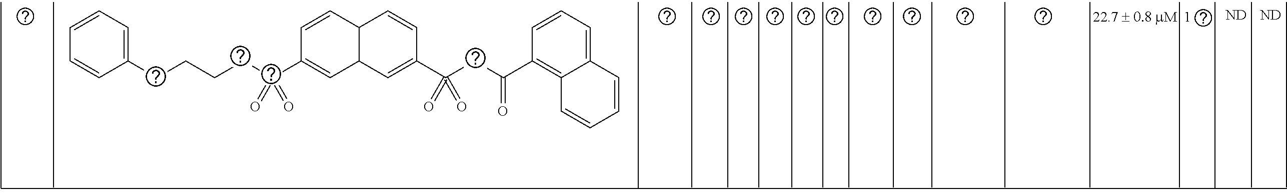

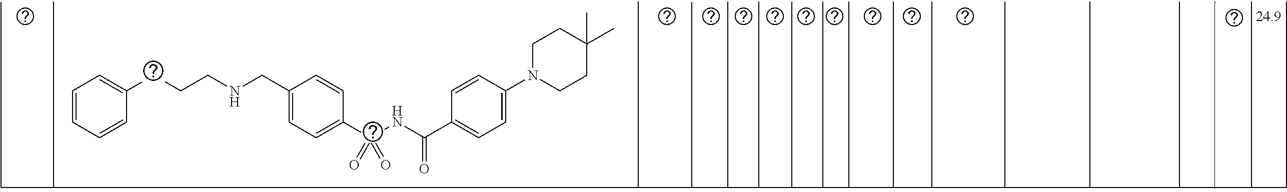

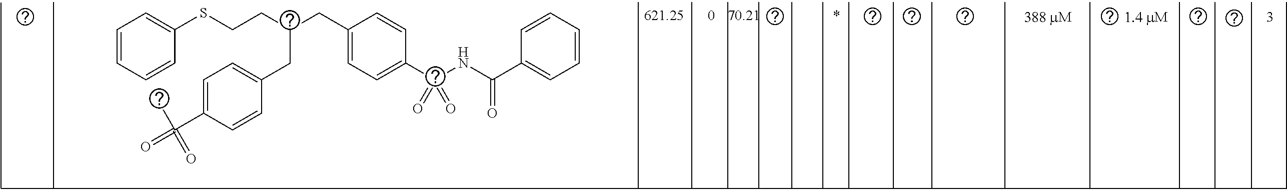

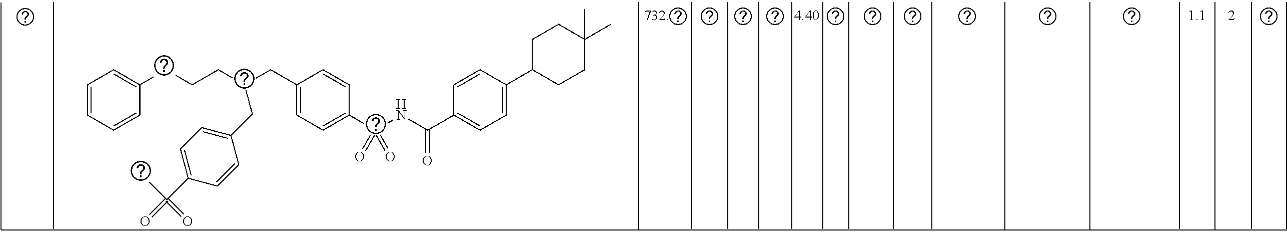

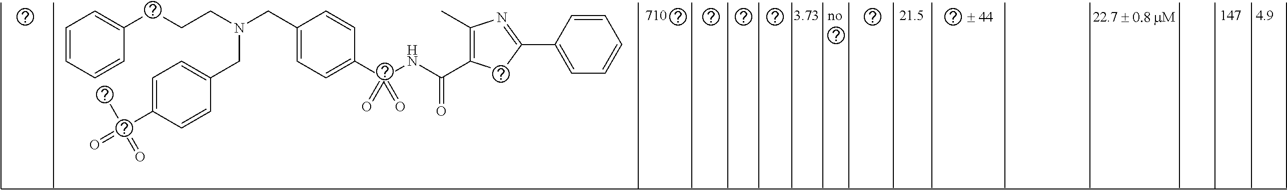

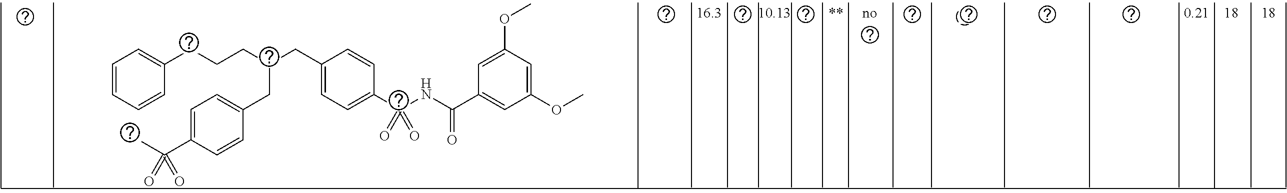

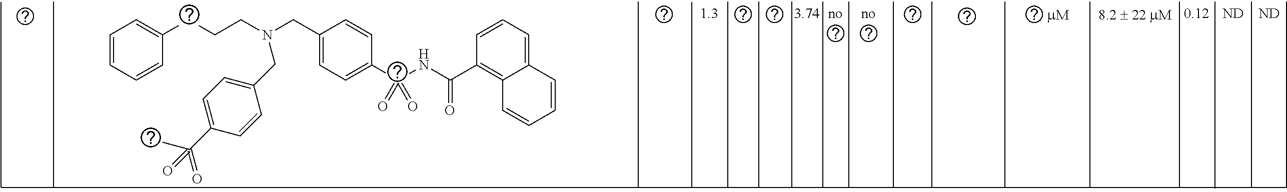

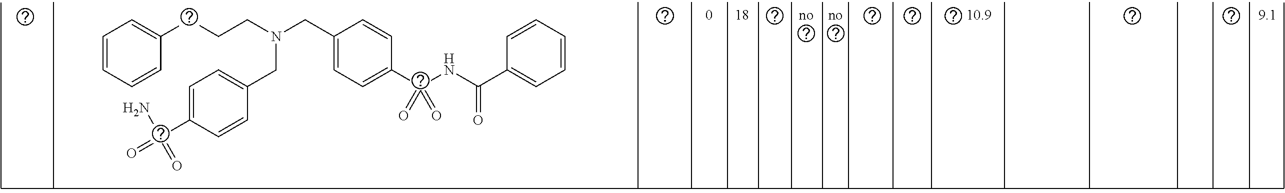

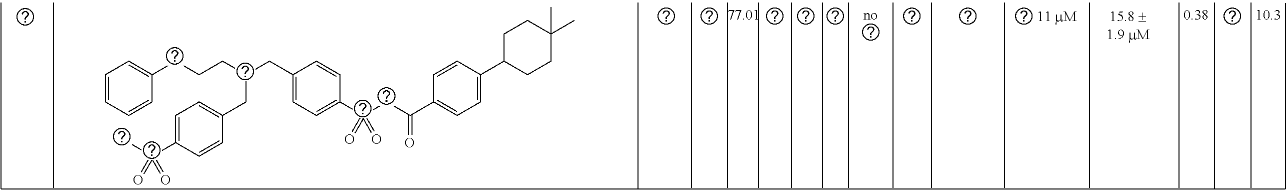

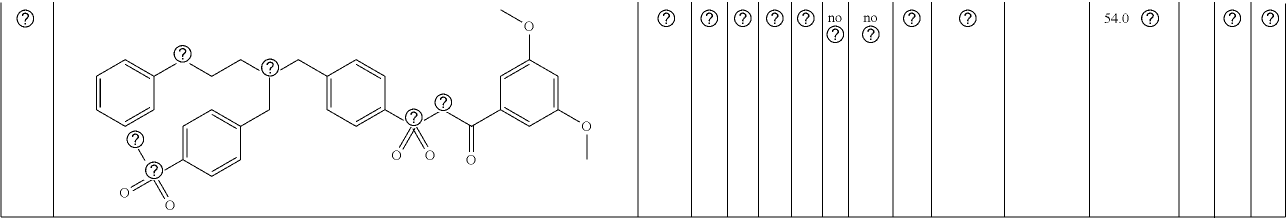

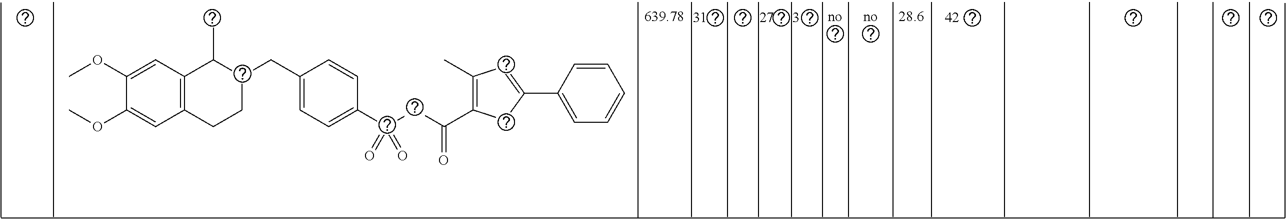

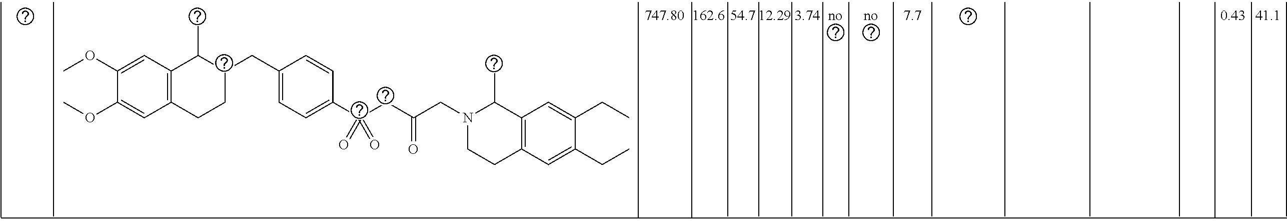

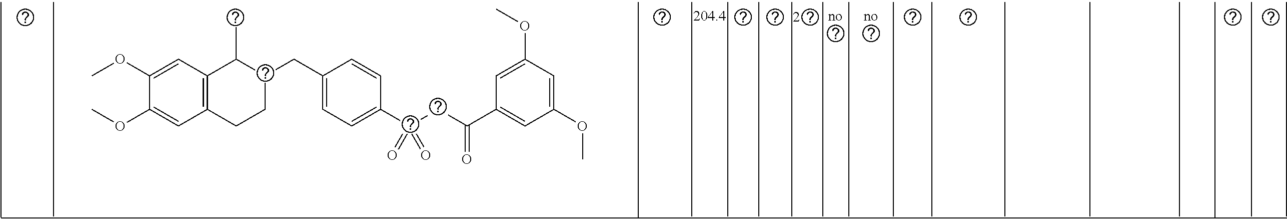

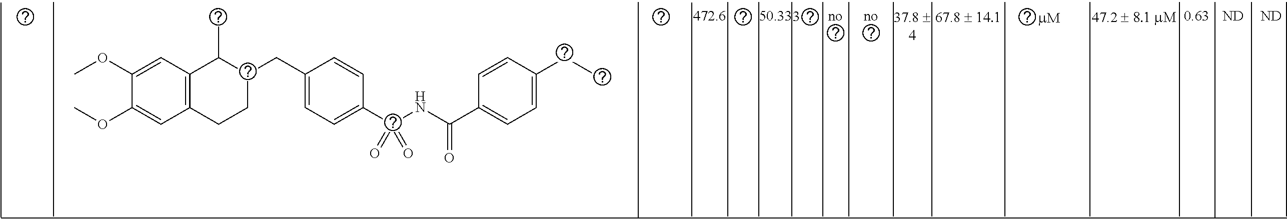

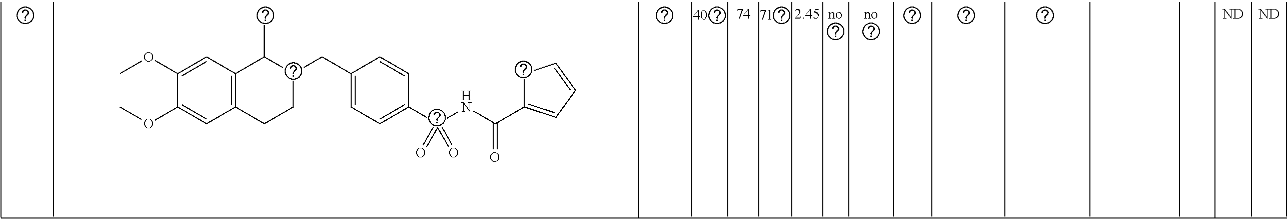

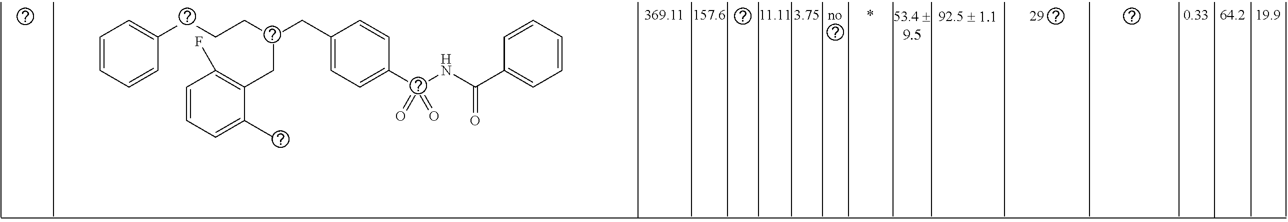

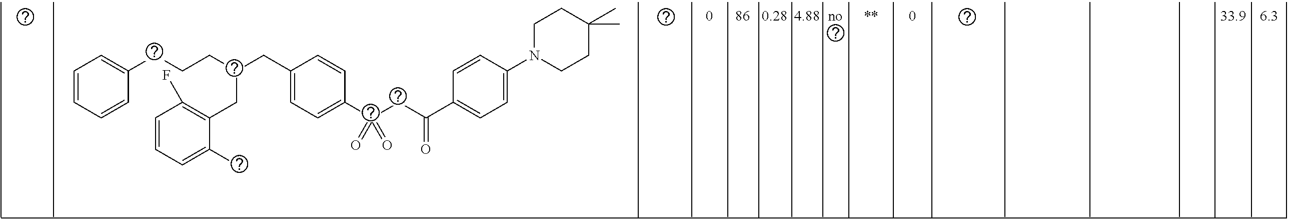

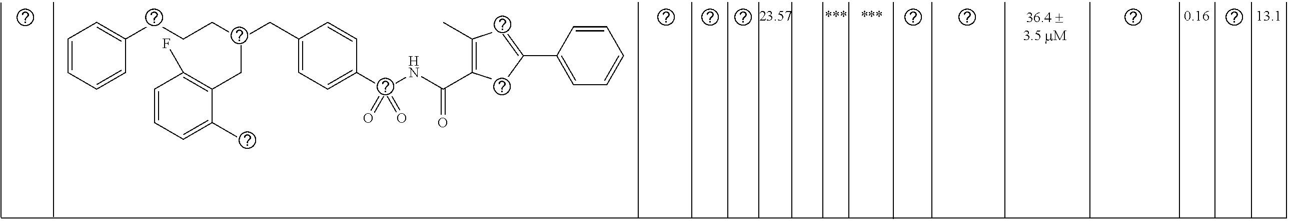

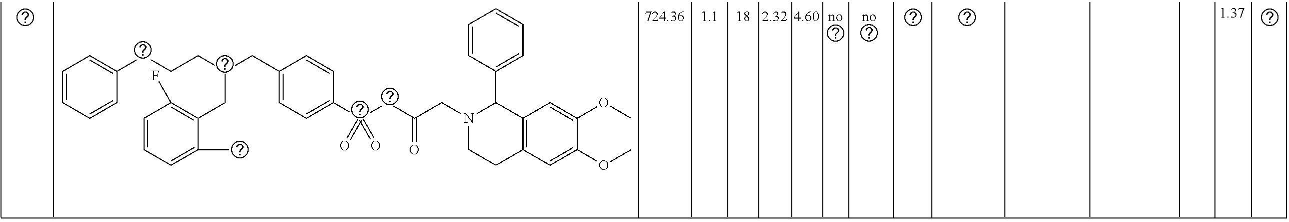

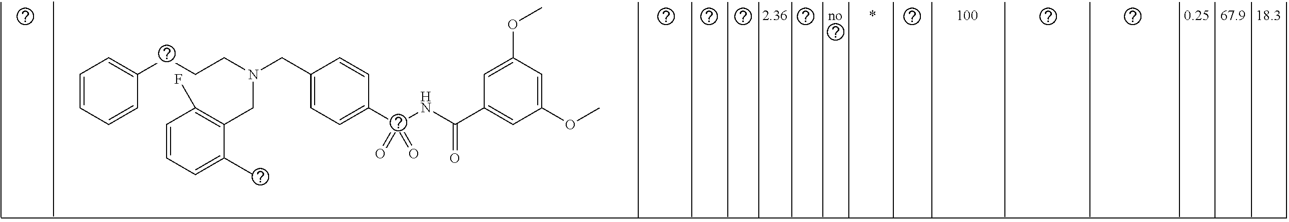

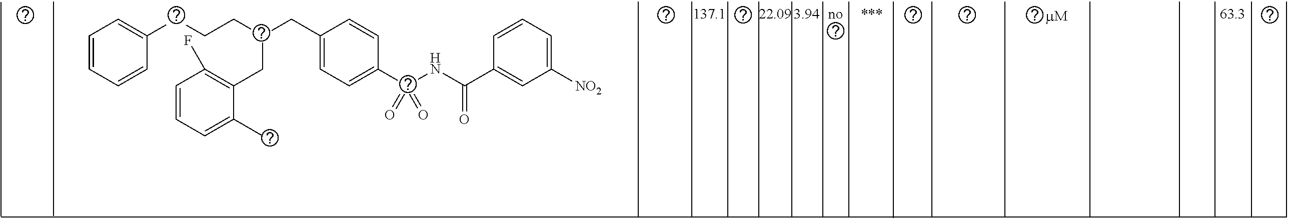

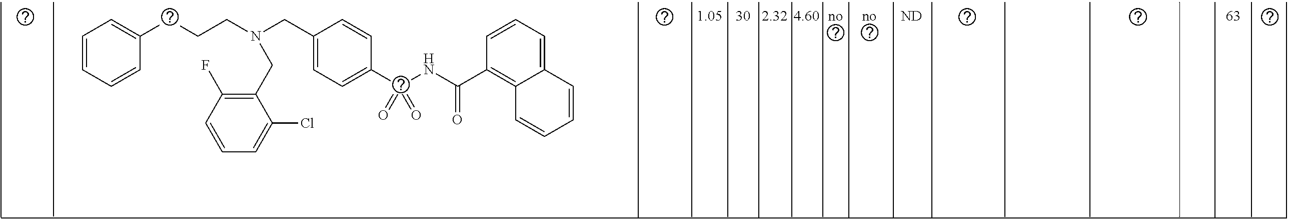

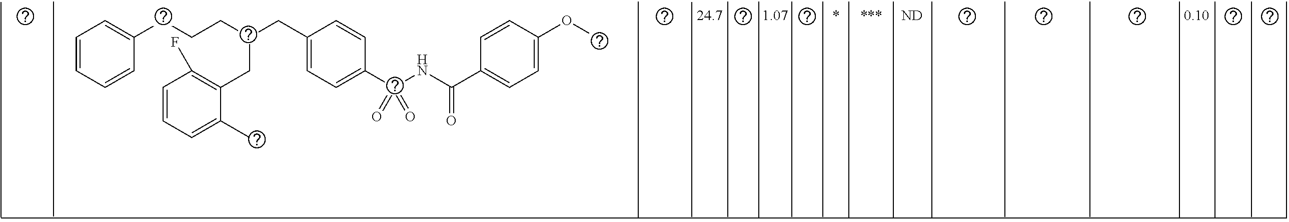

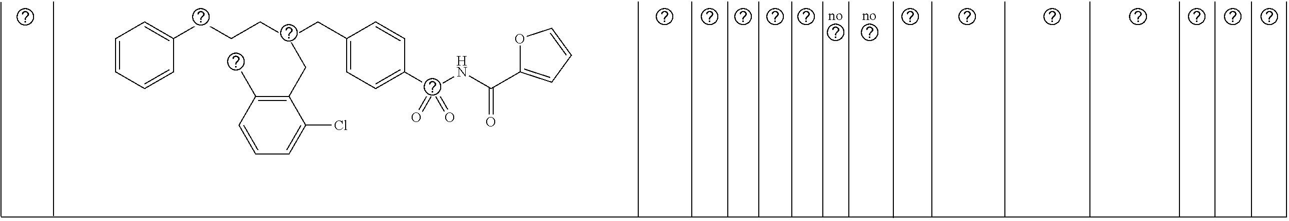

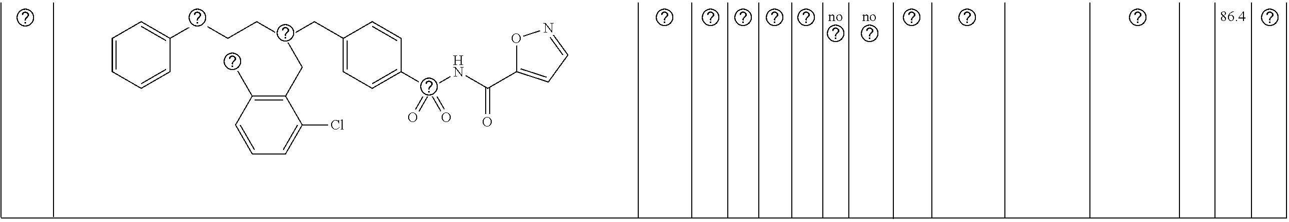

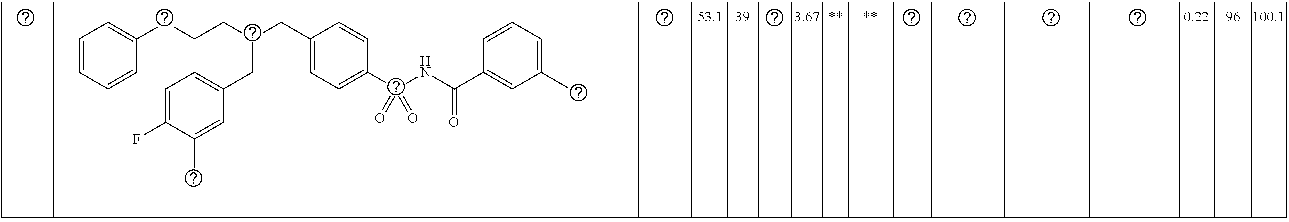

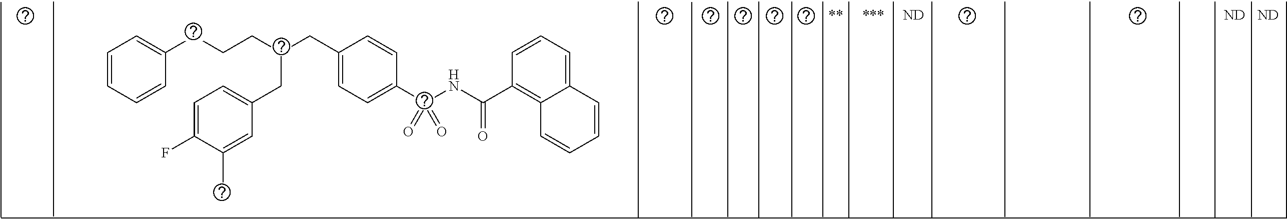

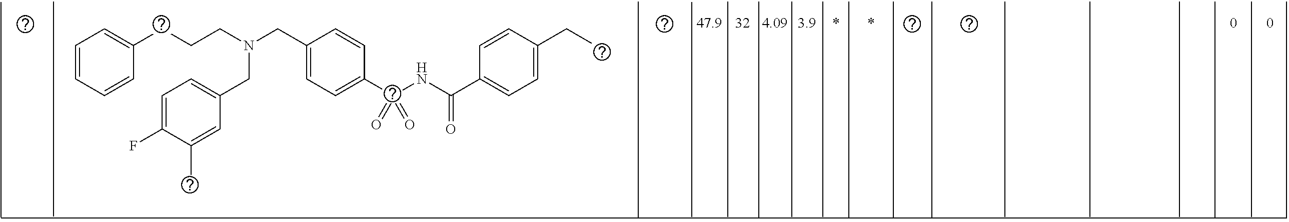

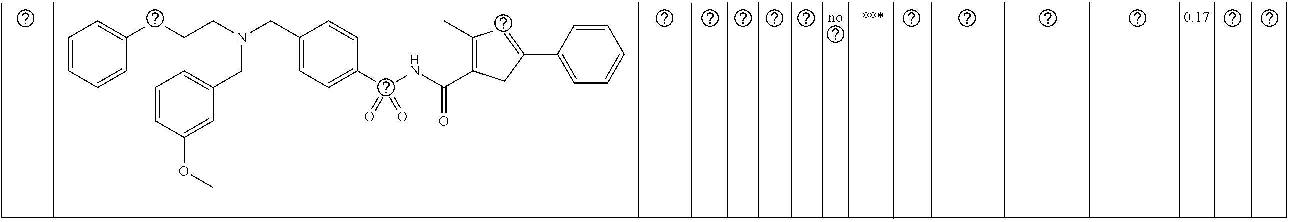

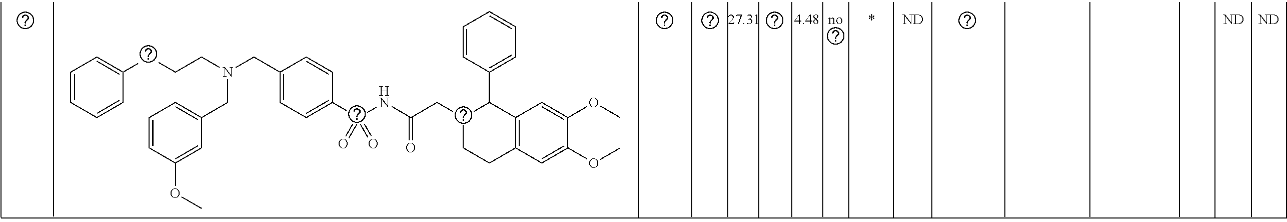

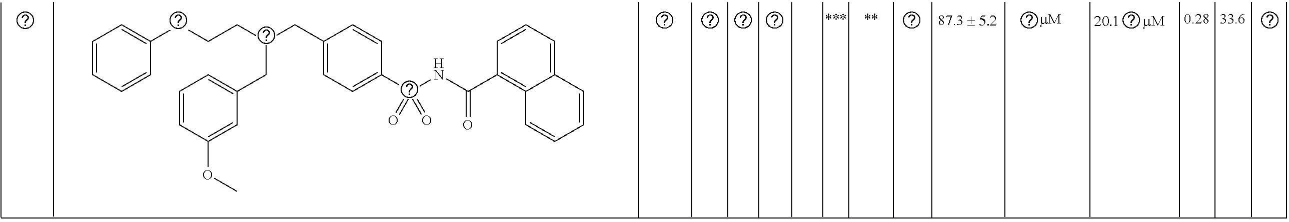

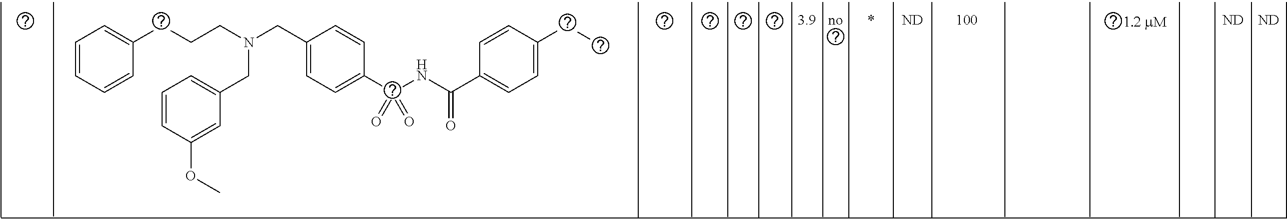

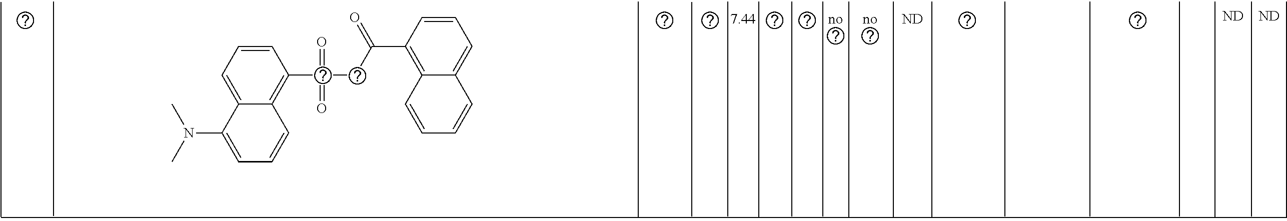

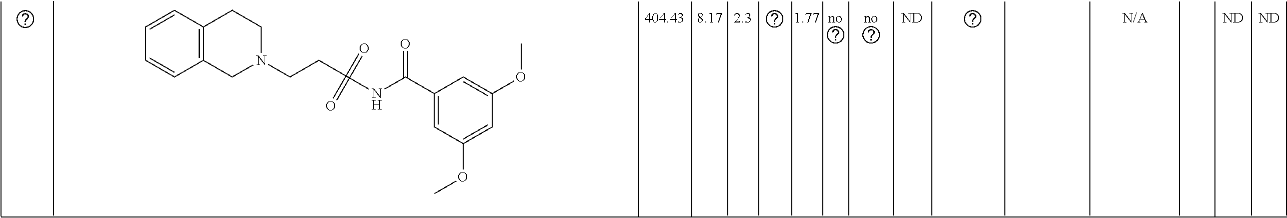

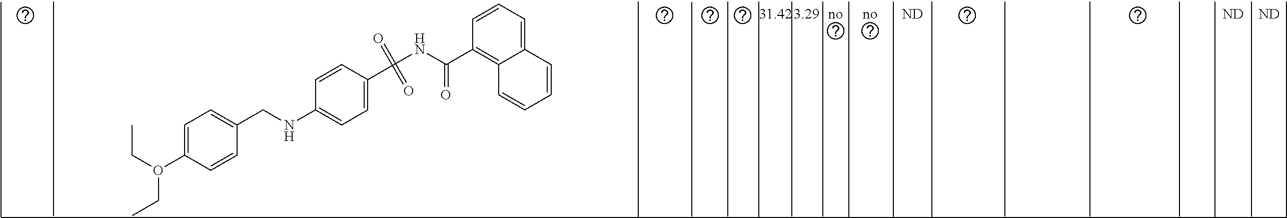

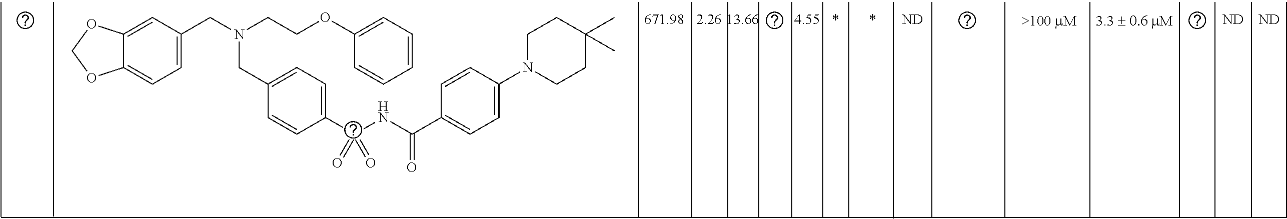

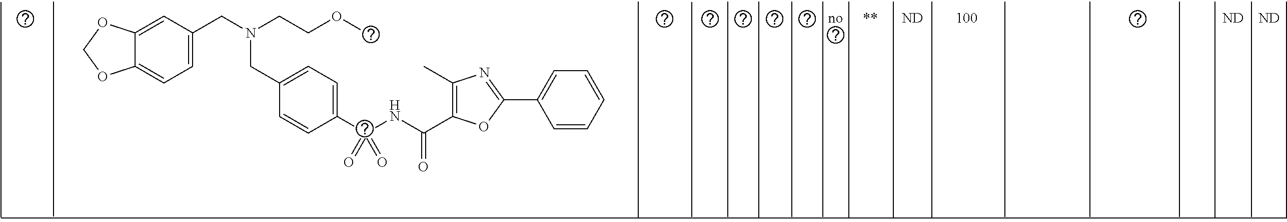

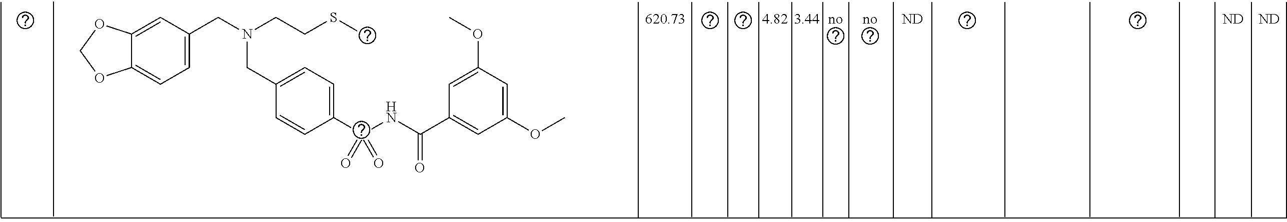

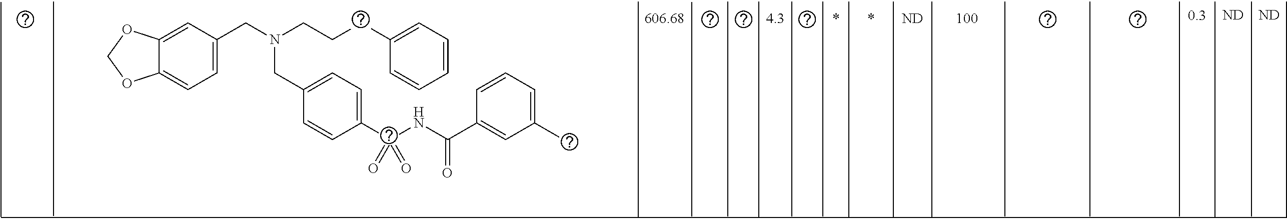

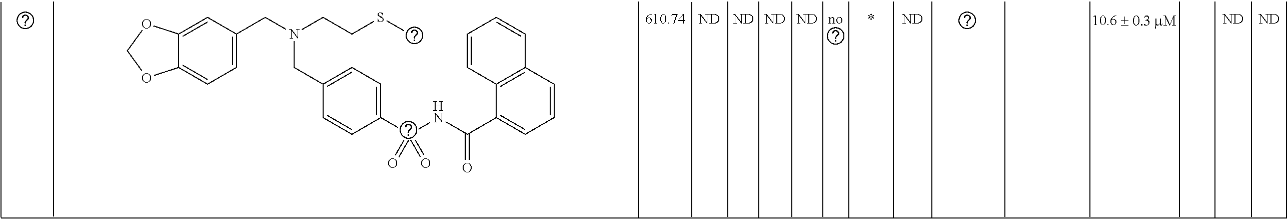

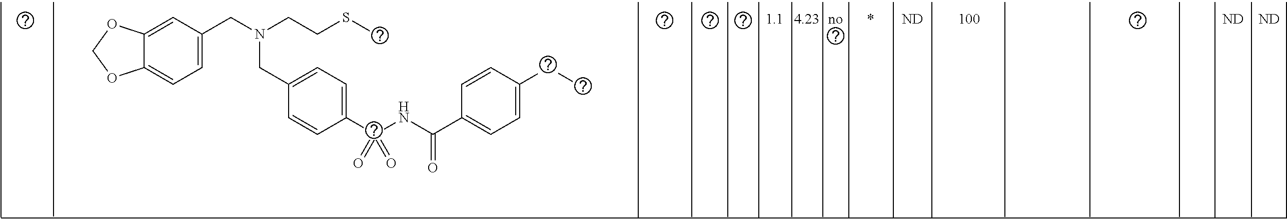

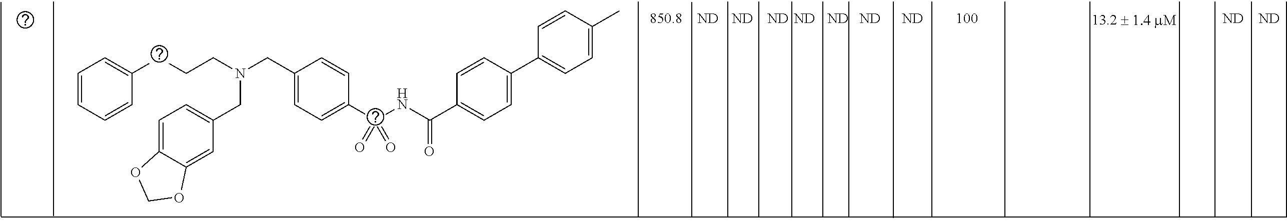

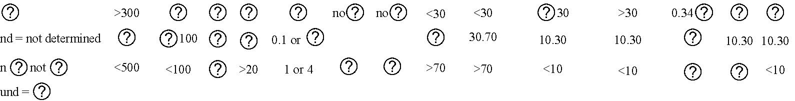

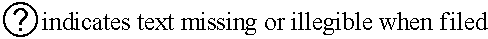

Kinetic TGS

| Amplification Factor | Stars |
|---|---|
| 2 to 4.9 | * |
| 5 to 9.9 | ** |
| 10 to 19.9 | *** |
| 20 and above | **** |

| | | | % Inhibition | |
|---|---|---|---|---|
| Name | Structure | Mol. Wt. | Bcl-XL | Mcl-1 |
| SZ1 | 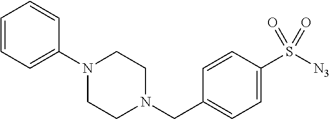 [SMILES: [N-]=[N+]=NS(=O)(=O)c1ccc(CN2CCN(c3ccccc3)CC2)cc1] | 357.43 | ND | ND |
| SZ2 | 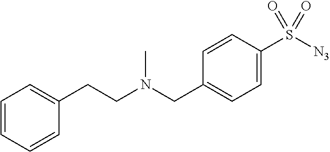 [SMILES: CN(CCc1ccccc1)Cc1ccc(S(=O)(=O)N=[N+]=[N-])cc1] | 330.4 | ND | ND |
| SZ3 | 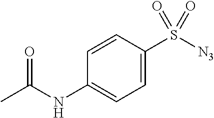 [SMILES: CC(=O)Nc1ccc(S(=O)(=O)N=[N+]=[N-])cc1] | 240.24 | 6.7 ± 4.5 | <1 |
| SZ4 | 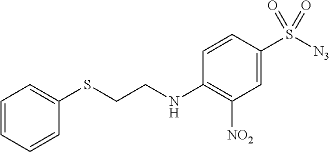 [SMILES: [N-]=[N+]=NS(=O)(=O)c1ccc(NCCSc2ccccc2)c([N+](=O)[O-])c1] | 379.41 | <1 | 23.4 ± 14.9 |
| SZ5 | 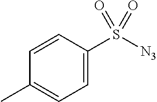 | 197.21 | 2.3 ± 1.1 | 1.2 ± 7.9 |
| SZ6 | 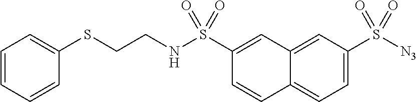 [SMILES: [N-]=[N+]=NS(=O)(=O)c1ccc2ccc(S(=O)(=O)NCCSc3ccccc3)cc2c1] | 448.54 | 4.6 ± 4.7 | 3.4 ± 0.2 |
| SZ8 | 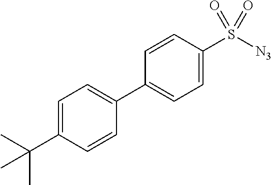 [SMILES: CC(C)(C)c1ccc(-c2ccc(S(=O)(=O)N=[N+]=[N-])cc2)cc1] | 315.39 | 2.2 ± 0.7 | <1 |
| SZ9 | 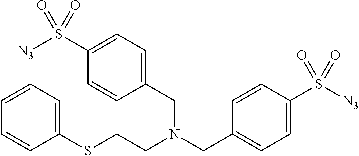 [SMILES: [N-]=[N+]=NS(=O)(=O)c1ccc(CN(CCSc2ccccc2)Cc2ccc(S(=O)(=O)N=[N+]=[N-])cc2)cc1] | 543.64 | 4.7 ± 4.0 | 13.0 ± 1.4 |
| SZ10 | 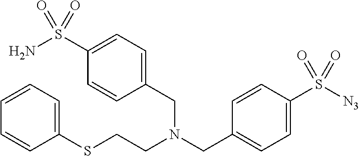 [SMILES: [N-]=[N+]=NS(=O)(=O)c1ccc(CN(CCSc2ccccc2)Cc2ccc(S(N)(=O)=O)cc2)cc1] | 517.64 | 1.9 ± 3.8 | <1 |
| SZ11 | 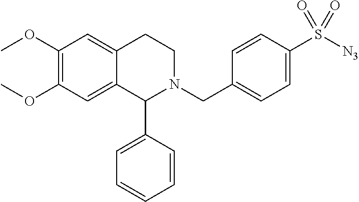 [SMILES: COc1cc2c(cc1OC)C(c1ccccc1)N(Cc1ccc(S(=O)(=O)N=[N+]=[N-])cc1)CC2] | 464.53 | <1 | <1 |
| SZ12 | 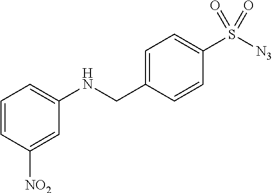 [SMILES: [N-]=[N+]=NS(=O)(=O)c1ccc(CNc2cccc([N+](=O)[O-])c2)cc1] | 333.32 | <1 | <1 |
| SZ13 | 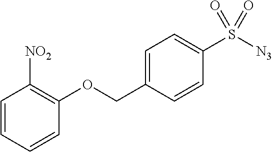 [SMILES: [N-]=[N+]=NS(=O)(=O)c1ccc(COc2ccccc2[N+](=O)[O-])cc1] | 334.3 | 3.8 ± 4.1 | <1 |
| SZ14 | 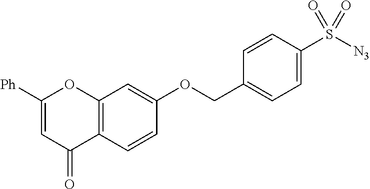 [SMILES: [N-]=[N+]=NS(=O)(=O)c1ccc(COc2ccc3c(=O)cc(-c4ccccc4)oc3c2)cc1] | 433.43 | 7.9 ± 5.8 | 2.1 ± 3.3 |
| SZ15 | 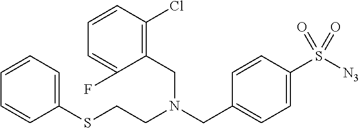 [SMILES: [N-]=[N+]=NS(=O)(=O)c1ccc(CN(CCSc2ccccc2)Cc2c(F)cccc2Cl)cc1] | 491 | 10.0 ± 0.9 | 25.1 ± 0.7 |
| SZ16B | 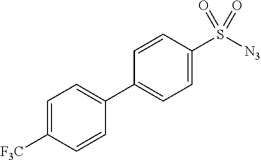 [SMILES: [N-]=[N+]=NS(=O)(=O)c1ccc(-c2ccc(C(F)(F)F)cc2)cc1] | 327.28 | 1.1 ± 1.2 | 1.3 ± 2.4 |
| SZ17 | 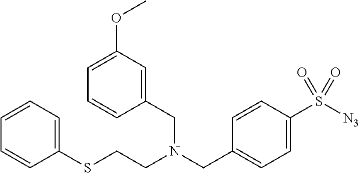 [SMILES: COc1cccc(CN(CCSc2ccccc2)Cc2ccc(S(=O)(=O)N=[N+]=[N-])cc2)c1] | 468.59 | 2.6 ± 7.8 | 4.0 ± 1.6 |
| SZ18 | 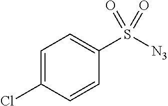 [SMILES: [N-]=[N+]=NS(=O)(=O)c1ccc(Cl)cc1] | 217.63 | <1 | <1 |
| SZ19 | 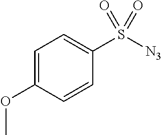 | 213.21 | <1 | <1 |
| SZ20 | 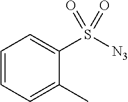 | 197.21 | 1.4 ± 1.6 | 6.2 ± 2.1 |
| SZ21 | 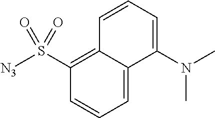 [SMILES: CN(C)c1cccc2c(S(=O)(=O)N=[N+]=[N-])cccc12] | 276.31 | 1.10 | 49.3 ± 3.3 |
| SZ22 | 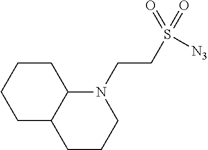 [SMILES: [N-]=[N+]=NS(=O)(=O)CCN1CCCC2CCCCC21] | 272.37 | <1 | 1.2 ± 1.2 |
| SZ23 | 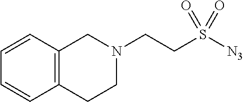 [SMILES: [N-]=[N+]=NS(=O)(=O)CCN1CCc2ccccc2C1] | 266.32 | 3.3 ± 2.1 | 3.6 ± 5.2 |
| SZ24 | 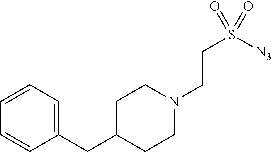 [SMILES: [N-]=[N+]=NS(=O)(=O)CCN1CCC(Cc2ccccc2)CC1] | 308.39 | <1 | 2.7 ± 7.5 |
| SZ25 | 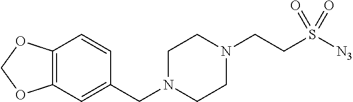 [SMILES: [N-]=[N+]=NS(=O)(=O)CCN1CCN(Cc2ccc3c(c2)OCO3)CC1] | 353.39 | <1 | <1 |
| SZ26 | 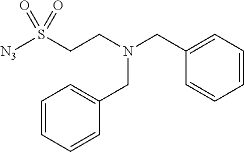 [SMILES: [N-]=[N+]=NS(=O)(=O)CCN(Cc1ccccc1)Cc1ccccc1] | 330.4 | <1 | <1 |
| SZ27 | 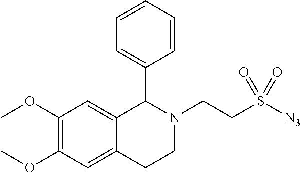 [SMILES: COc1cc2c(cc1OC)C(c1ccccc1)N(CCS(=O)(=O)N=[N+]=[N-])CC2] | 402.47 | 4.0 ± 0.1 | 13.9 ± 0.05 |
| SZ28 | 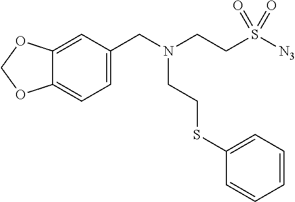 [SMILES: [N-]=[N+]=NS(=O)(=O)CCN(CCSc1ccccc1)Cc1ccc2c(c1)OCO2] | 420.5 | <1 | <1 |
| SZ29 | 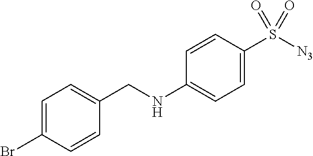 [SMILES: [N-]=[N+]=NS(=O)(=O)c1ccc(NCc2ccc(Br)cc2)cc1] | 367.22 | <1 | 5.7 ± 4.6 |
| SZ30 | 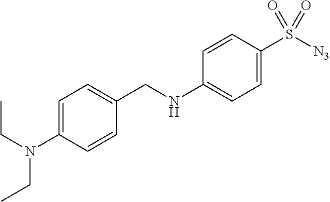 [SMILES: CCN(CC)c1ccc(CNc2ccc(S(=O)(=O)N=[N+]=[N-])cc2)cc1] | 359.44 | <1 | <1 |
| SZ31 | 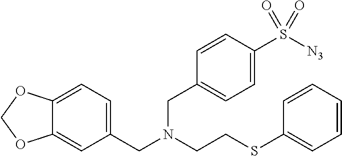 [SMILES: [N-]=[N+]=NS(=O)(=O)c1ccc(CN(CCSc2ccccc2)Cc2ccc3c(c2)OCO3)cc1] | 482.57 | <1 | <1 |
| TA1 | 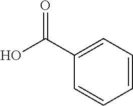 | 122.12 | <1 | <1 |
| TA2 | 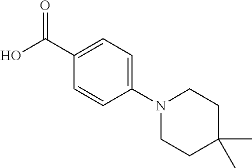 [SMILES: CC1(C)CCN(c2ccc(C(=O)O)cc2)CC1] | 233.3 | <1 | 6.8 ± 6.7 |
| TA3 | 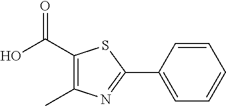 | 219.26 | <1 | 1.4 ± 3.6 |
| TA4 | 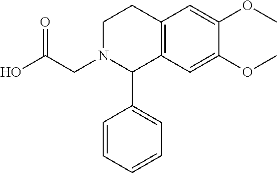 [SMILES: COc1cc2c(cc1OC)C(c1ccccc1)N(CC(=O)O)CC2] | 327.37 | <1 | 1.5 ± 0.7 |
| TA5 | 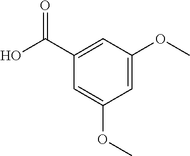 | 182.17 | <1 | 1.3 ± 13.4 |
| TA6 | 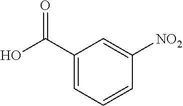 [SMILES: O=C(O)c1cccc([N+](=O)[O-])c1] | 167.11 | <1 | <1 |
| TA7 | 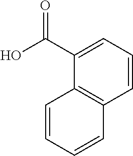 [SMILES: O=C(O)c1cccc2ccccc12] | 172.18 | <1 | 2.4 ± 6.8 |
| TA8 | 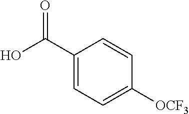 [SMILES: O=C(O)c1ccc(OC(F)(F)F)cc1] | 206.12 | 4.0 ± 1.2 | 3.6 ± 12.7 |
| TA9 | 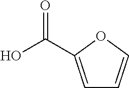 | 112.08 | <1 | <1 |
| TA10 | 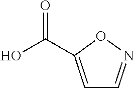 | 113.07 | 2.0 ± 1.7 | <1 |
| SK_2_170 | 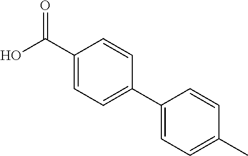 [SMILES: Cc1ccc(-c2ccc(C(=O)O)cc2)cc1] | 212.24 | 7.0 ± 13.0 | 6.8 ± 1.3 |

1. A compound for inhibiting a Bcl-2 family protein selected from one or more of Bcl-2, Bcl-X_{L}, Bcl-w, Mcl-1, and A1/Bfl-1 wherein the compound corresponds to Formula (3): 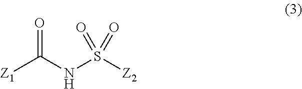
Z₁ is hydrocarbyl, substituted hydrocarbyl, heteroaryl, or heterocyclo; and
Z₂ is hydrocarbyl, substituted hydrocarbyl, heteroaryl, or heterocyclo.

2. The compound of claim 1 wherein the Bcl-2 family protein is Mcl-1.

3. A composition comprising a Bcl-2, Bcl-XL, and/or Bcl-w inhibitor; and an Mcl-1 and/or A1/Bfl-1 inhibitor, wherein at least one inhibitor is the compound of claim 1.

4. A method of treating or preventing cancer, the method comprising administering the compound of claim 1.

5. A compound comprising a first fragment selected from SZ1 to SZ31 and a second fragment selected from TA1 to TA15.

6. The compound of claim 5 having the formula selected from SZ31TA2, SZ15TA2, and SZ17TA2.

7. A method of screening for an inhibitor, as described herein.

8. The method of claim 7 comprising contacting a fragment library with a Bcl-2 family protein.

9. The method of claim 8 wherein the Bcl-2 family protein is selected from one or more of Bcl-2, Bcl-XL, Bcl-w, Mcl-1, and A1/Bfl-1.

10. The method of claim 9 wherein the Bcl-2 family protein is Mcl-1.

11. The compound of claim 1, wherein Z₁ is aryl, substituted aryl, or heteroaryl.

12. The compound of claim 1, wherein Z₁ has the formula: 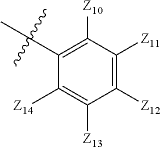
wherein
Z₁₀, Z₁₁, Z₁₂, Z₁₃, and Z₁₄ are independently hydrogen, hydroxyl, protected hydroxyl, halo, hydrocarbyl, substituted hydrocarbyl, heterocyclo, heteroaryl, alkoxy, alkenoxy, alkynoxy, aryloxy, arylalkoxy (heterocyclo)alkoxy, trihaloalkoxy, amino, amido, or cyano, or two of Z₁₀, Z₁₁, Z₁₂, Z₁₃, and Z₁₄, together with the carbon atoms to which they are attached, form a fused carbocyclic (e.g., napthyl) or heterocyclic ring.

13. The compound of claim 1, wherein Z₁₀, Z₁₁, Z₁₂, Z₁₃, and Z₁₄ are independently hydrogen, hydrocarbyl, substituted hydrocarbyl, amino, alkoxy, nitro, or trihalomethoxy.

14. The compound of claim 1, wherein Z₁ has the formula: 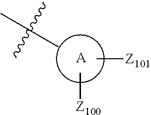
wherein
A is phenyl or a five- or six-membered aromatic carbocyclic or heterocyclic ring wherein from one to three carbon atoms may be replaced by a heteroatom selected from N, O, or S, and wherein A is substituted with Z₁₀₀ and Z₁₀₁ through ring carbon atoms or ring heteroatoms, and Z₁₀₀ and Z₁₀₁ are independently hydrogen, hydrocarbyl, substituted hydrocarbyl, heteroaryl, heterocyclo, alkoxy, alkenoxy, alkynoxy, aryloxy, heterocyclo(alkoxy), or halo.

15. The compound of claim 1, wherein Z₁ is substituted or unsubstituted furyl, thienyl, pyridyl, oxazolyl, isoxazolyl, imidazolyl, pyridyl, pyrimidyl, purinyl, triazolyl, or thiazolyl.

16. The compound of claim 1, wherein Z₁ is substituted or unsubstituted morpholino, pyran, tetrahydropyran, piperazinyl, piperidinyl, tetrahydropyridinyl, pyrrolidinyl, pyrrolinyl, 1,4-diazepanyl, or azepinyl.

17. The compound of claim 1, wherein Z₁ is —(CH₂)ₓ—Z₁₀₂ wherein Z₁₀₂ is hydrogen, hydrocarbyl, substituted hydrocarbyl, hydroxyl, protected hydroxyl, heteroaryl, heterocyclo, amino, amido, alkoxy, aryloxy, cyano, nitro, thiol, or an acetal, ketal, ester, ether, or thioether, and x is 1, 2, or 3.

18. The compound of claim 1, wherein Z₁, is hydrocarbyl, substituted hydrocarbyl, heteroaryl, heterocyclo, or has the formula: 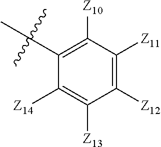
wherein
Z₁₀, Z₁₁, Z₁₂, Z₁₃, and Z₁₄ are independently hydrogen, amino, alkoxy, aryl, heteroaryl, heterocyclo, nitro, or trihalomethoxy (e.g., trifluoromethoxy); or Z₁ is —(CH₂)ₓ—Z₁₀₂ wherein Z₁₀₂ is hydrogen, alkyl, substituted alkyl, hydroxyl, protected hydroxyl, heteroaryl, heterocyclo, amino, amido, alkoxy, aryloxy, cyano, nitro, thiol, or an acetal, ketal, ester, ether, or thioether, and x is 1, 2, or 3.

19. The compound of claim 1, wherein Z₂ is substituted or unsubstituted alkyl, alkenyl, alkynyl, aryl, alkaryl, or aralkyl.

20. The compound of claim 1, wherein Z₂ has the formula: 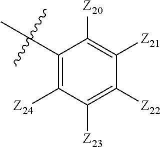
wherein
Z₂₀, Z₂₁, Z₂₂, Z₂₃, and Z₂₄ are independently hydrogen, halo, hydrocarbyl, substituted hydrocarbyl, alkoxy, alkenoxy, alkynoxy, aryloxy, nitro, cyano, amino, or amido, or two of Z₂₀, Z₂₁, Z₂₂, Z₂₃, and Z₂₄, together with the carbon atoms to which they are attached, form a fused carbocyclic or heterocyclic ring.

21. The compound of claim 1, Z₂₀, Z₂₁, Z₂₂, Z₂₃, and Z₂₄ are independently alkyl, substituted alkyl, amino, alkoxy, alkenoxy, alkynoxy, or aryloxy.

22. The compound of claim 1, wherein Z₂ is phenyl, substituted phenyl, napthyl, or substituted napthyl.

23. The compound of claim 1, wherein Z₂ is —(CH₂)ₓ—Z₂₀₀ wherein Z₂₀₀ is hydrogen, hydrocarbyl, substituted hydrocarbyl, hydroxyl, protected hydroxyl, heteroaryl, heterocyclo, amino, amido, alkoxy, aryloxy, cyano, nitro, thiol, or an acetal, ketal, ester, ether, or thioether, and x is 1, 2, or 3.

24. The compound is of claim 1, wherein Z₂ is —(CH₂)ₓ—Z₂₀₀ wherein x is 1, 2, or 3 and Z₂₀₀ is —N(Z₂₀₁)(Z₂₀₂), wherein Z₂₀₁ and Z₂₀₂ are independently hydrogen, alkyl, substituted alkyl, aryl, substituted aryl, or Z₂₀₁ and Z₂₀₂, together with the nitrogen atom to which they are attached, for a substituted or unsubstituted alicyclic, bicyclic, aryl, heteroaryl, or heterocyclic moiety.

25. The compound of claim 1, wherein Z₂ is substituted or unsubstituted furyl, thienyl, pyrrolyl, oxazolyl, imidazolyl, pyridyl, pyrimidyl, purinyl, triazolyl, or thiazolyl.

26. The compound of claim 1, wherein Z₂ is substituted or unsubstituted morpholino, pyran, tetrahydropyran, piperazinyl, piperidinyl, tetrahydropyridinyl, pyrrolidinyl, pyrrolinyl, 1,4-diazepanyl, or azepinyl.

27. The compound of claim 1, wherein the sulfonyl azide corresponds to Formula (2A) or (2B): 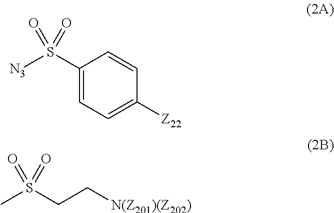
wherein Z₂₂ is hydrogen, halo, alkoxy, alkyl, or substituted alkyl; and
Z₂₀₁ and Z₂₀₂ are independently hydrogen, alkyl, substituted alkyl, aryl, substituted aryl, or Z₂₀₁ and Z₂₀₂ together with the nitrogen atom to which they are attached, form a substituted or unsubstituted alicyclic, bicyclic, aryl, heteroaryl, or heterocyclic moiety.

28. The compound of claim 6 having the formula SZ31TA2.

29. The compound of claim 1, wherein the compound has a selectivity index against Bcl-X_{L} and/or Mcl-1 of at least about 15.

30. The compound of claim 1, wherein the compound has a selectivity index against Bcl-X_{L} and/or Mcl-1 of at least about 30.

31. The compound of claim 1, wherein the compound has a selectivity index against Bcl-X_{L} and/or Mcl-1 of at least about 45.

32. The compound of claim 1, wherein the compound has a selectivity against Bcl-X_{L} and/or Mcl-1 of at least about 60.

33. The compound of claim 1, wherein the compound has a ligand efficiency of at least about 0.15.

34. The compound of claim 1, wherein the compound has a ligand efficiency of at least about 0.21.

35. The compound of claim 1, wherein the compound has a ligand efficiency of at least about 0.24.
